(19) United States
(12) Patent Application Publication
(10) Pub. No.: US 2023/0200218 A1
(43) Pub. Date: Jun. 22, 2023

(54) LIGHT-EMITTING DEVICE INCLUDING AMINE COMPOUND, ELECTRONIC APPARATUS INCLUDING THE LIGHT-EMITTING DEVICE, AND THE AMINE COMPOUND

(71) Applicant: Samsung Display Co., Ltd., Yongin-si (KR)

(72) Inventors: Hankyu Pak, Yongin-si (KR); Dongjun Kim, Yongin-si (KR); Chaeyeong Kim, Yongin-si (KR); Byeongwook Yoo, Yongin-si (KR); Seowon Cho, Yongin-si (KR); Sohee Jo, Yongin-si (KR)

(21) Appl. No.: 18/054,723

(22) Filed: Nov. 11, 2022

(30) Foreign Application Priority Data
Dec. 21, 2021 (KR) 10-2021-0184292

(51) Int. Cl. H01L 51/00; H01L 51/50; H01L 51/52; C07C 211/54; C07D 307/91; C07D 333/76; C07D 209/82; C07F 7/08

(52) U.S. Cl. CPC H01L 51/0059; H01L 51/006; H01L 51/0073; H01L 51/0074; H01L 51/0072; H01L 51/0094; H01L 51/5056; H01L 51/5012; H01L 51/52; C07C 211/54; C07D 307/91; C07D 333/76; C07D 209/82; C07F 7/0812

(57) ABSTRACT

Provided are an amine compound represented by Formula 1, a light-emitting device including the same, and an electronic apparatus including the light-emitting device. The light-emitting device includes: a first electrode; a second electrode facing the second electrode; an interlayer between the first electrode and the second electrode and including an emission layer; and at least one of the amine compound represented by Formula 1.

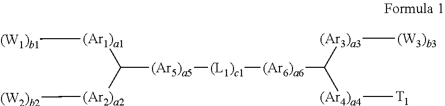

### CROSS-REFERENCE TO RELATED APPLICATION

This application claims priority to and the benefit of Korean Patent Application No. 10-2021-0184292, filed on Dec. 21, 2021, in the Korean Intellectual Property Office, the entire contents of which are hereby incorporated by reference.

### BACKGROUND

### 1. Field

One or more embodiments of the present disclosure relate to a light-emitting device including an amine compound, an electronic apparatus including the light-emitting device, and the amine compound.

### 2. Description of the Related Art

Self-emissive devices among light-emitting devices have wide viewing angles, high contrast ratios, short response times, and excellent characteristics in terms of luminance, driving voltage, and response speed, and produce full-color images, compared to other devices in the art.

In a light-emitting device, a first electrode is on a substrate, and a hole transport region, an emission layer, an electron transport region, and a second electrode are sequentially formed on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. These excitons transition from an excited state to a ground state, thereby generating light.

### SUMMARY

One or more embodiments of the present disclosure include a light-emitting device including an amine compound, an electronic apparatus including the light-emitting device, and the amine compound.

Additional aspects of embodiments will be set forth in part in the description which follows and, in part, will be apparent from the description, or may be learned by practice of the presented embodiments of the disclosure.

According to one or more embodiments, an amine compound represented by Formula 1 is provided.

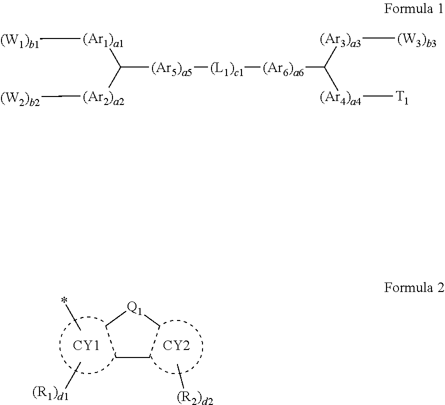

In Formulae 1 and 2,

L₁ may be *—C(X₁)(X₂)—*′,

X₁ and X₂ may each independently be:

hydrogen, deuterium, —F, —Cl, —Br, —I, a hydroxyl group, or a cyano group, or

a C₁-C₂₀ alkyl group unsubstituted or substituted with at least one R₁₀ₐ or a C₃-C₂₀ cycloalkyl group unsubstituted or substituted with at least one R₁₀ₐ,

X₁ and X₂ may optionally be linked to each other to form a C₃-C₂₀ cycloalkane group unsubstituted or substituted with at least one R₁₀ₐ,

c1 may be an integer from 1 to 3,

Ar₁ to Ar₆ may each independently be a single bond, a C₅-C₆₀ carbocyclic group unsubstituted or substituted with at least one R₁₀ₐ, or a C₁-C₆₀ heterocyclic group unsubstituted or substituted with at least one R₁₀ₐ,

a1 to a6 may each independently be an integer from 1 to 3,

W₁ to W₃ may each independently be a C₅-C₆₀ carbocyclic group unsubstituted or substituted with at least one R₁₀ₐ or a C₁-C₆₀ heterocyclic group unsubstituted or substituted with at least one R₁₀ₐ,

b1 to b3 may each independently be an integer from 1 to 10,

T₁ may be a group represented by Formula 2,

in Formula 2,

CY₁ and CY₂ may each independently be a C₅-C₆₀ carbocyclic group or a C₁-C₆₀ heterocyclic group,

Q₁ may be O, S, N(X₁₁), C(X₁₁)(X₁₂), or Si(X₁₁)(X₁₂),

R₁, R₂, X₁₁, and X₁₂ may each independently be hydrogen or the same as described in connection with R₁₀ₐ,

X₁₁ and X₁₂ may optionally be linked to each other to form a C₃-C₆₀ carbocyclic group or a C₁-C₆₀ heterocyclic group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, or any combination thereof,

d1 and d2 may each independently be an integer from 0 to 10,

in Formula 1, a group represented by *—(Ar₄)ₐ₄-T₁ may be different from a group represented by *—(Ar₁)ₐ₁—(W₁)_{b1} and a group represented by *—(Ar₂)ₐ₂—(W₂)_{b2},

* and *′ each indicate a binding site to a neighboring atom,

R₁₀ₐ may be:

deuterium, —F, —Cl, —Br, —I, a hydroxyl group, a cyano group, or a nitro group,

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and

Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments, a light-emitting device includes a first electrode,

a second electrode facing the first electrode,

an interlayer between the first electrode and the second electrode and including an emission layer, and

at least one of the amine compound represented by Formula 1.

According to one or more embodiments, an electronic apparatus includes the light-emitting device.

### BRIEF DESCRIPTION OF THE DRAWINGS

The above and other aspects and features of certain embodiments of the disclosure will be more apparent from the following description taken in conjunction with the accompanying drawings, in which:

FIG. 1 is a schematic view of a light-emitting device according to an embodiment; and

FIGS. 2 and 3 are each a schematic cross-sectional view of an electronic apparatus according to an embodiment.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of embodiments of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

An aspect of embodiments of the present disclosure provides an amine compound represented by Formula 1:

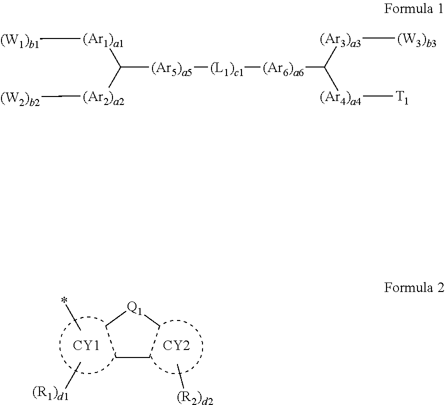

wherein, in Formulae 1 and 2,

L₁ may be *—C(X₁)(X₂)—*′,

X₁ and X₂ may each independently be:

hydrogen, deuterium, —F, —Cl, —Br, —I, a hydroxyl group, or a cyano group; or

a C₁-C₂₀ alkyl group unsubstituted or substituted with at least one R₁₀ₐ or a C₃-C₂₀ cycloalkyl group unsubstituted or substituted with at least one R₁₀ₐ,

X₁ and X₂ may optionally be linked to each other to form a C₃-C₂₀ cycloalkane group unsubstituted or substituted with at least one R₁₀ₐ, and

c1 may be an integer from 1 to 3.

In this regard, the term “cycloalkane group,” as used herein, includes not only a monocyclic group, such as a cyclopentane group or a cyclohexane group, but also a condensed cyclic unsaturated hydrocarbon group, such as an adamantane group (see, e.g., Compounds 25 and 29 below).

In an embodiment, X₁ and X₂ may each independently be: hydrogen; a C₁-C₁₀ alkyl group unsubstituted or substituted with deuterium, —F, a cyano group, or any combination thereof; or a C₃-C₁₀ cycloalkyl group unsubstituted or substituted with deuterium, —F, a cyano group, a C₁-C₁₀ alkyl group, or any combination thereof.

In one or more embodiments, X₁ and X₂ may each independently be: hydrogen; a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, or a sec-octyl group, each unsubstituted or substituted with deuterium, —F, a cyano group, or any combination thereof; or

a cyclopropyl group, a cyclobutyl group, a cyclopentyl group, a cyclohexyl group, a cycloheptyl group, a cyclooctyl group, an adamantanyl group, a norbornanyl group, a bicyclo[1.1.1]pentyl group, a bicyclo[2.1.1]hexyl group, or a bicyclo[2.2.2]octyl group, each unsubstituted or substituted with deuterium, —F, a cyano group, a C₁-C₁₀ alkyl group, or any combination thereof.

In one or more embodiments, X₁ and X₂ may each independently be hydrogen, a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, or a sec-octyl group.

In an embodiment, c1 in Formula 1 may be 1 or 2.

For example, c1 may be 1, but embodiments are not limited thereto.

In an embodiment, a group represented by *-(L₁)_{c1}-*′ in Formula 1 may be one selected from *—(CH₂)—*′, *—CH(CH₃)—*′, *—CH(CH₂CH₃)—*′, *—CH[(CH₂)₂CH₃]—*′, *—CH[(CH₂)₃CH₃]—*′, *—CH[(CH₂)₄CH₃]—*′, *—C(CH₃)₂—*′, *—C(CH₃)(CH₂CH₃)—*′, *—C(CH₃)[(CH₂)₂CH₃]—*′, *—C(CH₃)[(CH₂)₃CH₃]—*′, *—C(CH₃)[(CH₂)₄CH₃]—*′, *—C(CH₂CH₃)₂—*′ *—C(CH₂CH₃)[(CH₂)₂CH₃]—*′, *—C(CH₂CH₃)[(CH₂)₃CH₃]—*′, *—C(CH₂CH₃)[(CH₂)₄CH₃]—*′, and a group represented by one selected from Formulae L-1 to L-8, wherein * and *′ in Formulae L-1 to L-8 each indicate a binding site to a neighboring atom.

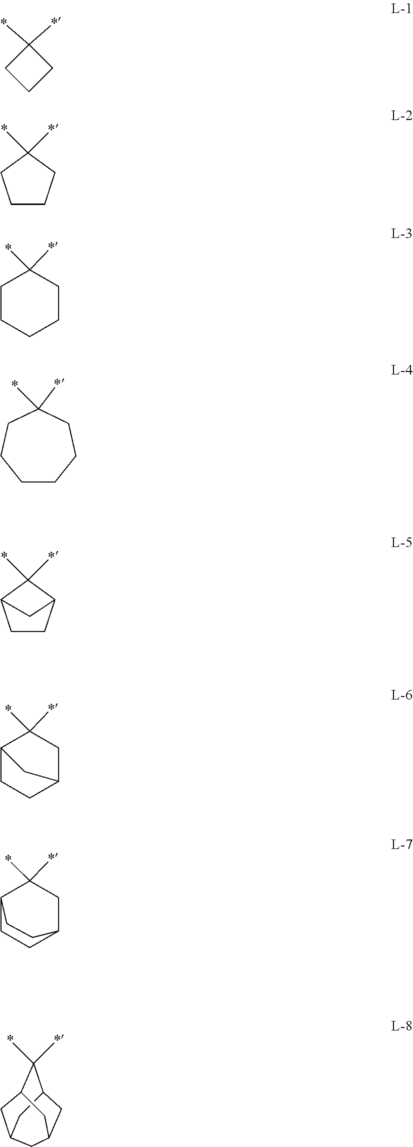

For example, the group represented by *-(L₁)_{c1}-*′ may be one selected from *—(CH₂)—*′, *—CH(CH₃)—*′, *—C(CH₃)₂—*′, *—C(CH₂CH₃)[(CH₂)₄CH₃]—*′, and a group represented by one selected from Formulae L-2, L-3, L-6, and L-8, but embodiments are not limited thereto.

In Formula 1, Ar₁ to Ar₆ may each independently be a single bond, a C₅-C₆₀ carbocyclic group unsubstituted or substituted with at least one R₁₀ₐ, or a C₁-C₆₀ heterocyclic group unsubstituted or substituted with at least one R₁₀ₐ, and

a1 to a6 may each independently be an integer from 1 to 3.

In an embodiment, Ar₁ to Ar₆ may each independently be: a single bond; or

a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluoren-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ, and

R₁₀ₐ is the same as described herein in connection with R₁₀ₐ.

In an embodiment, Ar₁ to Ar₆ in Formula 1 may each independently be one of a single bond and a group represented by one selected from Formulae 2-1 to 2-3, wherein * and *′ in Formulae 2-1 to 2-3 each indicate a binding site to a neighboring atom.

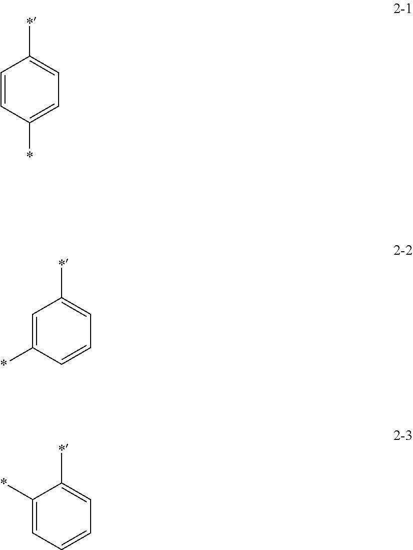

For example, Ar₁ to Ar₄ may each be a single bond, Ar₅ and Ar₆ may each independently be a group represented by one selected from Formulae 2-1 to 2-3, but embodiments are not limited thereto.

In an embodiment, a1 to a6 may each independently be 1 or 2.

For example, a1 to a6 may each be 1, but embodiments are not limited thereto.

W₁ to W₃ in Formula 1 may each independently be a C₅-C₆₀ carbocyclic group unsubstituted or substituted with at least one R₁₀ₐ or a C₁-C₆₀ heterocyclic group unsubstituted or substituted with at least one R₁₀ₐ.

In an embodiment, W₁ to W₃ may each independently be a C₅-C₃₀ carbocyclic group unsubstituted or substituted with at least one R₁₀ₐ or a C₁-C₃₀ heterocyclic group unsubstituted or substituted with at least one R₁₀ₐ.

In an embodiment, W₁ to W₃ may each independently be a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a benzoxazolyl group, a dibenzofuranyl group, a dibenzothiophenyl group, a benzocarbazolyl group, a dibenzocarbazolyl group, or a dibenzosilolyl group, each unsubstituted or substituted with deuterium, —F, —Cl, —Br, —I, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, a cyclopentyl group, a cyclohexyl group, a cycloheptyl group, a cyclooctyl group, an adamantanyl group, a norbornanyl group, a bicyclo[2.2.2]octyl group, a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, an indenyl group, a fluorenyl group, a spiro-bifluorenyl group, a benzofluorenyl group, a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzoimidazolyl group, a benzoxazolyl group, a benzothiazolyl group, a benzooxadiazolyl group, a benzothiadiazolyl group, an imidazopyridinyl group, an imidazopyrimidinyl group, an imidazotriazinyl group, an imidazopyrazinyl group, an imidazopyridazinyl group, —Si(Q₃₁)(Q₃₂)(Q₃₃), —N(Q₃₁)(Q₃₂), —B(Q₃₁)(Q₃₂), or any combination thereof, and

Q₃₁ to Q₃₃ are respectively the same as Q₃₁ to Q₃₃ described herein.

In one or more embodiments, W₁ to W₃ may each independently be a C₅-C₃₀ carbocyclic group or a C₁-C₃₀ heterocyclic group, each unsubstituted or substituted with deuterium, —F, —Cl, —Br, —I, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, a cyclopentyl group, a cyclohexyl group, a cycloheptyl group, a cyclooctyl group, an adamantanyl group, a norbornanyl group, a bicyclo[2.2.2]octyl group, a phenyl group, a naphthyl group, or any combination thereof, wherein the C₅-C₃₀ carbocyclic group and the C₁-C₃₀ heterocyclic group may each be a monocyclic group or a bicyclic group, such as a phenyl group, a naphthyl group, a pyridinyl group, a triazinyl group, a pyrrolyl group, a thiophenyl group, a furanyl group, a benzosilolyl group, a benzothiophenyl group, or a benzofuranyl group, but embodiments are not limited thereto.

When the amine compound has one tricyclic group such as a group represented by Formula 2, a refractive index thereof may be small compared to a case in which the amine compound has two or more tricyclic groups (for example, see Compound E of Comparative Example 6 provided herein), and thus, low refractive properties obtained through an alkane group linking group (see *-(L₁)_{c1}-*′ of Formula 1) may not be impaired or reduced.

In an embodiment, W₁ to W₃ may each independently be a phenyl group or a naphthyl group, each unsubstituted or substituted with deuterium, —F, a cyano group, a nitro group, a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a cyclopentyl group, a cyclohexyl group, a cycloheptyl group, a cyclooctyl group, an adamantanyl group, a norbornanyl group, a bicyclo[1.1.1]pentyl group, a bicyclo[2.1.1]hexyl group, a bicyclo[2.2.2]octyl group, or any combination thereof.

For example, W₁ to W₃ may each independently be a phenyl group unsubstituted or substituted with deuterium, —F, a cyano group, a nitro group, a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a cyclopentyl group, a cyclohexyl group, a cycloheptyl group, a cyclooctyl group, an adamantanyl group, a norbornanyl group, a bicyclo[1.1.1]pentyl group, a bicyclo[2.1.1]hexyl group, a bicyclo[2.2.2]octyl group, or any combination thereof, but embodiments are not limited thereto.

In Formula 1, b1 to b3 may each independently be an integer from 1 to 10.

In an embodiment, b1 to b3 in Formula 1 may each independently be an integer from 1 to 5.

For example, b1 to b3 in Formula 1 may each independently be 1 or 2.

In one or more embodiments, b1 to b3 may each be 1, but embodiments are not limited thereto.

In one or more embodiments, a group represented by (W₁)_{b1}—(Ar₁)ₐ₁—*, a group represented by (W₂)_{b2}—(Ar₂)ₐ₂—*, and a group represented by (W₃)_{b3}—(Ar₃)ₐ₃—* may each independently be a group represented by one selected from Formulae 3-1 to 3-4:

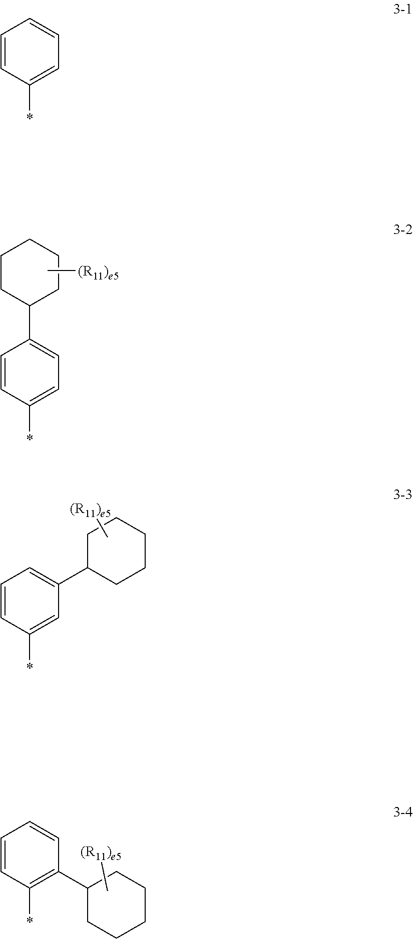

wherein, in Formulae 3-1 to 3-4,

R₁₁ may be: hydrogen, deuterium, —F, or a cyano group; or a C₁-C₁₀ alkyl group or a C₁-C₁₀ alkoxy group, each unsubstituted or substituted with deuterium, —F, a cyano group, or any combination thereof,

e5 may be an integer from 0 to 5, and

* indicates a binding site to a neighboring atom.

For example, i) the group represented by (W₁)_{b1}—(Ar₁)ₐ₁—*, the group represented by (W₂)_{b2}—(Ar₂)ₐ₂—*, and the group represented by (W₃)_{b3}—(Ar₃)ₐ₃—* may each be a group represented by Formula 3-1, or ii) at least one selected from the group represented by (W₁)_{b1}—(Ar₁)ₐ₁—*, the group represented by (W₂)_{b2}—(Ar₂)ₐ₂—*, and the group represented by (W₃)_{b3}—(Ar₃)ₐ₃—* may be a group represented by Formula 3-2, and the remaining groups that are not the group represented by Formula 3-2 may each be a group represented by Formula 3-1, but embodiments are not limited thereto.

T₁ in Formula 1 may be a group represented by Formula 2, and

CY₁ and CY₂ in Formula 2 may each independently be a C₅-C₆₀ carbocyclic group or a C₁-C₆₀ heterocyclic group.

In an embodiment, CY₁ and CY₂ may each independently be a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluoren-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group.

In an embodiment, CY₁ and CY₂ may each independently be a benzene group or a naphthalene group.

For example, CY₁ and CY₂ may each be a benzene group, but embodiments are not limited thereto.

In Formulae 1 and 2, Q₁ may be O, S, N(X₁₁), C(X₁₁)(X₁₂), or Si(X₁₁)(X₁₂),

R₁, R₂, X₁₁, and X₁₂ may each independently be hydrogen or the same as described herein in connection with R₁₀ₐ, and

X₁₁ and X₁₂ may optionally be linked to each other to form a C₃-C₆₀ carbocyclic group or a C₁-C₆₀ heterocyclic group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, or any combination thereof.

In this regard, when Q₁ is C(X₁₁)(X₁₂), X₁₁ and X₁₂ may optionally be linked to each other, so that *—C(X₁₁)(X₁₂)—*′ may form a carbocyclic group or a heterocyclic group (for example, see Compound 449, etc.), and when Q₁ is Si(X₁₁)(X₁₂), X₁₁ and X₁₂ may optionally be linked to each other, so that *—Si(X₁₁)(X₁₂)—*′ may form a heterocyclic group.

In an embodiment, X₁₁ and X₁₂ may each independently be: hydrogen; a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, or a sec-octyl group, each unsubstituted or substituted with deuterium, —F, a cyano group, or any combination thereof; or

a cyclopentyl group, a cyclohexyl group, a cycloheptyl group, a cyclooctyl group, an adamantanyl group, a norbornanyl group, a bicyclo[1.1.1]pentyl group, a bicyclo[2.1.1]hexyl group, a bicyclo[2.2.2]octyl group, a phenyl group, or a naphthyl group, each unsubstituted or substituted with deuterium, —F, a cyano group, a C₁-C₁₀ alkyl group, or any combination thereof.

In one or more embodiments, when Q₁ is C(X₁₁)(X₁₂), X₁₁ and X₁₂ may optionally be linked to each other, so that *—C(X₁₁)(X₁₂)—*′ may form a C₃-C₂₀ cycloalkane group unsubstituted or substituted with deuterium, —F, —Cl, —Br, —I, a hydroxyl group, a cyano group, a nitro group, a C₁-C₁₀ alkyl group, or any combination thereof.

In this regard, the term “cycloalkane group,” as used herein, includes not only a monocyclic group, such as a cyclopentane group or a cyclohexane group, but also a condensed cyclic unsaturated hydrocarbon group, such as an adamantane group.

In one or more embodiments, when Q₁ is C(X₁₁)(X₁₂), X₁₁ and X₁₂ may optionally be linked to each other, so that *—C(X₁₁)(X₁₂)—*′ may form a group represented by one selected from Formulae L-1 to L-8, wherein * and *′ in Formulae L-1 to L-8 each indicate a binding site to a neighboring atom.

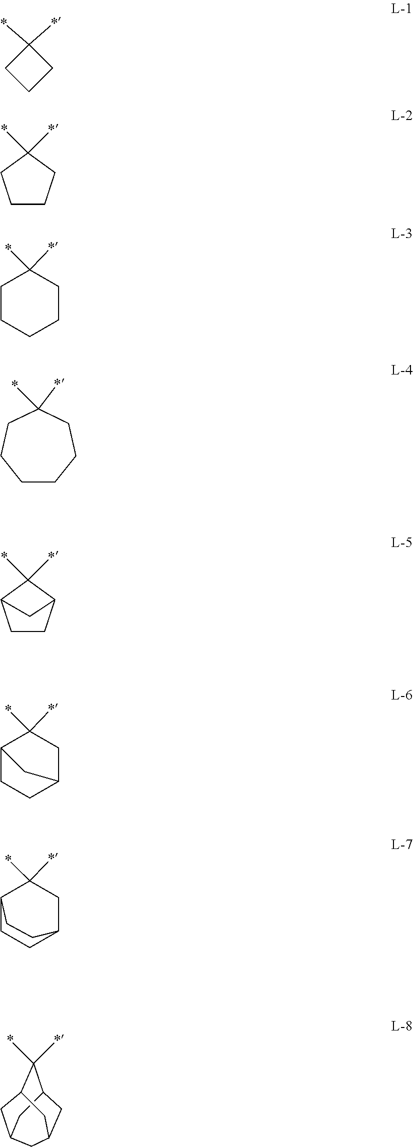

For example, when Q₁ is C(X₁₁)(X₁₂), X₁₁ and X₁₂ may optionally be linked to each other, so that *—C(X₁₁)(X₁₂)—*′ may form a cycloalkane group selected from groups represented by Formulae L-1 to L-4.

In an embodiment, R₁ and R₂ in Formula 2 may each independently be: hydrogen, deuterium, —F, a cyano group, or a nitro group; or a C₁-C₁₀ alkyl group, a C₃-C₁₀ cycloalkyl group, or a C₁-C₁₀ alkoxy group, each unsubstituted or substituted with deuterium, —F, a cyano group, a nitro group, or any combination thereof.

In Formula 2, d1 and d2 may each independently be an integer from 0 to 10.

In an embodiment, d1 and d2 may each independently be an integer from 0 to 5.

In an embodiment, T₁ may be a group represented by one selected from Formulae 4-1 to 4-4:

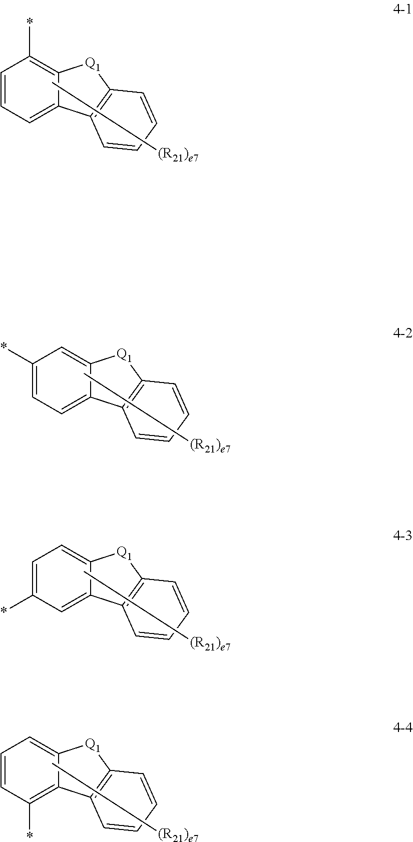

wherein, in Formulae 4-1 to 4-4,

Q₁ is the same as Q₁ described with respect to Formula 2,

R₂₁ is the same as described in connection with R₁,

e7 may be an integer from 0 to 7, and

* indicates a binding site to a neighboring atom.

In Formula 1, a group represented by *—(Ar₄)ₐ₄-T₁ may be different from a group represented by *—(Ar₁)ₐ₁—(W₁)_{b1} and a group represented by *—(Ar₂)ₐ₂—(W₂)_{b2}, and * and *′ each indicate a binding site to a neighboring atom.

In an embodiment, the group represented by (W₁)_{b1}—(Ar₁)ₐ₁—*, the group represented by (W₂)_{b2}—(Ar₂)ₐ₂—*, and the group represented by (W₃)_{b3}—(Ar₃)ₐ₃—* may each independently be a group represented by one selected from Formulae 3-1 to 3-4, and T₁ may be a group represented by one selected from Formulae 4-1 to 4-4.

In an embodiment, the amine compound may be represented by Formula 1A:

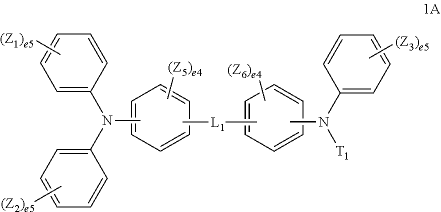

wherein, in Formula 1A,

L₁ and T₁ are respectively the same as L₁ and T₁ described with respect to Formula 1,

Z₁ to Z₃, Z₅, and Z₆ are each same as described herein in connection with R₁₀ₐ,

e4 may be an integer from 0 to 4, and

e5 may be an integer from 0 to 5.

For example, Z₁ to Z₃, Z₅, and Z₆ in Formula 1A may each independently be: hydrogen, deuterium, —F, a cyano group, or a nitro group; or a C₁-Cia alkyl group, a C₃-C₁₀ cycloalkyl group, or a C₁-C₁₀ alkoxy group, each unsubstituted or substituted with deuterium, —F, a cyano group, a nitro group, or any combination thereof.

The term “R₁₀ₐ,” as used herein, may be:

deuterium, —F, —Cl, —Br, —I, a hydroxyl group, a cyano group, or a nitro group;

a C₁-C₆₀ alkyl group, a C₂-C₆₀ to alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C h-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and

Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, the amine compound represented by Formula 1 may be one selected from Compounds 1 to 584, but embodiments are not limited thereto:

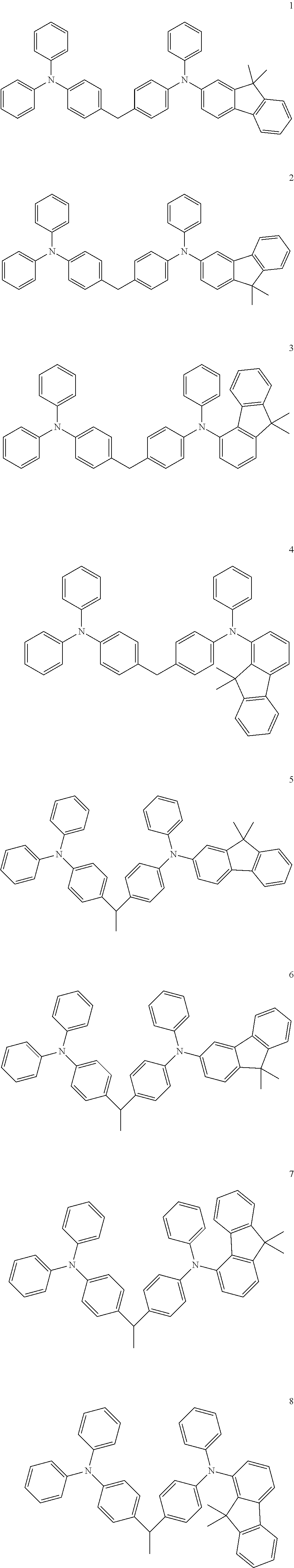

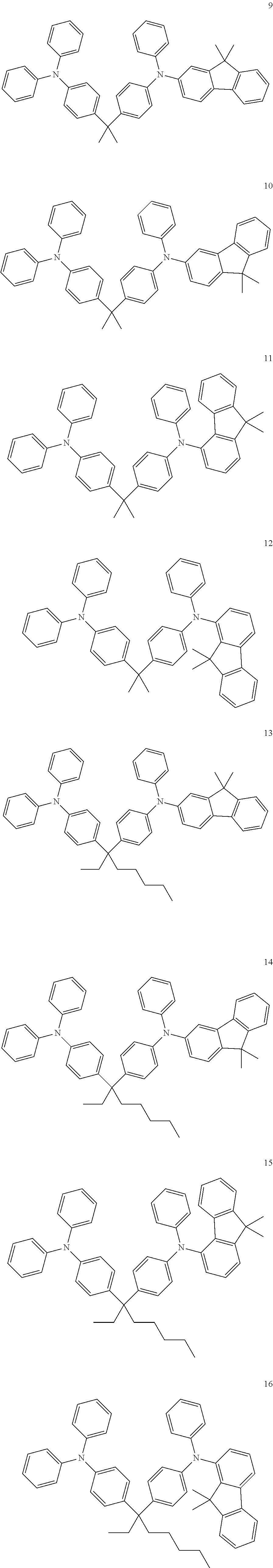

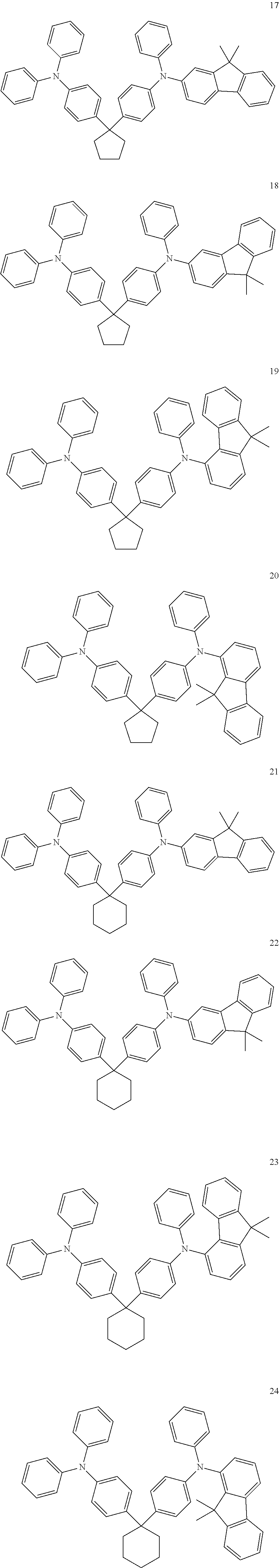

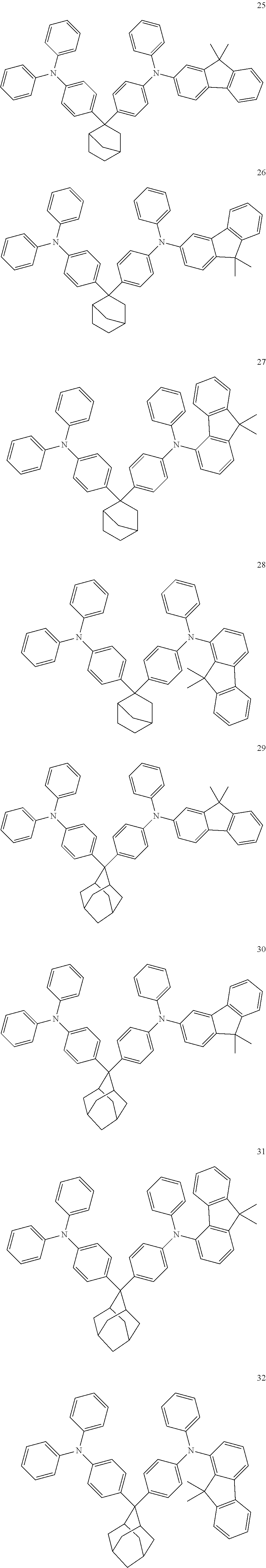

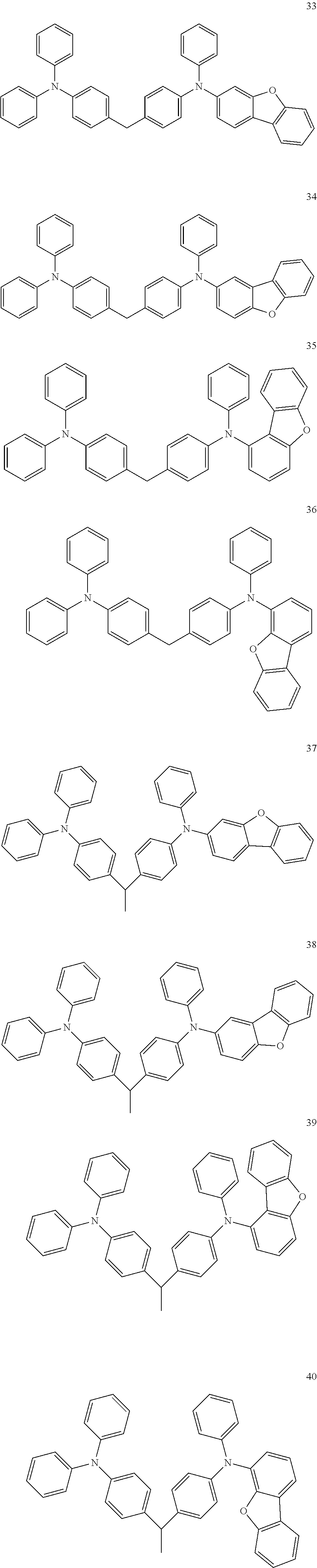

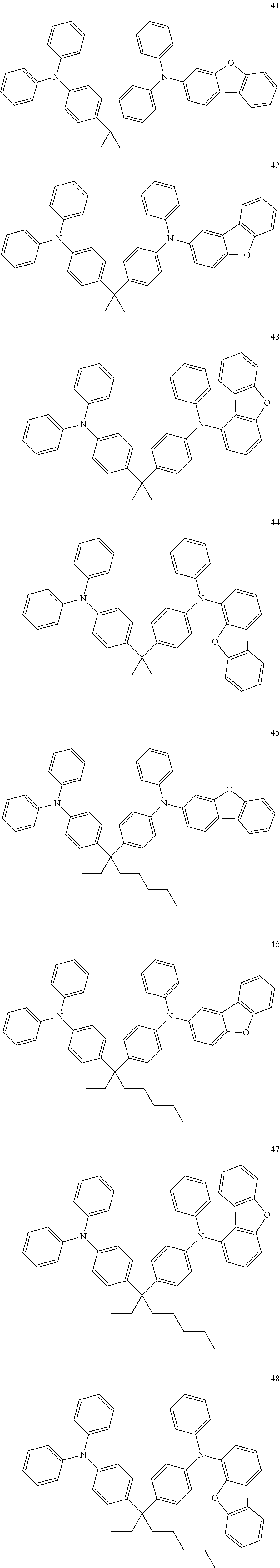

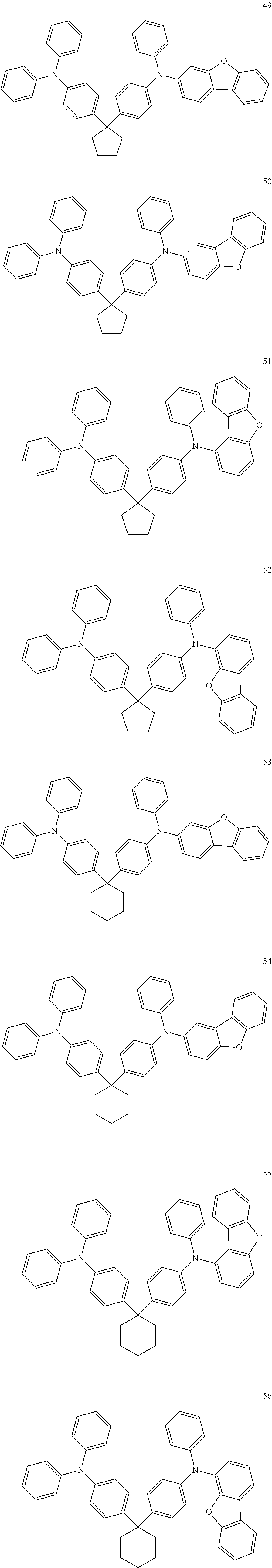

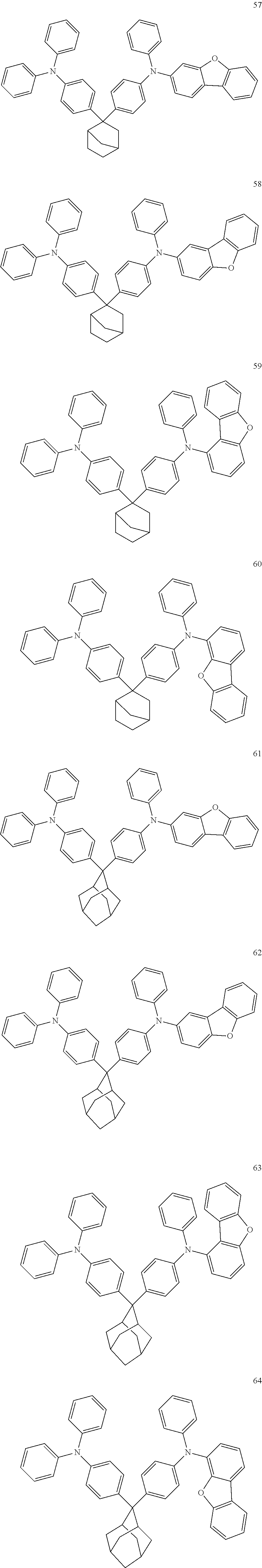

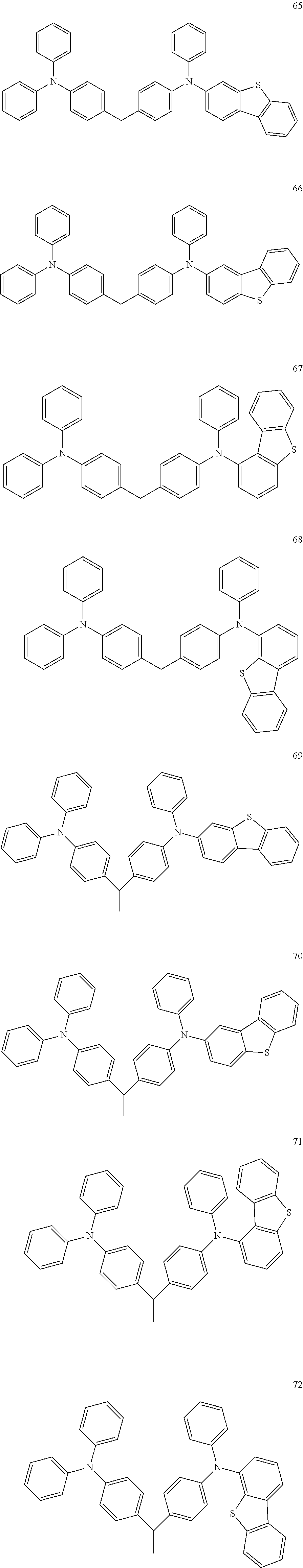

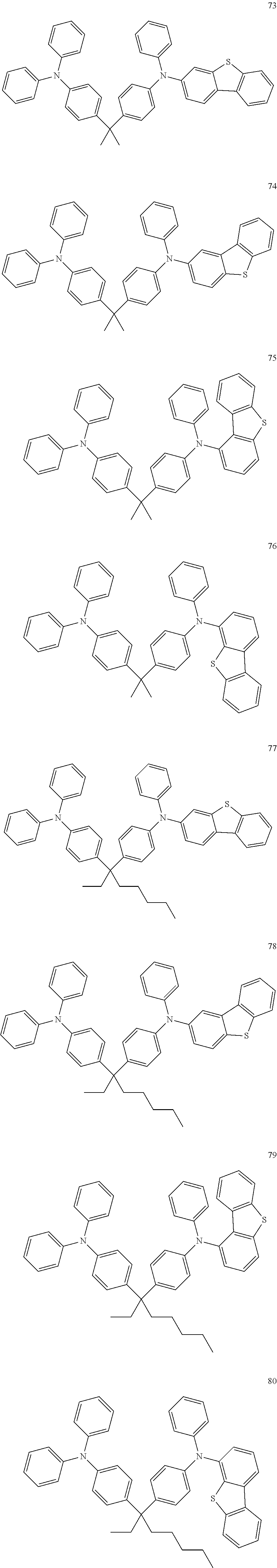

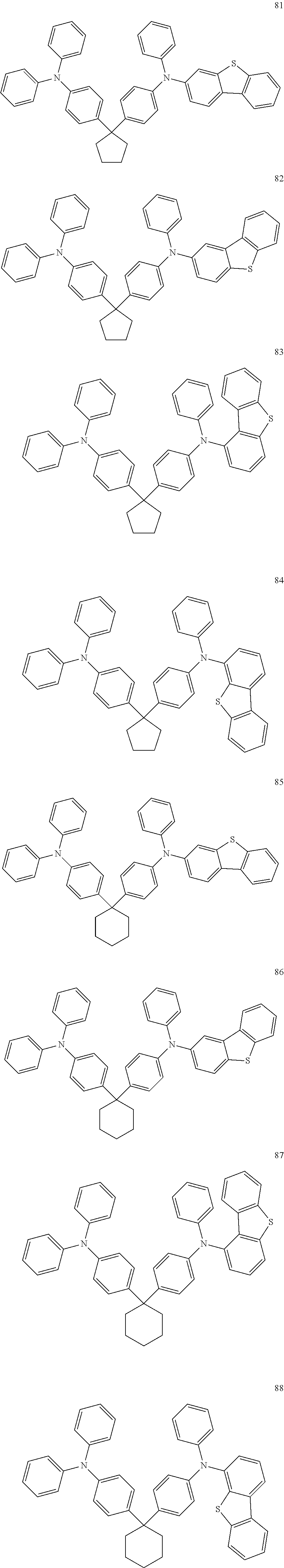

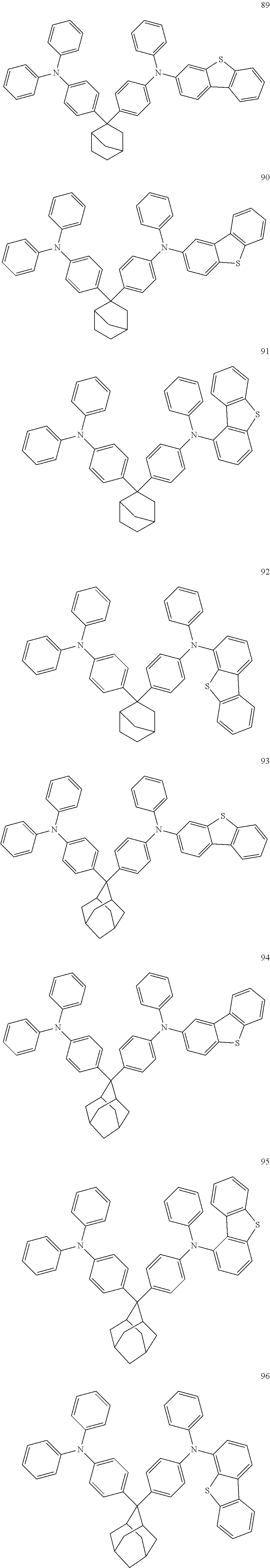

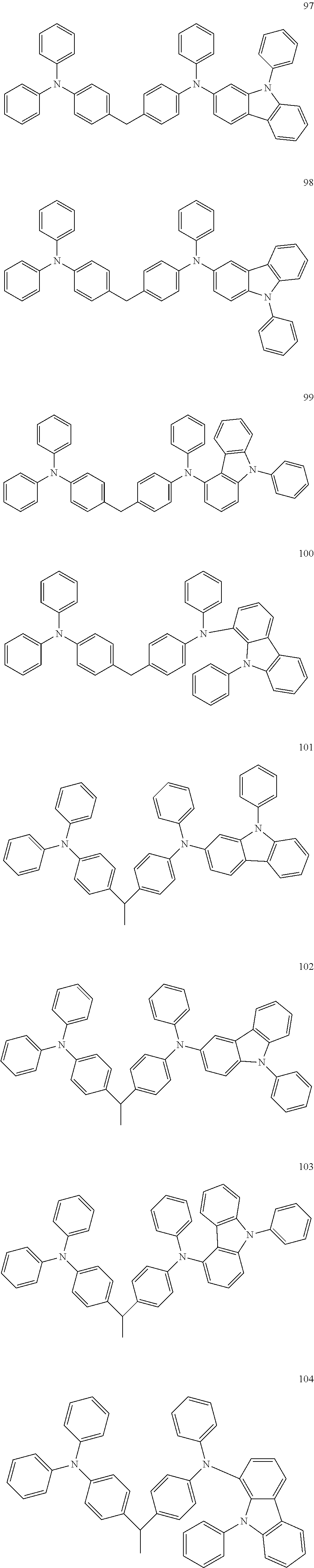

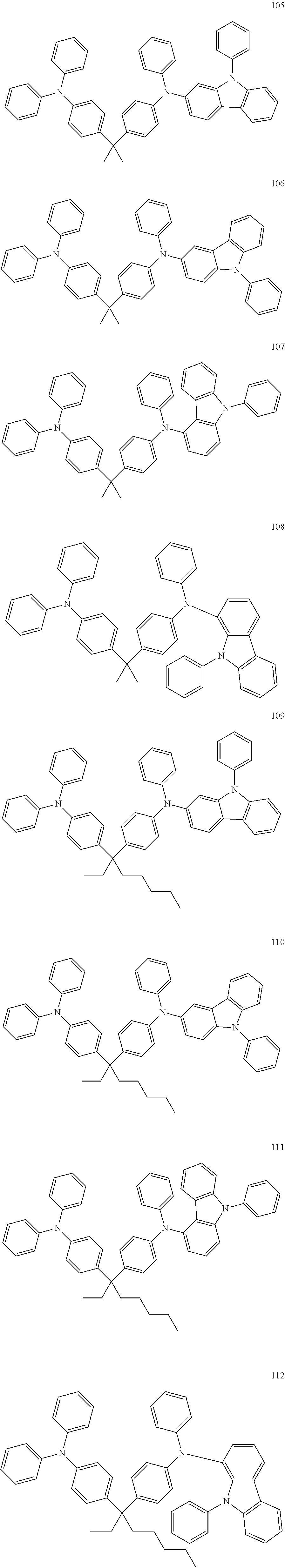

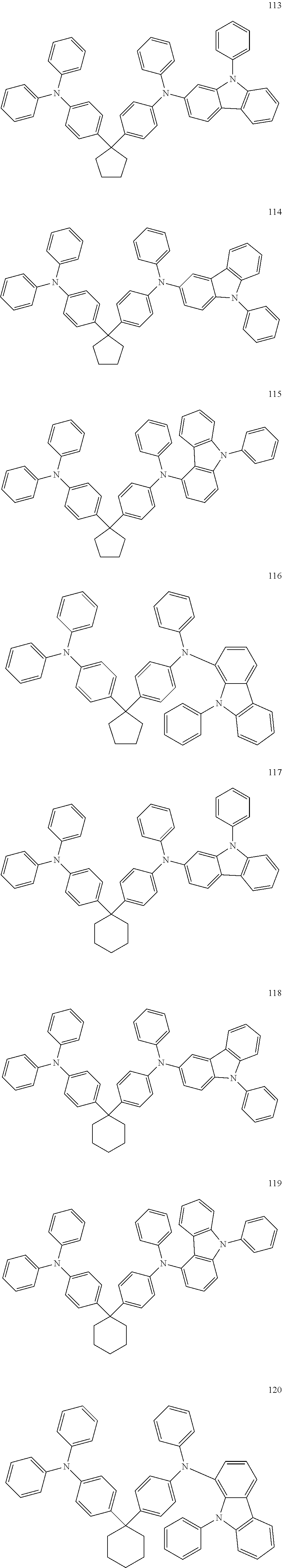

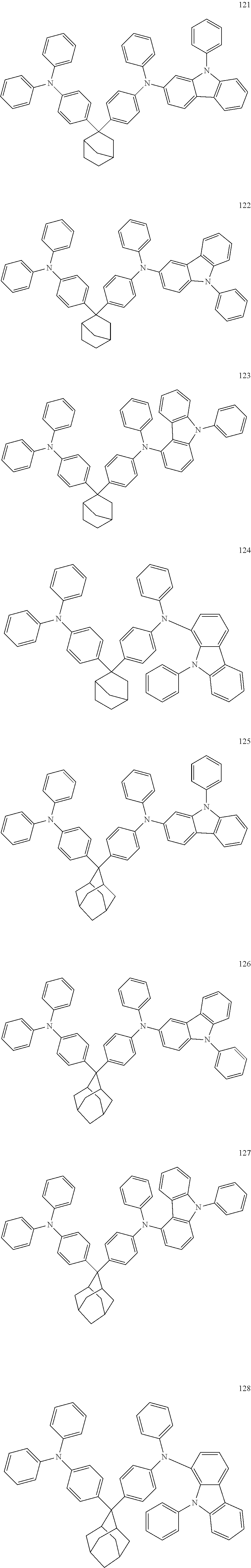

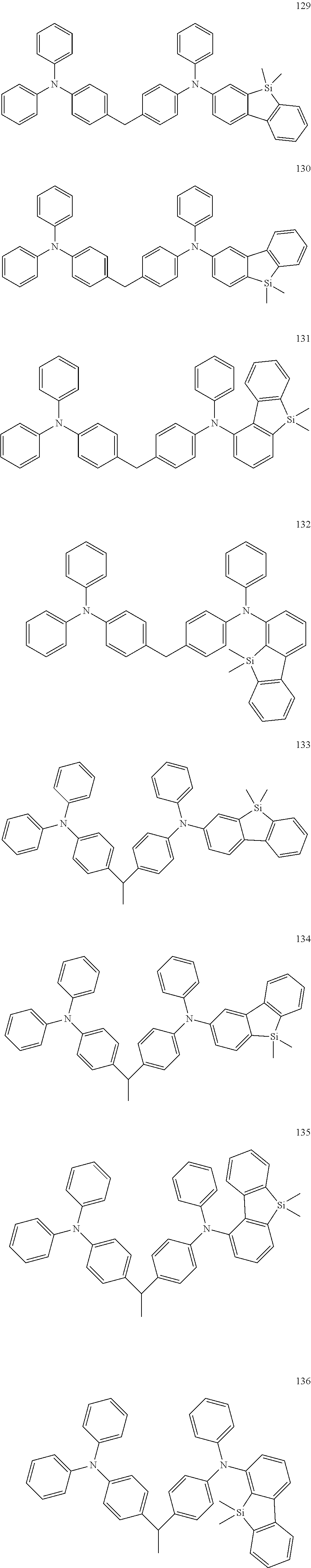

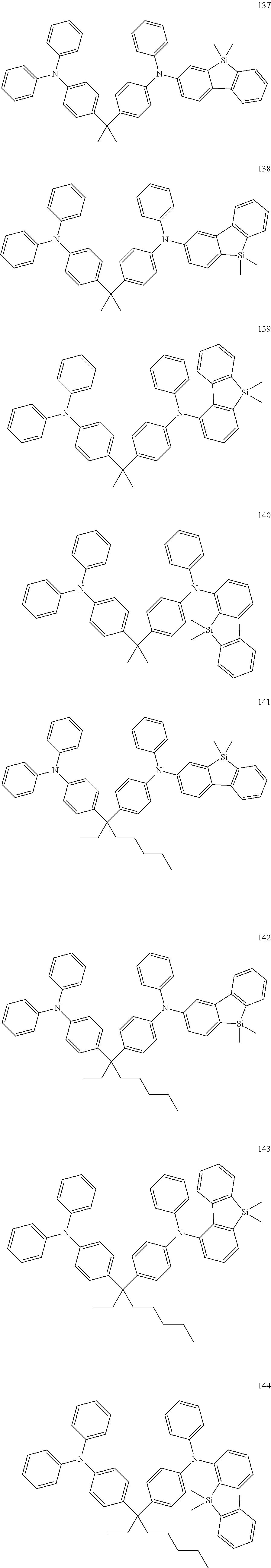

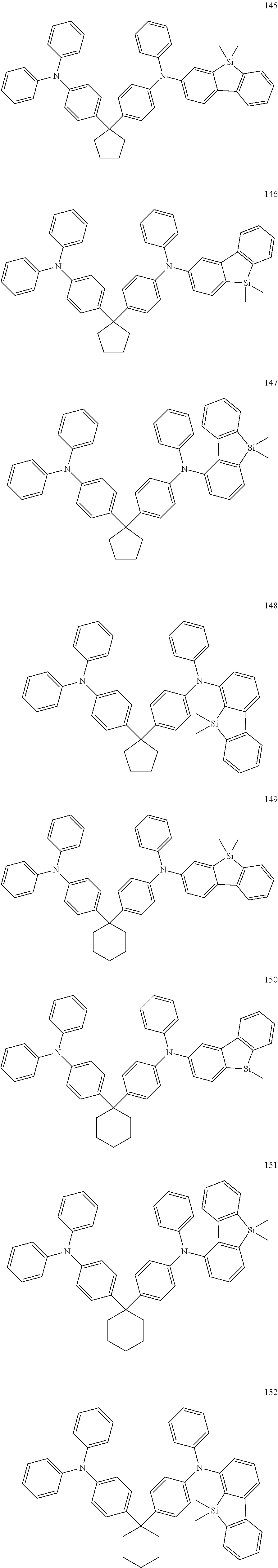

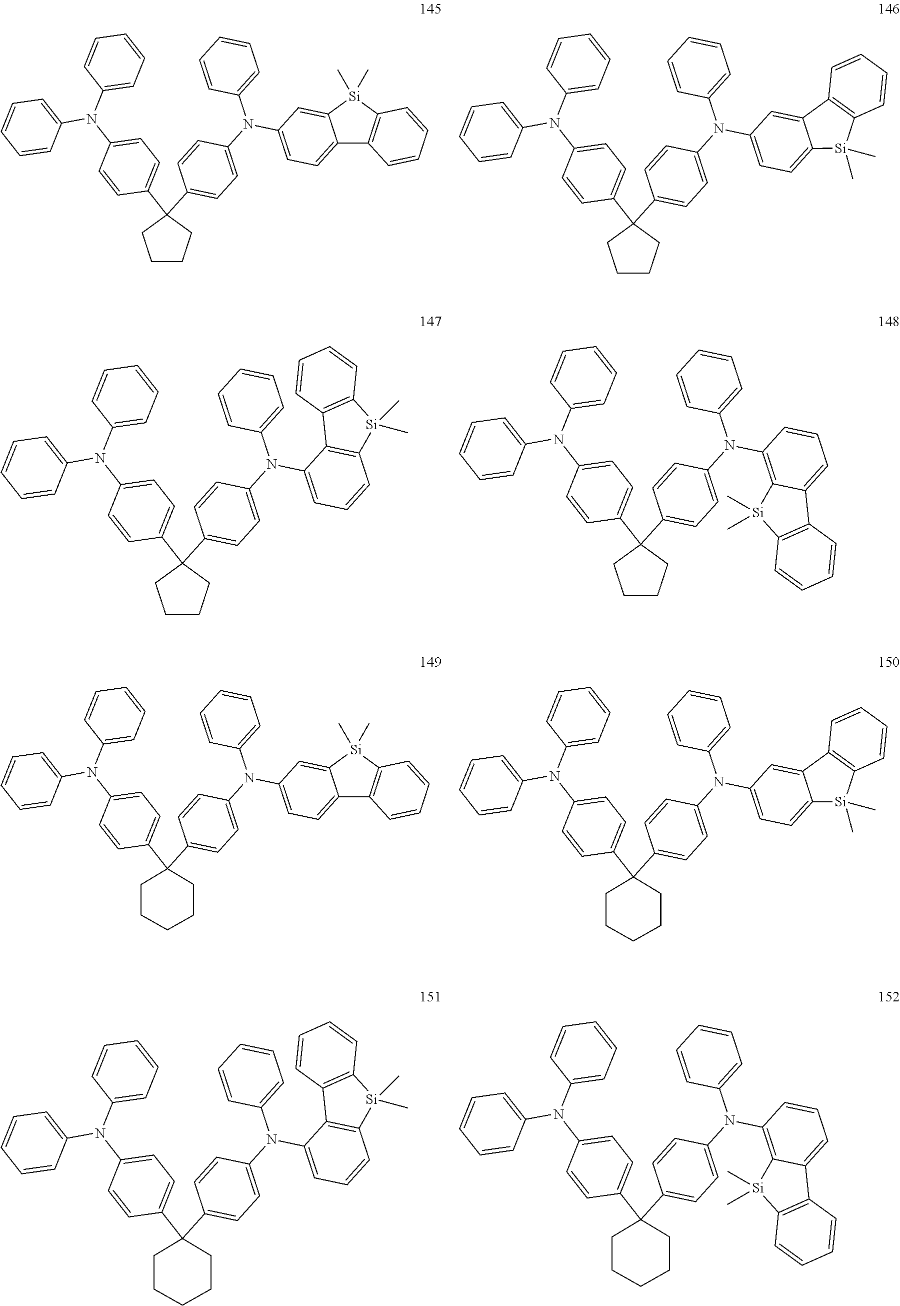

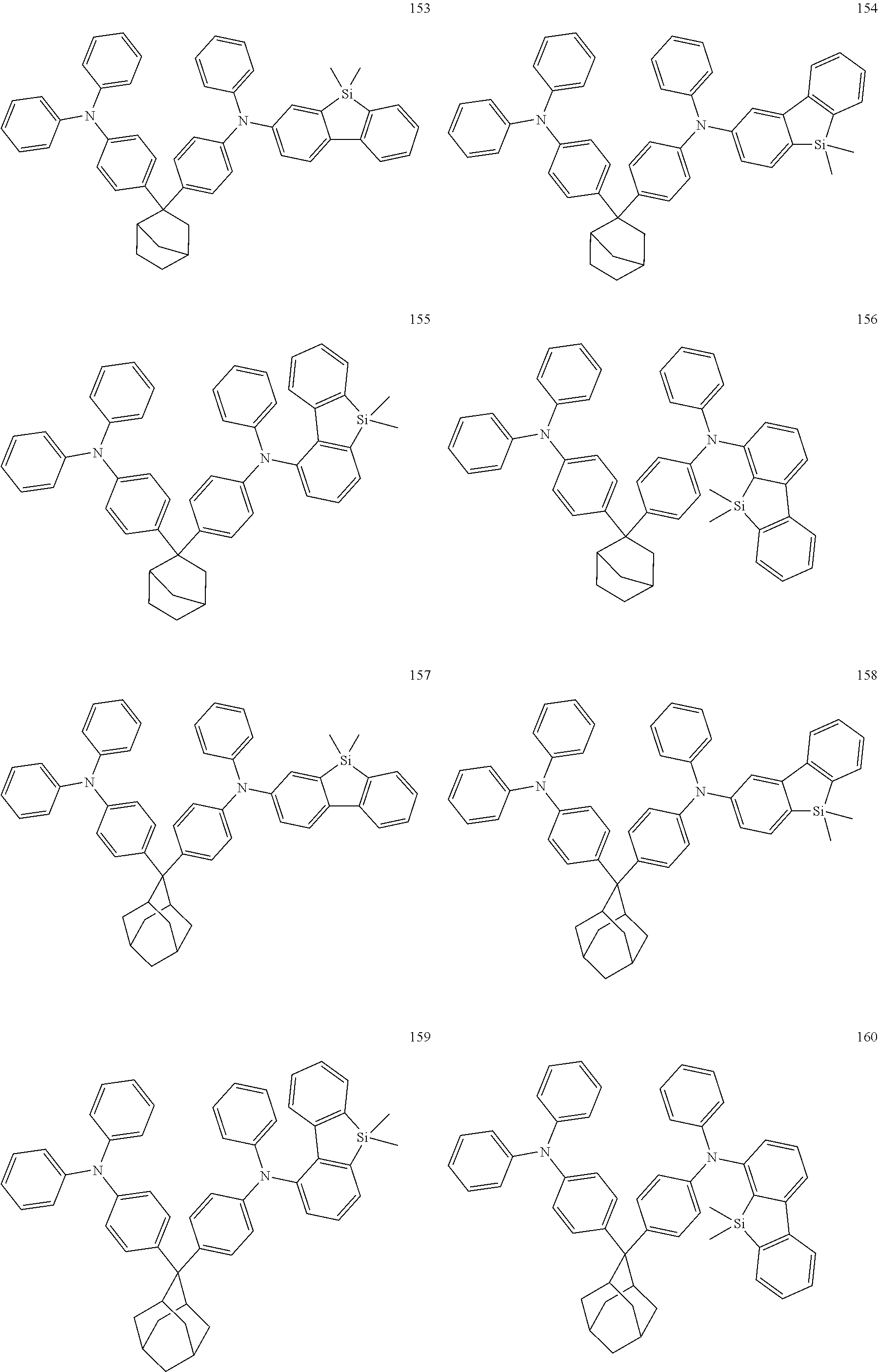

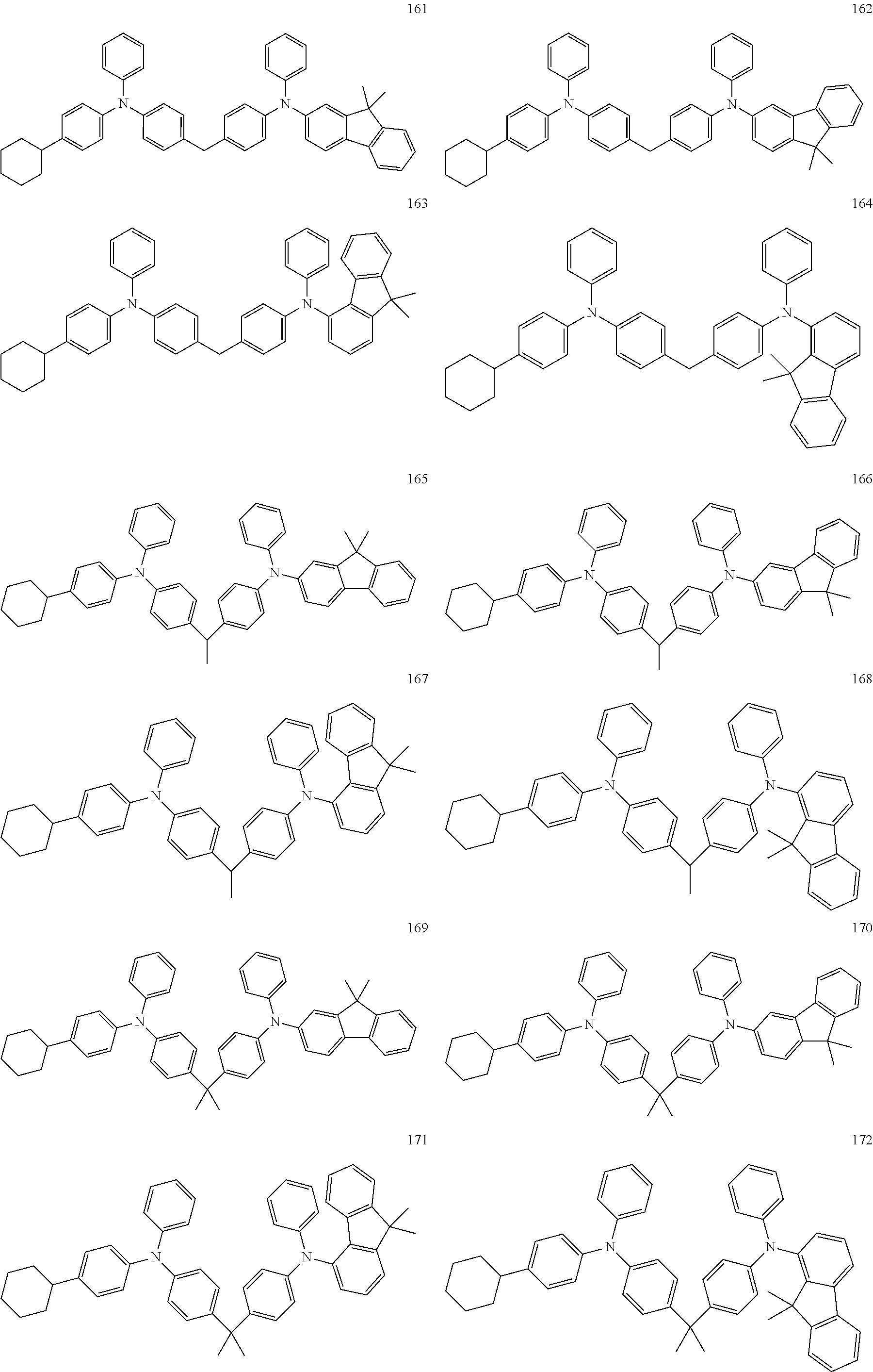

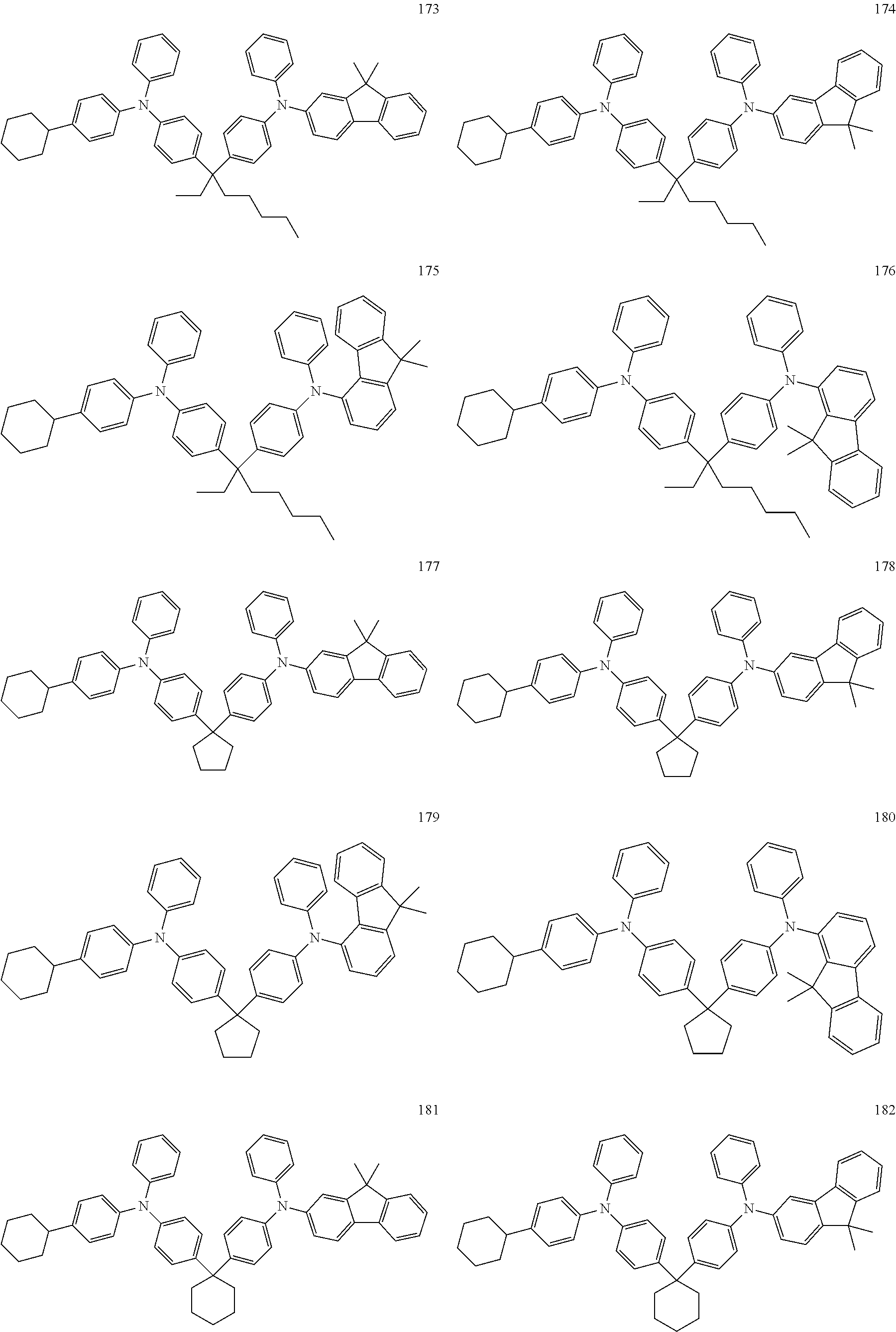

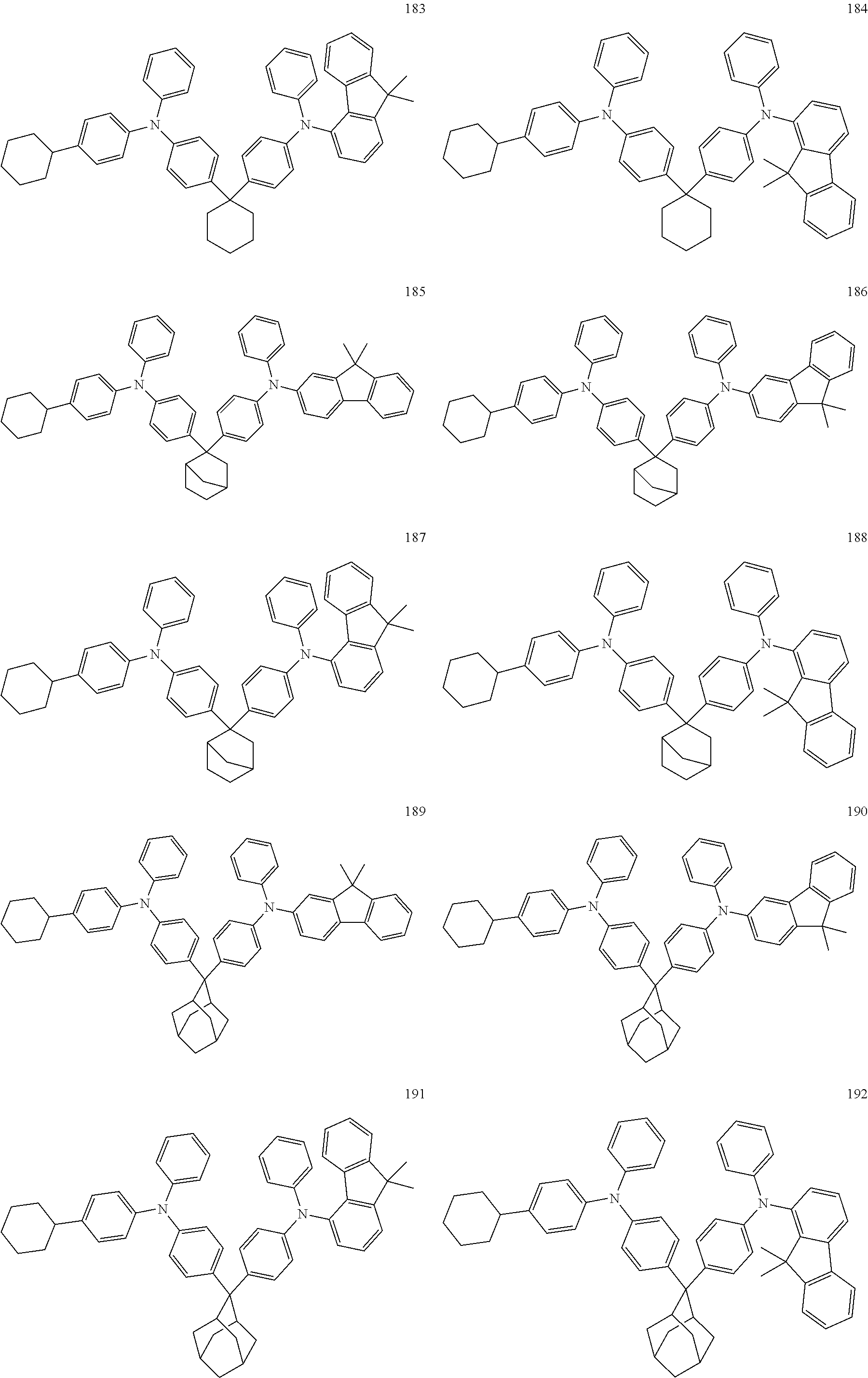

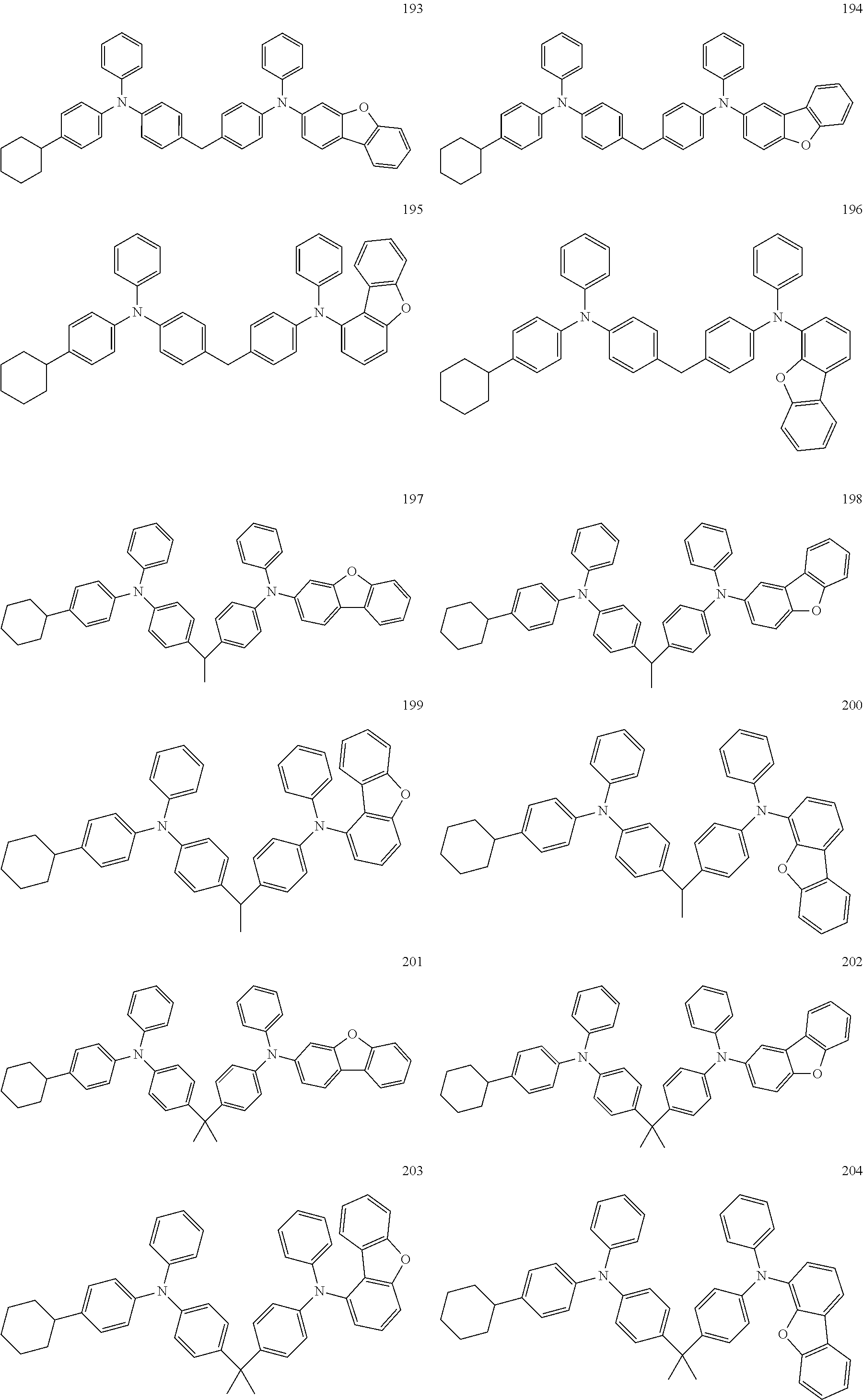

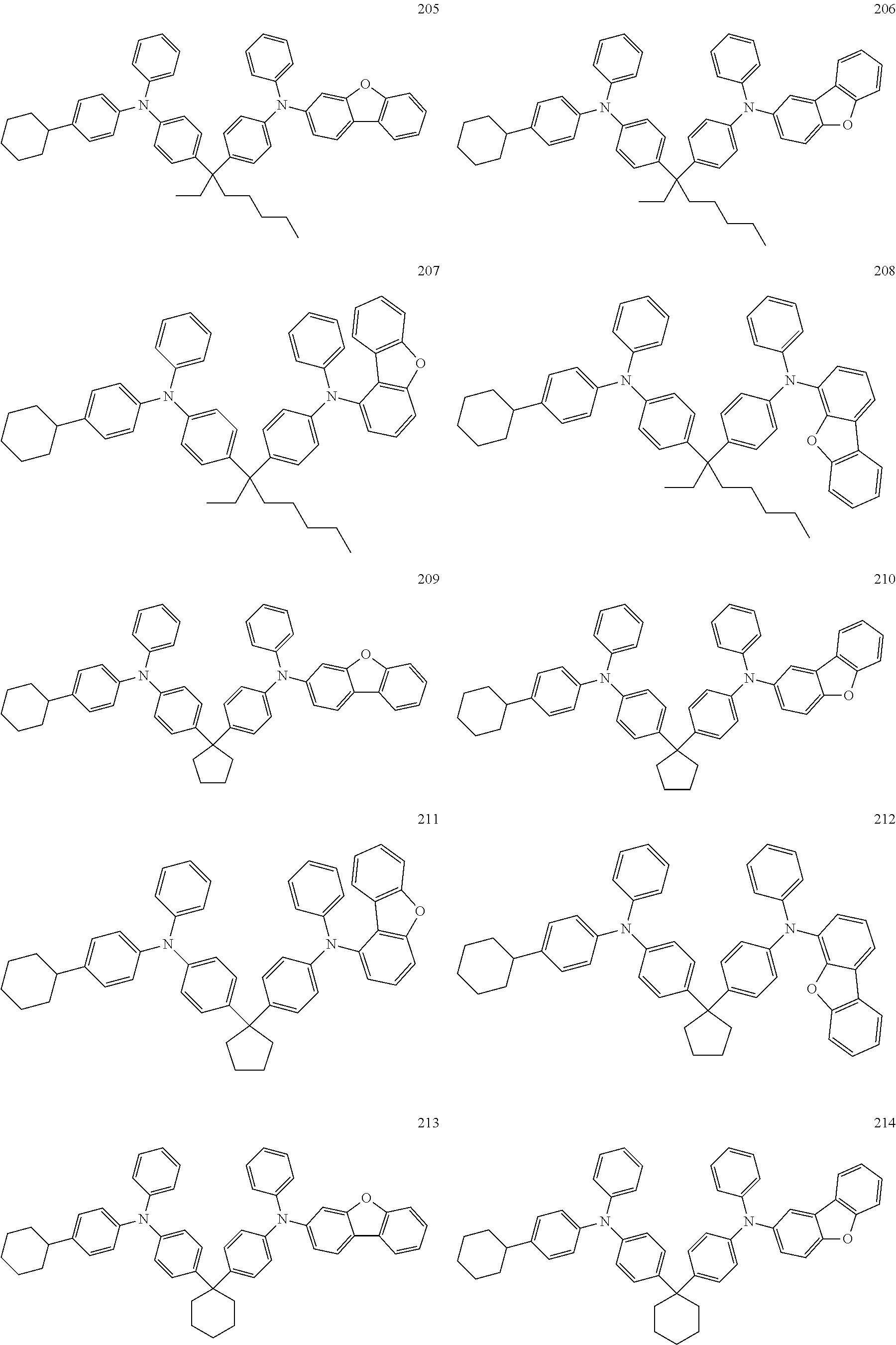

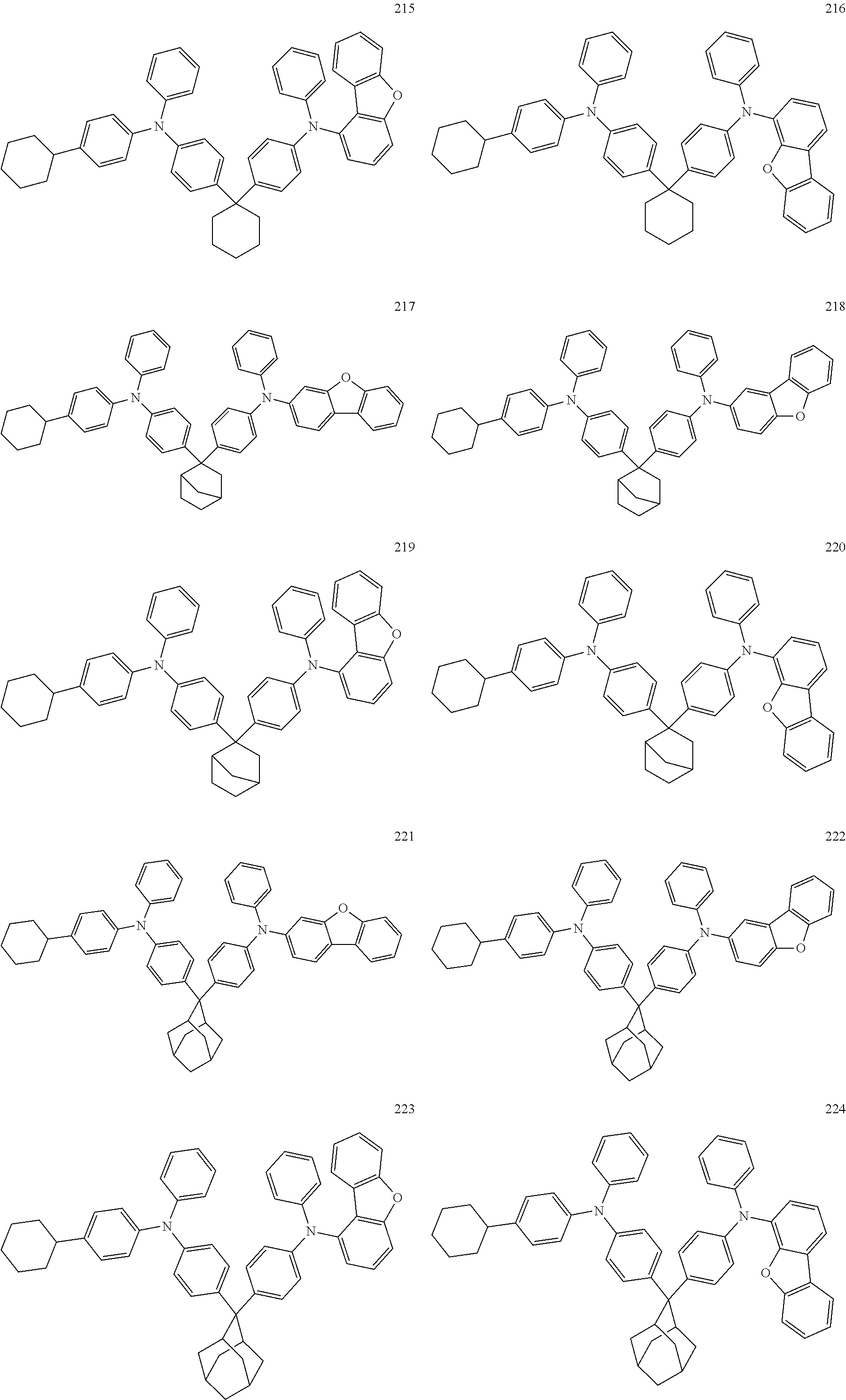

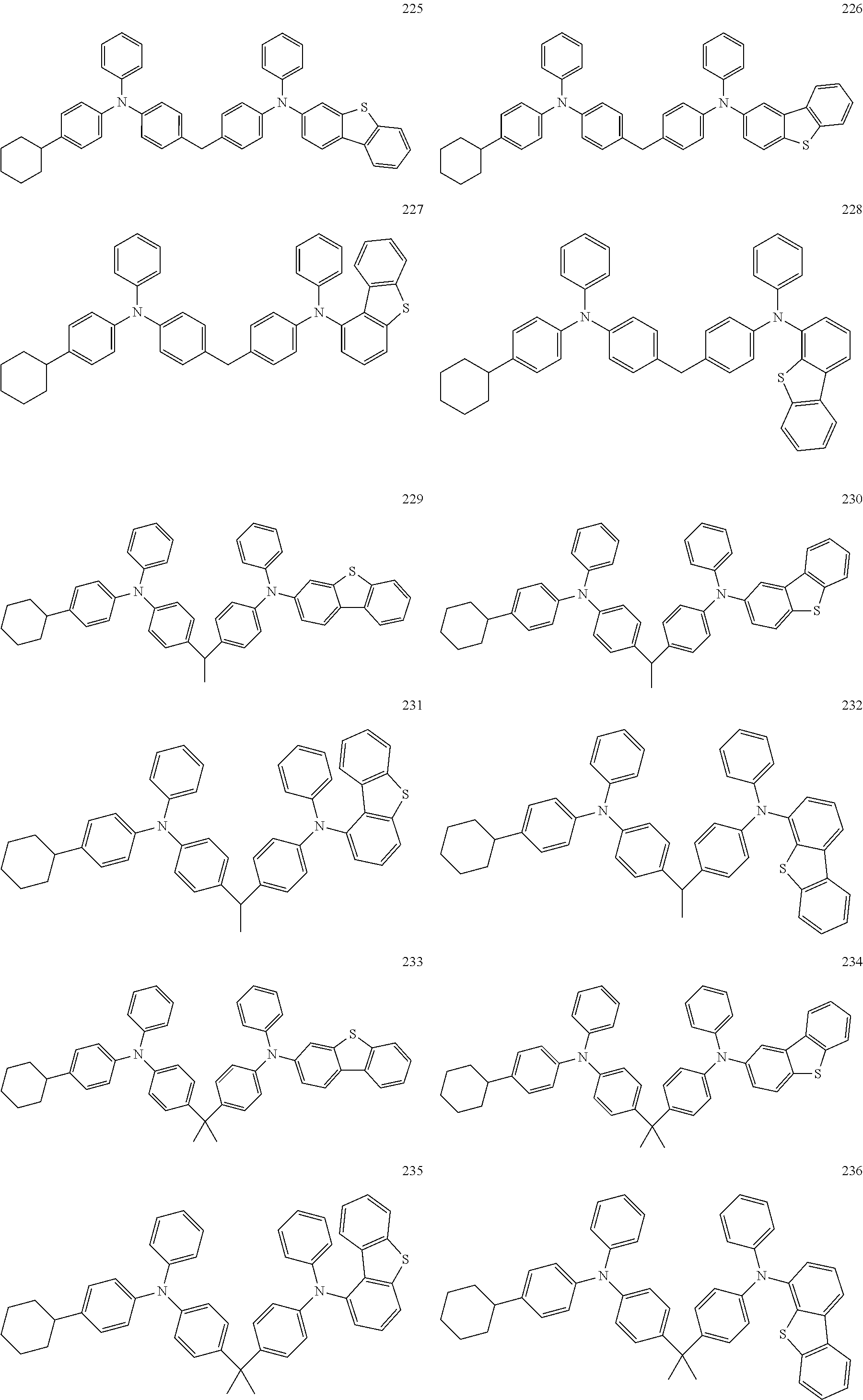

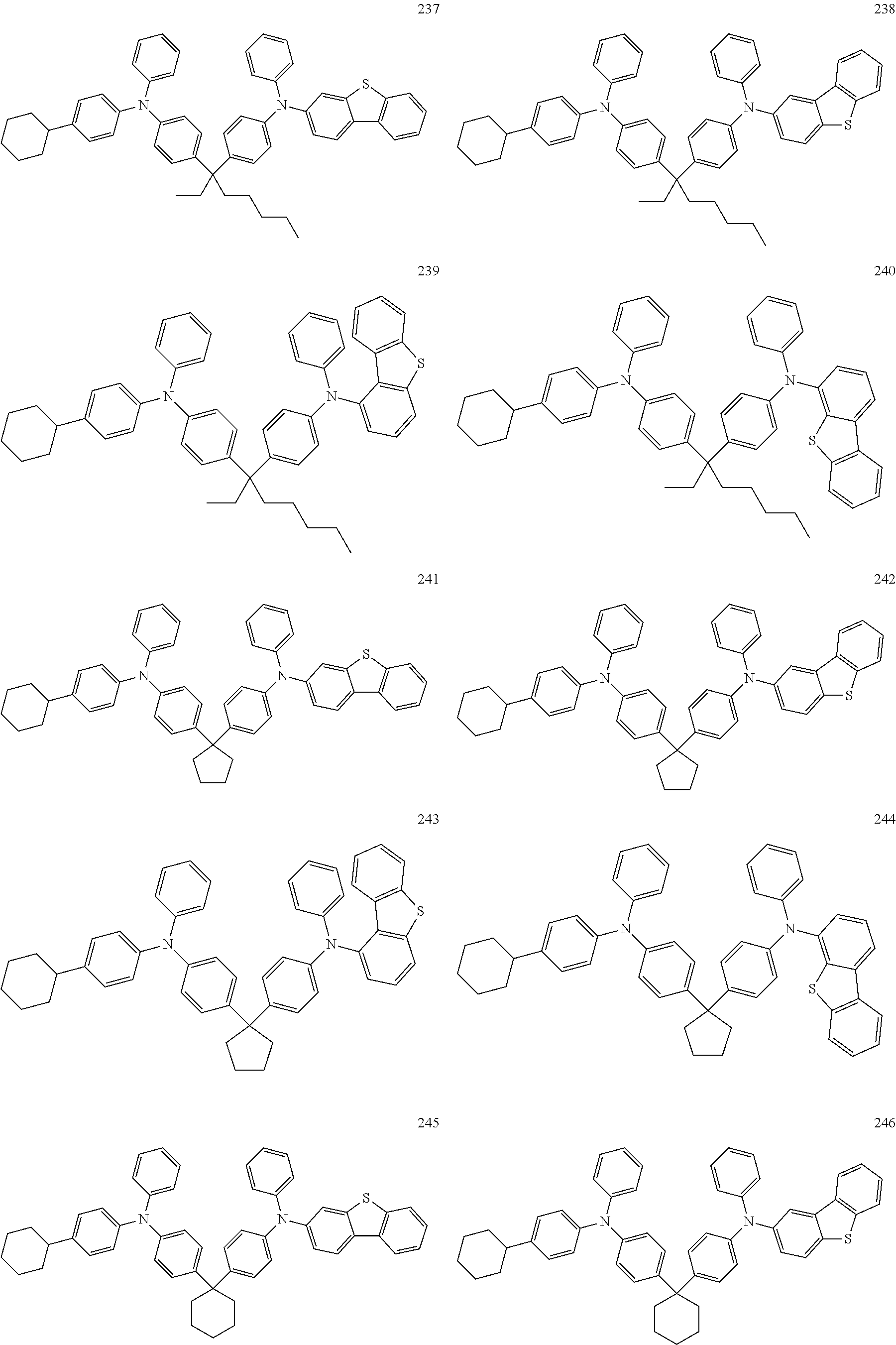

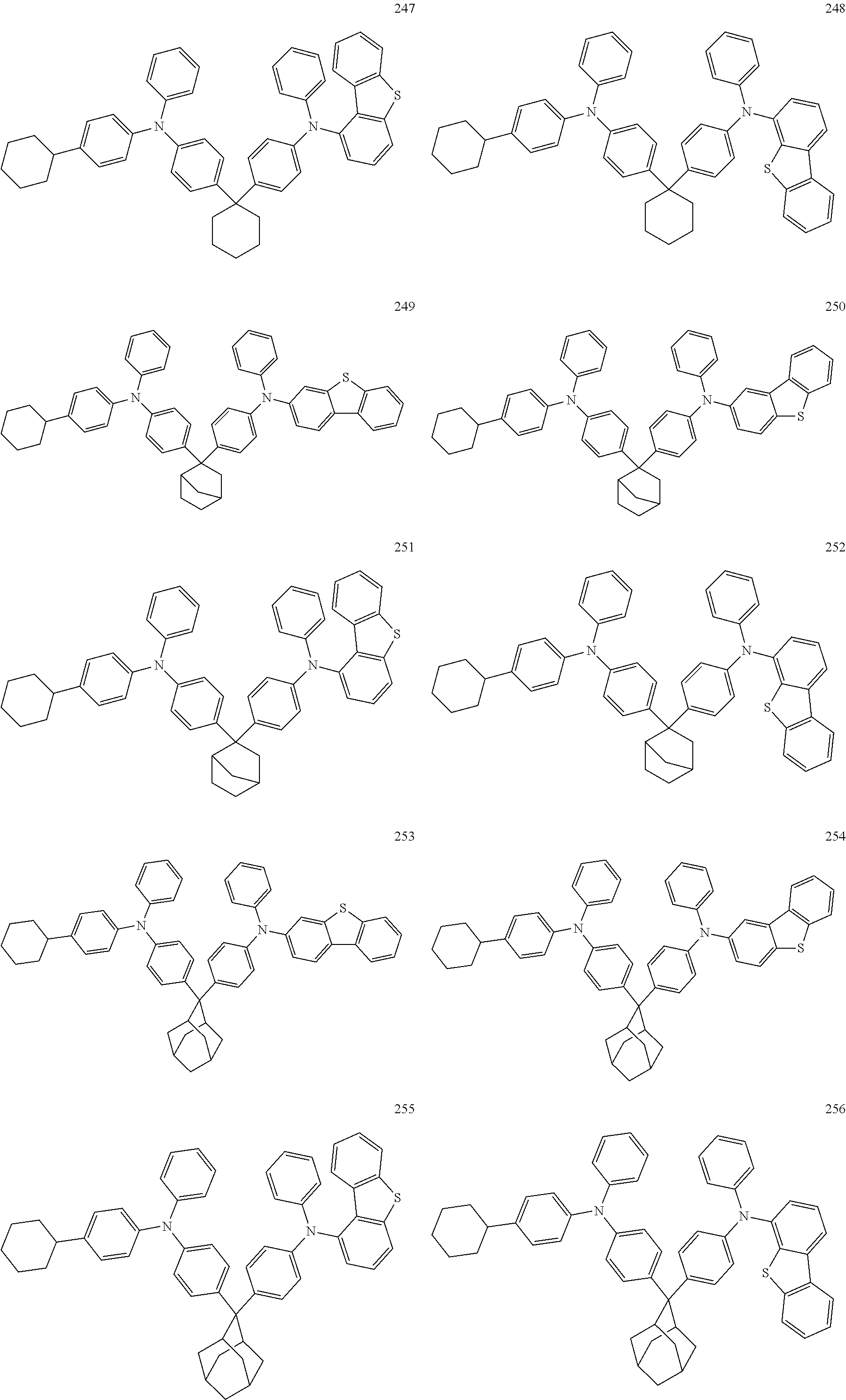

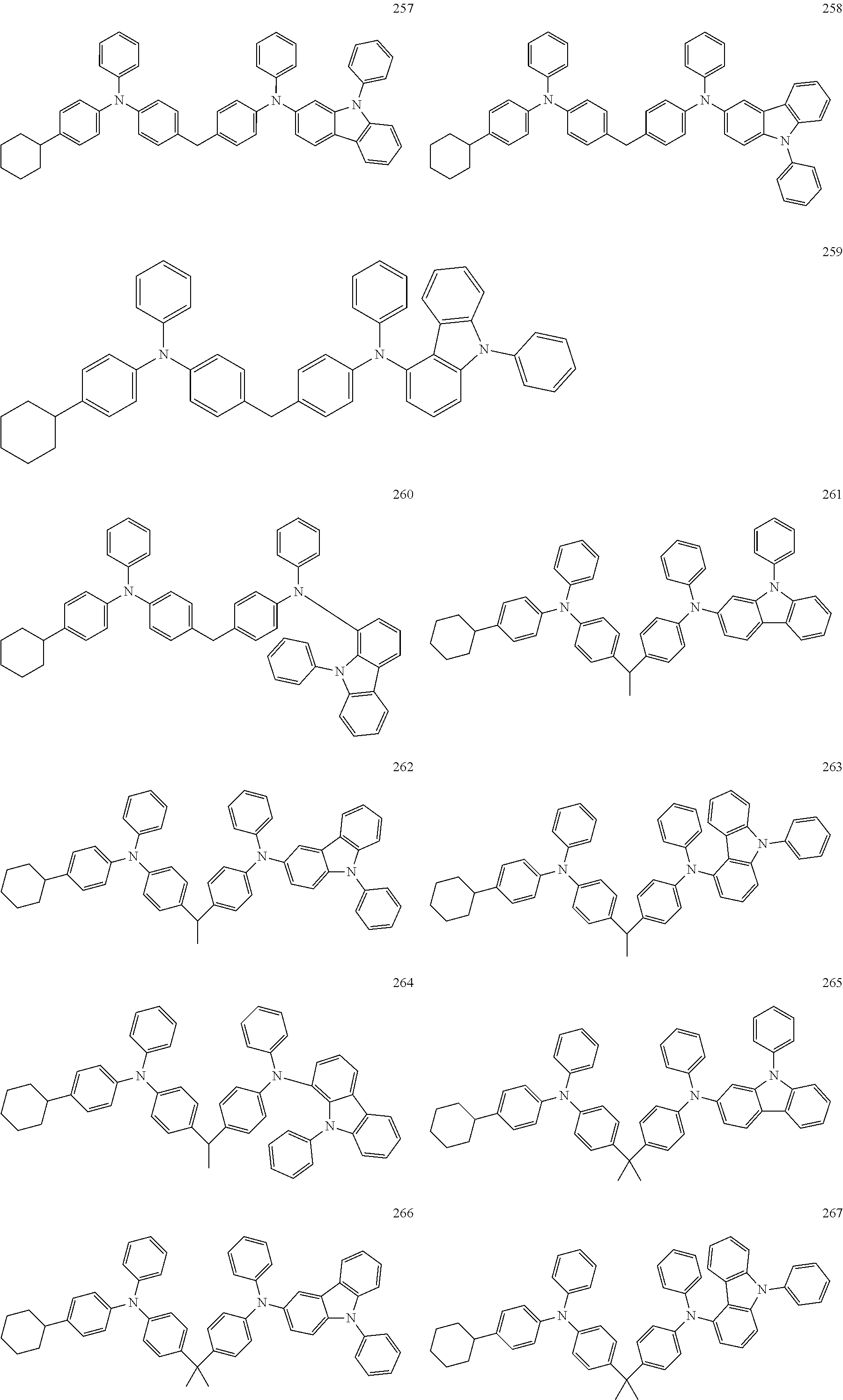

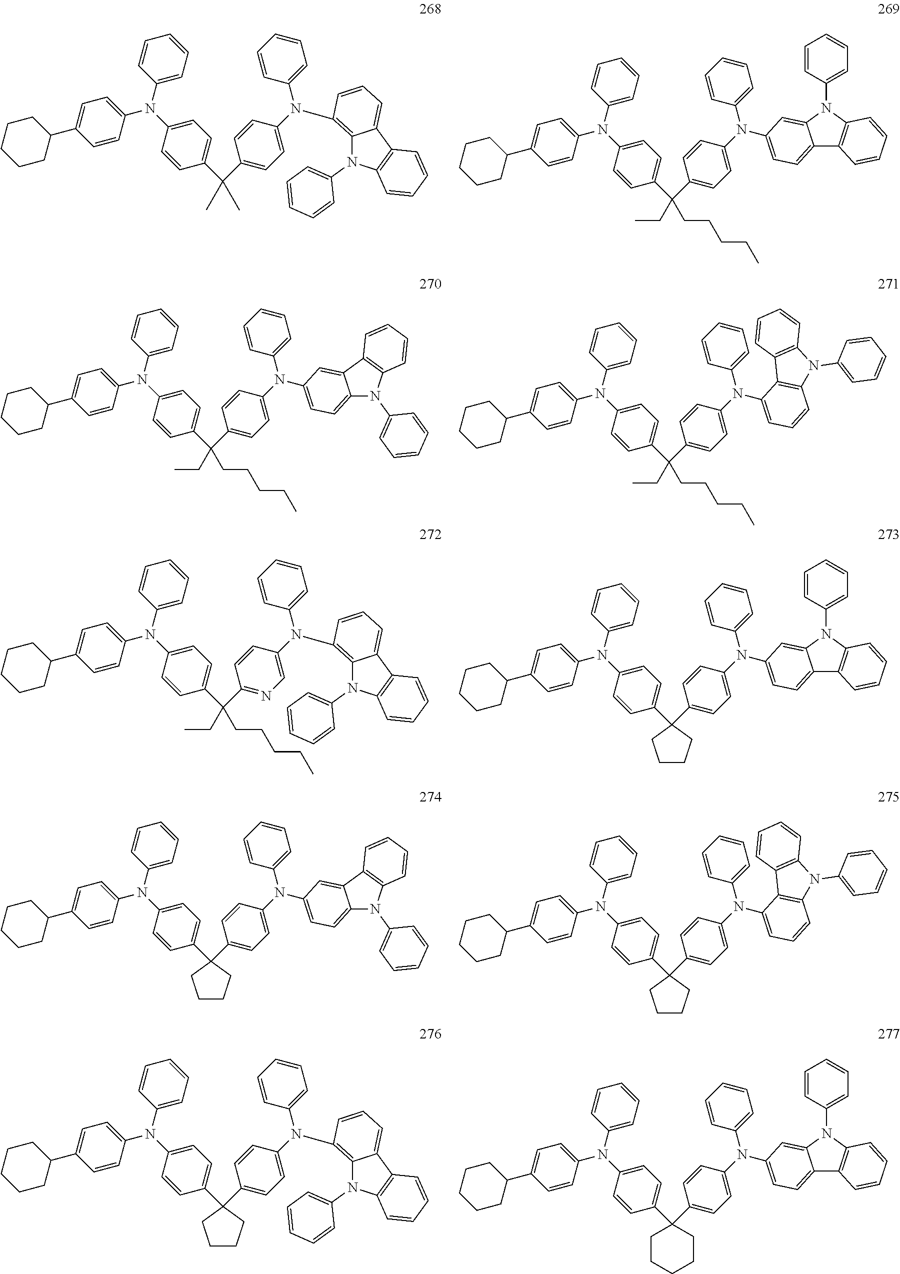

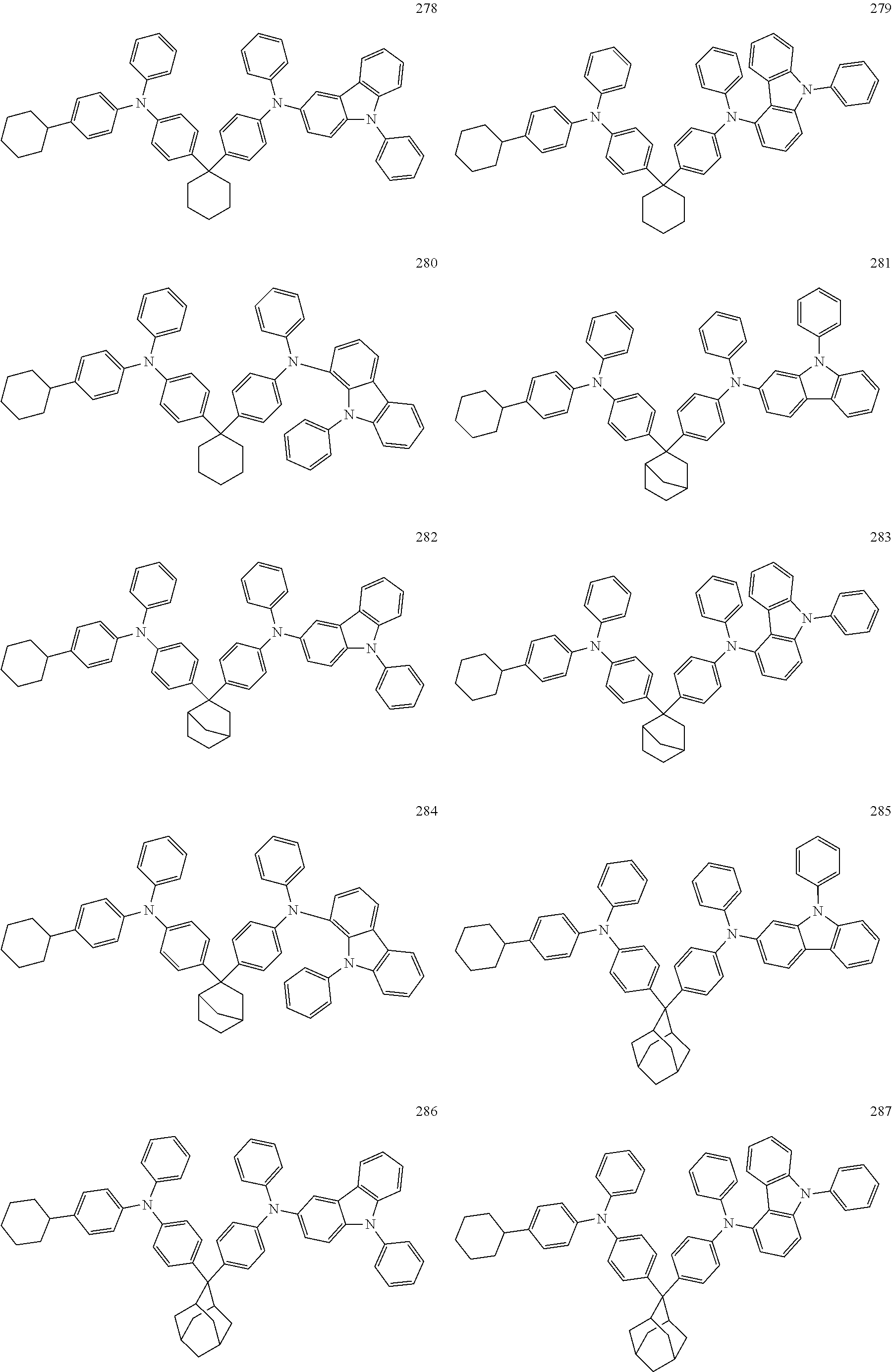

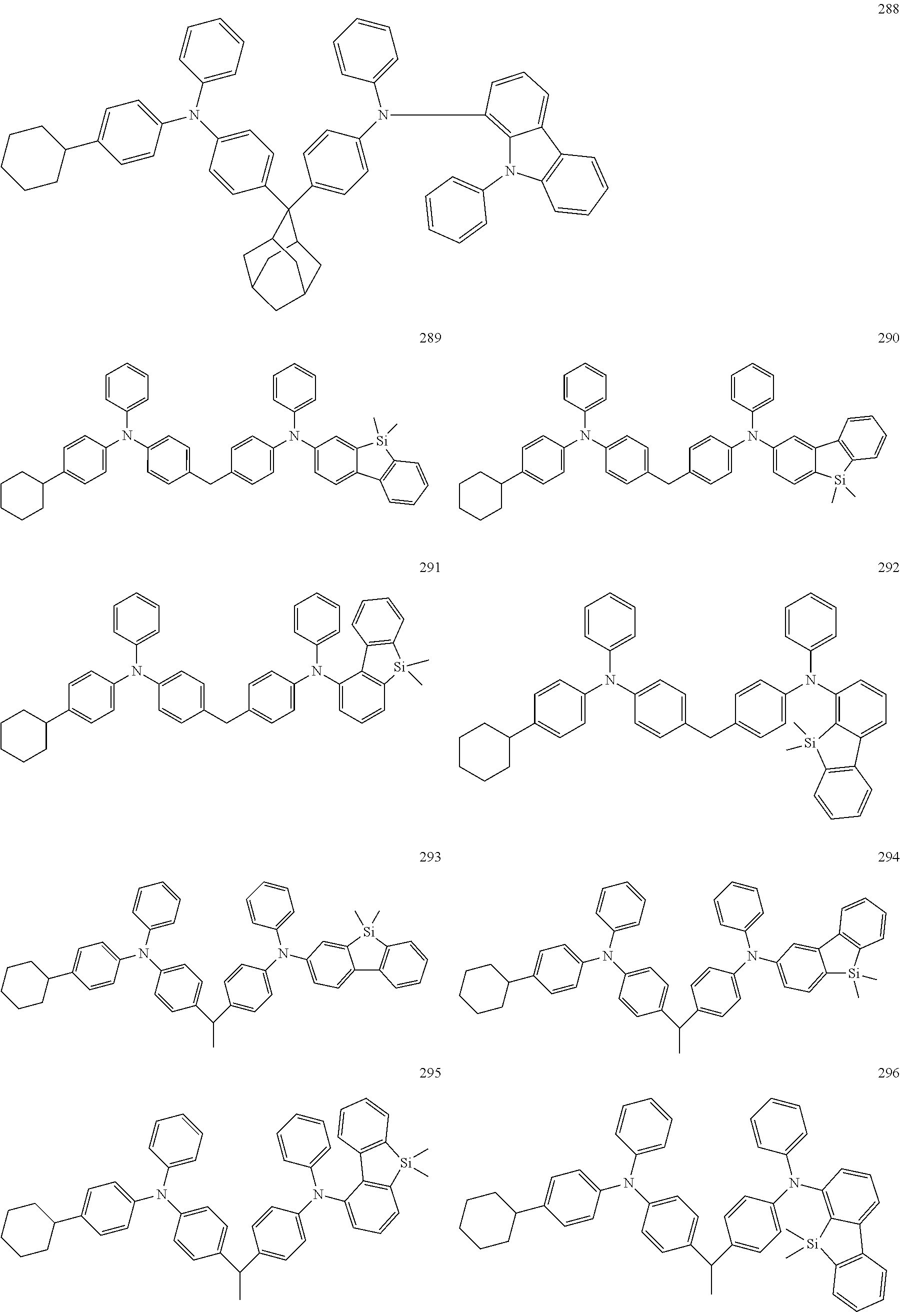

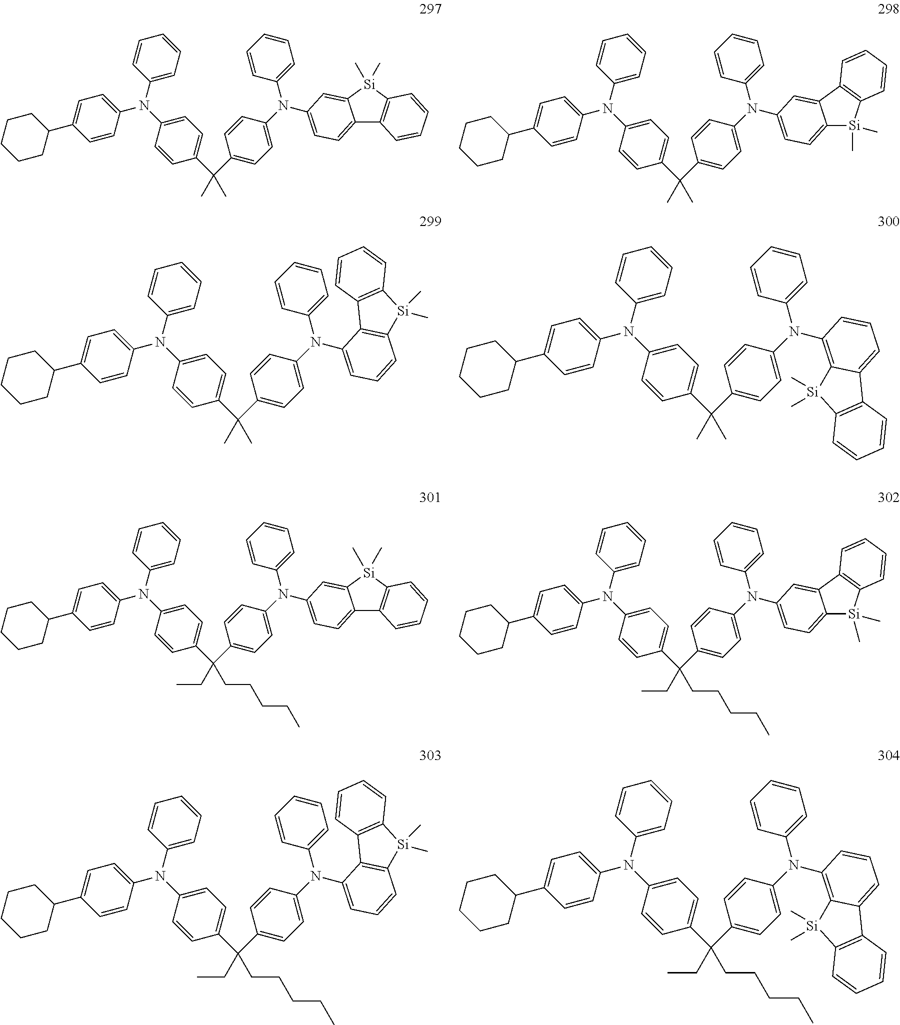

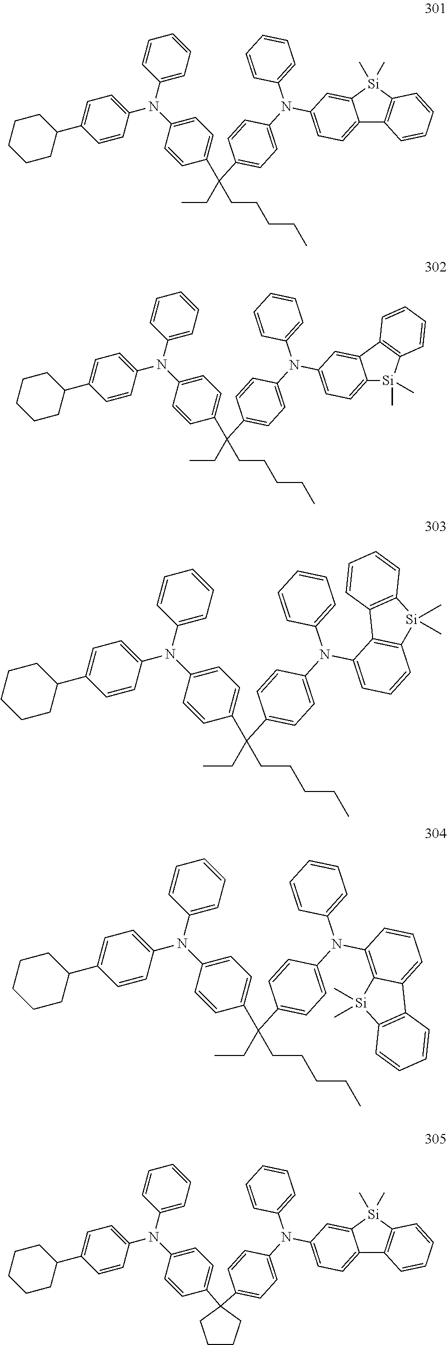

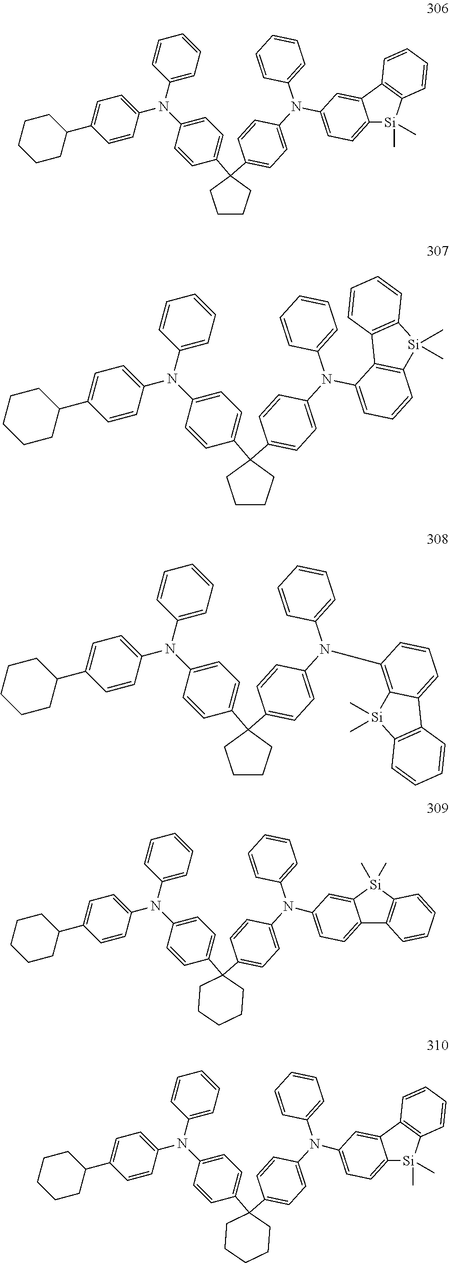

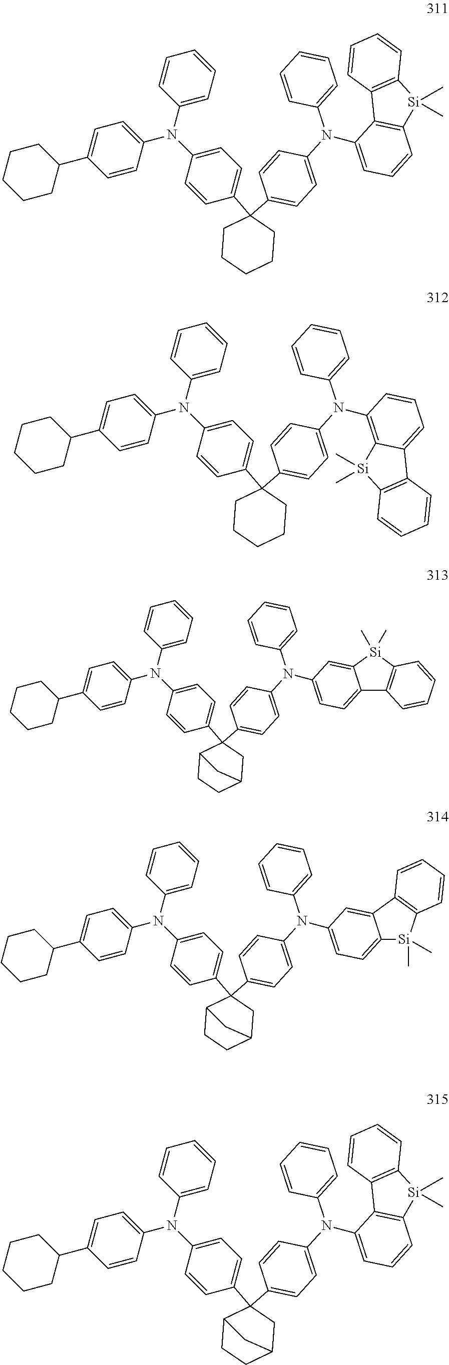

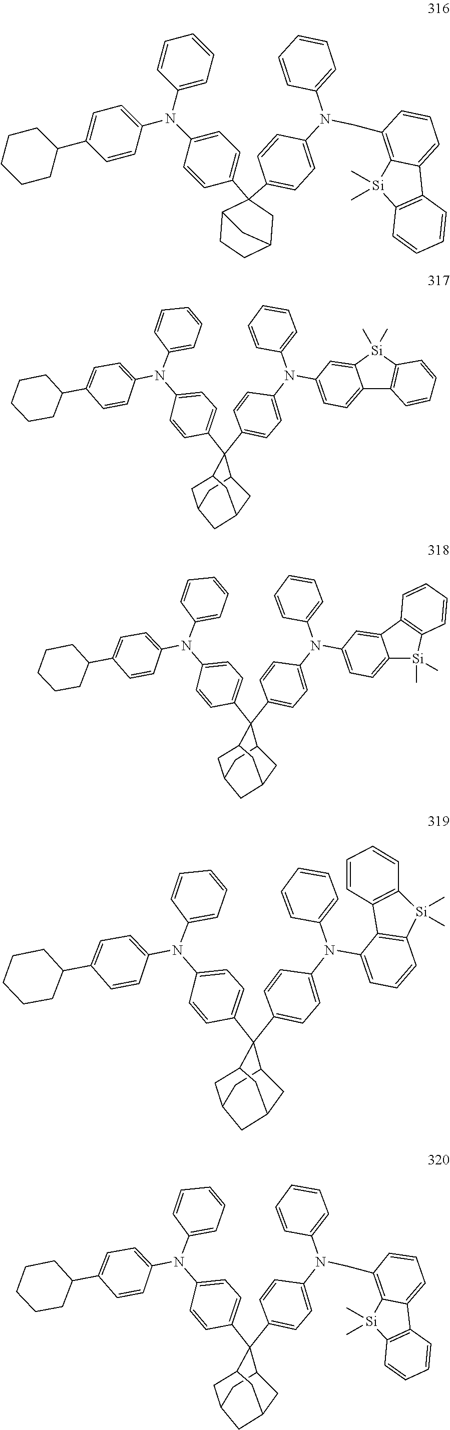

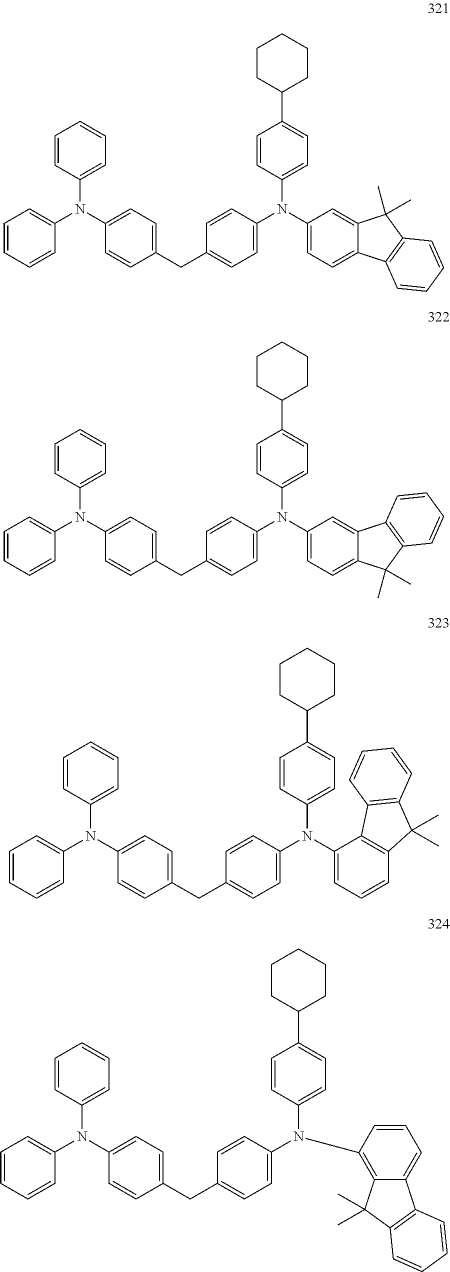 [SMILES: CC1(C)c2ccccc2-c2ccc(N(c3ccc(Cc4ccc(N(c5ccccc5)c5ccccc5)cc4)cc3)c3ccc(C4CCCCC4)cc3)cc21 ; CC1(C)c2ccccc2-c2cc(N(c3ccc(Cc4ccc(N(c5ccccc5)c5ccccc5)cc4)cc3)c3ccc(C4CCCCC4)cc3)ccc21 ; CC1(C)c2ccccc2-c2c(N(c3ccc(Cc4ccc(N(c5ccccc5)c5ccccc5)cc4)cc3)c3ccc(C4CCCCC4)cc3)cccc21 ; CC1(C)c2ccccc2-c2cccc(CN(c3ccc(Cc4ccc(N(c5ccccc5)c5ccccc5)cc4)cc3)c3ccc(C4CCCCC4)cc3)c21]

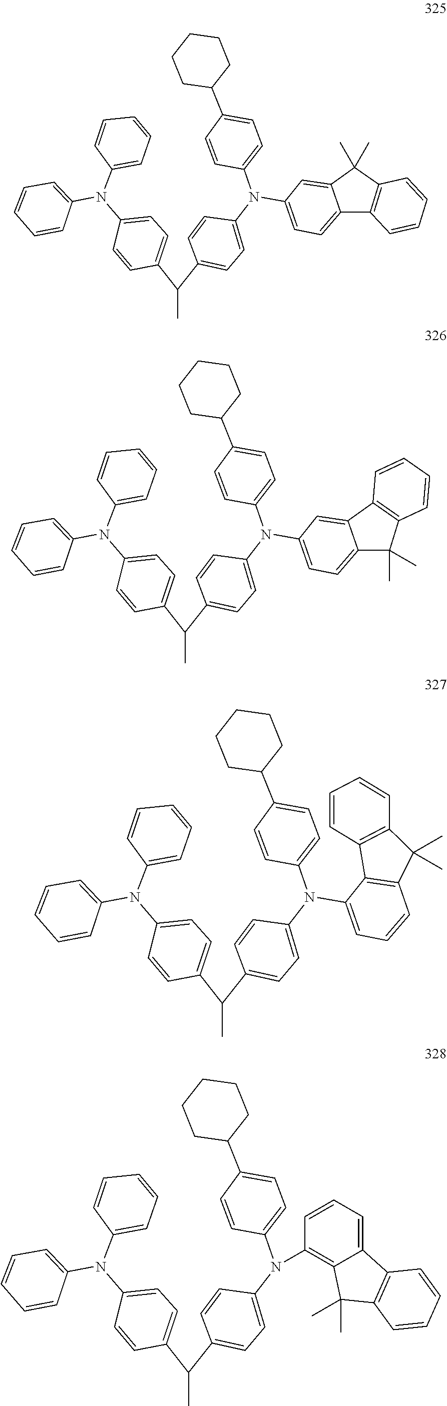 [SMILES: CC(c1ccc(N(c2ccccc2)c2ccccc2)cc1)c1ccc(N(c2ccc(C3CCCCC3)cc2)c2ccc3c(c2)C(C)(C)c2ccccc2-3)cc1 ; CC(c1ccc(N(c2ccccc2)c2ccccc2)cc1)c1ccc(N(c2ccc(C3CCCCC3)cc2)c2ccc3c(c2)-c2ccccc2C3(C)C)cc1 ; CC(c1ccc(N(c2ccccc2)c2ccccc2)cc1)c1ccc(N(c2ccc(C3CCCCC3)cc2)c2cccc3c2-c2ccccc2C3(C)C)cc1 ; CC(c1ccc(N(c2ccccc2)c2ccccc2)cc1)c1ccc(N(c2ccc(C3CCCCC3)cc2)c2cccc3c2C(C)(C)c2ccccc2-3)cc1]

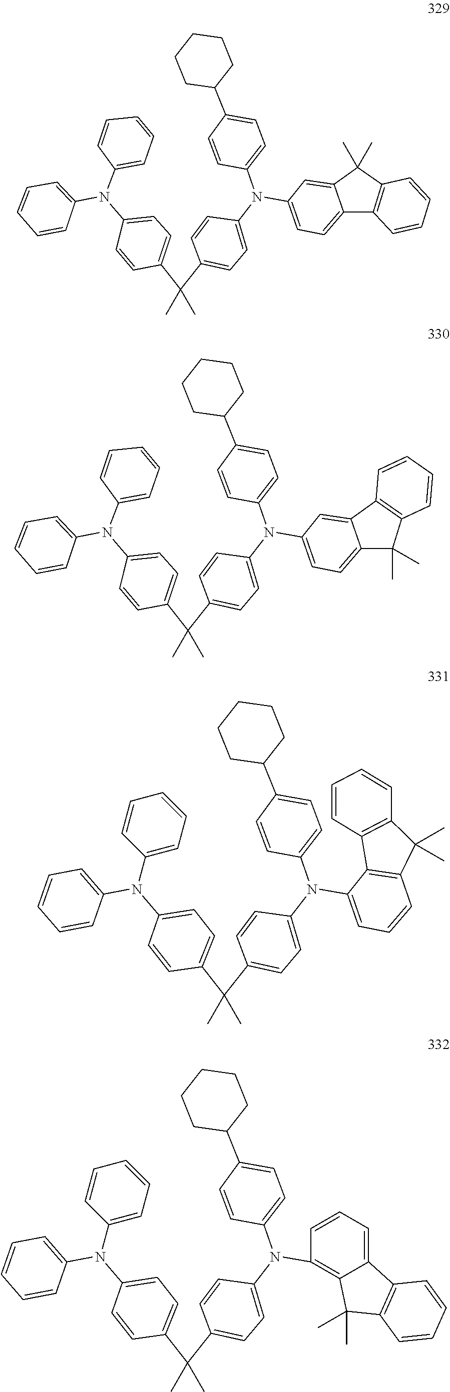 [SMILES: CC(C)(c1ccc(N(c2ccccc2)c2ccccc2)cc1)c1ccc(N(c2ccc(C3CCCCC3)cc2)c2ccc3c(c2)C(C)(C)c2ccccc2-3)cc1 ; CC(C)(c1ccc(N(c2ccccc2)c2ccccc2)cc1)c1ccc(N(c2ccc(C3CCCCC3)cc2)c2ccc3c(c2)-c2ccccc2C3(C)C)cc1 ; CC(C)(c1ccc(N(c2ccccc2)c2ccccc2)cc1)c1ccc(N(c2ccc(C3CCCCC3)cc2)c2cccc3c2-c2ccccc2C3(C)C)cc1 ; CC(C)(c1ccc(N(c2ccccc2)c2ccccc2)cc1)c1ccc(N(c2ccc(C3CCCCC3)cc2)c2cccc3c2C(C)(C)c2ccccc2-3)cc1]

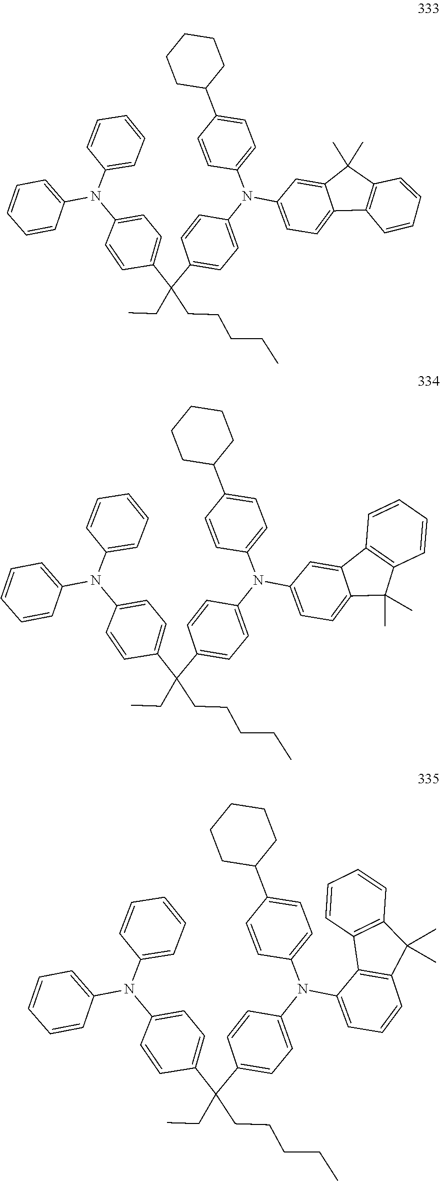 [SMILES: CCCCCC(CC)(c1ccc(N(c2ccccc2)c2ccccc2)cc1)c1ccc(N(c2ccc(C3CCCCC3)cc2)c2ccc3c(c2)C(C)(C)c2ccccc2-3)cc1 ; CCCCCC(CC)(c1ccc(N(c2ccccc2)c2ccccc2)cc1)c1ccc(N(c2ccc(C3CCCCC3)cc2)c2ccc3c(c2)-c2ccccc2C3(C)C)cc1 ; CCCCCC(CC)(c1ccc(N(c2ccccc2)c2ccccc2)cc1)c1ccc(N(c2ccc(C3CCCCC3)cc2)c2cccc3c2-c2ccccc2C3(C)C)cc1]

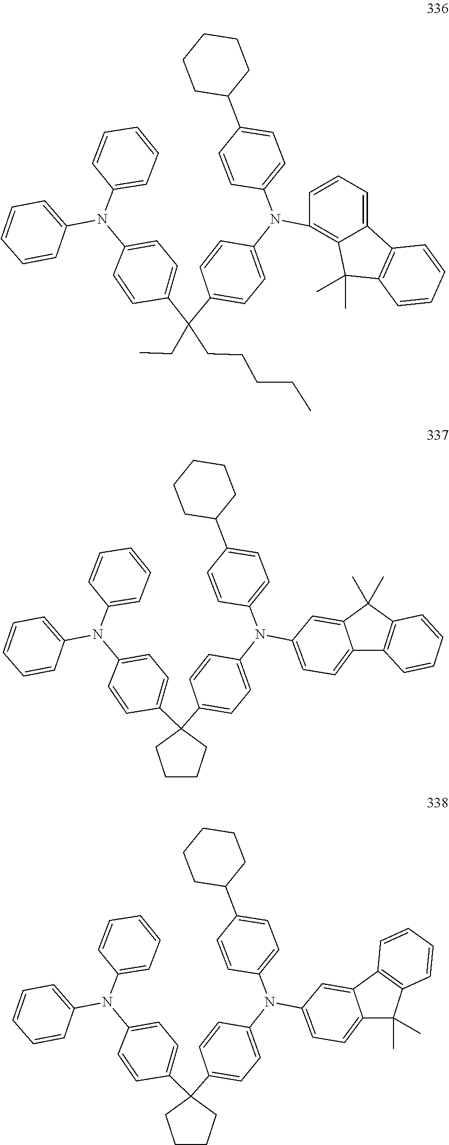 [SMILES: CCCCCC(CC)(c1ccc(N(c2ccccc2)c2ccccc2)cc1)c1ccc(N(c2ccc(C3CCCCC3)cc2)c2cccc3c2C(C)(C)c2ccccc2-3)cc1 ; CC1(C)c2ccccc2-c2ccc(N(c3ccc(C4CCCCC4)cc3)c3ccc(C4(c5ccc(N(c6ccccc6)c6ccccc6)cc5)CCCC4)cc3)cc21 ; CC1(C)c2ccccc2-c2cc(N(c3ccc(C4CCCCC4)cc3)c3ccc(C4(c5ccc(N(c6ccccc6)c6ccccc6)cc5)CCCC4)cc3)ccc21]

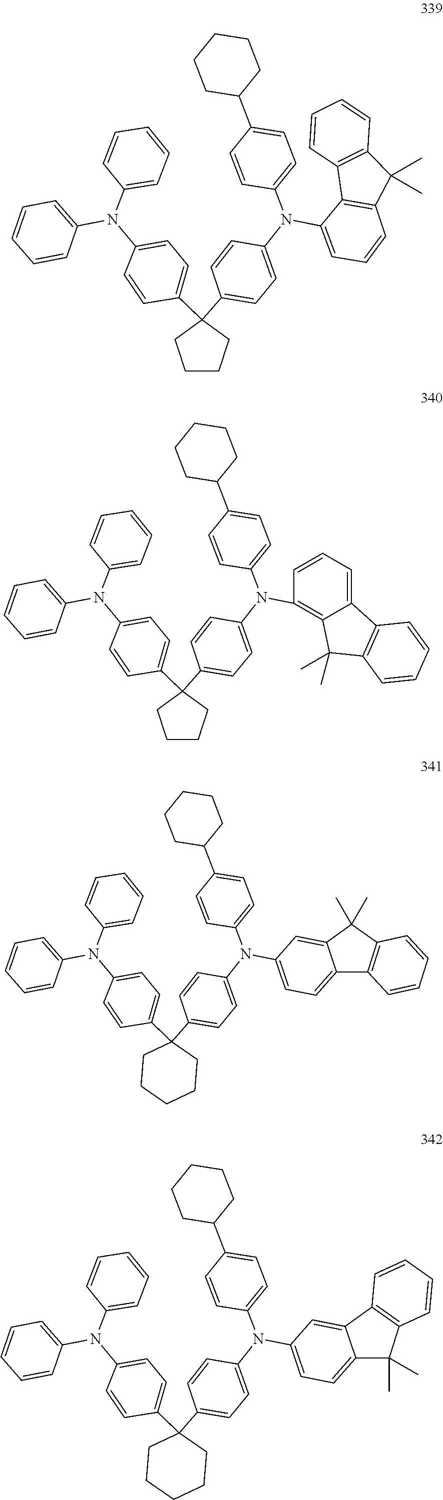 [SMILES: CC1(C)c2ccccc2-c2c(N(c3ccc(C4CCCCC4)cc3)c3ccc(C4(c5ccc(N(c6ccccc6)c6ccccc6)cc5)CCCC4)cc3)cccc21 ; CC1(C)c2ccccc2-c2cccc(N(c3ccc(C4CCCCC4)cc3)c3ccc(C4(c5ccc(N(c6ccccc6)c6ccccc6)cc5)CCCC4)cc3)c21 ; CC1(C)c2ccccc2-c2ccc(N(c3ccc(C4CCCCC4)cc3)c3ccc(C4(c5ccc(N(c6ccccc6)c6ccccc6)cc5)CCCCC4)cc3)cc21 ; CC1(C)c2ccccc2-c2cc(N(c3ccc(C4CCCCC4)cc3)c3ccc(C4(c5ccc(N(c6ccccc6)c6ccccc6)cc5)CCCCC4)cc3)ccc21]

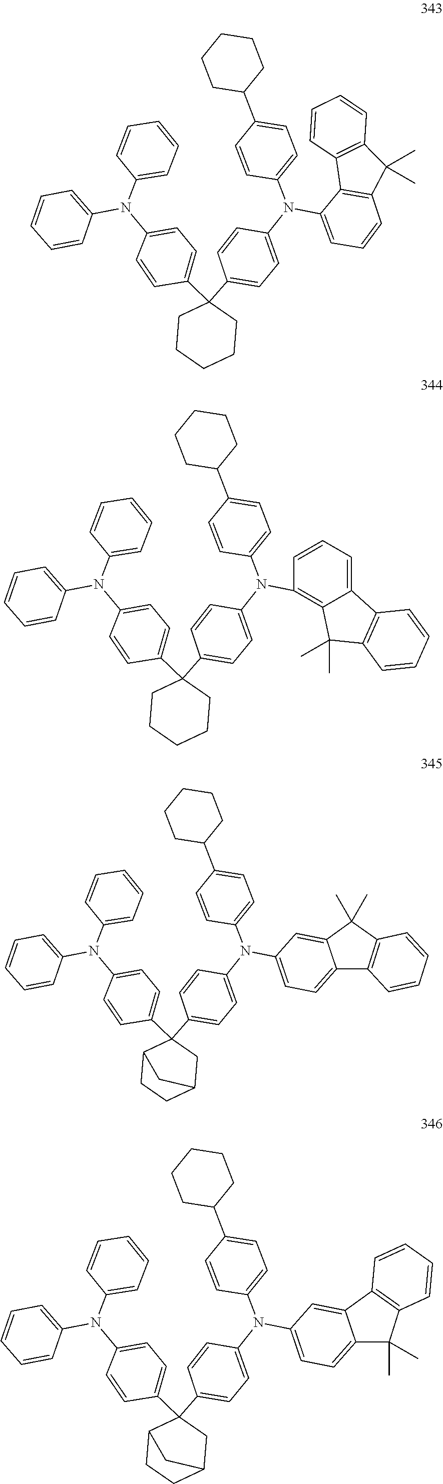

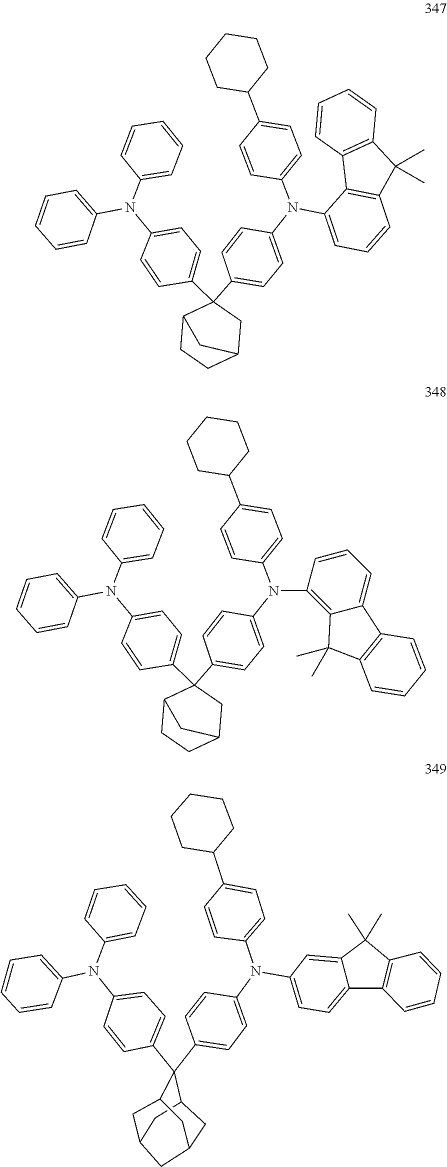

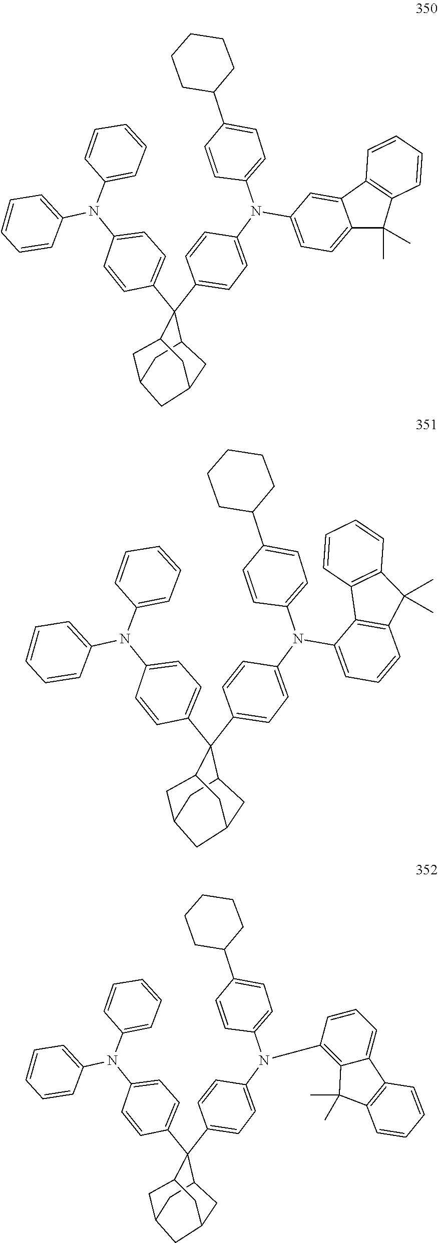 [SMILES: CC1(C)c2ccccc2-c2cc(N(c3ccc(C4CCCCC4)cc3)c3ccc(C4(c5ccc(N(c6ccccc6)c6ccccc6)cc5)C5CC6CC(C5)CC4C6)cc3)ccc21 ; CC1(C)c2ccccc2-c2c(N(c3ccc(C4CCCCC4)cc3)c3ccc(C4(c5ccc(N(c6ccccc6)c6ccccc6)cc5)C5CC6CC(C5)CC4C6)cc3)cccc21 ; CC1(C)c2ccccc2-c2cccc(CN(c3ccc(C4CCCCC4)cc3)c3ccc(C4(c5ccc(N(c6ccccc6)c6ccccc6)cc5)C5CC6CC(C5)CC4C6)cc3)c21]

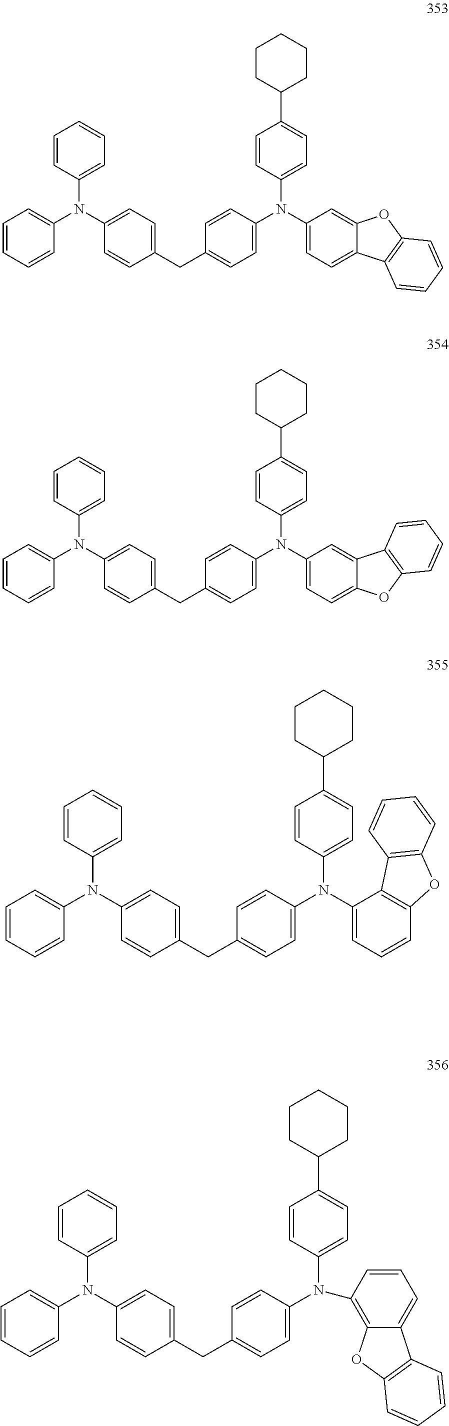 [SMILES: c1ccc(N(c2ccccc2)c2ccc(Cc3ccc(N(c4ccc(C5CCCCC5)cc4)c4ccc5c(c4)oc4ccccc45)cc3)cc2)cc1 ; c1ccc(N(c2ccccc2)c2ccc(Cc3ccc(N(c4ccc(C5CCCCC5)cc4)c4ccc5oc6ccccc6c5c4)cc3)cc2)cc1 ; c1ccc(N(c2ccccc2)c2ccc(Cc3ccc(N(c4ccc(C5CCCCC5)cc4)c4cccc5oc6ccccc6c45)cc3)cc2)cc1 ; c1ccc(N(c2ccccc2)c2ccc(Cc3ccc(N(c4ccc(C5CCCCC5)cc4)c4cccc5c4oc4ccccc45)cc3)cc2)cc1]

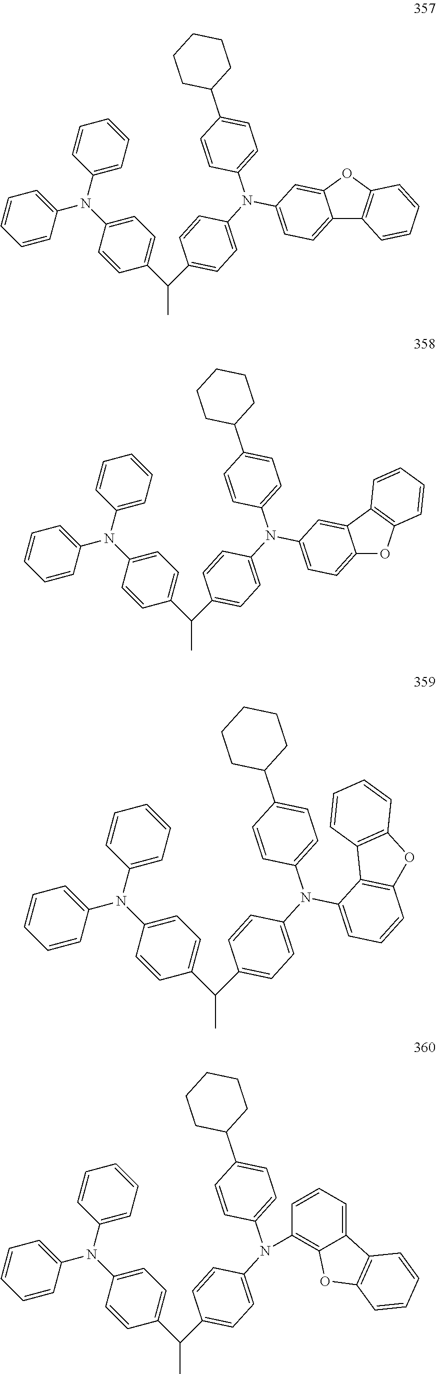 [SMILES: CC(c1ccc(N(c2ccccc2)c2ccccc2)cc1)c1ccc(N(c2ccc(C3CCCCC3)cc2)c2ccc3c(c2)oc2ccccc23)cc1 ; CC(c1ccc(N(c2ccccc2)c2ccccc2)cc1)c1ccc(N(c2ccc(C3CCCCC3)cc2)c2ccc3oc4ccccc4c3c2)cc1 ; CC(c1ccc(N(c2ccccc2)c2ccccc2)cc1)c1ccc(N(c2ccc(C3CCCCC3)cc2)c2cccc3oc4ccccc4c23)cc1 ; CC(c1ccc(N(c2ccccc2)c2ccccc2)cc1)c1ccc(N(c2ccc(C3CCCCC3)cc2)c2cccc3c2oc2ccccc23)cc1]

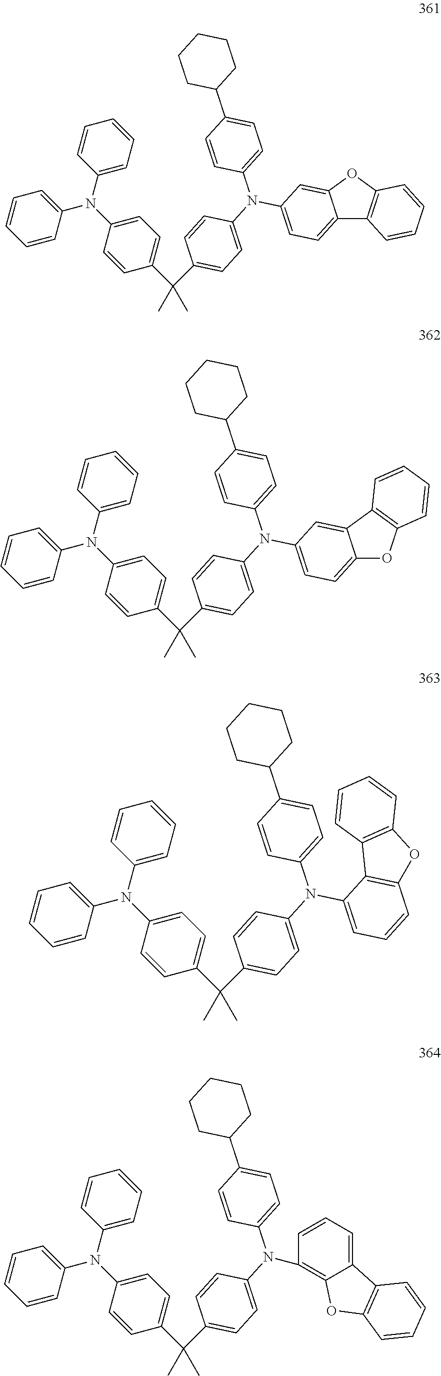 [SMILES: CC(C)(c1ccc(N(c2ccccc2)c2ccccc2)cc1)c1ccc(N(c2ccc(C3CCCCC3)cc2)c2ccc3c(c2)oc2ccccc23)cc1 ; CC(C)(c1ccc(N(c2ccccc2)c2ccccc2)cc1)c1ccc(N(c2ccc(C3CCCCC3)cc2)c2ccc3oc4ccccc4c3c2)cc1 ; CC(C)(c1ccc(N(c2ccccc2)c2ccccc2)cc1)c1ccc(N(c2ccc(C3CCCCC3)cc2)c2cccc3oc4ccccc4c23)cc1 ; CC(C)(c1ccc(N(c2ccccc2)c2ccccc2)cc1)c1ccc(N(c2ccc(C3CCCCC3)cc2)c2cccc3c2oc2ccccc23)cc1]

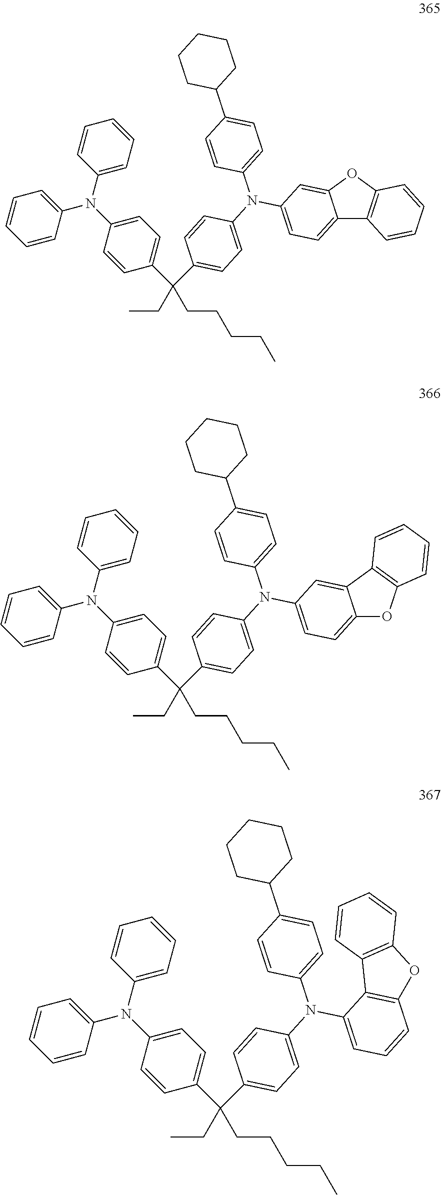 [SMILES: CCCCCC(CC)(c1ccc(N(c2ccccc2)c2ccccc2)cc1)c1ccc(N(c2ccc(C3CCCCC3)cc2)c2ccc3c(c2)oc2ccccc23)cc1 ; CCCCCC(CC)(c1ccc(N(c2ccccc2)c2ccccc2)cc1)c1ccc(N(c2ccc(C3CCCCC3)cc2)c2ccc3oc4ccccc4c3c2)cc1 ; CCCCCC(CC)(c1ccc(N(c2ccccc2)c2ccccc2)cc1)c1ccc(N(c2ccc(C3CCCCC3)cc2)c2cccc3oc4ccccc4c23)cc1]

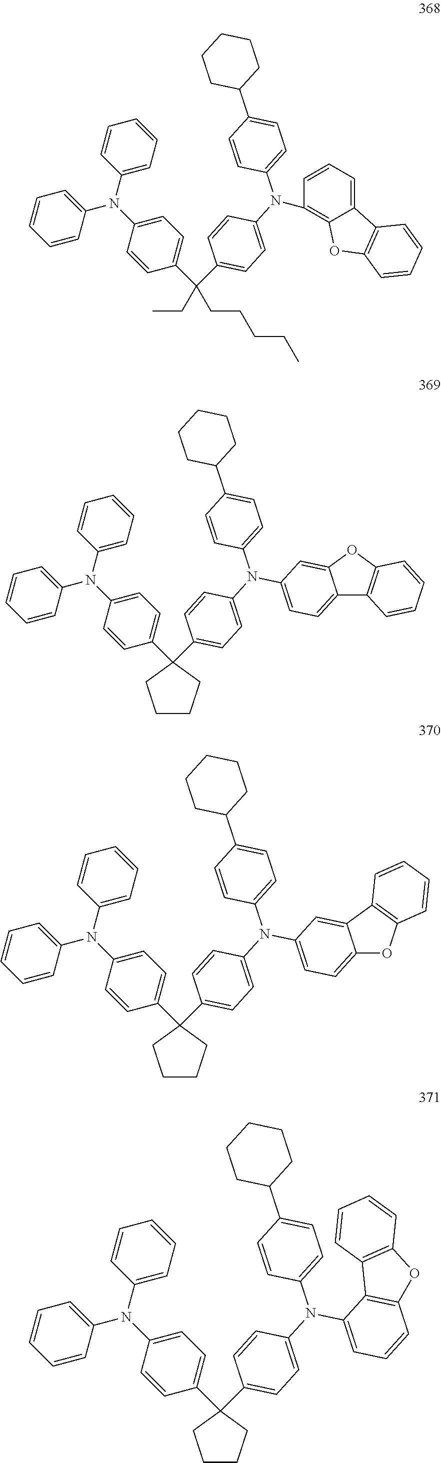 [SMILES: CCCCCC(CC)(c1ccc(N(c2ccccc2)c2ccccc2)cc1)c1ccc(N(c2ccc(C3CCCCC3)cc2)c2cccc3c2oc2ccccc23)cc1 ; c1ccc(N(c2ccccc2)c2ccc(C3(c4ccc(N(c5ccc(C6CCCCC6)cc5)c5ccc6c(c5)oc5ccccc56)cc4)CCCC3)cc2)cc1 ; c1ccc(N(c2ccccc2)c2ccc(C3(c4ccc(N(c5ccc(C6CCCCC6)cc5)c5ccc6oc7ccccc7c6c5)cc4)CCCC3)cc2)cc1 ; c1ccc(N(c2ccccc2)c2ccc(C3(c4ccc(N(c5ccc(C6CCCCC6)cc5)c5cccc6oc7ccccc7c56)cc4)CCCC3)cc2)cc1]

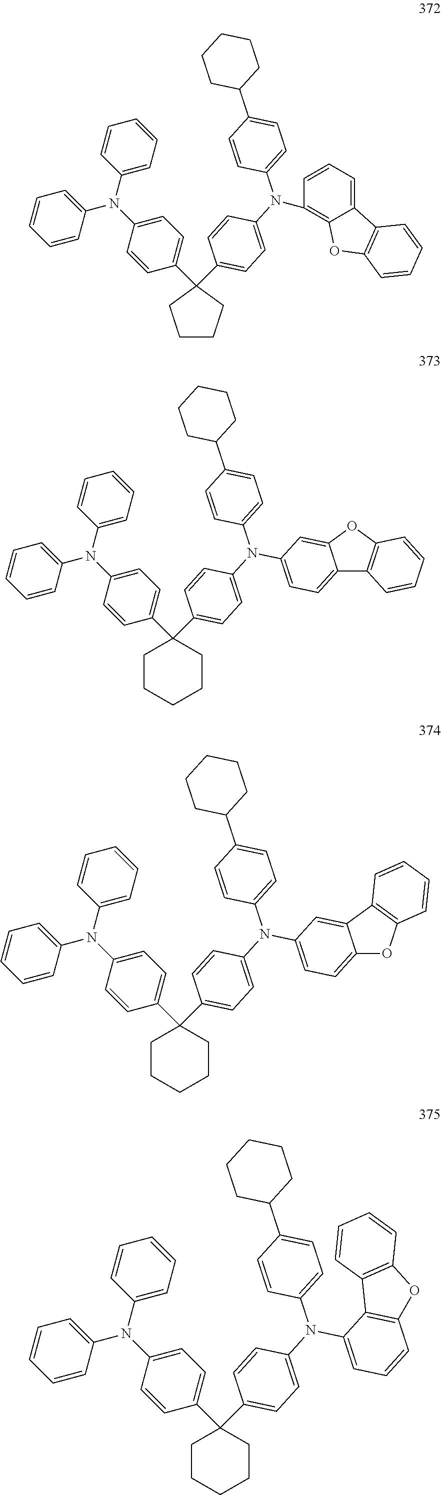 [SMILES: c1ccc(N(c2ccccc2)c2ccc(C3(c4ccc(N(c5ccc(C6CCCCC6)cc5)c5cccc6c5oc5ccccc56)cc4)CCCC3)cc2)cc1 ; c1ccc(N(c2ccccc2)c2ccc(C3(c4ccc(N(c5ccc(C6CCCCC6)cc5)c5ccc6c(c5)oc5ccccc56)cc4)CCCCC3)cc2)cc1 ; c1ccc(N(c2ccccc2)c2ccc(C3(c4ccc(N(c5ccc(C6CCCCC6)cc5)c5ccc6oc7ccccc7c6c5)cc4)CCCCC3)cc2)cc1 ; c1ccc(N(c2ccccc2)c2ccc(C3(c4ccc(N(c5ccc(C6CCCCC6)cc5)c5cccc6oc7ccccc7c56)cc4)CCCCC3)cc2)cc1]

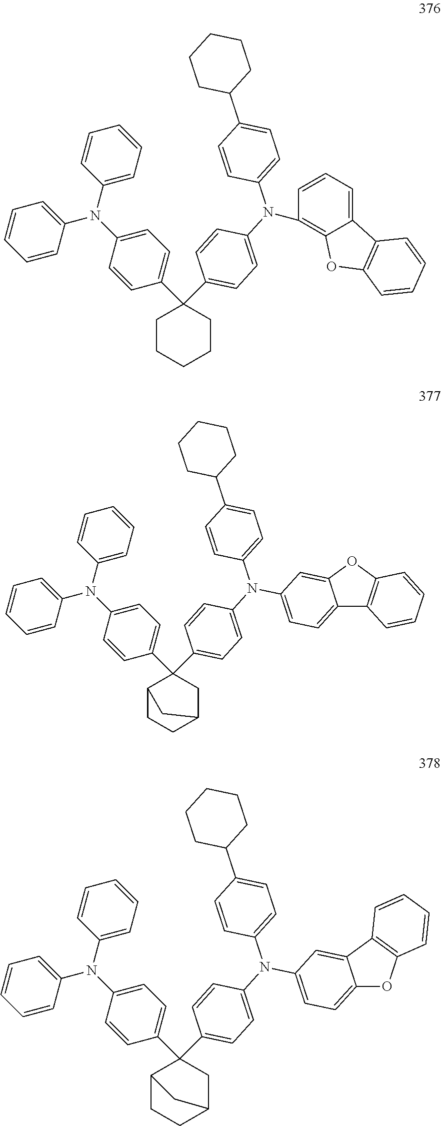 [SMILES: c1ccc(N(c2ccccc2)c2ccc(C3(c4ccc(N(c5ccc(C6CCCCC6)cc5)c5cccc6c5oc5ccccc56)cc4)CCCCC3)cc2)cc1 ; c1ccc(N(c2ccccc2)c2ccc(C3(c4ccc(N(c5ccc(C6CCCCC6)cc5)c5ccc6c(c5)oc5ccccc56)cc4)CC4CCC3C4)cc2)cc1 ; c1ccc(N(c2ccccc2)c2ccc(C3(c4ccc(N(c5ccc(C6CCCCC6)cc5)c5ccc6oc7ccccc7c6c5)cc4)CC4CCC3C4)cc2)cc1]

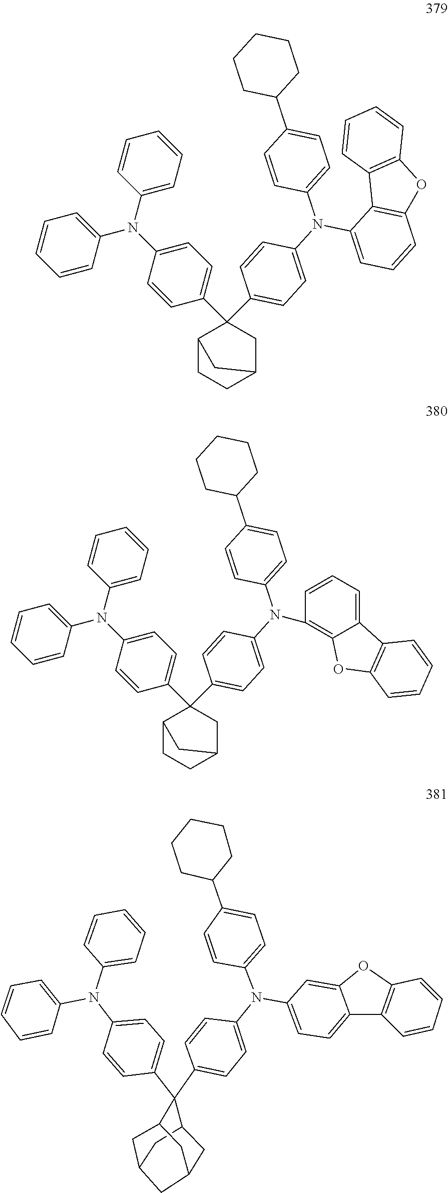 [SMILES: c1ccc(N(c2ccccc2)c2ccc(C3(c4ccc(N(c5ccc(C6CCCCC6)cc5)c5cccc6oc7ccccc7c56)cc4)CC4CCC3C4)cc2)cc1 ; c1ccc(N(c2ccccc2)c2ccc(C3(c4ccc(N(c5ccc(C6CCCCC6)cc5)c5cccc6c5oc5ccccc56)cc4)CC4CCC3C4)cc2)cc1 ; c1ccc(N(c2ccccc2)c2ccc(C3(c4ccc(N(c5ccc(C6CCCCC6)cc5)c5ccc6c(c5)oc5ccccc56)cc4)C4CC5CC(C4)CC3C5)cc2)cc1]

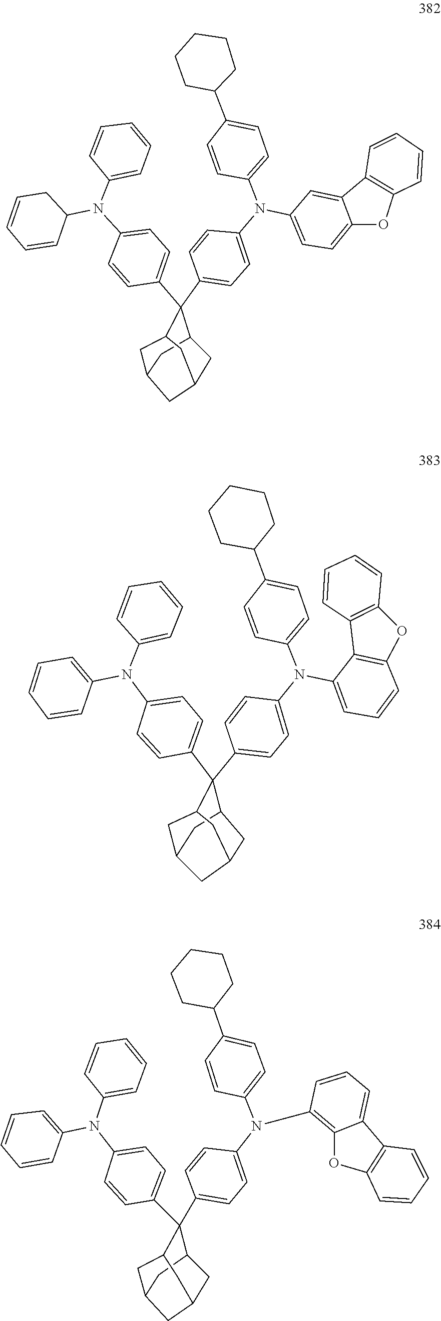 [SMILES: C1=CCC(N(c2ccccc2)c2ccc(C3(c4ccc(N(c5ccc(C6CCCCC6)cc5)c5ccc6oc7ccccc7c6c5)cc4)C4CC5CC(C4)CC3C5)cc2)C=C1 ; c1ccc(N(c2ccccc2)c2ccc(C3(c4ccc(N(c5ccc(C6CCCCC6)cc5)c5cccc6oc7ccccc7c56)cc4)C4CC5CC(C4)CC3C5)cc2)cc1 ; c1ccc(N(c2ccccc2)c2ccc(C3(c4ccc(N(c5ccc(C6CCCCC6)cc5)c5cccc6c5oc5ccccc56)cc4)C4CC5CC(C4)CC3C5)cc2)cc1]

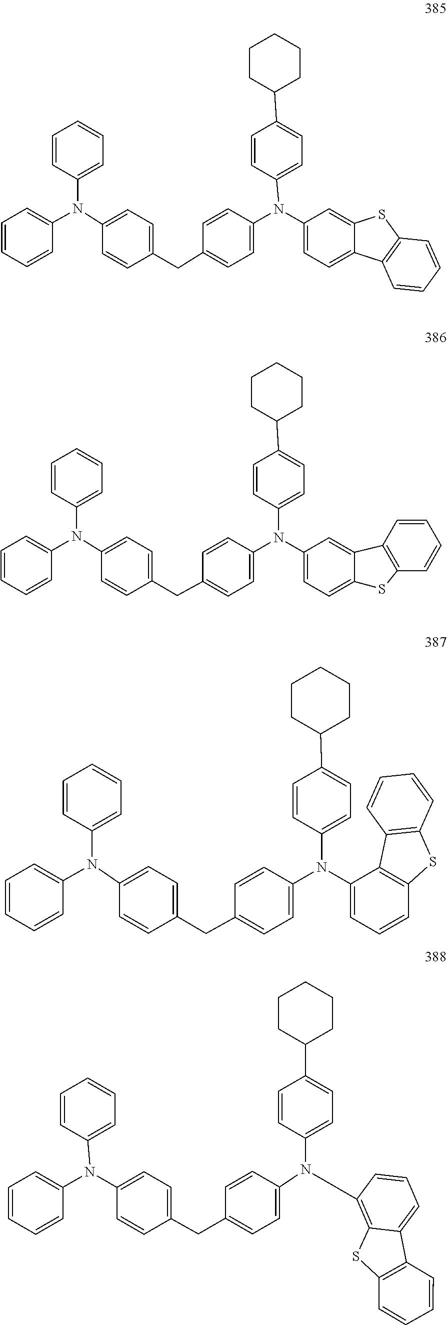 [SMILES: c1ccc(N(c2ccccc2)c2ccc(Cc3ccc(N(c4ccc(C5CCCCC5)cc4)c4ccc5c(c4)sc4ccccc45)cc3)cc2)cc1 ; c1ccc(N(c2ccccc2)c2ccc(Cc3ccc(N(c4ccc(C5CCCCC5)cc4)c4ccc5sc6ccccc6c5c4)cc3)cc2)cc1 ; c1ccc(N(c2ccccc2)c2ccc(Cc3ccc(N(c4ccc(C5CCCCC5)cc4)c4cccc5sc6ccccc6c45)cc3)cc2)cc1 ; c1ccc(N(c2ccccc2)c2ccc(Cc3ccc(N(c4ccc(C5CCCCC5)cc4)c4cccc5c4sc4ccccc45)cc3)cc2)cc1]

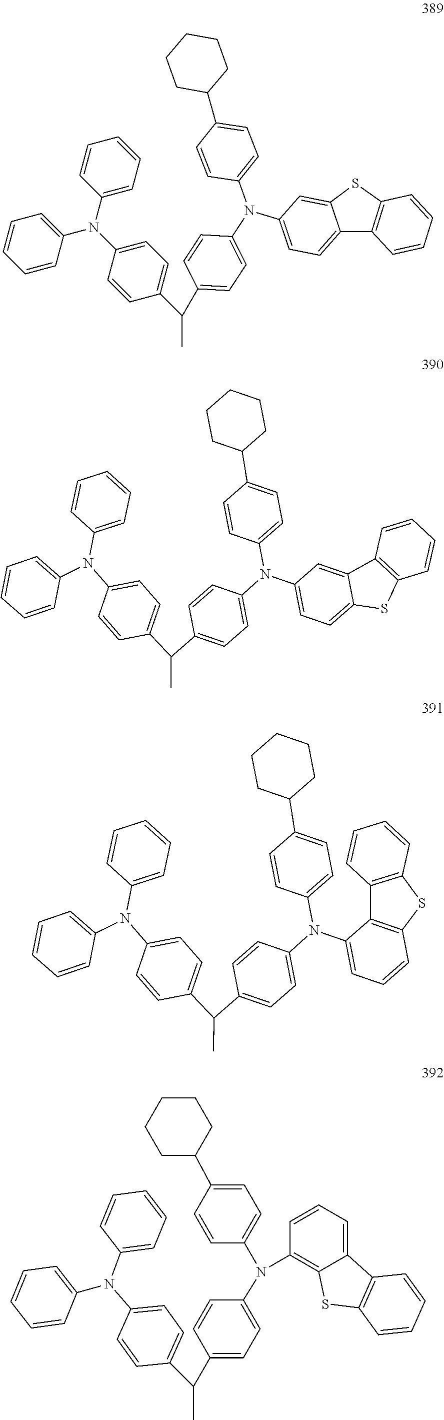 [SMILES: CC(c1ccc(N(c2ccccc2)c2ccccc2)cc1)c1ccc(N(c2ccc(C3CCCCC3)cc2)c2ccc3c(c2)sc2ccccc23)cc1 ; CC(c1ccc(N(c2ccccc2)c2ccccc2)cc1)c1ccc(N(c2ccc(C3CCCCC3)cc2)c2ccc3sc4ccccc4c3c2)cc1 ; CC(c1ccc(N(c2ccccc2)c2ccccc2)cc1)c1ccc(N(c2ccc(C3CCCCC3)cc2)c2cccc3sc4ccccc4c23)cc1 ; CC(c1ccc(N(c2ccccc2)c2ccccc2)cc1)c1ccc(N(c2ccc(C3CCCCC3)cc2)c2cccc3c2sc2ccccc23)cc1]

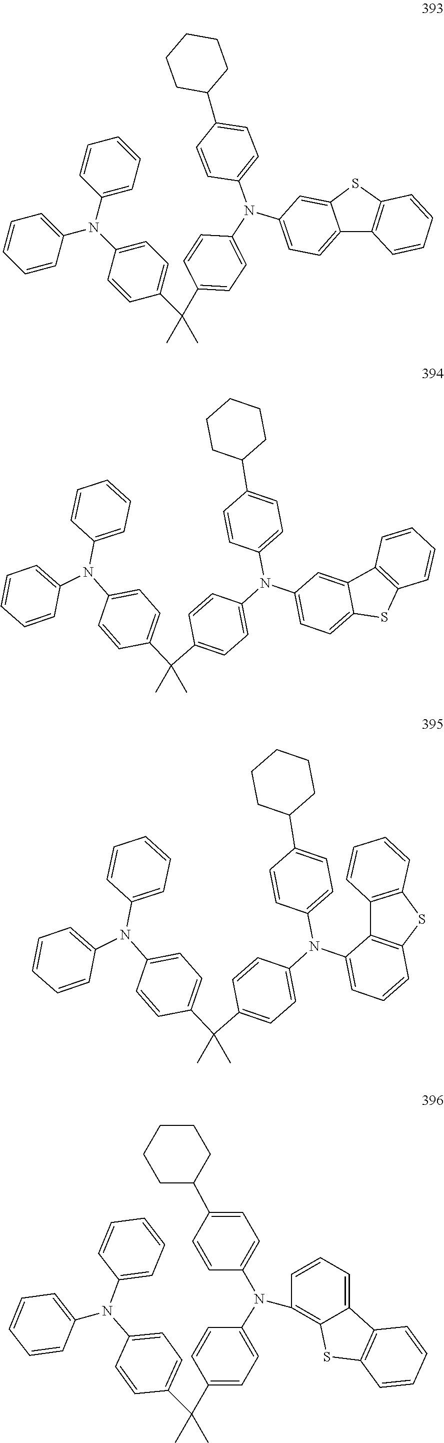 [SMILES: CC(C)(c1ccc(N(c2ccccc2)c2ccccc2)cc1)c1ccc(N(c2ccc(C3CCCCC3)cc2)c2ccc3c(c2)sc2ccccc23)cc1 ; CC(C)(c1ccc(N(c2ccccc2)c2ccccc2)cc1)c1ccc(N(c2ccc(C3CCCCC3)cc2)c2ccc3sc4ccccc4c3c2)cc1 ; CC(C)(c1ccc(N(c2ccccc2)c2ccccc2)cc1)c1ccc(N(c2ccc(C3CCCCC3)cc2)c2cccc3sc4ccccc4c23)cc1 ; CC(C)(c1ccc(N(c2ccccc2)c2ccccc2)cc1)c1ccc(N(c2ccc(C3CCCCC3)cc2)c2cccc3c2sc2ccccc23)cc1]

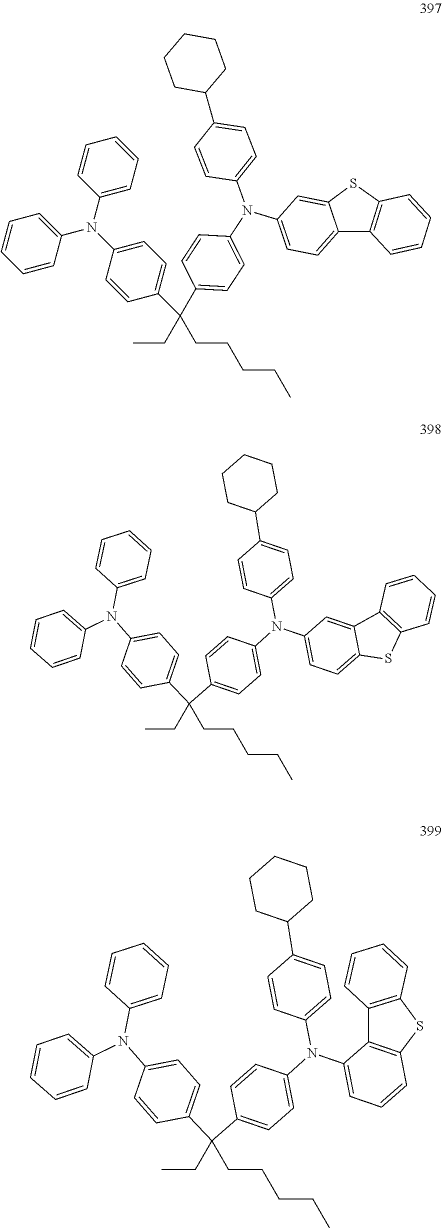 [SMILES: CCCCCC(CC)(c1ccc(N(c2ccccc2)c2ccccc2)cc1)c1ccc(N(c2ccc(C3CCCCC3)cc2)c2ccc3c(c2)sc2ccccc23)cc1 ; CCCCCC(CC)(c1ccc(N(c2ccccc2)c2ccccc2)cc1)c1ccc(N(c2ccc(C3CCCCC3)cc2)c2ccc3sc4ccccc4c3c2)cc1 ; CCCCCC(CC)(c1ccc(N(c2ccccc2)c2ccccc2)cc1)c1ccc(N(c2ccc(C3CCCCC3)cc2)c2cccc3sc4ccccc4c23)cc1]

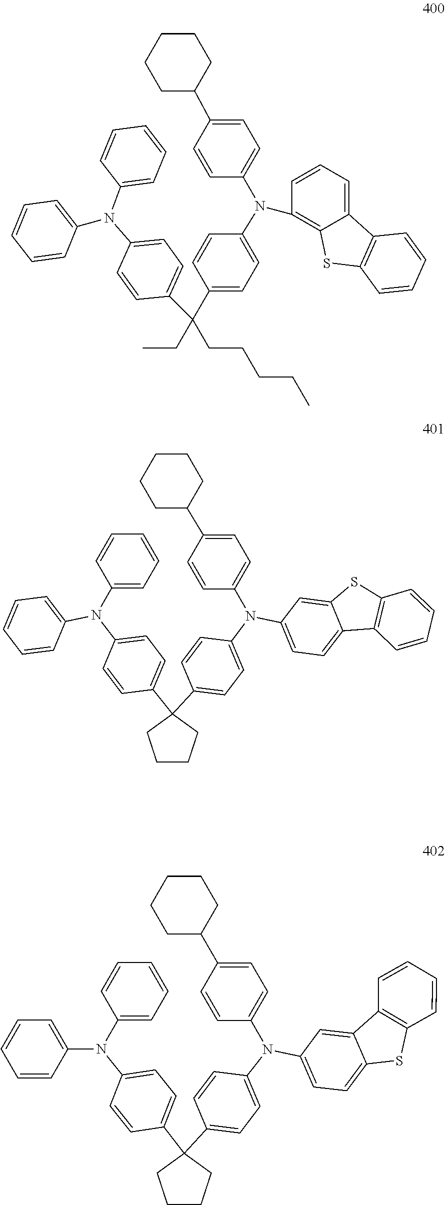 [SMILES: CCCCCC(CC)(c1ccc(N(c2ccccc2)c2ccccc2)cc1)c1ccc(N(c2ccc(C3CCCCC3)cc2)c2cccc3c2sc2ccccc23)cc1 ; c1ccc(N(c2ccccc2)c2ccc(C3(c4ccc(N(c5ccc(C6CCCCC6)cc5)c5ccc6c(c5)sc5ccccc56)cc4)CCCC3)cc2)cc1 ; c1ccc(N(c2ccccc2)c2ccc(C3(c4ccc(N(c5ccc(C6CCCCC6)cc5)c5ccc6sc7ccccc7c6c5)cc4)CCCC3)cc2)cc1]

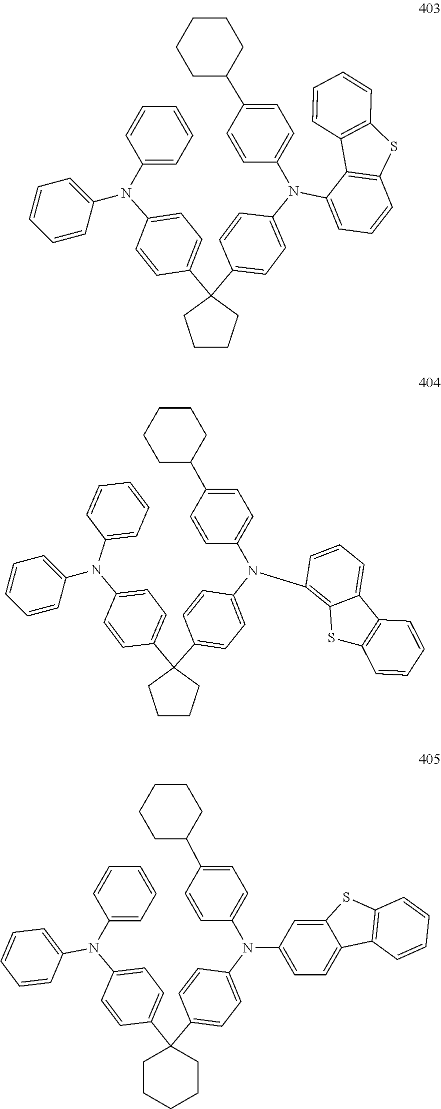 [SMILES: c1ccc(N(c2ccccc2)c2ccc(C3(c4ccc(N(c5ccc(C6CCCCC6)cc5)c5cccc6sc7ccccc7c56)cc4)CCCC3)cc2)cc1 ; c1ccc(N(c2ccccc2)c2ccc(C3(c4ccc(N(c5ccc(C6CCCCC6)cc5)c5cccc6c5sc5ccccc56)cc4)CCCC3)cc2)cc1 ; c1ccc(N(c2ccccc2)c2ccc(C3(c4ccc(N(c5ccc(C6CCCCC6)cc5)c5ccc6c(c5)sc5ccccc56)cc4)CCCCC3)cc2)cc1]

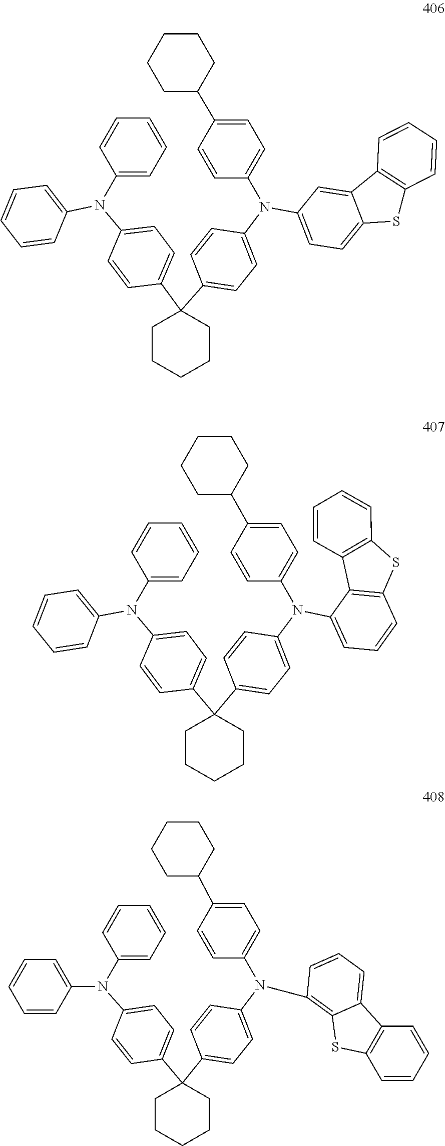

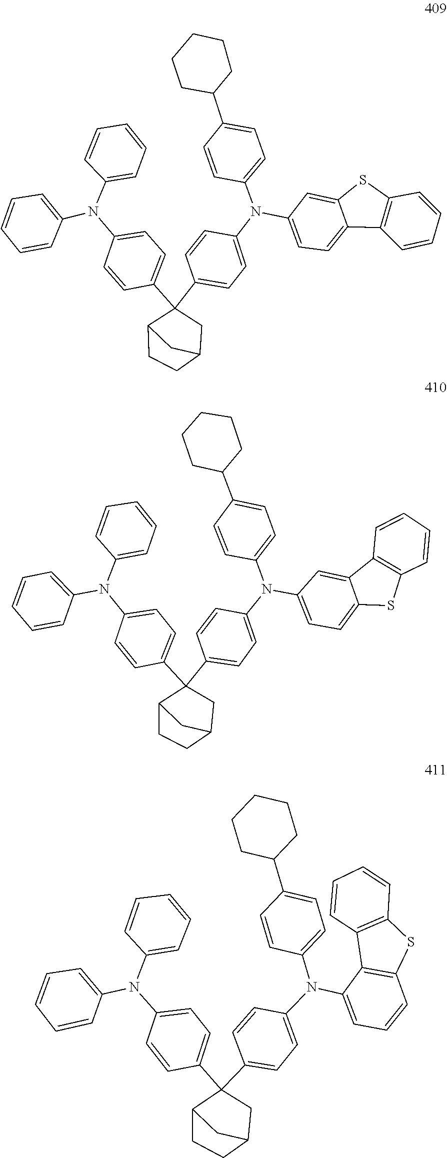 [SMILES: c1ccc(N(c2ccccc2)c2ccc(C3(c4ccc(N(c5ccc(C6CCCCC6)cc5)c5ccc6c(c5)sc5ccccc56)cc4)CC4CCC3C4)cc2)cc1 ; c1ccc(N(c2ccccc2)c2ccc(C3(c4ccc(N(c5ccc(C6CCCCC6)cc5)c5ccc6sc7ccccc7c6c5)cc4)CC4CCC3C4)cc2)cc1 ; c1ccc(N(c2ccccc2)c2ccc(C3(c4ccc(N(c5ccc(C6CCCCC6)cc5)c5cccc6sc7ccccc7c56)cc4)CC4CCC3C4)cc2)cc1]

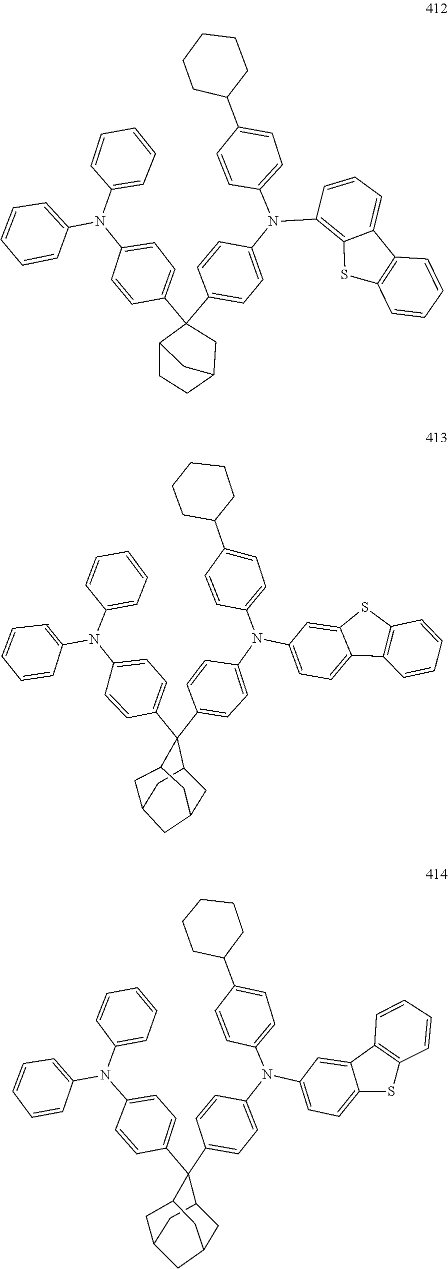 [SMILES: c1ccc(N(c2ccccc2)c2ccc(C3(c4ccc(N(c5ccc(C6CCCCC6)cc5)c5cccc6c5sc5ccccc56)cc4)CC4CCC3C4)cc2)cc1 ; Cc1ccccc1N(c1ccccc1)c1ccc(C2(c3ccc(N(c4ccc(C5CCCCC5)cc4)c4ccc5c(c4)sc4ccccc45)cc3)C3CC4CC(C3)CC2C4)cc1 ; c1ccc(N(c2ccccc2)c2ccc(C3(c4ccc(N(c5ccc(C6CCCCC6)cc5)c5ccc6sc7ccccc7c6c5)cc4)C4CC5CC(C4)CC3C5)cc2)cc1]

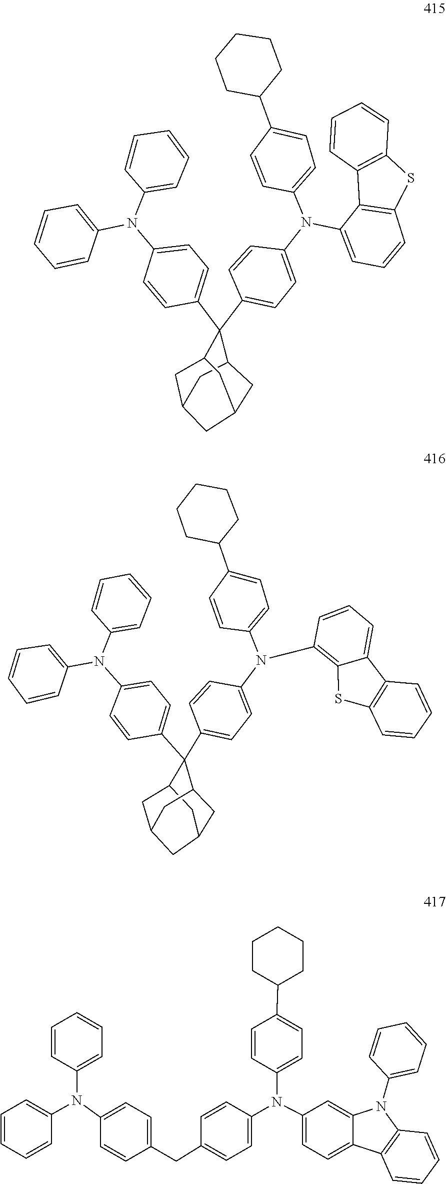 [SMILES: c1ccc(N(c2ccccc2)c2ccc(C3(c4ccc(N(c5ccc(C6CCCCC6)cc5)c5cccc6sc7ccccc7c56)cc4)C4CC5CC(C4)CC3C5)cc2)cc1 ; c1ccc(N(c2ccccc2)c2ccc(C3(c4ccc(N(c5ccc(C6CCCCC6)cc5)c5cccc6c5sc5ccccc56)cc4)C4CC5CC(C4)CC3C5)cc2)cc1 ; c1ccc(N(c2ccccc2)c2ccc(Cc3ccc(N(c4ccc(C5CCCCC5)cc4)c4ccc5c6ccccc6n(-c6ccccc6)c5c4)cc3)cc2)cc1]

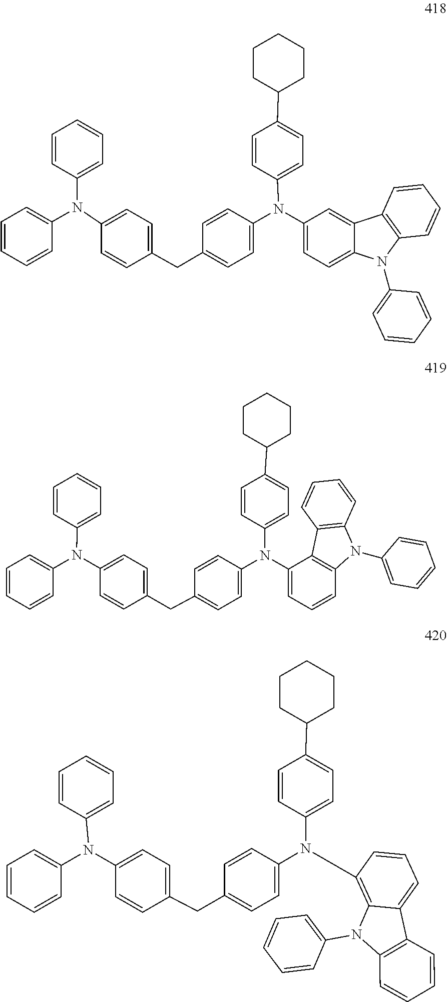 [SMILES: c1ccc(N(c2ccccc2)c2ccc(Cc3ccc(N(c4ccc(C5CCCCC5)cc4)c4ccc5c(c4)c4ccccc4n5-c4ccccc4)cc3)cc2)cc1 ; c1ccc(N(c2ccccc2)c2ccc(Cc3ccc(N(c4ccc(C5CCCCC5)cc4)c4cccc5c4c4ccccc4n5-c4ccccc4)cc3)cc2)cc1 ; c1ccc(N(c2ccccc2)c2ccc(Cc3ccc(N(c4ccc(C5CCCCC5)cc4)c4cccc5c6ccccc6n(-c6ccccc6)c45)cc3)cc2)cc1]

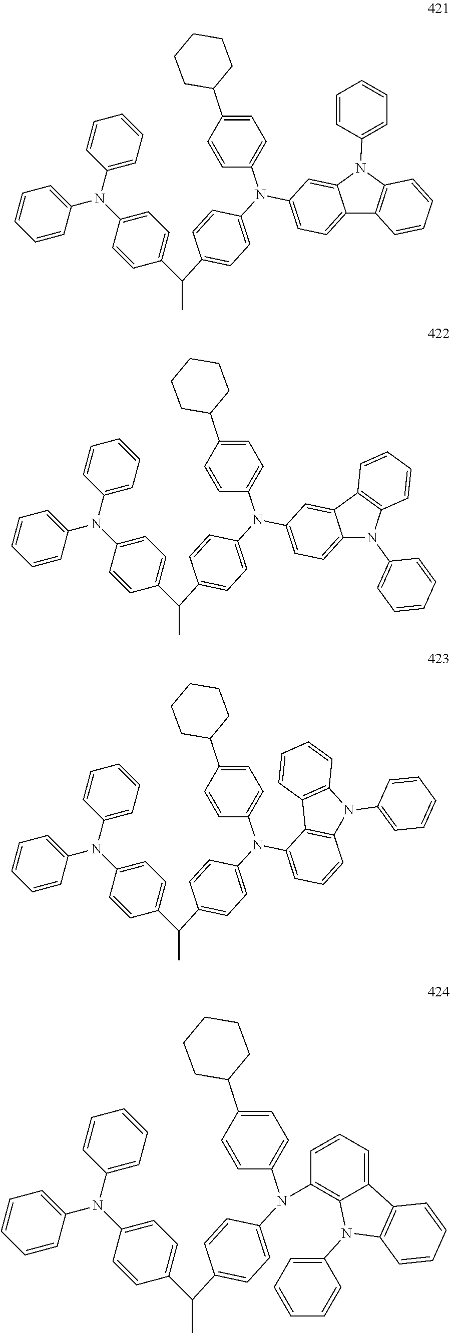 [SMILES: CC(c1ccc(N(c2ccccc2)c2ccccc2)cc1)c1ccc(N(c2ccc(C3CCCCC3)cc2)c2ccc3c4ccccc4n(-c4ccccc4)c3c2)cc1 ; CC(c1ccc(N(c2ccccc2)c2ccccc2)cc1)c1ccc(N(c2ccc(C3CCCCC3)cc2)c2ccc3c(c2)c2ccccc2n3-c2ccccc2)cc1 ; CC(c1ccc(N(c2ccccc2)c2ccccc2)cc1)c1ccc(N(c2ccc(C3CCCCC3)cc2)c2cccc3c2c2ccccc2n3-c2ccccc2)cc1 ; CC(c1ccc(N(c2ccccc2)c2ccccc2)cc1)c1ccc(N(c2ccc(C3CCCCC3)cc2)c2cccc3c4ccccc4n(-c4ccccc4)c23)cc1]

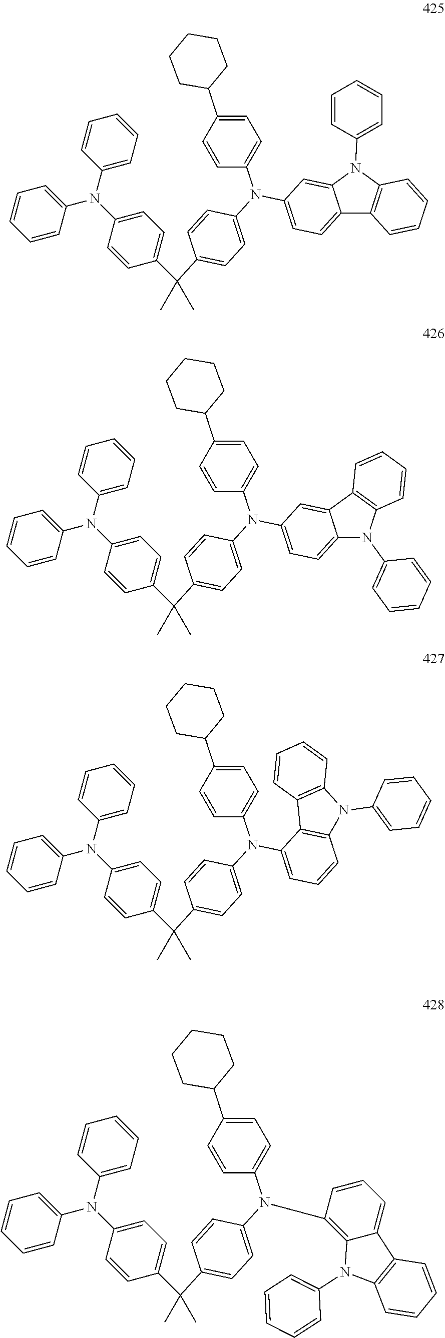 [SMILES: CC(C)(c1ccc(N(c2ccccc2)c2ccccc2)cc1)c1ccc(N(c2ccc(C3CCCCC3)cc2)c2ccc3c4ccccc4n(-c4ccccc4)c3c2)cc1 ; CC(C)(c1ccc(N(c2ccccc2)c2ccccc2)cc1)c1ccc(N(c2ccc(C3CCCCC3)cc2)c2ccc3c(c2)c2ccccc2n3-c2ccccc2)cc1 ; CC(C)(c1ccc(N(c2ccccc2)c2ccccc2)cc1)c1ccc(N(c2ccc(C3CCCCC3)cc2)c2cccc3c2c2ccccc2n3-c2ccccc2)cc1 ; CC(C)(c1ccc(N(Cc2cccc3c4ccccc4n(-c4ccccc4)c23)c2ccc(C3CCCCC3)cc2)cc1)c1ccc(N(c2ccccc2)c2ccccc2)cc1]

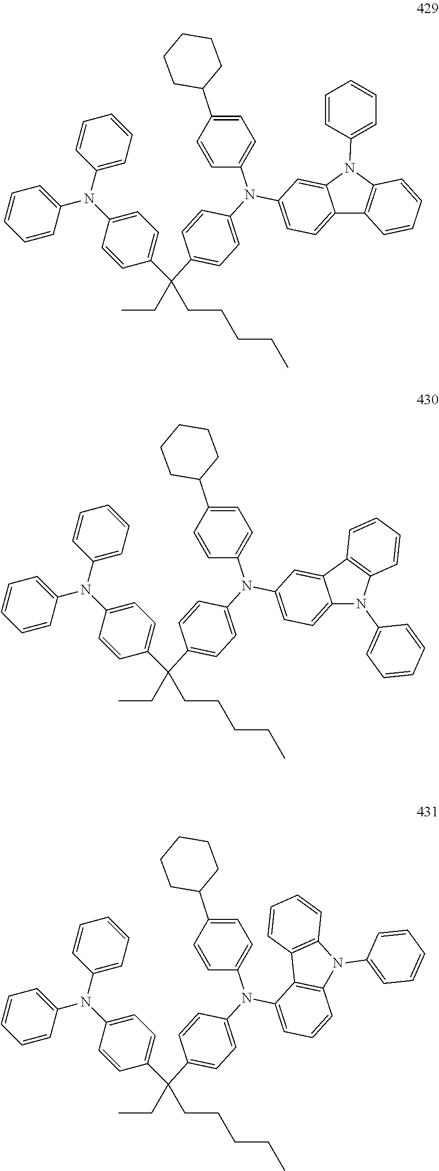 [SMILES: CCCCCC(CC)(c1ccc(N(c2ccccc2)c2ccccc2)cc1)c1ccc(N(c2ccc(C3CCCCC3)cc2)c2ccc3c4ccccc4n(-c4ccccc4)c3c2)cc1 ; CCCCCC(CC)(c1ccc(N(c2ccccc2)c2ccccc2)cc1)c1ccc(N(c2ccc(C3CCCCC3)cc2)c2ccc3c(c2)c2ccccc2n3-c2ccccc2)cc1 ; CCCCCC(CC)(c1ccc(N(c2ccccc2)c2ccccc2)cc1)c1ccc(N(c2ccc(C3CCCCC3)cc2)c2cccc3c2c2ccccc2n3-c2ccccc2)cc1]

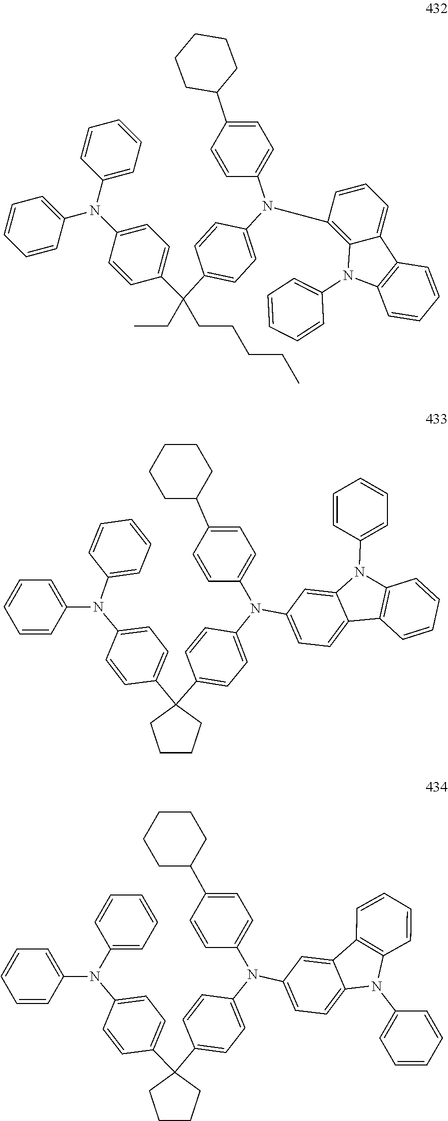 [SMILES: CCCCCC(CC)(c1ccc(N(Cc2cccc3c4ccccc4n(-c4ccccc4)c23)c2ccc(C3CCCCC3)cc2)cc1)c1ccc(N(c2ccccc2)c2ccccc2)cc1 ; c1ccc(N(c2ccccc2)c2ccc(C3(c4ccc(N(c5ccc(C6CCCCC6)cc5)c5ccc6c7ccccc7n(-c7ccccc7)c6c5)cc4)CCCC3)cc2)cc1 ; c1ccc(N(c2ccccc2)c2ccc(C3(c4ccc(N(c5ccc(C6CCCCC6)cc5)c5ccc6c(c5)c5ccccc5n6-c5ccccc5)cc4)CCCC3)cc2)cc1]

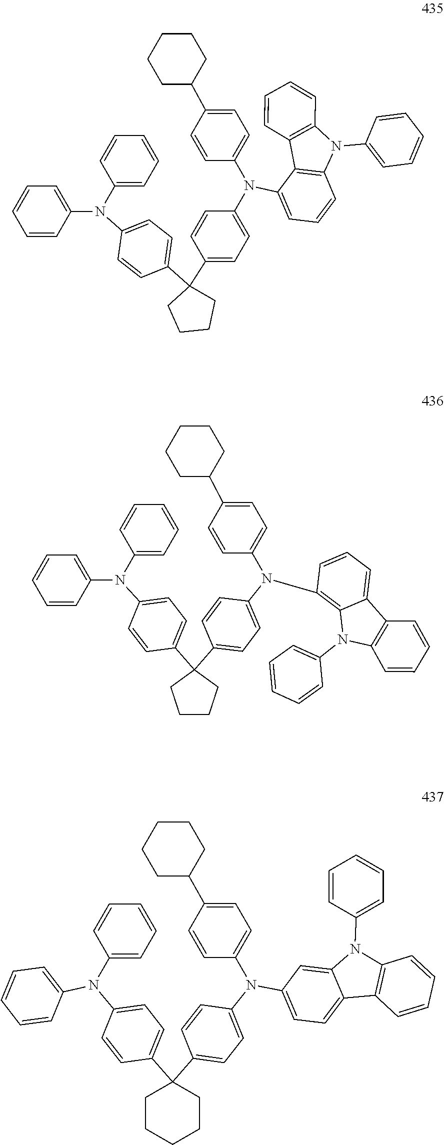 [SMILES: c1ccc(N(c2ccccc2)c2ccc(C3(c4ccc(N(c5ccc(C6CCCCC6)cc5)c5cccc6c5c5ccccc5n6-c5ccccc5)cc4)CCCC3)cc2)cc1 ; c1ccc(N(c2ccccc2)c2ccc(C3(c4ccc(N(c5ccc(C6CCCCC6)cc5)c5cccc6c7ccccc7n(-c7ccccc7)c56)cc4)CCCC3)cc2)cc1 ; c1ccc(N(c2ccccc2)c2ccc(C3(c4ccc(N(c5ccc(C6CCCCC6)cc5)c5ccc6c7ccccc7n(-c7ccccc7)c6c5)cc4)CCCCC3)cc2)cc1]

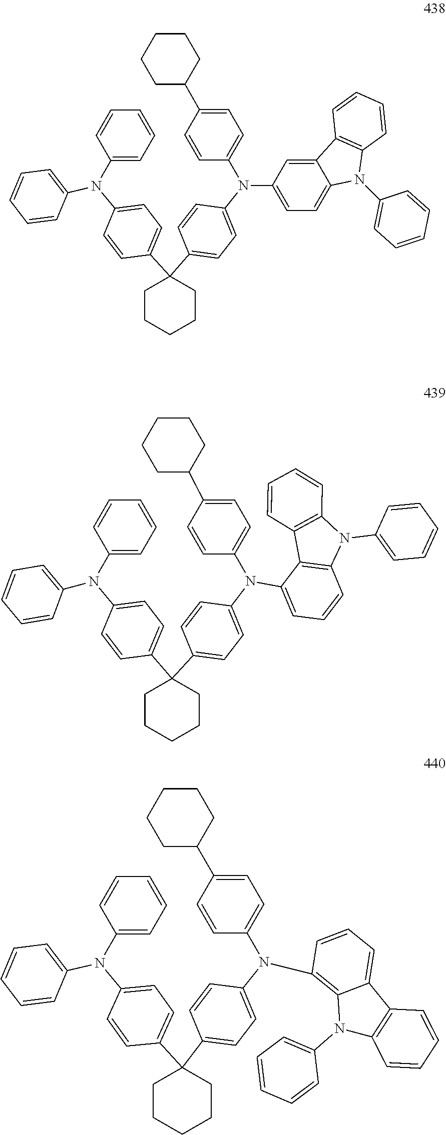 [SMILES: c1ccc(N(c2ccccc2)c2ccc(C3(c4ccc(N(c5ccc(C6CCCCC6)cc5)c5ccc6c(c5)c5ccccc5n6-c5ccccc5)cc4)CCCCC3)cc2)cc1 ; c1ccc(N(c2ccccc2)c2ccc(C3(c4ccc(N(c5ccc(C6CCCCC6)cc5)c5cccc6c5c5ccccc5n6-c5ccccc5)cc4)CCCCC3)cc2)cc1 ; c1ccc(N(c2ccccc2)c2ccc(C3(c4ccc(N(c5ccc(C6CCCCC6)cc5)c5cccc6c7ccccc7n(-c7ccccc7)c56)cc4)CCCCC3)cc2)cc1]

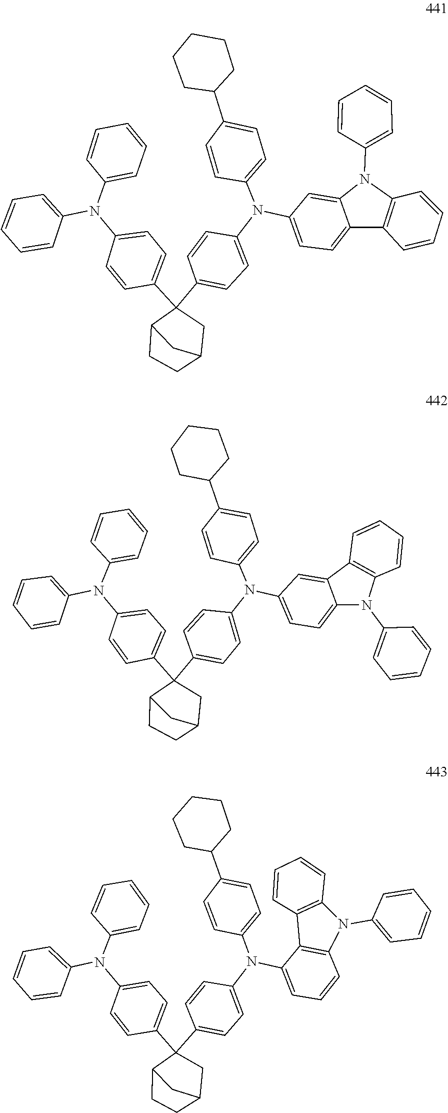 [SMILES: c1ccc(N(c2ccccc2)c2ccc(C3(c4ccc(N(c5ccc(C6CCCCC6)cc5)c5ccc6c7ccccc7n(-c7ccccc7)c6c5)cc4)CC4CCC3C4)cc2)cc1 ; c1ccc(N(c2ccccc2)c2ccc(C3(c4ccc(N(c5ccc(C6CCCCC6)cc5)c5ccc6c(c5)c5ccccc5n6-c5ccccc5)cc4)CC4CCC3C4)cc2)cc1 ; c1ccc(N(c2ccccc2)c2ccc(C3(c4ccc(N(c5ccc(C6CCCCC6)cc5)c5cccc6c5c5ccccc5n6-c5ccccc5)cc4)CC4CCC3C4)cc2)cc1]

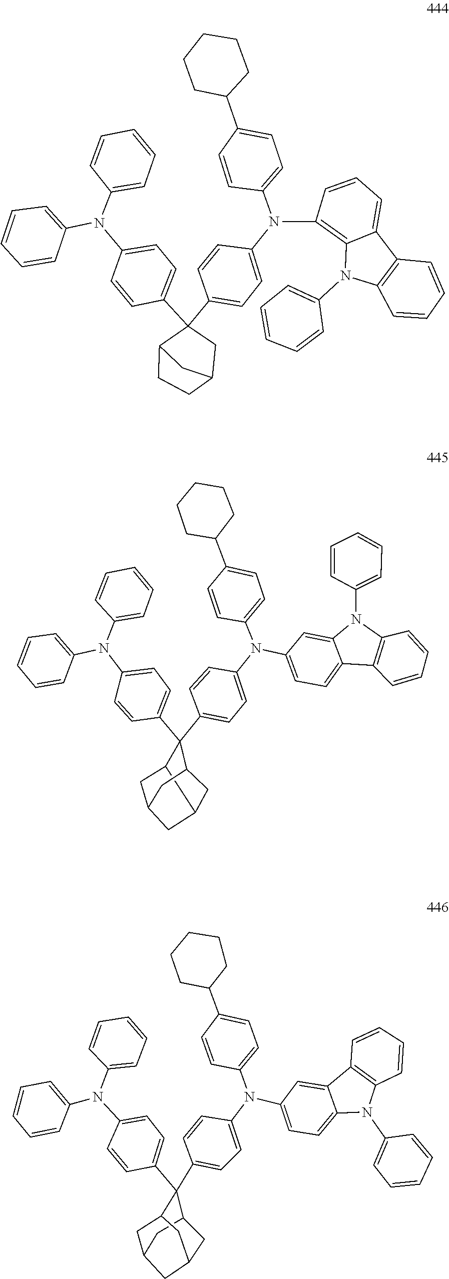 [SMILES: c1ccc(N(c2ccccc2)c2ccc(C3(c4ccc(N(c5ccc(C6CCCCC6)cc5)c5cccc6c7ccccc7n(-c7ccccc7)c56)cc4)CC4CCC3C4)cc2)cc1 ; c1ccc(N(c2ccccc2)c2ccc(C3(c4ccc(N(c5ccc(C6CCCCC6)cc5)c5ccc6c7ccccc7n(-c7ccccc7)c6c5)cc4)C4CC5CC(C4)C3C5)cc2)cc1 ; c1ccc(N(c2ccccc2)c2ccc(C3(c4ccc(N(c5ccc(C6CCCCC6)cc5)c5ccc6c(c5)c5ccccc5n6-c5ccccc5)cc4)C4CC5CC(C4)CC3C5)cc2)cc1]

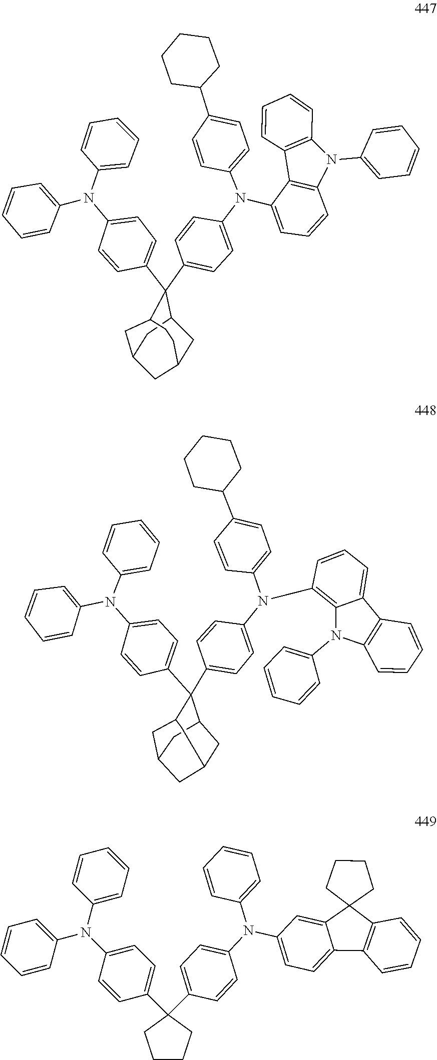 [SMILES: c1ccc(N(c2ccccc2)c2ccc(C3(c4ccc(N(c5ccc(C6CCCCC6)cc5)c5cccc6c5c5ccccc5n6-c5ccccc5)cc4)C4CCC5CC(C4)CC3C5)cc2)cc1 ; c1ccc(N(c2ccccc2)c2ccc(C3(c4ccc(N(c5ccc(C6CCCCC6)cc5)c5cccc6c7ccccc7n(-c7ccccc7)c56)cc4)C4CC5CC(C4)CC3C5)cc2)cc1 ; c1ccc(N(c2ccccc2)c2ccc(C3(c4ccc(N(c5ccccc5)c5ccc6c(c5)C5(CCCC5)c5ccccc5-6)cc4)CCCC3)cc2)cc1]

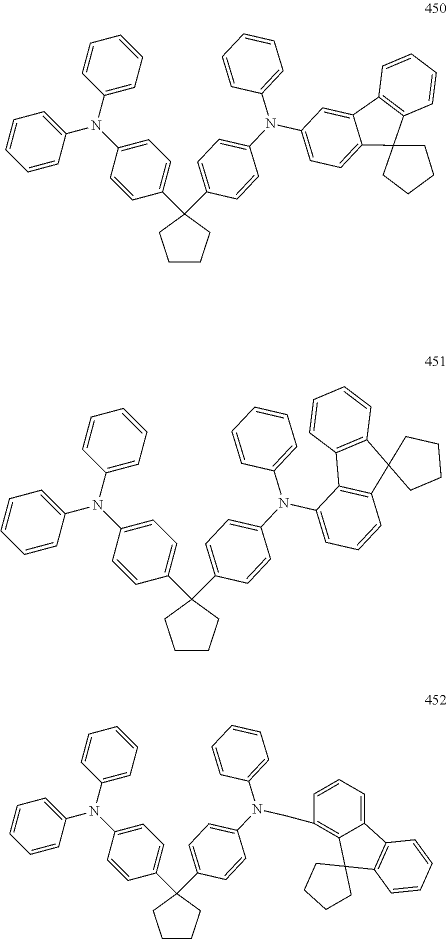 [SMILES: c1ccc(N(c2ccccc2)c2ccc(C3(c4ccc(N(c5ccccc5)c5ccc6c(c5)-c5ccccc5C65CCCC5)cc4)CCCC3)cc2)cc1 ; c1ccc(N(c2ccccc2)c2ccc(C3(c4ccc(N(c5ccccc5)c5cccc6c5-c5ccccc5C65CCCC5)cc4)CCCC3)cc2)cc1 ; c1ccc(N(Cc2cccc3c2C2(CCCC2)c2ccccc2-3)c2ccc(C3(c4ccc(N(c5ccccc5)c5ccccc5)cc4)CCCC3)cc2)cc1]

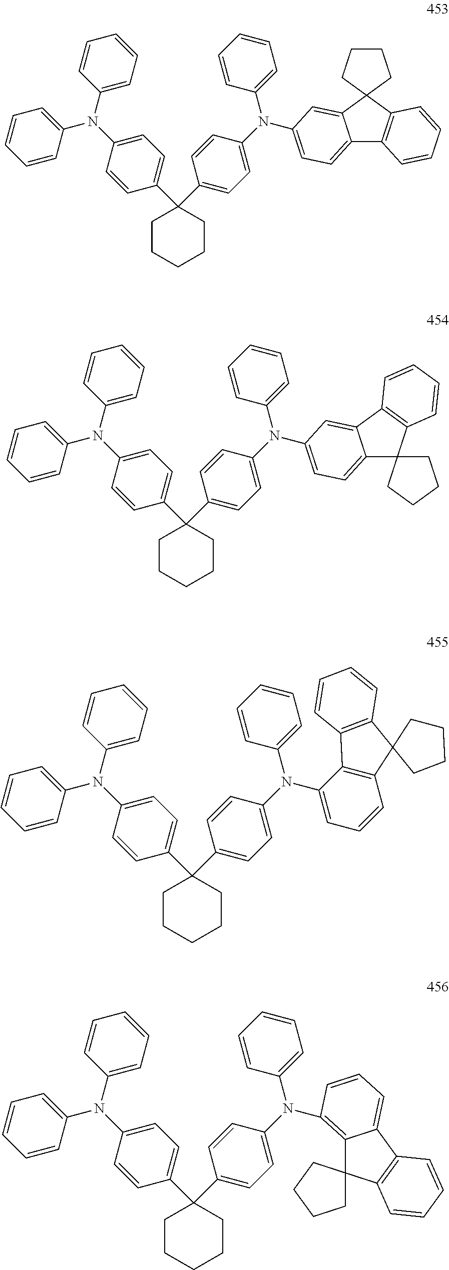 [SMILES: c1ccc(N(c2ccccc2)c2ccc(C3(c4ccc(N(c5ccccc5)c5ccc6c(c5)C5(CCCC5)c5ccccc5-6)cc4)CCCCC3)cc2)cc1 ; c1ccc(N(c2ccccc2)c2ccc(C3(c4ccc(N(c5ccccc5)c5ccc6c(c5)-c5ccccc5C65CCCC5)cc4)CCCCC3)cc2)cc1 ; c1ccc(N(c2ccccc2)c2ccc(C3(c4ccc(N(c5ccccc5)c5cccc6c5-c5ccccc5C65CCCC5)cc4)CCCCC3)cc2)cc1 ; c1ccc(N(c2ccccc2)c2ccc(C3(c4ccc(N(c5ccccc5)c5cccc6c5C5(CCCC5)c5ccccc5-6)cc4)CCCCC3)cc2)cc1]

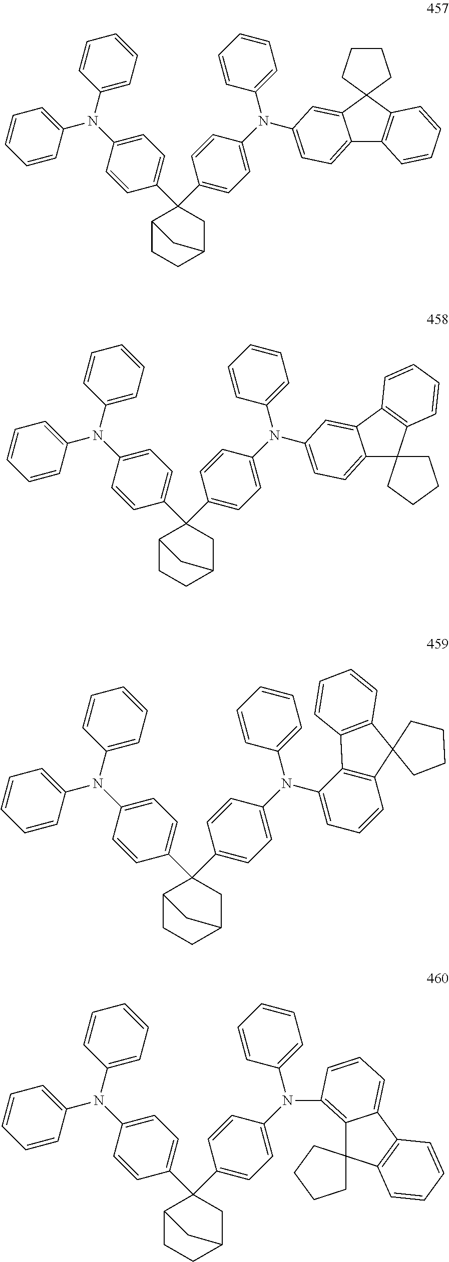 [SMILES: c1ccc(N(c2ccccc2)c2ccc(C3(c4ccc(N(c5ccccc5)c5ccc6c(c5)C5(CCCC5)c5ccccc5-6)cc4)CC4CCC3C4)cc2)cc1 ; c1ccc(N(c2ccccc2)c2ccc(C3(c4ccc(N(c5ccccc5)c5ccc6c(c5)-c5ccccc5C65CCCC5)cc4)CC4CCC3C4)cc2)cc1 ; c1ccc(N(c2ccccc2)c2ccc(C3(c4ccc(N(c5ccccc5)c5cccc6c5-c5ccccc5C65CCCC5)cc4)CC4CCC3C4)cc2)cc1 ; c1ccc(N(c2ccccc2)c2ccc(C3(c4ccc(N(c5ccccc5)c5cccc6c5C5(CCCC5)c5ccccc5-6)cc4)CC4CCC3C4)cc2)cc1]

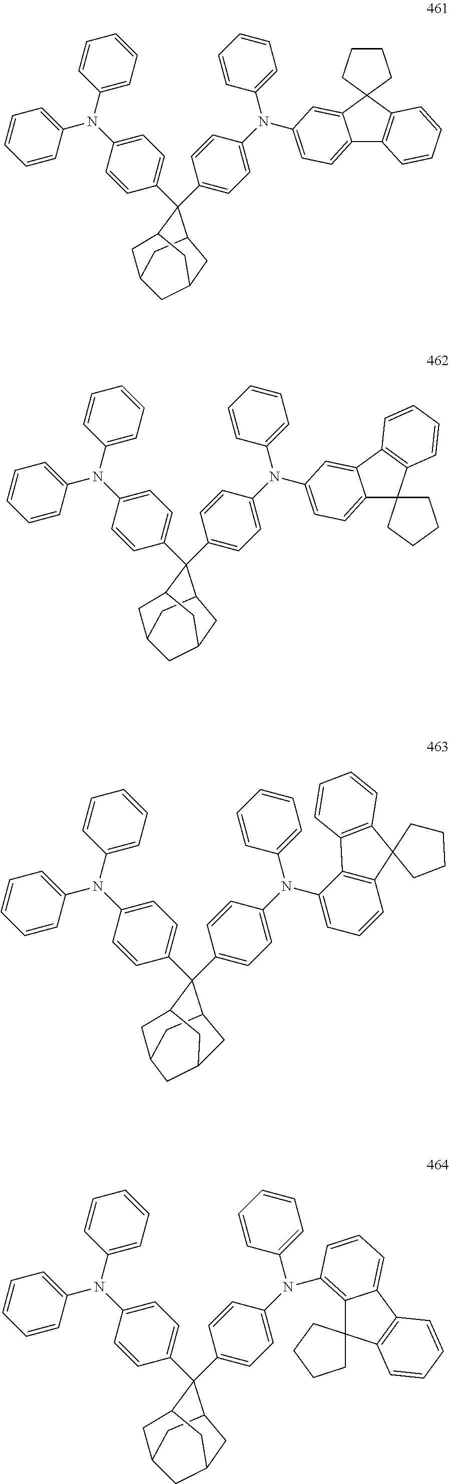 [SMILES: c1ccc(N(c2ccccc2)c2ccc(C3(c4ccc(N(c5ccccc5)c5ccc6c(c5)C5(CCCC5)c5ccccc5-6)cc4)C4CC5CC(C4)CC3C5)cc2)cc1 ; c1ccc(N(c2ccccc2)c2ccc(C3(c4ccc(N(c5ccccc5)c5ccc6c(c5)-c5ccccc5C65CCCC5)cc4)C4CC5CC(C4)CC3C5)cc2)cc1 ; c1ccc(N(c2ccccc2)c2ccc(C3(c4ccc(N(c5ccccc5)c5cccc6c5-c5ccccc5C65CCCC5)cc4)C4CC5CC(C4)CC3C5)cc2)cc1 ; c1ccc(N(c2ccccc2)c2ccc(C3(c4ccc(N(c5ccccc5)c5cccc6c5C5(CCCC5)c5ccccc5-6)cc4)C4CC5CC(C4)CC3C5)cc2)cc1]

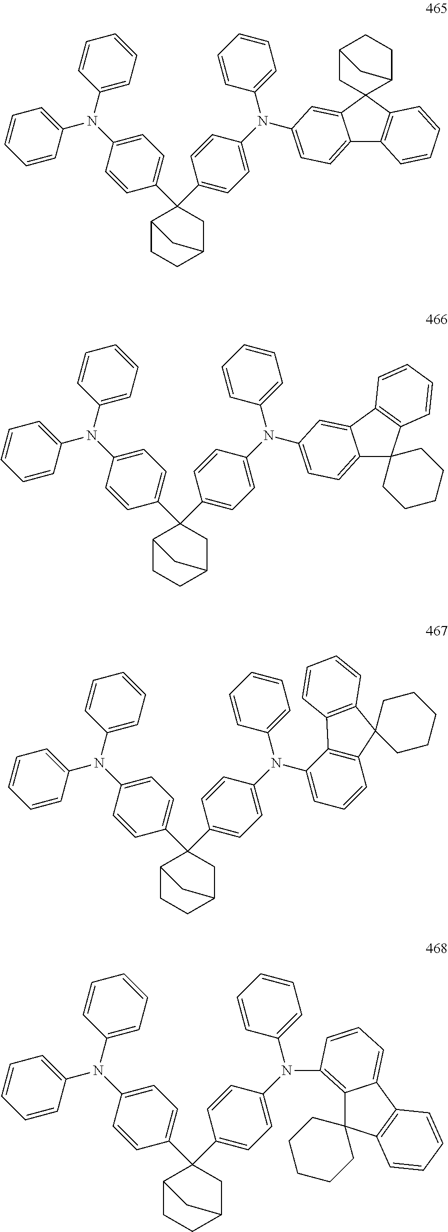 [SMILES: c1ccc(N(c2ccccc2)c2ccc(C3(c4ccc(N(c5ccccc5)c5ccc6c(c5)C5(CC7CCC5C7)c5ccccc5-6)cc4)CC4CCC3C4)cc2)cc1 ; c1ccc(N(c2ccccc2)c2ccc(C3(c4ccc(N(c5ccccc5)c5ccc6c(c5)-c5ccccc5C65CCCCC5)cc4)CC4CCC3C4)cc2)cc1 ; c1ccc(N(c2ccccc2)c2ccc(C3(c4ccc(N(c5ccccc5)c5cccc6c5-c5ccccc5C65CCCCC5)cc4)CC4CCC3C4)cc2)cc1 ; c1ccc(N(c2ccccc2)c2ccc(C3(c4ccc(N(c5ccccc5)c5cccc6c5C5(CCCCC5)c5ccccc5-6)cc4)CC4CCC3C4)cc2)cc1]

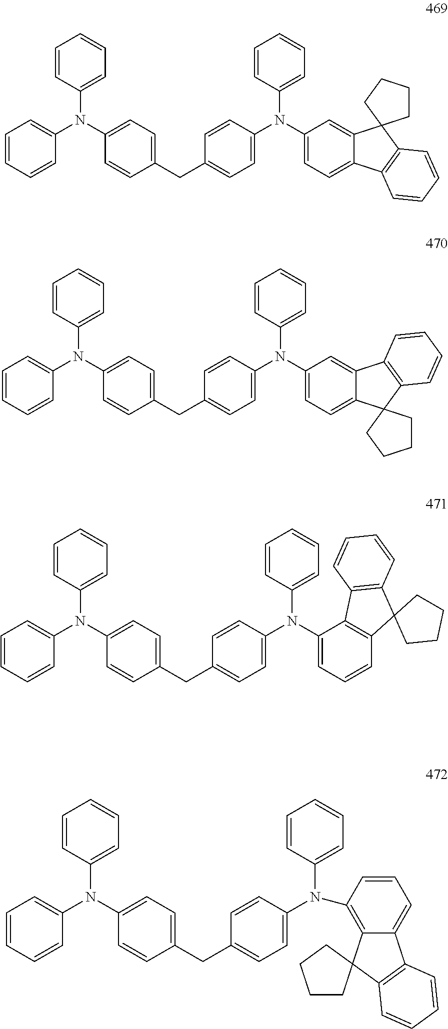 [SMILES: c1ccc(N(c2ccccc2)c2ccc(Cc3ccc(N(c4ccccc4)c4ccc5c(c4)C4(CCCC4)c4ccccc4-5)cc3)cc2)cc1 ; c1ccc(N(c2ccccc2)c2ccc(Cc3ccc(N(c4ccccc4)c4ccc5c(c4)-c4ccccc4C54CCCC4)cc3)cc2)cc1 ; c1ccc(N(c2ccccc2)c2ccc(Cc3ccc(N(c4ccccc4)c4cccc5c4-c4ccccc4C54CCCC4)cc3)cc2)cc1 ; c1ccc(N(c2ccccc2)c2ccc(Cc3ccc(N(c4ccccc4)c4cccc5c4C4(CCCC4)c4ccccc4-5)cc3)cc2)cc1]

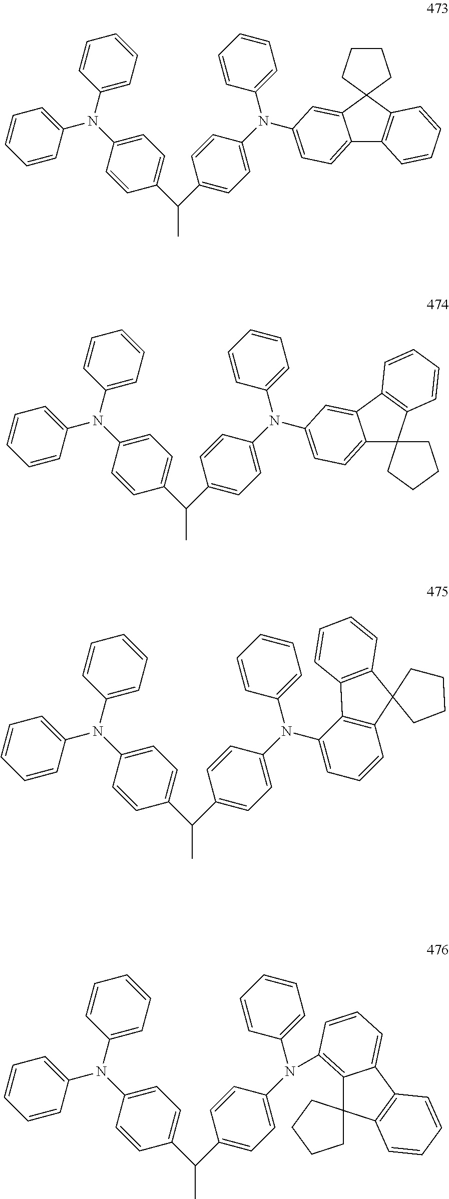 [SMILES: CC(c1ccc(N(c2ccccc2)c2ccccc2)cc1)c1ccc(N(c2ccccc2)c2ccc3c(c2)C2(CCCC2)c2ccccc2-3)cc1 ; CC(c1ccc(N(c2ccccc2)c2ccccc2)cc1)c1ccc(N(c2ccccc2)c2ccc3c(c2)-c2ccccc2C32CCCC2)cc1 ; CC(c1ccc(N(c2ccccc2)c2ccccc2)cc1)c1ccc(N(c2ccccc2)c2cccc3c2-c2ccccc2C32CCCC2)cc1 ; CC(c1ccc(N(c2ccccc2)c2ccccc2)cc1)c1ccc(N(c2ccccc2)c2cccc3c2C2(CCCC2)c2ccccc2-3)cc1]

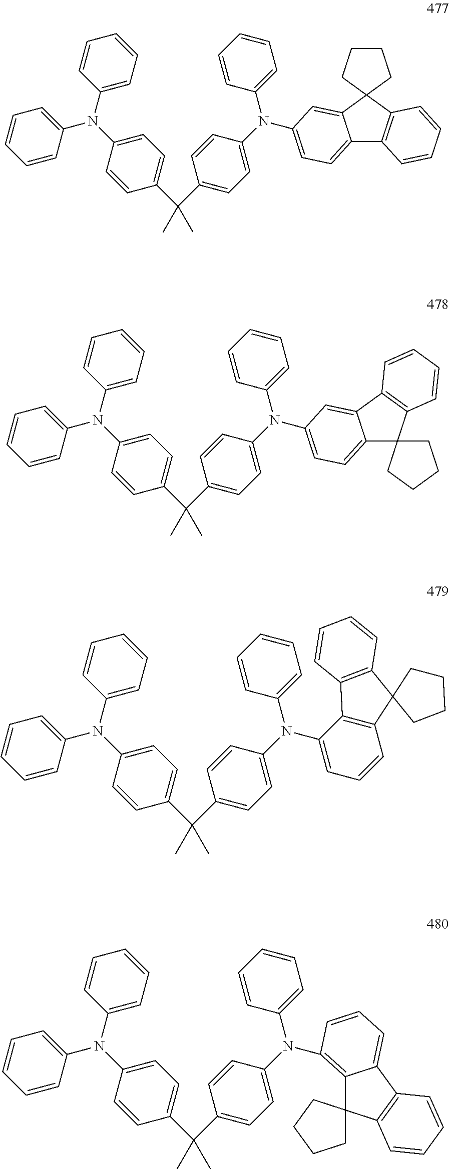 [SMILES: CC(C)(c1ccc(N(c2ccccc2)c2ccccc2)cc1)c1ccc(N(c2ccccc2)c2ccc3c(c2)C2(CCCC2)c2ccccc2-3)cc1 ; CC(C)(c1ccc(N(c2ccccc2)c2ccccc2)cc1)c1ccc(N(c2ccccc2)c2ccc3c(c2)-c2ccccc2C32CCCC2)cc1 ; CC(C)(c1ccc(N(c2ccccc2)c2ccccc2)cc1)c1ccc(N(c2ccccc2)c2cccc3c2-c2ccccc2C32CCCC2)cc1 ; CC(C)(c1ccc(N(c2ccccc2)c2ccccc2)cc1)c1ccc(N(c2ccccc2)c2cccc3c2C2(CCCC2)c2ccccc2-3)cc1]

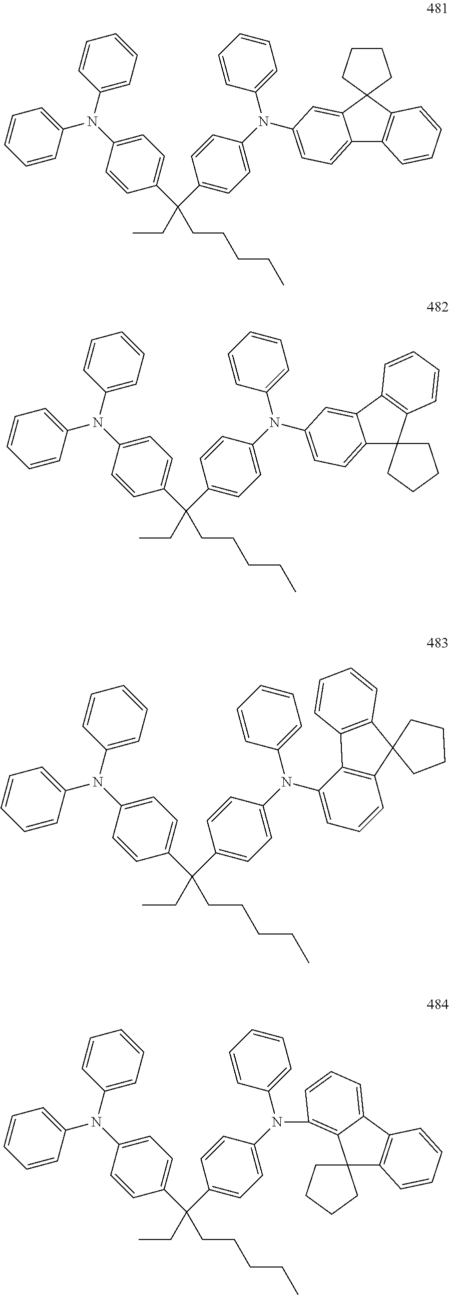 [SMILES: CCCCCC(CC)(c1ccc(N(c2ccccc2)c2ccccc2)cc1)c1ccc(N(c2ccccc2)c2ccc3c(c2)C2(CCCC2)c2ccccc2-3)cc1 ; CCCCCC(CC)(c1ccc(N(c2ccccc2)c2ccccc2)cc1)c1ccc(N(c2ccccc2)c2ccc3c(c2)-c2ccccc2C32CCCC2)cc1 ; CCCCCC(CC)(c1ccc(N(c2ccccc2)c2ccccc2)cc1)c1ccc(N(c2ccccc2)c2cccc3c2-c2ccccc2C32CCCC2)cc1 ; CCCCCC(CC)(c1ccc(N(c2ccccc2)c2ccccc2)cc1)c1ccc(N(c2ccccc2)c2cccc3c2C2(CCCC2)c2ccccc2-3)cc1]

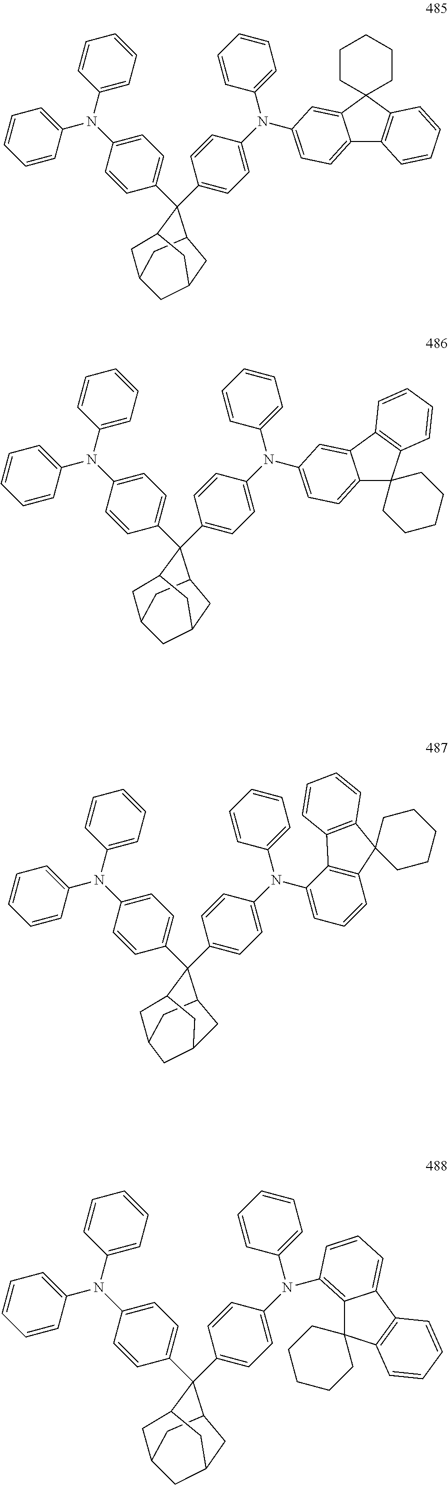 [SMILES: c1ccc(N(c2ccccc2)c2ccc(C3(c4ccc(N(c5ccccc5)c5ccc6c(c5)C5(CCCCC5)c5ccccc5-6)cc4)C4CC5CC(C4)CC3C5)cc2)cc1 ; c1ccc(N(c2ccccc2)c2ccc(C3(c4ccc(N(c5ccccc5)c5ccc6c(c5)-c5ccccc5C65CCCCC5)cc4)C4CC5CC(C4)CC3C5)cc2)cc1 ; c1ccc(N(c2ccccc2)c2ccc(C3(c4ccc(N(c5ccccc5)c5cccc6c5-c5ccccc5C65CCCCC5)cc4)C4CC5CC(C4)CC3C5)cc2)cc1 ; c1ccc(N(c2ccccc2)c2ccc(C3(c4ccc(N(c5ccccc5)c5cccc6c5C5(CCCCC5)c5ccccc5-6)cc4)C4CC5CC(C4)CC3C5)cc2)cc1]

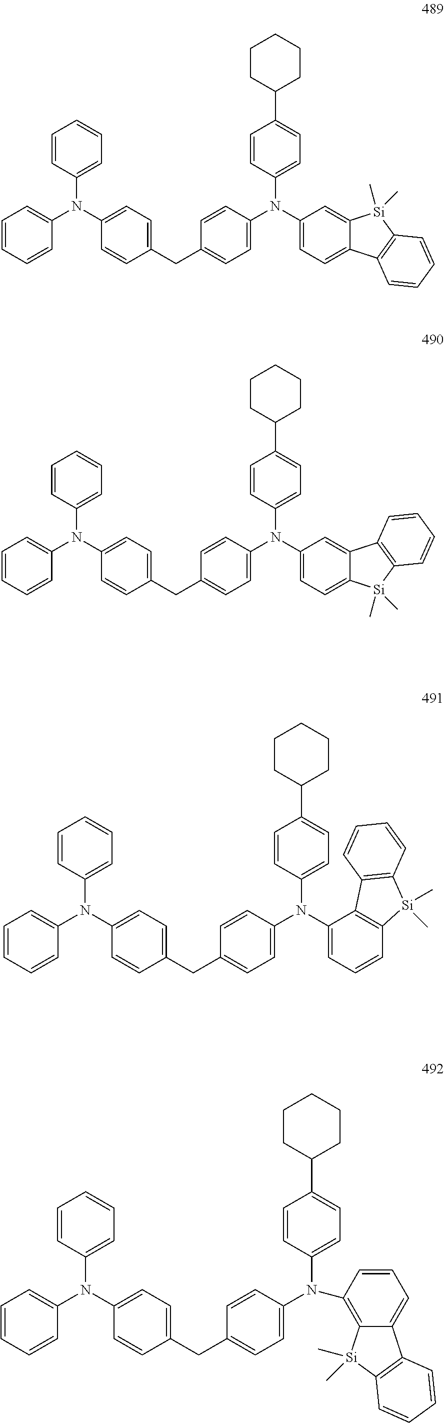 [SMILES: C[Si]1(C)c2ccccc2-c2ccc(N(c3ccc(Cc4ccc(N(c5ccccc5)c5ccccc5)cc4)cc3)c3ccc(C4CCCCC4)cc3)cc21 ; C[Si]1(C)c2ccccc2-c2cc(N(c3ccc(Cc4ccc(N(c5ccccc5)c5ccccc5)cc4)cc3)c3ccc(C4CCCCC4)cc3)ccc21 ; C[Si]1(C)c2ccccc2-c2c(N(c3ccc(Cc4ccc(N(c5ccccc5)c5ccccc5)cc4)cc3)c3ccc(C4CCCCC4)cc3)cccc21 ; C[Si]1(C)c2ccccc2-c2cccc(N(c3ccc(Cc4ccc(N(c5ccccc5)c5ccccc5)cc4)cc3)c3ccc(C4CCCCC4)cc3)c21]

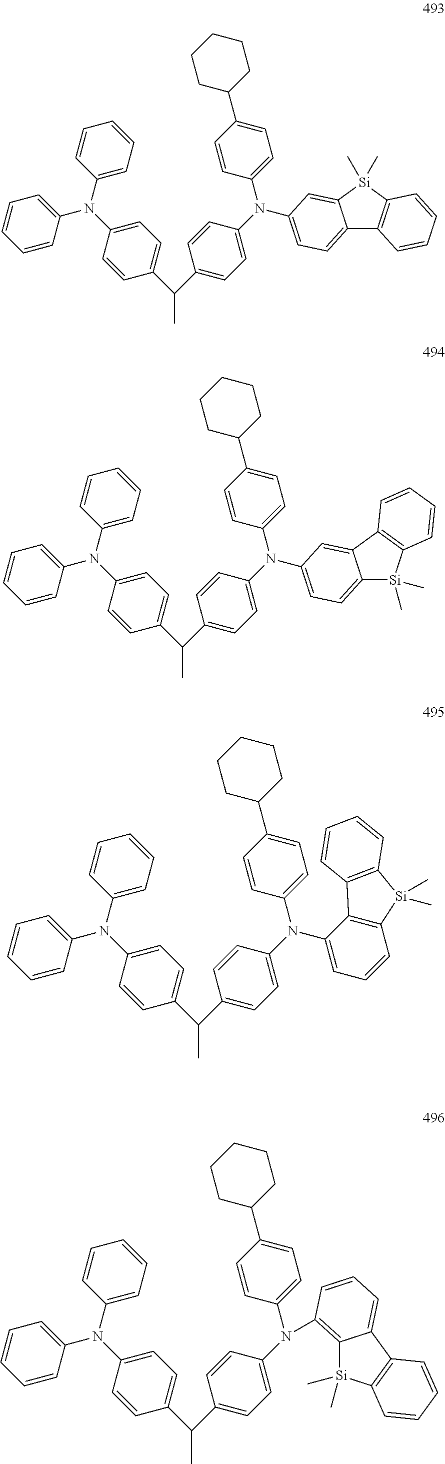 [SMILES: CC(c1ccc(N(c2ccccc2)c2ccccc2)cc1)c1ccc(N(c2ccc(C3CCCCC3)cc2)c2ccc3c(c2)[Si](C)(C)c2ccccc2-3)cc1 ; CC(c1ccc(N(c2ccccc2)c2ccccc2)cc1)c1ccc(N(c2ccc(C3CCCCC3)cc2)c2ccc3c(c2)-c2ccccc2[Si]3(C)C)cc1 ; CC(c1ccc(N(c2ccccc2)c2ccccc2)cc1)c1ccc(N(c2ccc(C3CCCCC3)cc2)c2cccc3c2-c2ccccc2[Si]3(C)C)cc1 ; CC(c1ccc(N(c2ccccc2)c2ccccc2)cc1)c1ccc(N(c2ccc(C3CCCCC3)cc2)c2cccc3c2[Si](C)(C)c2ccccc2-3)cc1]

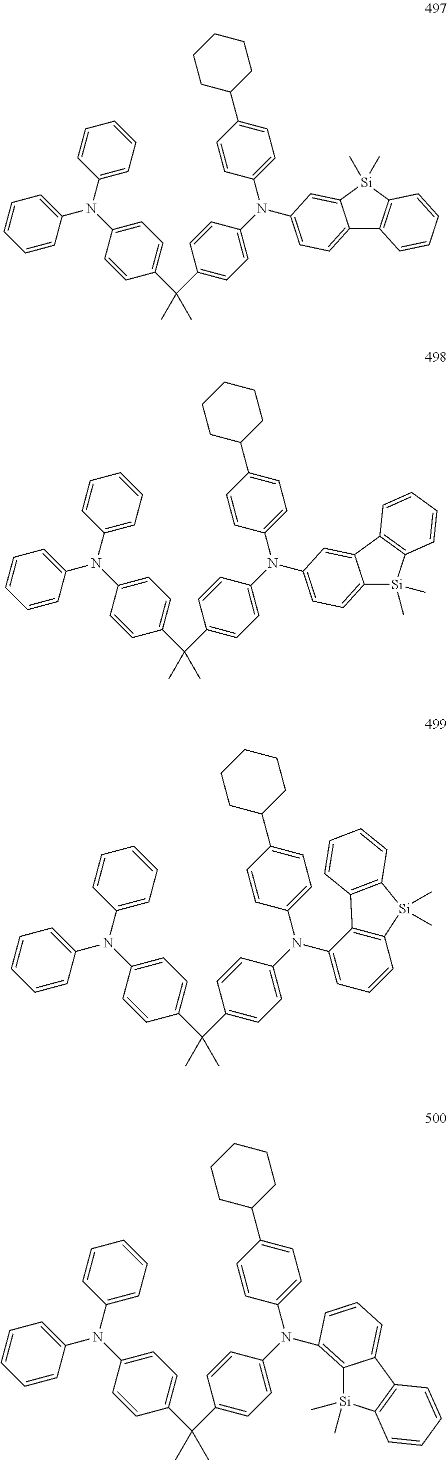 [SMILES: CC(C)(c1ccc(N(c2ccccc2)c2ccccc2)cc1)c1ccc(N(c2ccc(C3CCCCC3)cc2)c2ccc3c(c2)[Si](C)(C)c2ccccc2-3)cc1 ; CC(C)(c1ccc(N(c2ccccc2)c2ccccc2)cc1)c1ccc(N(c2ccc(C3CCCCC3)cc2)c2ccc3c(c2)-c2ccccc2[Si]3(C)C)cc1 ; CC(C)(c1ccc(N(c2ccccc2)c2ccccc2)cc1)c1ccc(N(c2ccc(C3CCCCC3)cc2)c2cccc3c2-c2ccccc2[Si]3(C)C)cc1 ; CC(C)(c1ccc(N(c2ccccc2)c2ccccc2)cc1)c1ccc(N(c2ccc(C3CCCCC3)cc2)c2cccc3c2[Si](C)(C)c2ccccc2-3)cc1]

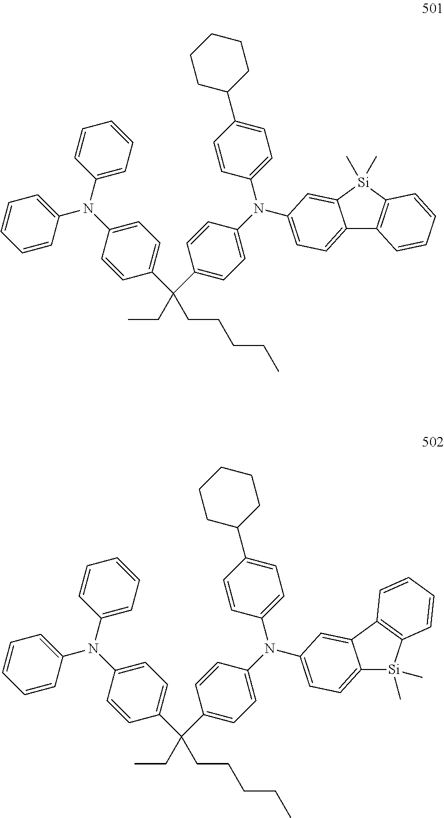 [SMILES: CCCCCC(CC)(c1ccc(N(c2ccccc2)c2ccccc2)cc1)c1ccc(N(c2ccc(C3CCCCC3)cc2)c2ccc3c(c2)[Si](C)(C)c2ccccc2-3)cc1 ; CCCCCC(CC)(c1ccc(N(c2ccccc2)c2ccccc2)cc1)c1ccc(N(c2ccc(C3CCCCC3)cc2)c2ccc3c(c2)-c2ccccc2[Si]3(C)C)cc1]

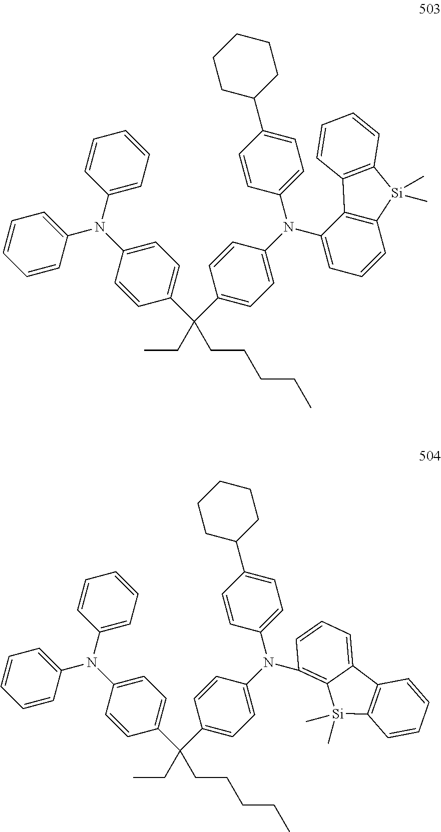 [SMILES: CCCCCC(CC)(c1ccc(N(c2ccccc2)c2ccccc2)cc1)c1ccc(N(c2ccc(C3CCCCC3)cc2)c2cccc3c2-c2ccccc2[Si]3(C)C)cc1 ; CCCCCC(CC)(c1ccc(N(c2ccccc2)c2ccccc2)cc1)c1ccc(N(c2ccc(C3CCCCC3)cc2)c2cccc3c2[Si](C)(C)c2ccccc2-3)cc1]

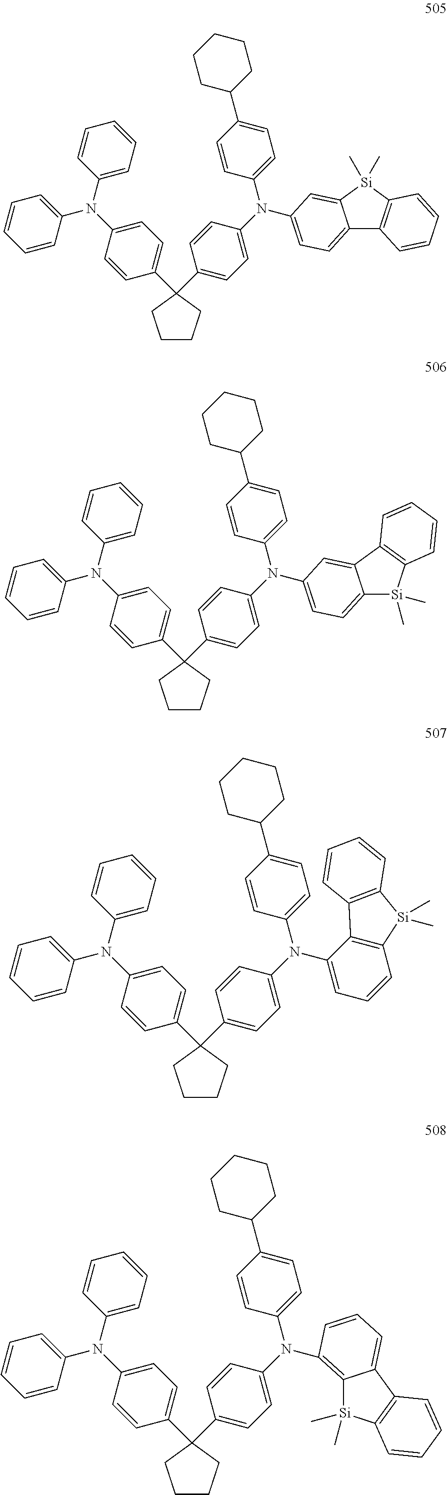 [SMILES: C[Si]1(C)c2ccccc2-c2ccc(N(c3ccc(C4CCCCC4)cc3)c3ccc(C4(c5ccc(N(c6ccccc6)c6ccccc6)cc5)CCCC4)cc3)cc21 ; C[Si]1(C)c2ccccc2-c2cc(N(c3ccc(C4CCCCC4)cc3)c3ccc(C4(c5ccc(N(c6ccccc6)c6ccccc6)cc5)CCCC4)cc3)ccc21 ; C[Si]1(C)c2ccccc2-c2c(N(c3ccc(C4CCCCC4)cc3)c3ccc(C4(c5ccc(N(c6ccccc6)c6ccccc6)cc5)CCCC4)cc3)cccc21 ; C[Si]1(C)c2ccccc2-c2cccc(N(c3ccc(C4CCCCC4)cc3)c3ccc(C4(c5ccc(N(c6ccccc6)c6ccccc6)cc5)CCCC4)cc3)c21]

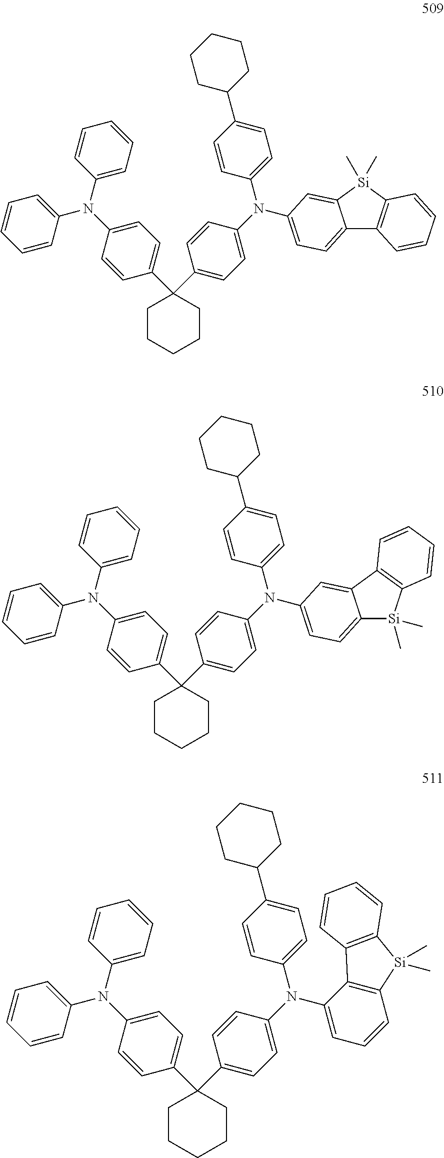 [SMILES: C[Si]1(C)c2ccccc2-c2ccc(N(c3ccc(C4CCCCC4)cc3)c3ccc(C4(c5ccc(N(c6ccccc6)c6ccccc6)cc5)CCCCC4)cc3)cc21 ; C[Si]1(C)c2ccccc2-c2cc(N(c3ccc(C4CCCCC4)cc3)c3ccc(C4(c5ccc(N(c6ccccc6)c6ccccc6)cc5)CCCCC4)cc3)ccc21 ; C[Si]1(C)c2ccccc2-c2c(N(c3ccc(C4CCCCC4)cc3)c3ccc(C4(c5ccc(N(c6ccccc6)c6ccccc6)cc5)CCCCC4)cc3)cccc21]

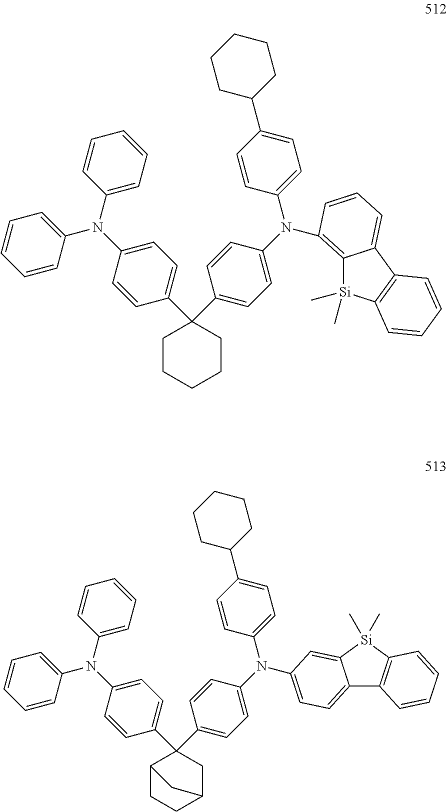 [SMILES: C[Si]1(C)c2ccccc2-c2cccc(N(c3ccc(C4CCCCC4)cc3)c3ccc(C4(c5ccc(N(c6ccccc6)c6ccccc6)cc5)CCCCC4)cc3)c21 ; C[Si]1(C)c2ccccc2-c2ccc(N(c3ccc(C4CCCCC4)cc3)c3ccc(C4(c5ccc(N(c6ccccc6)c6ccccc6)cc5)CC5CCC4C5)cc3)cc21]

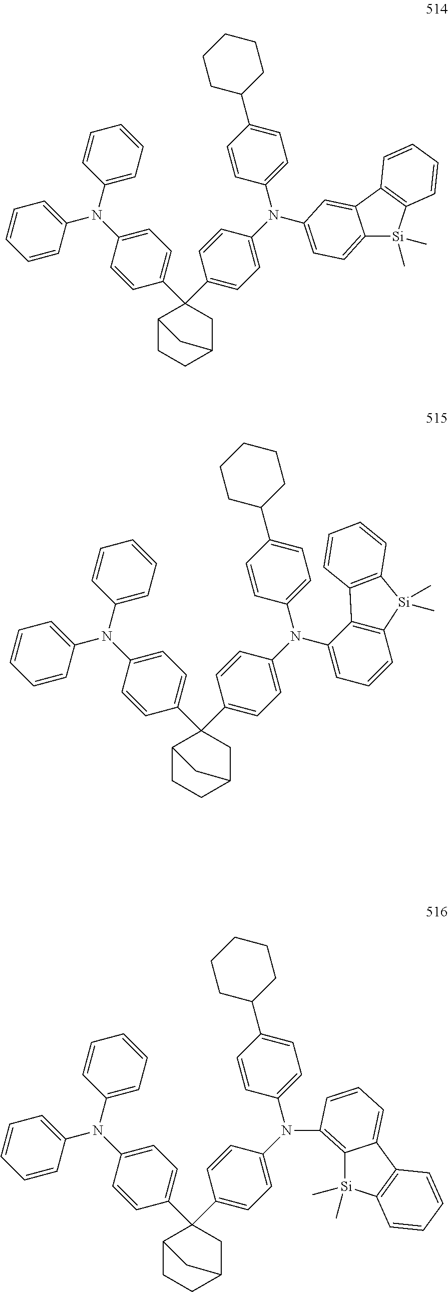 [SMILES: C[Si]1(C)c2ccccc2-c2cc(N(c3ccc(C4CCCCC4)cc3)c3ccc(C4(c5ccc(N(c6ccccc6)c6ccccc6)cc5)CC5CCC4C5)cc3)ccc21 ; C[Si]1(C)c2ccccc2-c2c(N(c3ccc(C4CCCCC4)cc3)c3ccc(C4(c5ccc(N(c6ccccc6)c6ccccc6)cc5)CC5CCC4C5)cc3)cccc21 ; C[Si]1(C)c2ccccc2-c2cccc(N(c3ccc(C4CCCCC4)cc3)c3ccc(C4(c5ccc(N(c6ccccc6)c6ccccc6)cc5)CC5CCC4C5)cc3)c21]

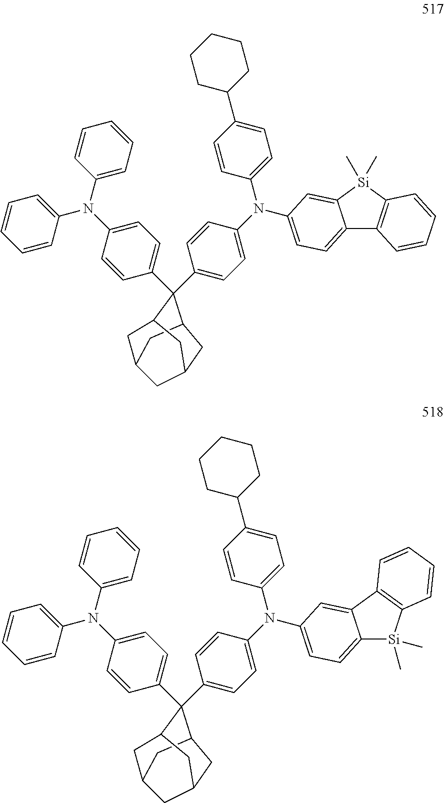 [SMILES: C[Si]1(C)c2ccccc2-c2ccc(N(c3ccc(C4CCCCC4)cc3)c3ccc(C4(c5ccc(N(c6ccccc6)c6ccccc6)cc5)C5CC6CC(C5)CC4C6)cc3)cc21 ; C[Si]1(C)c2ccccc2-c2cc(N(c3ccc(C4CCCCC4)cc3)c3ccc(C4(c5ccc(N(c6ccccc6)c6ccccc6)cc5)C5CC6CC(C5)CC4C6)cc3)ccc21]

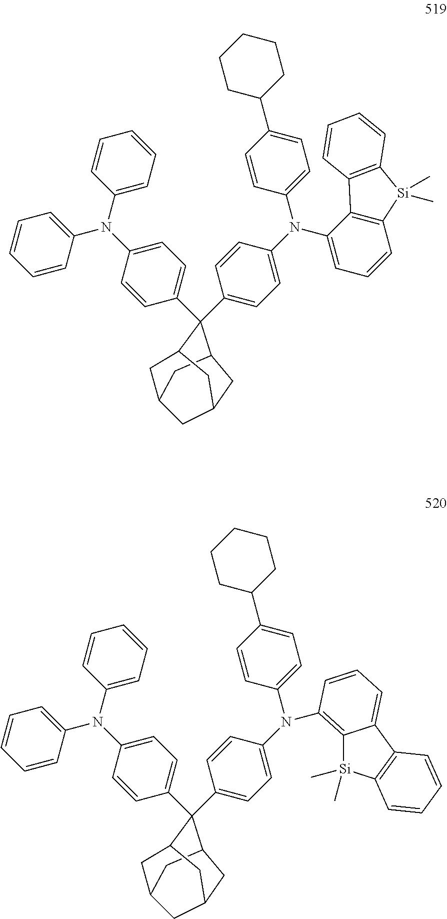 [SMILES: C[Si]1(C)c2ccccc2-c2c(N(c3ccc(C4CCCCC4)cc3)c3ccc(C4(c5ccc(N(c6ccccc6)c6ccccc6)cc5)C5CC6CC(C5)CC4C6)cc3)cccc21 ; C[Si]1(C)c2ccccc2-c2cccc(N(c3ccc(C4CCCCC4)cc3)c3ccc(C4(c5ccc(N(c6ccccc6)c6ccccc6)cc5)C5CC6CC(C5)CC4C6)cc3)c21]

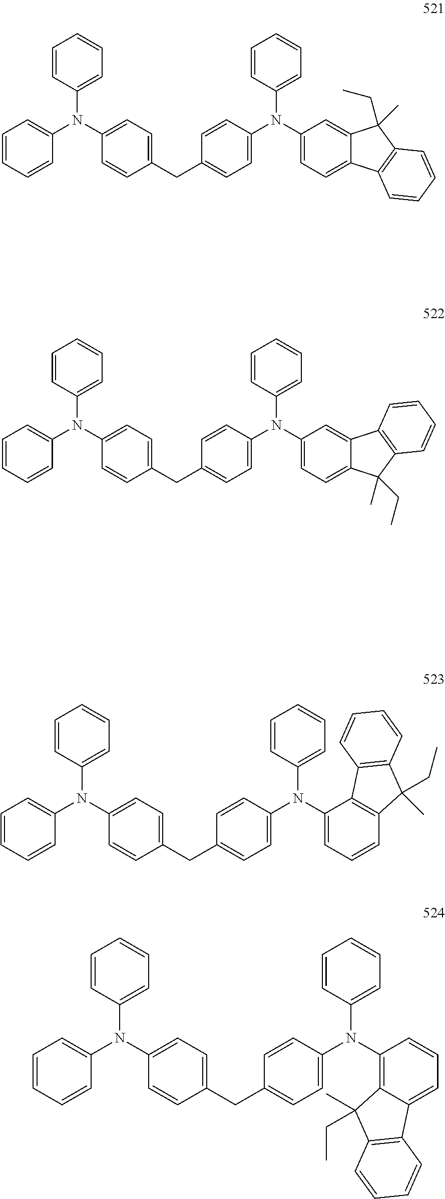 [SMILES: CCC1(C)c2ccccc2-c2ccc(N(c3ccccc3)c3ccc(Cc4ccc(N(c5ccccc5)c5ccccc5)cc4)cc3)cc21 ; CCC1(C)c2ccccc2-c2cc(N(c3ccccc3)c3ccc(Cc4ccc(N(c5ccccc5)c5ccccc5)cc4)cc3)ccc21 ; CCC1(C)c2ccccc2-c2c(N(c3ccccc3)c3ccc(Cc4ccc(N(c5ccccc5)c5ccccc5)cc4)cc3)cccc21 ; CCC1(C)c2ccccc2-c2cccc(N(c3ccccc3)c3ccc(Cc4ccc(N(c5ccccc5)c5ccccc5)cc4)cc3)c21]

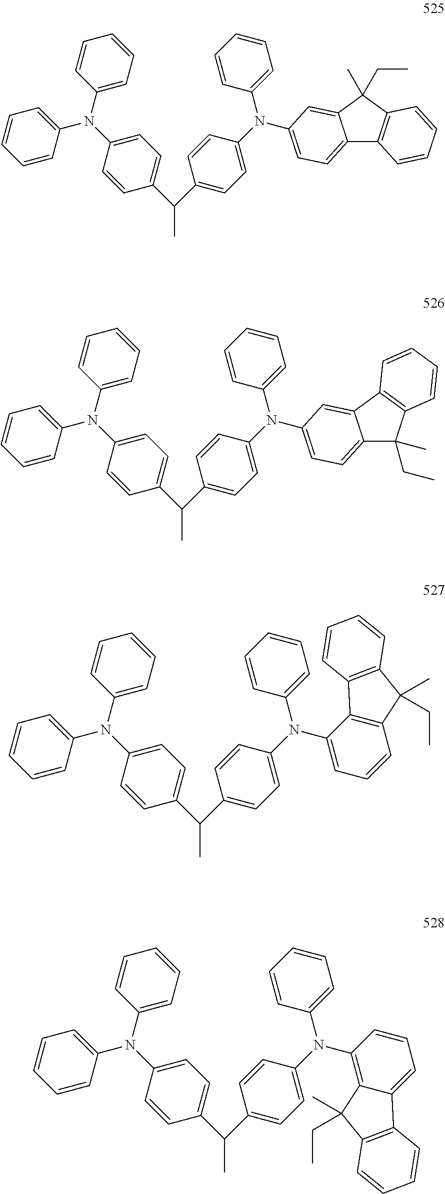 [SMILES: CCC1(C)c2ccccc2-c2ccc(N(c3ccccc3)c3ccc(C(C)c4ccc(N(c5ccccc5)c5ccccc5)cc4)cc3)cc21 ; CCC1(C)c2ccccc2-c2cc(N(c3ccccc3)c3ccc(C(C)c4ccc(N(c5ccccc5)c5ccccc5)cc4)cc3)ccc21 ; CCC1(C)c2ccccc2-c2c(N(c3ccccc3)c3ccc(C(C)c4ccc(N(c5ccccc5)c5ccccc5)cc4)cc3)cccc21 ; CCC1(C)c2ccccc2-c2cccc(N(c3ccccc3)c3ccc(C(C)c4ccc(N(c5ccccc5)c5ccccc5)cc4)cc3)c21]

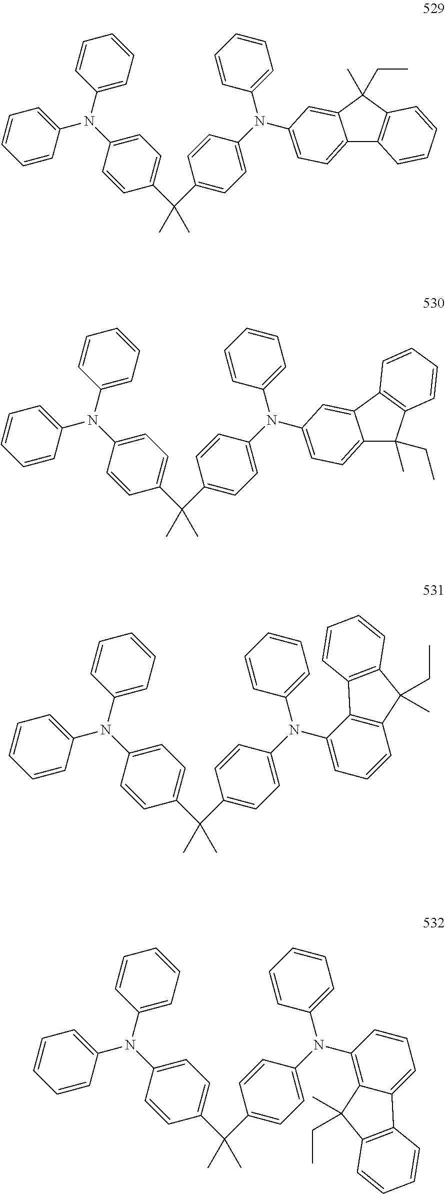 [SMILES: CCC1(C)c2ccccc2-c2ccc(N(c3ccccc3)c3ccc(C(C)(C)c4ccc(N(c5ccccc5)c5ccccc5)cc4)cc3)cc21 ; CCC1(C)c2ccccc2-c2cc(N(c3ccccc3)c3ccc(C(C)(C)c4ccc(N(c5ccccc5)c5ccccc5)cc4)cc3)ccc21 ; CCC1(C)c2ccccc2-c2c(N(c3ccccc3)c3ccc(C(C)(C)c4ccc(N(c5ccccc5)c5ccccc5)cc4)cc3)cccc21 ; CCC1(C)c2ccccc2-c2cccc(N(c3ccccc3)c3ccc(C(C)(C)c4ccc(N(c5ccccc5)c5ccccc5)cc4)cc3)c21]

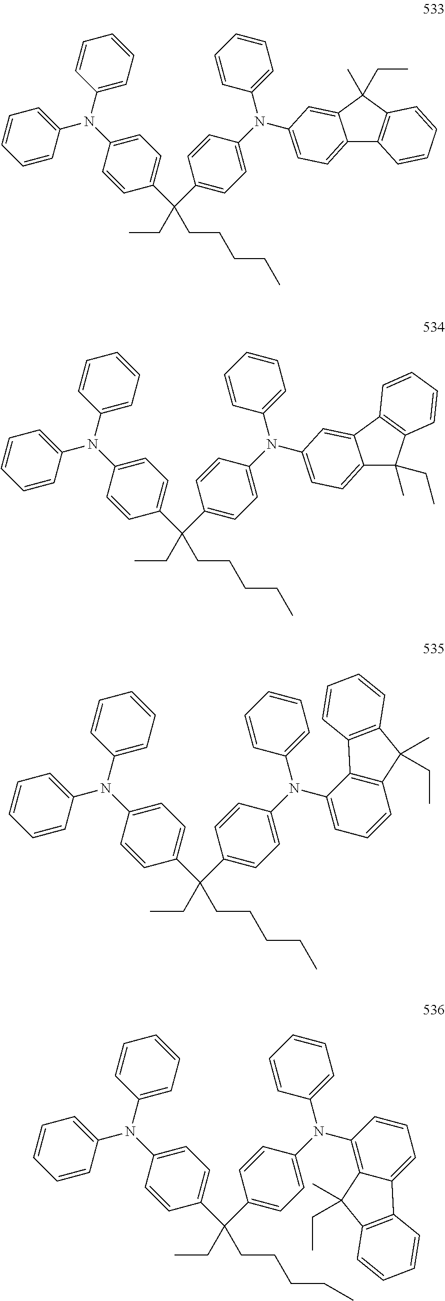

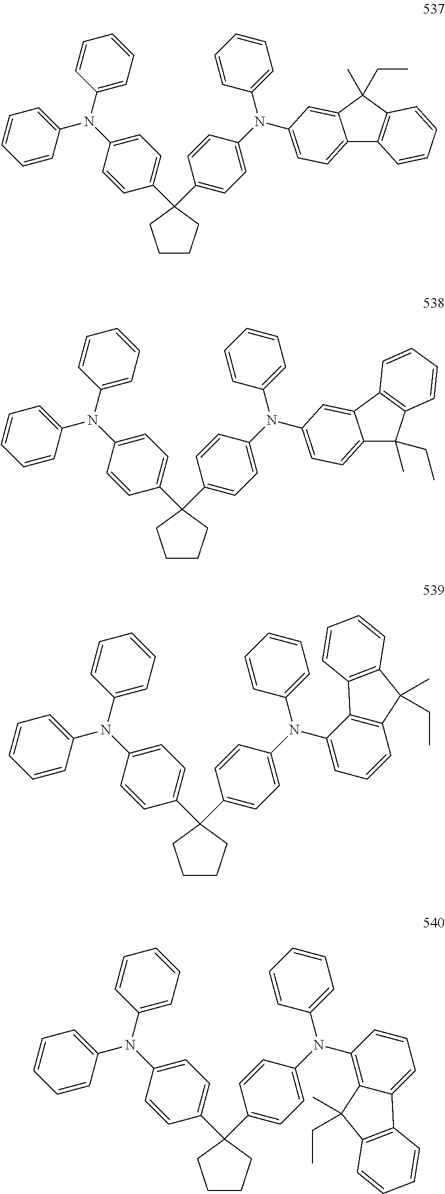 [SMILES: CCC1(C)c2ccccc2-c2ccc(N(c3ccccc3)c3ccc(C4(c5ccc(N(c6ccccc6)c6ccccc6)cc5)CCCC4)cc3)cc21 ; CCC1(C)c2ccccc2-c2cc(N(c3ccccc3)c3ccc(C4(c5ccc(N(c6ccccc6)c6ccccc6)cc5)CCCC4)cc3)ccc21 ; CCC1(C)c2ccccc2-c2c(N(c3ccccc3)c3ccc(C4(c5ccc(N(c6ccccc6)c6ccccc6)cc5)CCCC4)cc3)cccc21 ; CCC1(C)c2ccccc2-c2cccc(N(c3ccccc3)c3ccc(C4(c5ccc(N(c6ccccc6)c6ccccc6)cc5)CCCC4)cc3)c21]

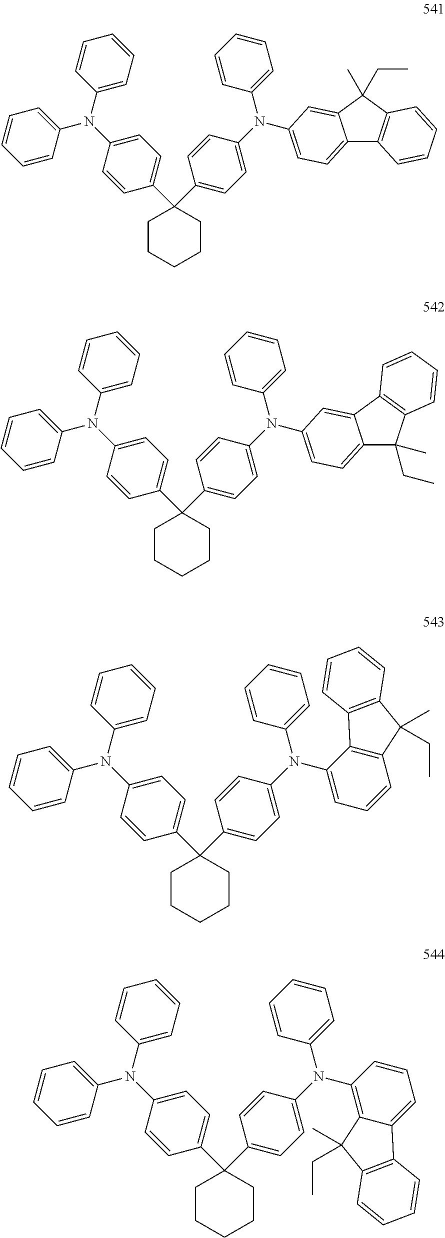 [SMILES: CCC1(C)c2ccccc2-c2ccc(N(c3ccccc3)c3ccc(C4(c5ccc(N(c6ccccc6)c6ccccc6)cc5)CCCCC4)cc3)cc21 ; CCC1(C)c2ccccc2-c2cc(N(c3ccccc3)c3ccc(C4(c5ccc(N(c6ccccc6)c6ccccc6)cc5)CCCCC4)cc3)ccc21 ; CCC1(C)c2ccccc2-c2c(N(c3ccccc3)c3ccc(C4(c5ccc(N(c6ccccc6)c6ccccc6)cc5)CCCCC4)cc3)cccc21 ; CCC1(C)c2ccccc2-c2cccc(N(c3ccccc3)c3ccc(C4(c5ccc(N(c6ccccc6)c6ccccc6)cc5)CCCCC4)cc3)c21]

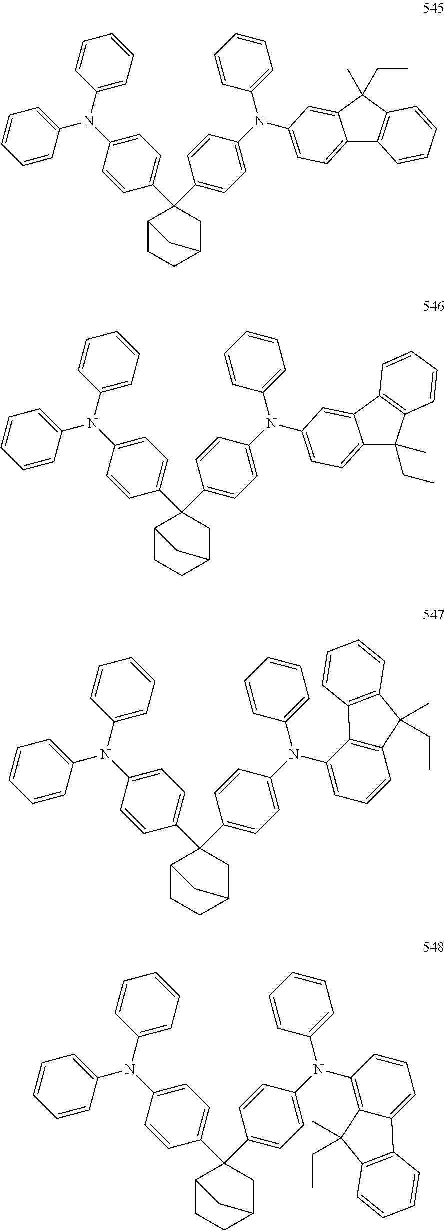 [SMILES: CCC1(C)c2ccccc2-c2ccc(N(c3ccccc3)c3ccc(C4(c5ccc(N(c6ccccc6)c6ccccc6)cc5)CC5CCC4C5)cc3)cc21 ; CCC1(C)c2ccccc2-c2cc(N(c3ccccc3)c3ccc(C4(c5ccc(N(c6ccccc6)c6ccccc6)cc5)CC5CCC4C5)cc3)ccc21 ; CCC1(C)c2ccccc2-c2c(N(c3ccccc3)c3ccc(C4(c5ccc(N(c6ccccc6)c6ccccc6)cc5)CC5CCC4C5)cc3)cccc21 ; CCC1(C)c2ccccc2-c2cccc(N(c3ccccc3)c3ccc(C4(c5ccc(N(c6ccccc6)c6ccccc6)cc5)CC5CCC4C5)cc3)c21]

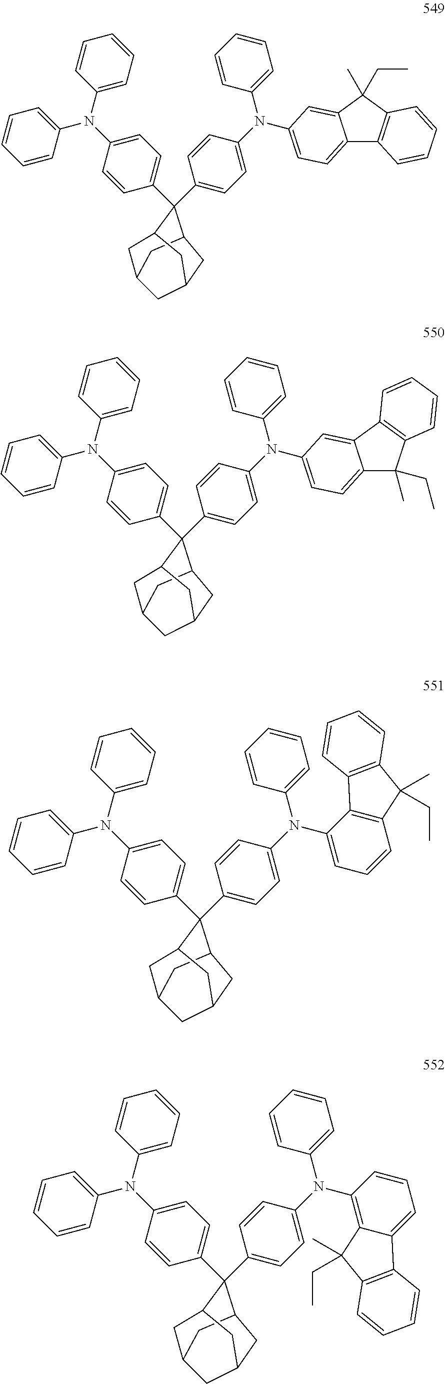 [SMILES: CCC1(C)c2ccccc2-c2ccc(N(c3ccccc3)c3ccc(C4(c5ccc(N(c6ccccc6)c6ccccc6)cc5)C5CC6CC(C5)CC4C6)cc3)cc21 ; CCC1(C)c2ccccc2-c2cc(N(c3ccccc3)c3ccc(C4(c5ccc(N(c6ccccc6)c6ccccc6)cc5)C5CC6CC(C5)CC4C6)cc3)ccc21 ; CCC1(C)c2ccccc2-c2c(N(c3ccccc3)c3ccc(C4(c5ccc(N(c6ccccc6)c6ccccc6)cc5)C5CC6CC(C5)CC4C6)cc3)cccc21 ; CCC1(C)c2ccccc2-c2cccc(N(c3ccccc3)c3ccc(C4(c5ccc(N(c6ccccc6)c6ccccc6)cc5)C5CC6CC(C5)CC4C6)cc3)c21]

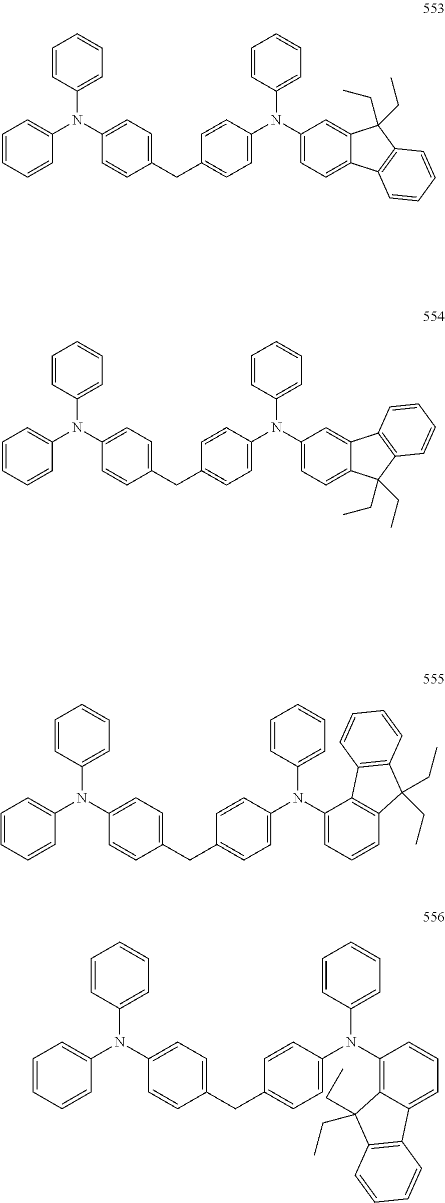 [SMILES: CCC1(CC)c2ccccc2-c2ccc(N(c3ccccc3)c3ccc(Cc4ccc(N(c5ccccc5)c5ccccc5)cc4)cc3)cc21 ; CCC1(CC)c2ccccc2-c2cc(N(c3ccccc3)c3ccc(Cc4ccc(N(c5ccccc5)c5ccccc5)cc4)cc3)ccc21 ; CCC1(CC)c2ccccc2-c2c(N(c3ccccc3)c3ccc(Cc4ccc(N(c5ccccc5)c5ccccc5)cc4)cc3)cccc21 ; CCC1(CC)c2ccccc2-c2cccc(N(c3ccccc3)c3ccc(Cc4ccc(N(c5ccccc5)c5ccccc5)cc4)cc3)c21]

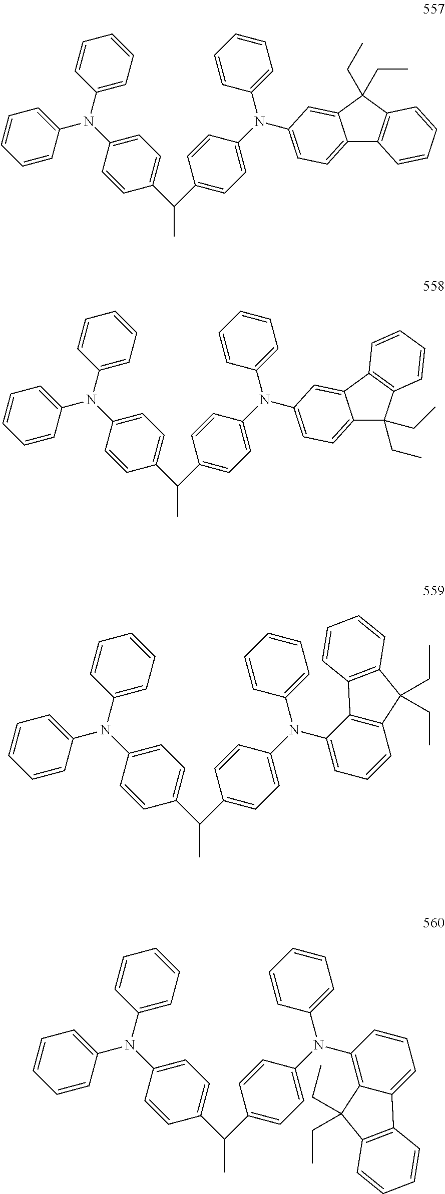 [SMILES: CCC1(CC)c2ccccc2-c2ccc(N(c3ccccc3)c3ccc(C(C)c4ccc(N(c5ccccc5)c5ccccc5)cc4)cc3)cc21 ; CCC1(CC)c2ccccc2-c2cc(N(c3ccccc3)c3ccc(C(C)c4ccc(N(c5ccccc5)c5ccccc5)cc4)cc3)ccc21 ; CCC1(CC)c2ccccc2-c2c(N(c3ccccc3)c3ccc(C(C)c4ccc(N(c5ccccc5)c5ccccc5)cc4)cc3)cccc21 ; CCC1(CC)c2ccccc2-c2cccc(N(c3ccccc3)c3ccc(C(C)c4ccc(N(c5ccccc5)c5ccccc5)cc4)cc3)c21]

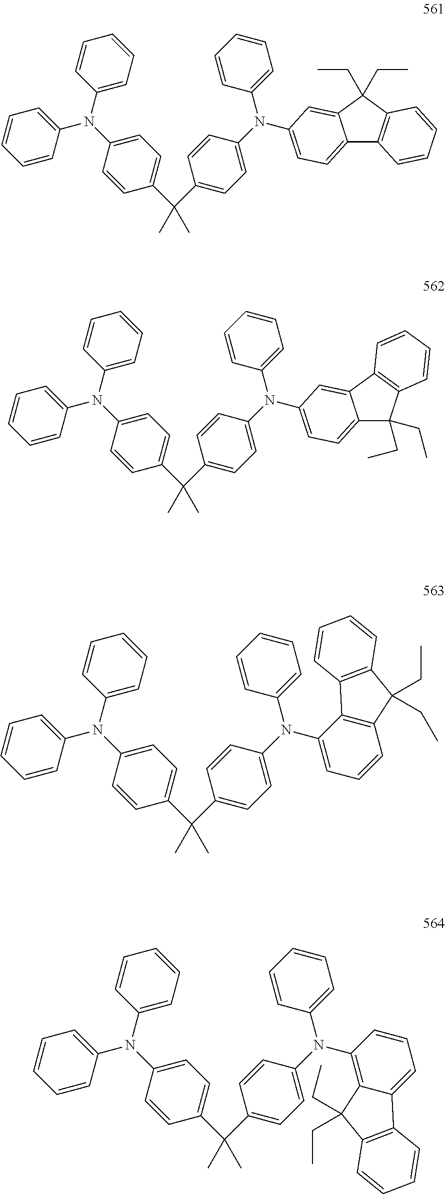 [SMILES: CCC1(CC)c2ccccc2-c2ccc(N(c3ccccc3)c3ccc(C(C)(C)c4ccc(N(c5ccccc5)c5ccccc5)cc4)cc3)cc21 ; CCC1(CC)c2ccccc2-c2cc(N(c3ccccc3)c3ccc(C(C)(C)c4ccc(N(c5ccccc5)c5ccccc5)cc4)cc3)ccc21 ; CCC1(CC)c2ccccc2-c2c(N(c3ccccc3)c3ccc(C(C)(C)c4ccc(N(c5ccccc5)c5ccccc5)cc4)cc3)cccc21 ; CCC1(CC)c2ccccc2-c2cccc(N(c3ccccc3)c3ccc(C(C)(C)c4ccc(N(c5ccccc5)c5ccccc5)cc4)cc3)c21]

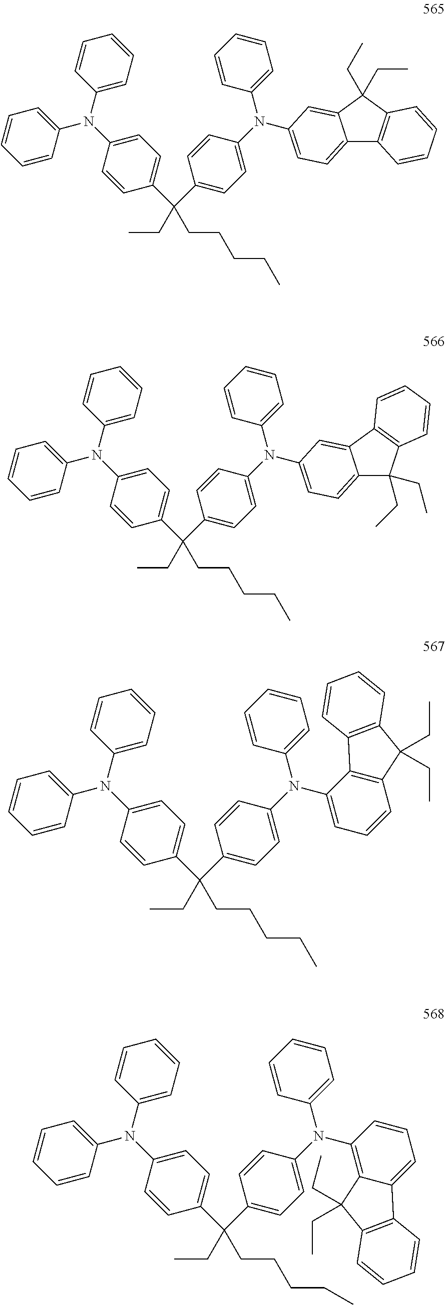 [SMILES: CCCCCC(CC)(c1ccc(N(c2ccccc2)c2ccccc2)cc1)c1ccc(N(c2ccccc2)c2ccc3c(c2)C(CC)(CC)c2ccccc2-3)cc1 ; CCCCCC(CC)(c1ccc(N(c2ccccc2)c2ccccc2)cc1)c1ccc(N(c2ccccc2)c2ccc3c(c2)-c2ccccc2C3(CC)CC)cc1 ; CCCCCC(CC)(c1ccc(N(c2ccccc2)c2ccccc2)cc1)c1ccc(N(c2ccccc2)c2cccc3c2-c2ccccc2C3(CC)CC)cc1 ; CCCCCC(CC)(c1ccc(N(c2ccccc2)c2ccccc2)cc1)c1ccc(N(c2ccccc2)c2cccc3c2C(CC)(CC)c2ccccc2-3)cc1]

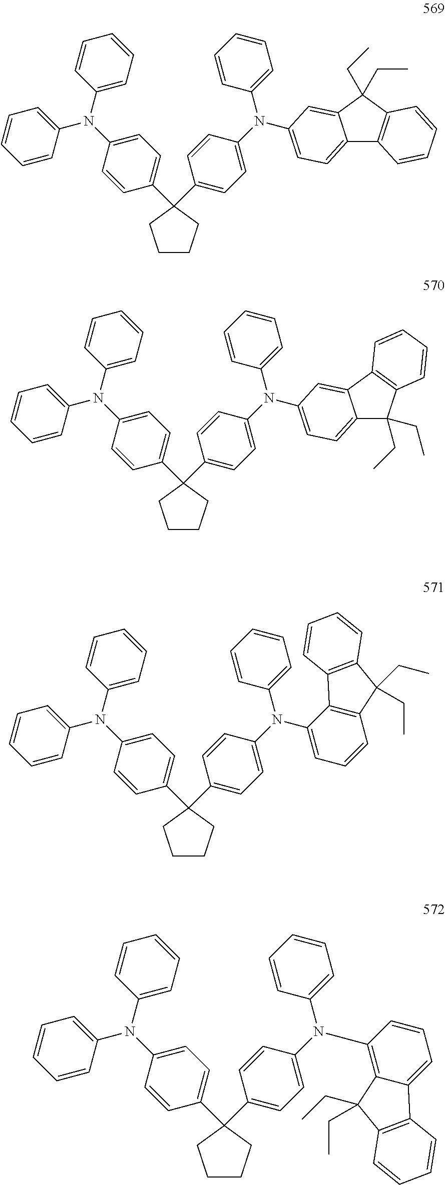

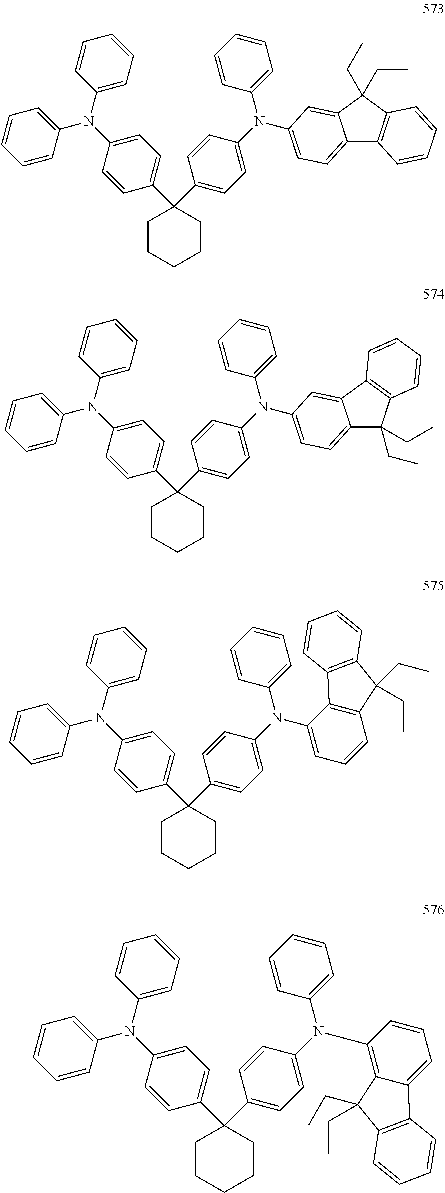 [SMILES: CCC1(CC)c2ccccc2-c2ccc(N(c3ccccc3)c3ccc(C4(c5ccc(N(c6ccccc6)c6ccccc6)cc5)CCCCC4)cc3)cc21 ; CCC1(CC)c2ccccc2-c2cc(N(c3ccccc3)c3ccc(C4(c5ccc(N(c6ccccc6)c6ccccc6)cc5)CCCCC4)cc3)ccc21 ; CCC1(CC)c2ccccc2-c2c(N(c3ccccc3)c3ccc(C4(c5ccc(N(c6ccccc6)c6ccccc6)cc5)CCCCC4)cc3)cccc21 ; CCC1(CC)c2ccccc2-c2cccc(N(c3ccccc3)c3ccc(C4(c5ccc(N(c6ccccc6)c6ccccc6)cc5)CCCCC4)cc3)c21]

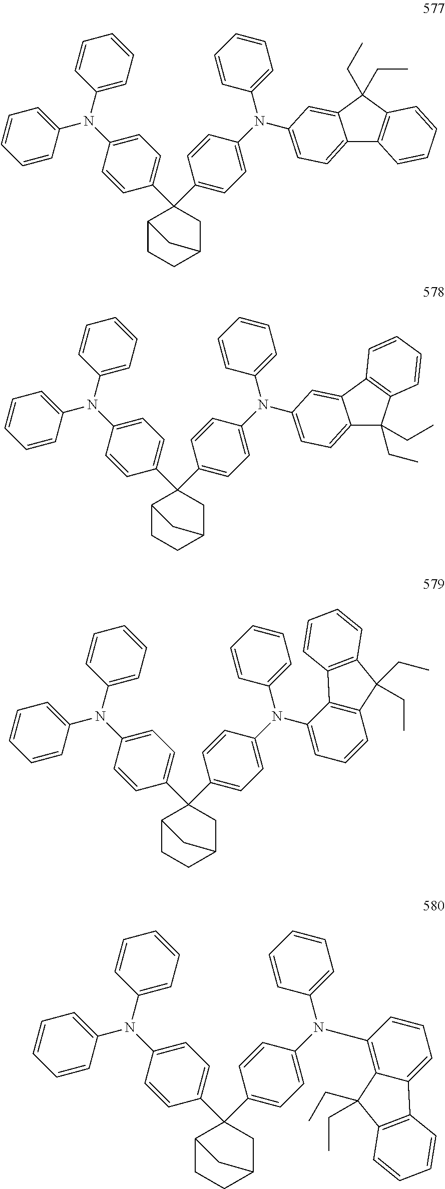 [SMILES: CCC1(CC)c2ccccc2-c2ccc(N(c3ccccc3)c3ccc(C4(c5ccc(N(c6ccccc6)c6ccccc6)cc5)CC5CCC4C5)cc3)cc21 ; CCC1(CC)c2ccccc2-c2cc(N(c3ccccc3)c3ccc(C4(c5ccc(N(c6ccccc6)c6ccccc6)cc5)CC5CCC4C5)cc3)ccc21 ; CCC1(CC)c2ccccc2-c2c(N(c3ccccc3)c3ccc(C4(c5ccc(N(c6ccccc6)c6ccccc6)cc5)CC5CCC4C5)cc3)cccc21 ; CCC1(CC)c2ccccc2-c2cccc(N(c3ccccc3)c3ccc(C4(c5ccc(N(c6ccccc6)c6ccccc6)cc5)CC5CCC4C5)cc3)c21]

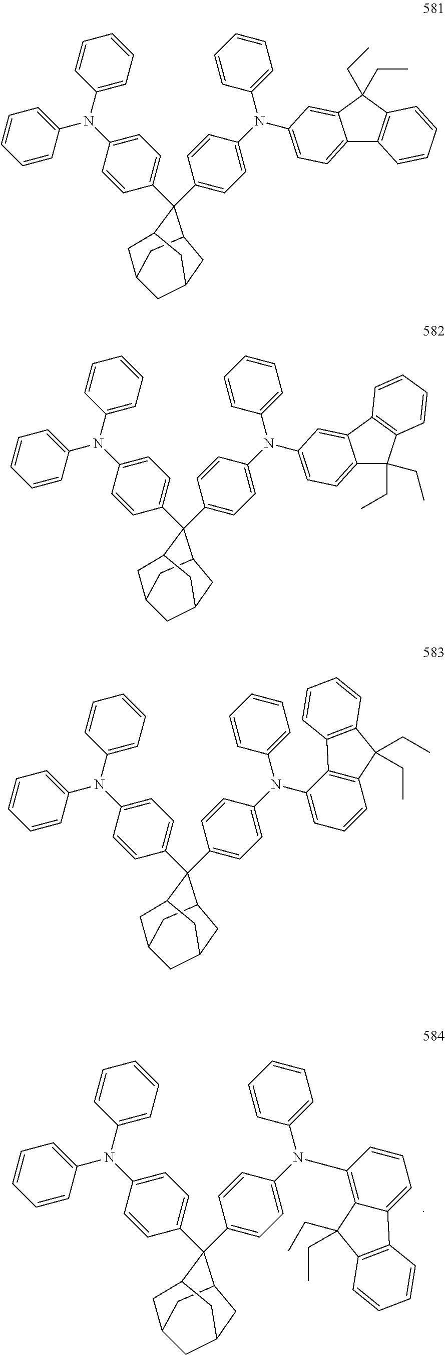

In the amine compound represented by Formula 1, a band gap may be widened by applying a structure in which two amines are linked, and a glass transition temperature may be significantly increased by increasing a molecular weight of the amine compound. In addition, because a linking group linking the two amines includes an alkane group (see the group represented by *-(L₁)_{c1}-*′ in Formula 1), the amine compound represented by Formula 1 may have low refractive characteristics.

In addition, in the amine compound, physical and/or chemical properties may be controlled by changing substituents. Because the amine compound has a tricyclic group, such as a fluorene group, a carbazole group, or the like (see T₁ in Formula 1 and the group represented by Formula 2), polarons may be stabilized to provide a long lifespan in a light-emitting device, and a hopping rate of charges may be increased to improve charge mobility.

In addition, because the two amines linked via the alkane group have an asymmetric structure (in Formula 1, the group represented by *—(Ar₄)ₐ₄-T₁ is different from the group represented by *—(Ar₁)ₐ₁—(W₁)_{b1} and the group represented by *—(Ar₂)ₐ₂—(W₂)_{b2}), hole injection characteristics may be improved through an increase in energy disorder, compared to a case in which the two amines have a symmetric structure (for example, see Compounds B to F provided herein).

Accordingly, by using the amine compound represented by Formula 1, an electronic device (for example, an organic light-emitting device) having high efficiency, low voltage, high luminance, and/or long lifespan may be implemented.

Synthesis methods of the amine compound represented by Formula 1 may be recognizable by one of ordinary skill in the art by referring to Synthesis Examples and/or Examples provided below.

At least one amine compound represented by Formula 1 may be used in a light-emitting device (for example, an organic light-emitting device).

In an embodiment, provided is a light-emitting device including: a first electrode; a second electrode facing the first electrode; an interlayer between the first electrode and the second electrode and including an emission layer; and the amine compound represented by Formula 1 as described herein.

In an embodiment,

the first electrode of the light-emitting device may be an anode,

the second electrode of the light-emitting device may be a cathode,

the interlayer may further include a hole transport region between the first electrode and the emission layer and an electron transport region between the emission layer and the second electrode,

the hole transport region may include a hole injection layer, a hole transport layer, an emission auxiliary layer, an electron blocking layer, or any combination thereof, and

the electron transport region may include a buffer layer, a hole blocking layer, an electron control layer, an electron transport layer, an electron injection layer, or any combination thereof.

In an embodiment, the amine compound may be included between the first electrode and the second electrode of the light-emitting device. The amine compound may be included in the interlayer of the light-emitting device, for example, in at least one selected from the hole transport region, the electron transport region, and the emission layer of the interlayer.

In an embodiment, the amine compound represented by Formula 1 may be included in the hole transport region of the interlayer.

In an embodiment, the hole transport region may include at least one selected from a hole injection layer, a hole transport layer, and an electron blocking layer, and

the at least one selected from the hole injection layer, the hole transport layer, and the electron blocking layer may include the amine compound represented by Formula 1.

For example, the hole transport layer may include the amine compound represented by Formula 1.

In an embodiment, the hole transport region of the light-emitting device may further include a p-dopant having a lowest unoccupied molecular orbital (LUMO) energy level of −3.5 eV or less.

For example, the p-dopant may include at least one selected from a quinone derivative, a metal oxide, and a cyano group-containing compound.

For example, the hole transport region may include a hole transport layer, and the hole transport layer may include the p-dopant, or the hole transport region may include a hole injection layer, and the hole injection layer may include the p-dopant.

In an embodiment, the emission layer may include a host and a dopant. For example, the host may include an anthracene compound, but is not limited thereto, and the dopant may include an arylamine compound and/or a styrylaryl compound, but is not limited thereto. In this case, an amount of the host may be greater than an amount of the dopant. For example, the amount of the dopant in the emission layer may be in a range of about 0.01 part by weight to about 5 parts by weight, or about 0.01 part by weight to about 3 parts by weight, based on 100 parts by weight of the total weight of the host and the dopant, but embodiments are not limited thereto.

The emission layer may emit red light, green light, blue light, and/or white light. For example, the emission layer may emit blue light. The blue light may have a maximum emission wavelength in a range of, for example, about 400 nm to about 490 nm, or about 430 nm to about 490 nm.

In one or more embodiments, the light-emitting device may include a capping layer outside the first electrode and/or outside the second electrode.

For example, the light-emitting device may further include at least one selected from a first capping layer outside the first electrode and a second capping layer outside the second electrode, and at least one selected from the first capping layer and the second capping layer may include the amine compound represented by Formula 1. The first capping layer and/or the second capping layer are respectively the same as those described herein.

In an embodiment, the light-emitting device may include:

a first capping layer outside the first electrode and including the amine compound represented by Formula 1;

a second capping layer outside the second electrode and including the amine compound represented by Formula 1; or

the first capping layer and the second capping layer.

The wording “(interlayer and/or capping layer) includes an amine compound,” as used herein, may be understood as “(interlayer and/or capping layer) may include one kind of amine compound represented by Formula 1 or two different kinds of amine compounds, each represented by Formula 1”.

In an embodiment, the interlayer and/or the capping layer may include Compound 1 only as the amine compound. In this regard, Compound 1 may be present in the emission layer of the light-emitting device. In one or more embodiments, the interlayer may include, as the amine compound, Compound 1 and Compound 2. In this regard, Compound 1 and Compound 2 may be present in the same layer (for example, both Compound 1 and Compound 2 may be present in the emission layer), or may be present in different layers (for example, Compound 1 may be present in the emission layer, and Compound 2 may be present in the hole transport region).

The term “interlayer,” as used herein, refers to a single layer and/or all of a plurality of layers between the first electrode and the second electrode of the light-emitting device.

Another aspect of embodiments of the present disclosure provides an electronic apparatus including the light-emitting device. The electronic apparatus may further include a thin-film transistor. For example, the electronic apparatus may further include a thin-film transistor including a source electrode and a drain electrode, wherein the first electrode of the light-emitting device may be electrically connected to the source electrode or the drain electrode. In one or more embodiments, the electronic apparatus may further include a color filter, a color conversion layer, a touch screen layer, a polarizing layer, or any combination thereof. The electronic apparatus is the same as described herein.

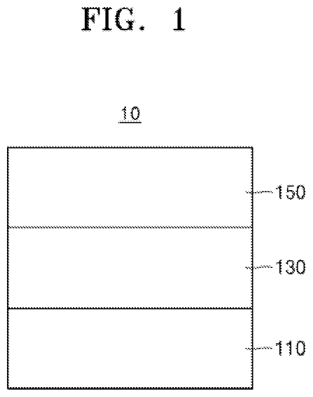

### Description of FIG. 1

FIG. 1 is a schematic cross-sectional view of a light-emitting device 10 according to an embodiment. The light-emitting device 10 includes a first electrode 110, an interlayer 130, and a second electrode 150.

Hereinafter, the structure of the light-emitting device 10 according to an embodiment and a method of manufacturing the light-emitting device 10 will be described with reference to FIG. 1.

### First Electrode 110

In FIG. 1, a substrate may be additionally under the first electrode 110 and/or on the second electrode 150. As the substrate, a glass substrate and/or a plastic substrate may be used. In one or more embodiments, the substrate may be a flexible substrate, and may include plastics having excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing and/or sputtering a material for forming the first electrode 110 on the substrate. When the first electrode 110 is an anode, the material for forming the first electrode 110 may be a high work function material that facilitates injection of holes.

The first electrode 110 may be a reflective electrode, a semi-transmissive electrode, or a transmissive electrode. When the first electrode 110 is a transmissive electrode, the material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, the material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure consisting of a single layer or a multi-layered structure including a plurality of layers. For example, the first electrode 110 may have a three-layered structure of ITO/Ag/ITO.

### Interlayer 130

The interlayer 130 is on the first electrode 110. The interlayer 130 includes an emission layer.

The interlayer 130 may further include a hole transport region between the first electrode 110 and the emission layer and an electron transport region between the emission layer and the second electrode 150.

The interlayer 130 may further include, in addition to various suitable organic materials, a metal-containing compound, such as an organometallic compound, an inorganic material, such as a quantum dot, and/or the like.

In one or more embodiments, interlayer 130 may include, i) two or more emitting units sequentially stacked between the first electrode 110 and the second electrode 150, and ii) a charge generation layer between the two or more emitting units. When the interlayer 130 includes the emitting units and the charge generation layer as described above, the light-emitting device 10 may be a tandem light-emitting device.

### Hole Transport Region in Interlayer 130

The hole transport region may have i) a single-layered structure consisting of a single layer consisting of a single material, ii) a single-layered structure consisting of a single layer consisting of a plurality of different materials, or iii) a multi-layered structure including a plurality of layers including different materials.

The hole transport region may include a hole injection layer, a hole transport layer, an emission auxiliary layer, an electron blocking layer, or any combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, the layers of each structure being stacked sequentially from the first electrode 110.

The hole transport region may include a compound represented by Formula 201, a compound represented by Formula 202, or any combination thereof:

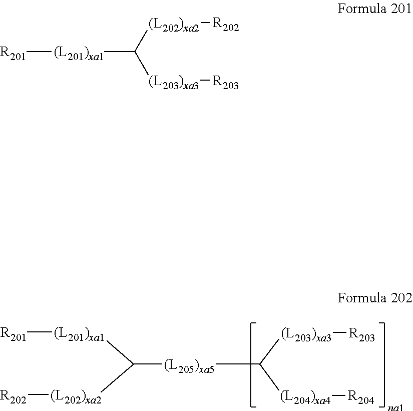

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently be an integer from 0 to 5,

xa5 may be an integer from 1 to 10,

R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (for example, a carbazole group, etc.) unsubstituted or substituted with at least one R₁₀ₐ (for example, see Compound HT16),

R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and

na1 may be an integer from 1 to 4.

For example, each of Formulae 201 and 202 may include at least one selected from groups represented by Formulae CY201 to CY217:

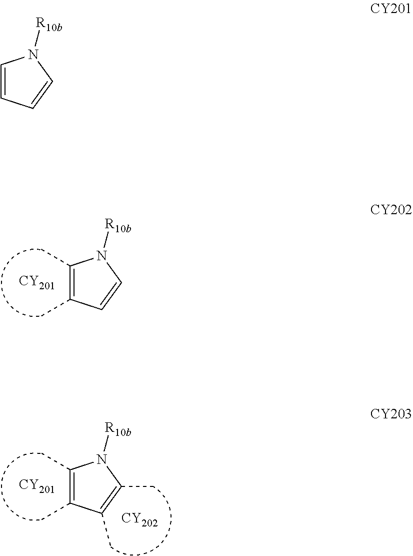

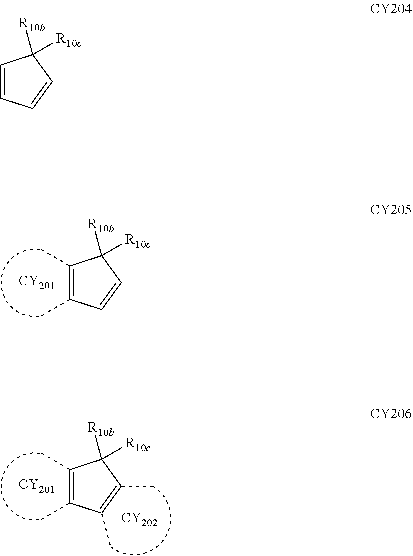

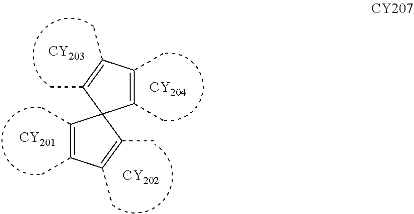

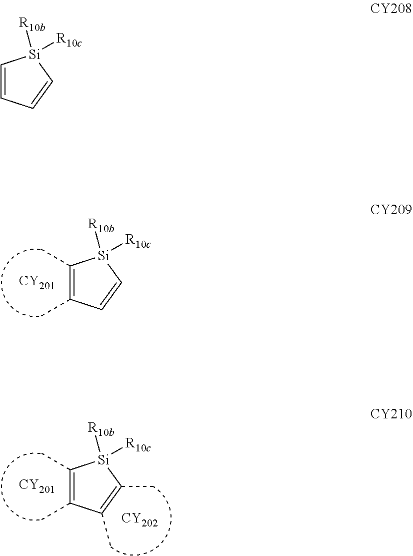

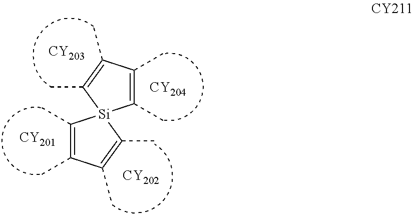

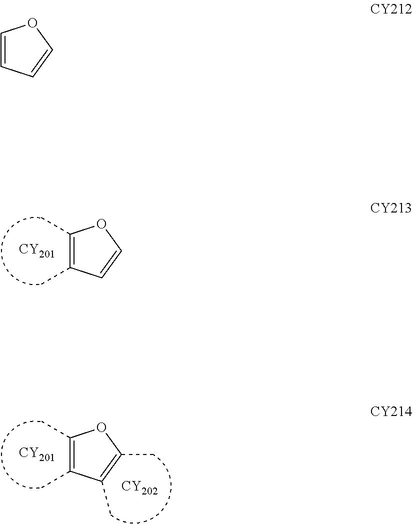

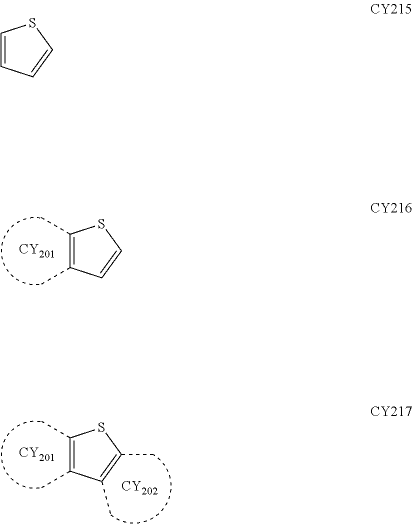

wherein, in Formulae CY201 to CY217, R_{10b} and R_{10c} are each the same as described in connection with R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described herein.

In an embodiment, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one selected from groups represented by Formulae CY201 to CY203.

In one or more embodiments, Formula 201 may include at least one selected from the groups represented by Formulae CY201 to CY203 and at least one selected from the groups represented by Formulae CY204 to CY217.

In one or more embodiments, in Formula 201, xa1 may be 1, R₂₀₁ may be a group represented by one selected from Formulae CY201 to CY203, xa2 may be 0, and R₂₀₂ may be a group represented by one selected from Formulae CY204 to CY207.

In one or more embodiments, each of Formulae 201 and 202 may not include a group represented by one selected from Formulae CY201 to CY203.

In one or more embodiments, each of Formulae 201 and 202 may not include a group represented by one selected from Formulae CY201 to CY203, and may include at least one selected from the groups represented by Formulae CY204 to CY217.

In one or more embodiments, each of Formulae 201 and 202 may not include a group represented by one selected from Formulae CY201 to CY217.

For example, the hole transport region may include one of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

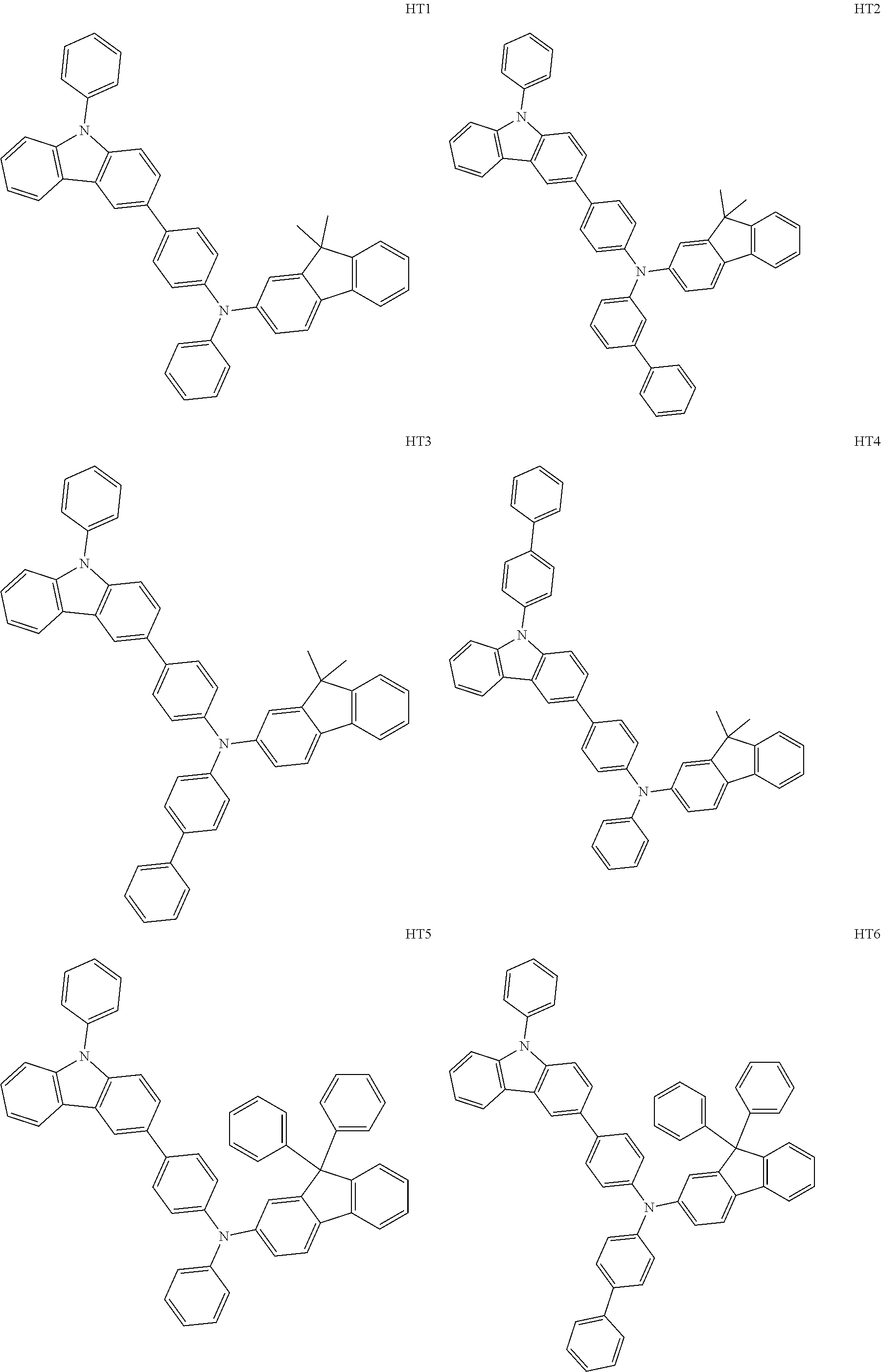

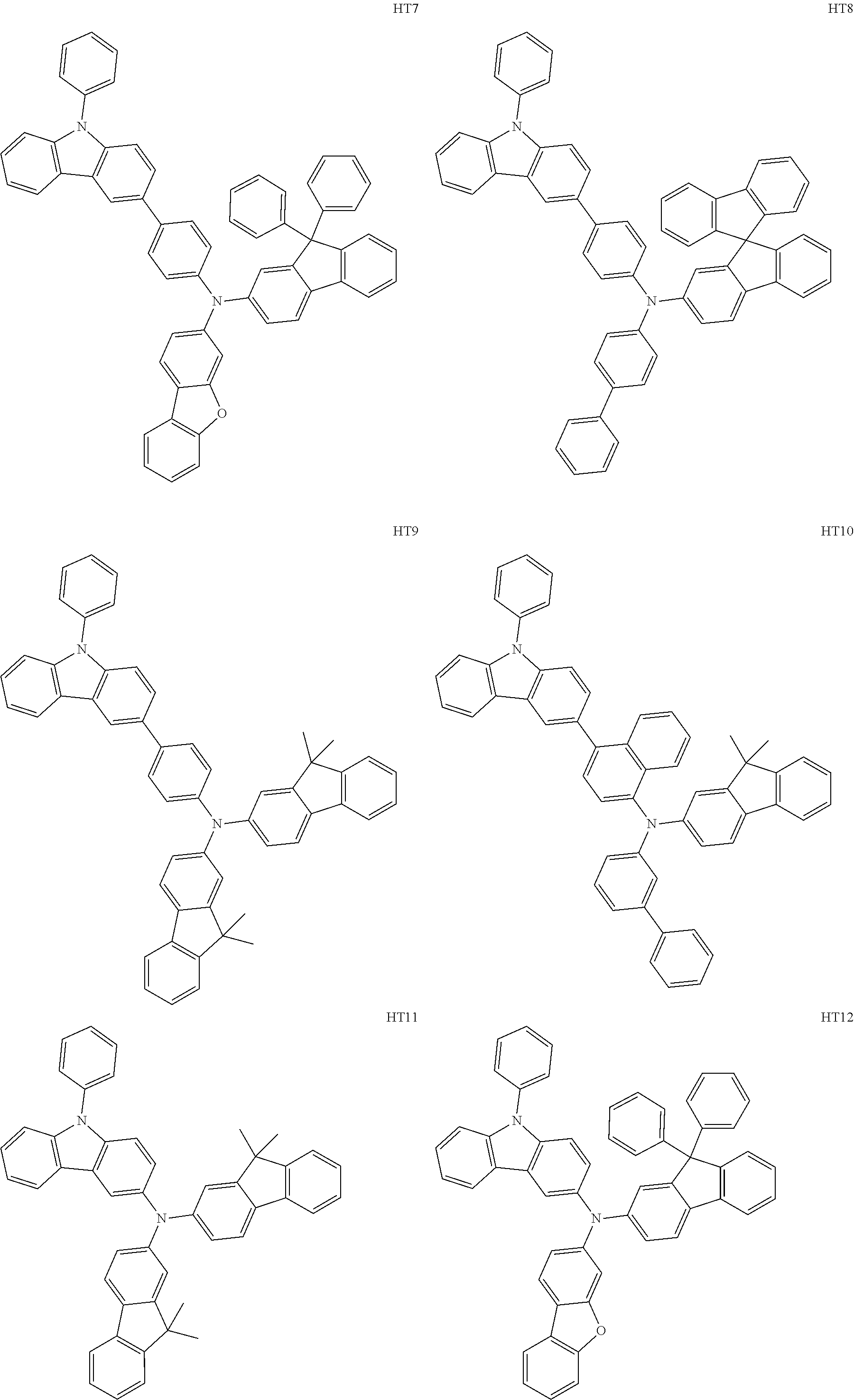

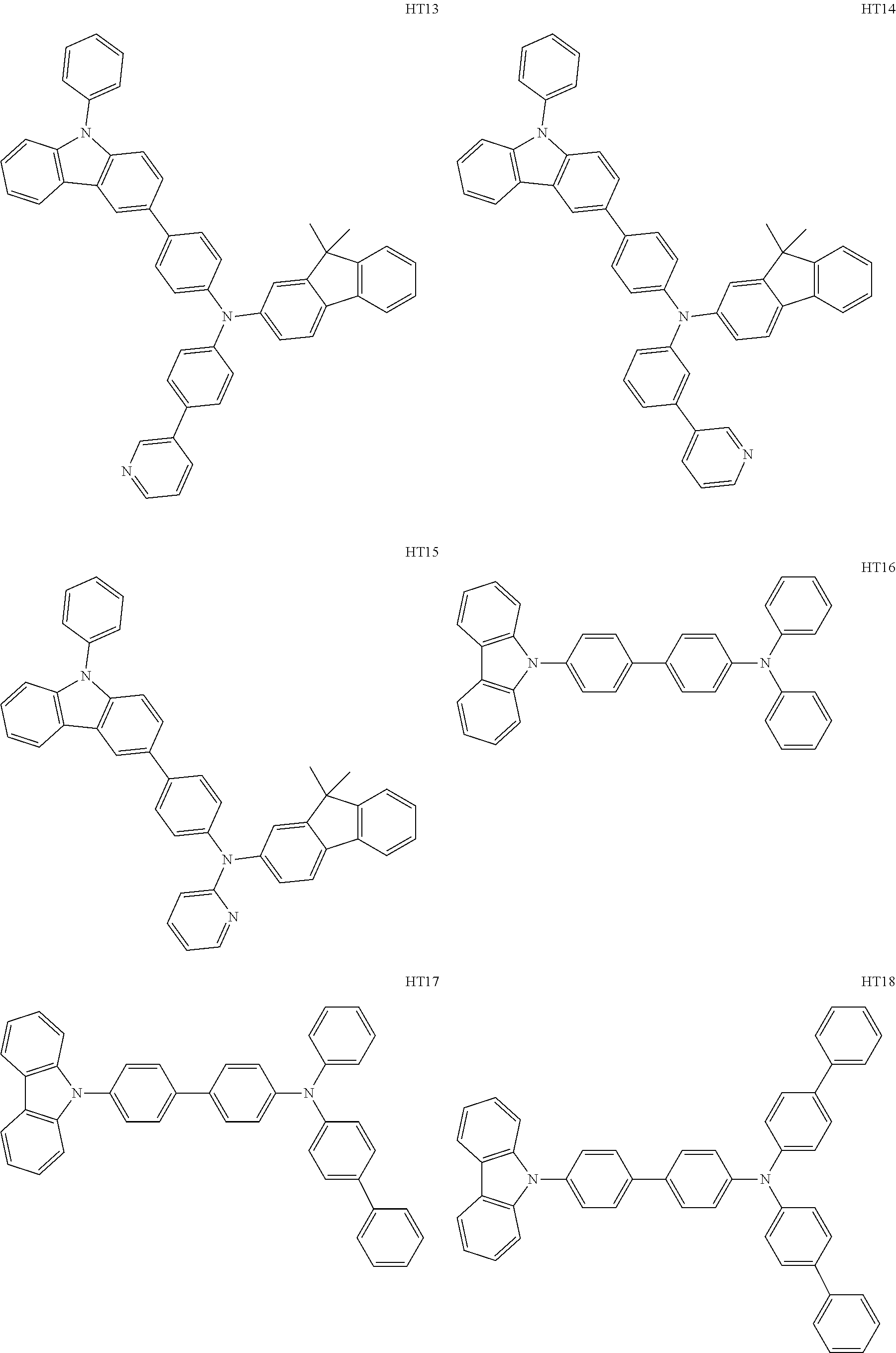

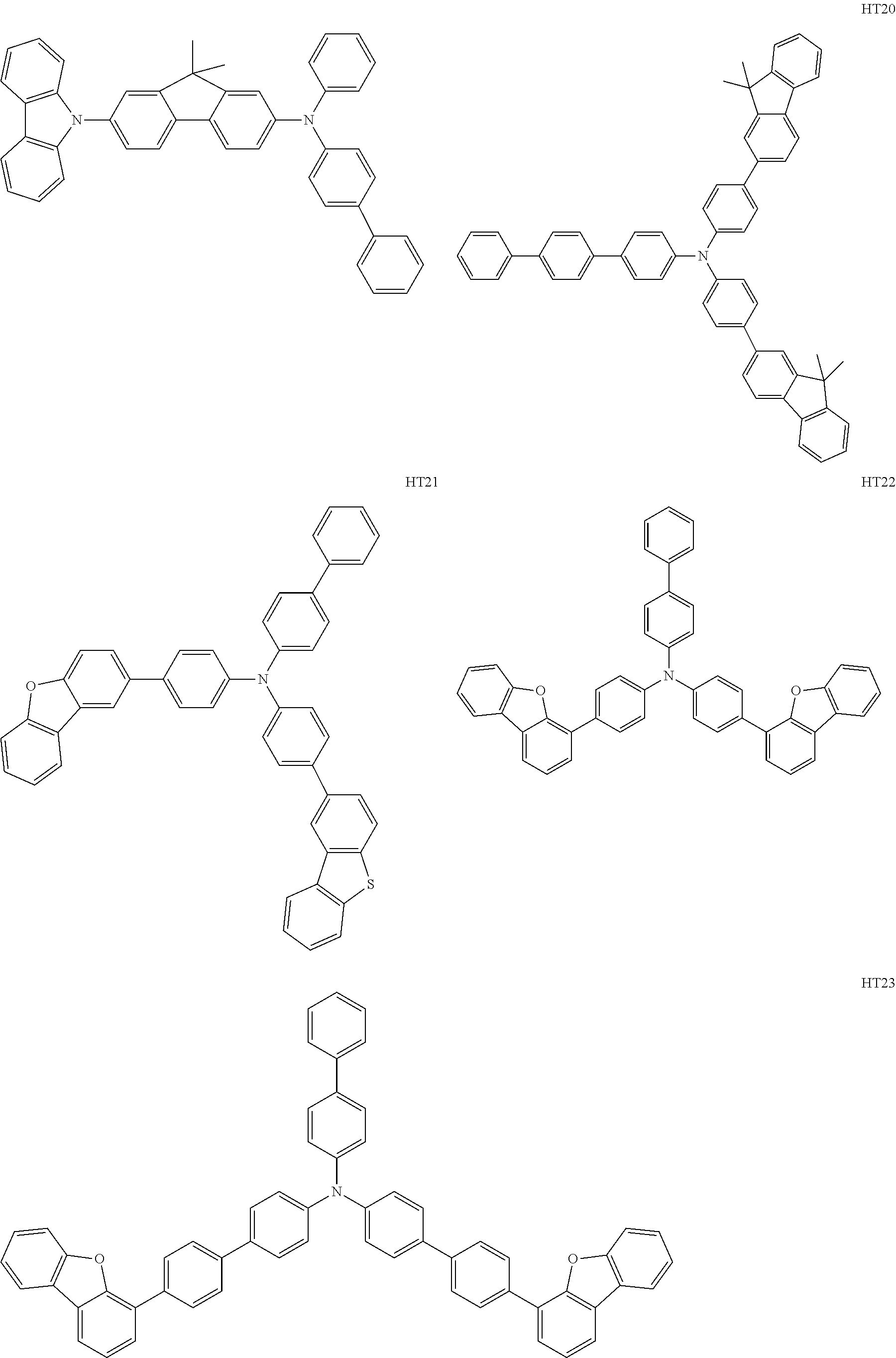

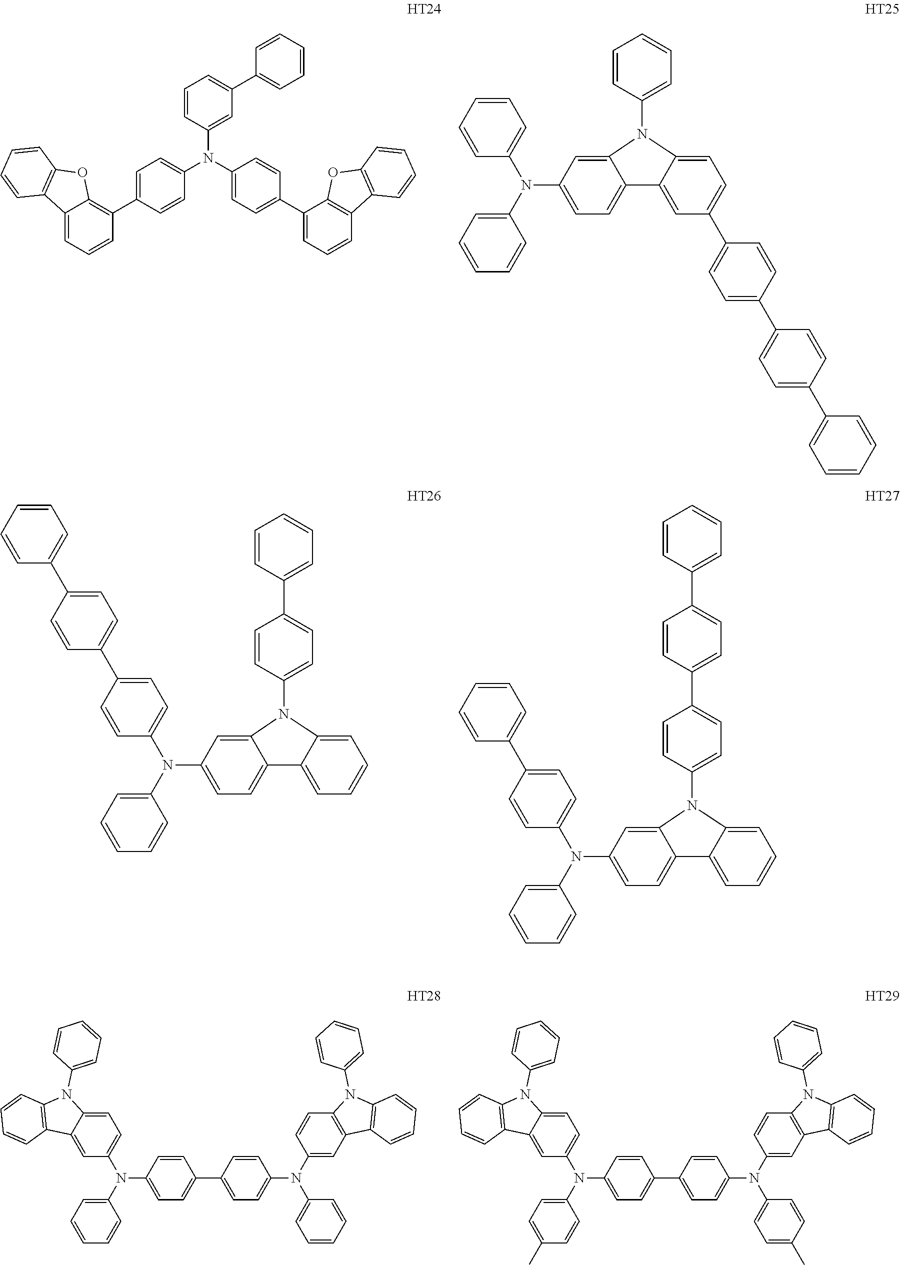

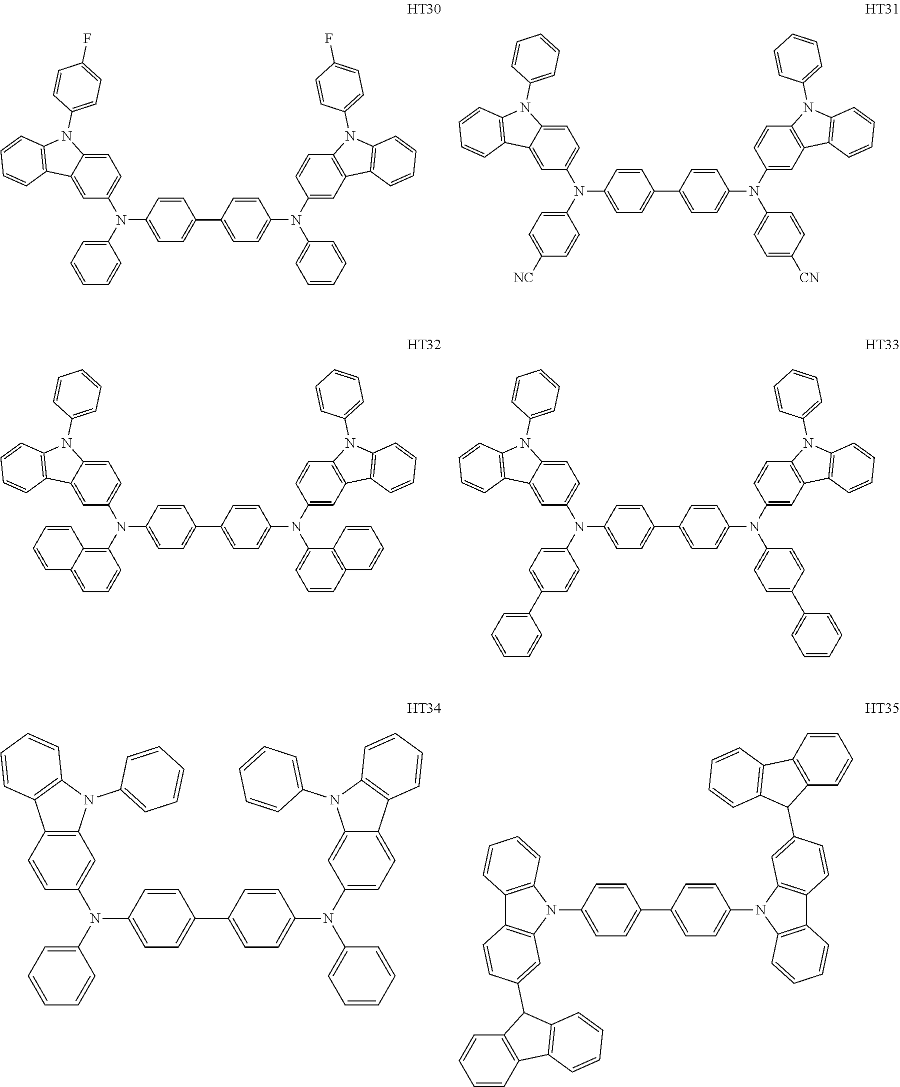

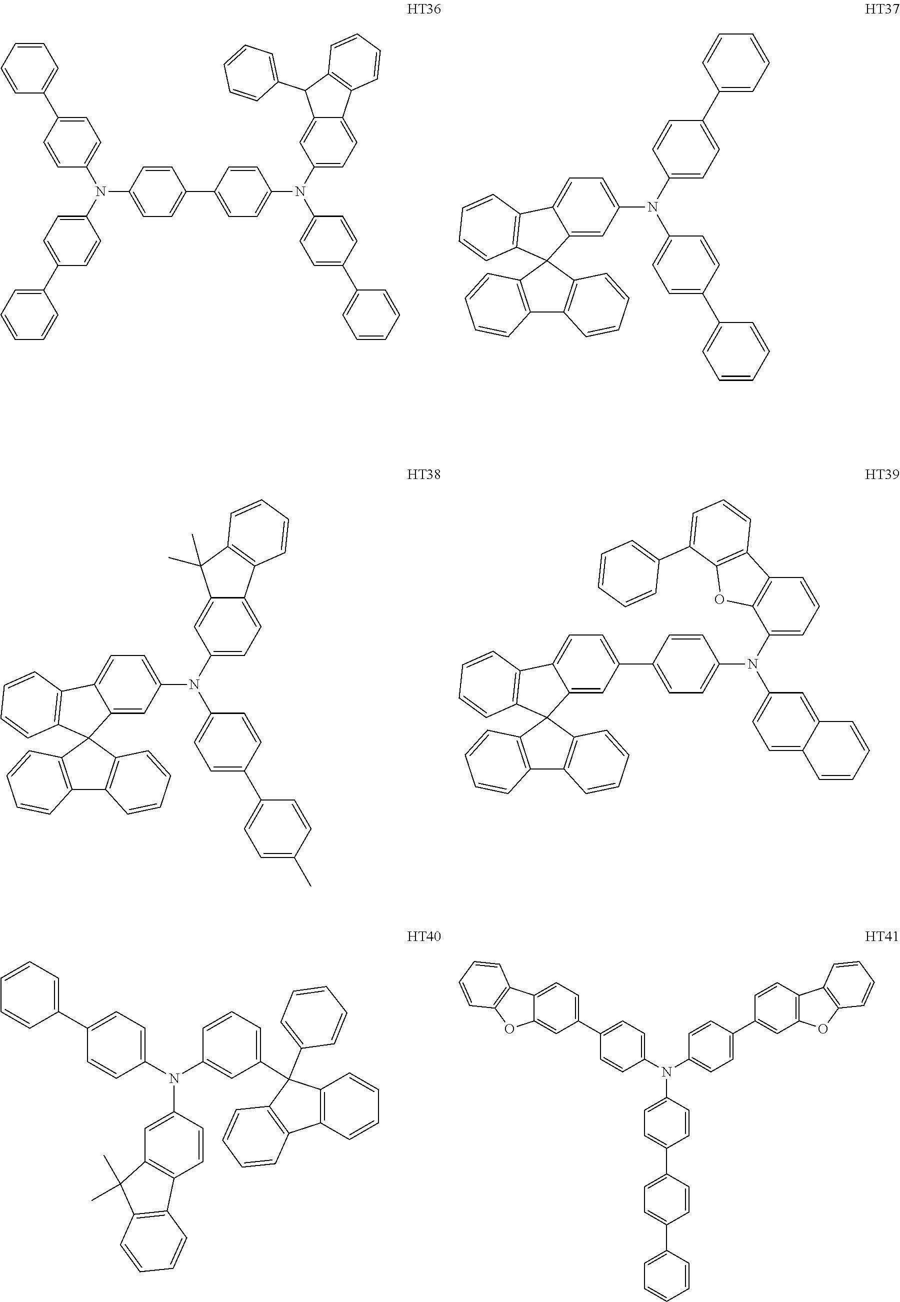

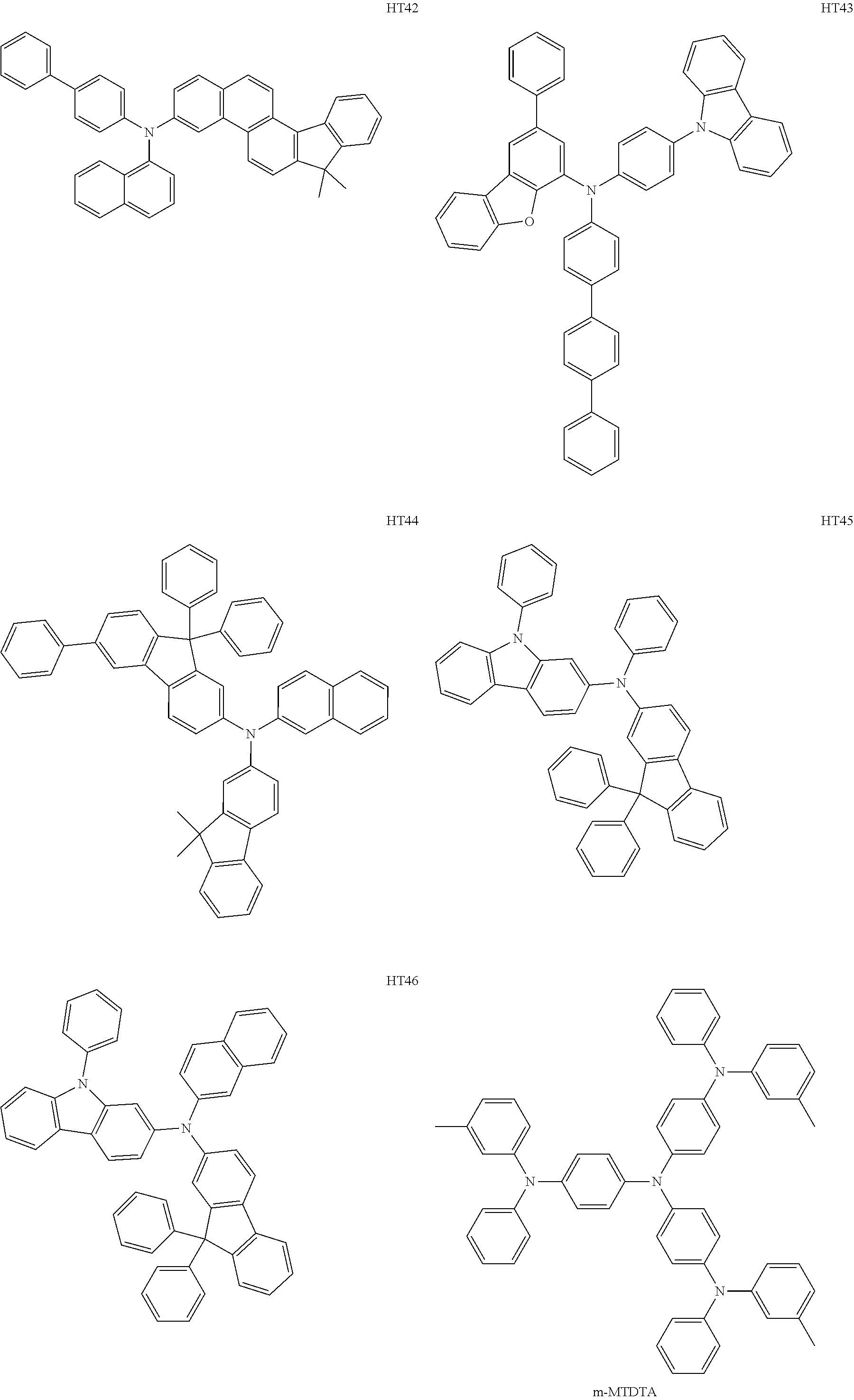

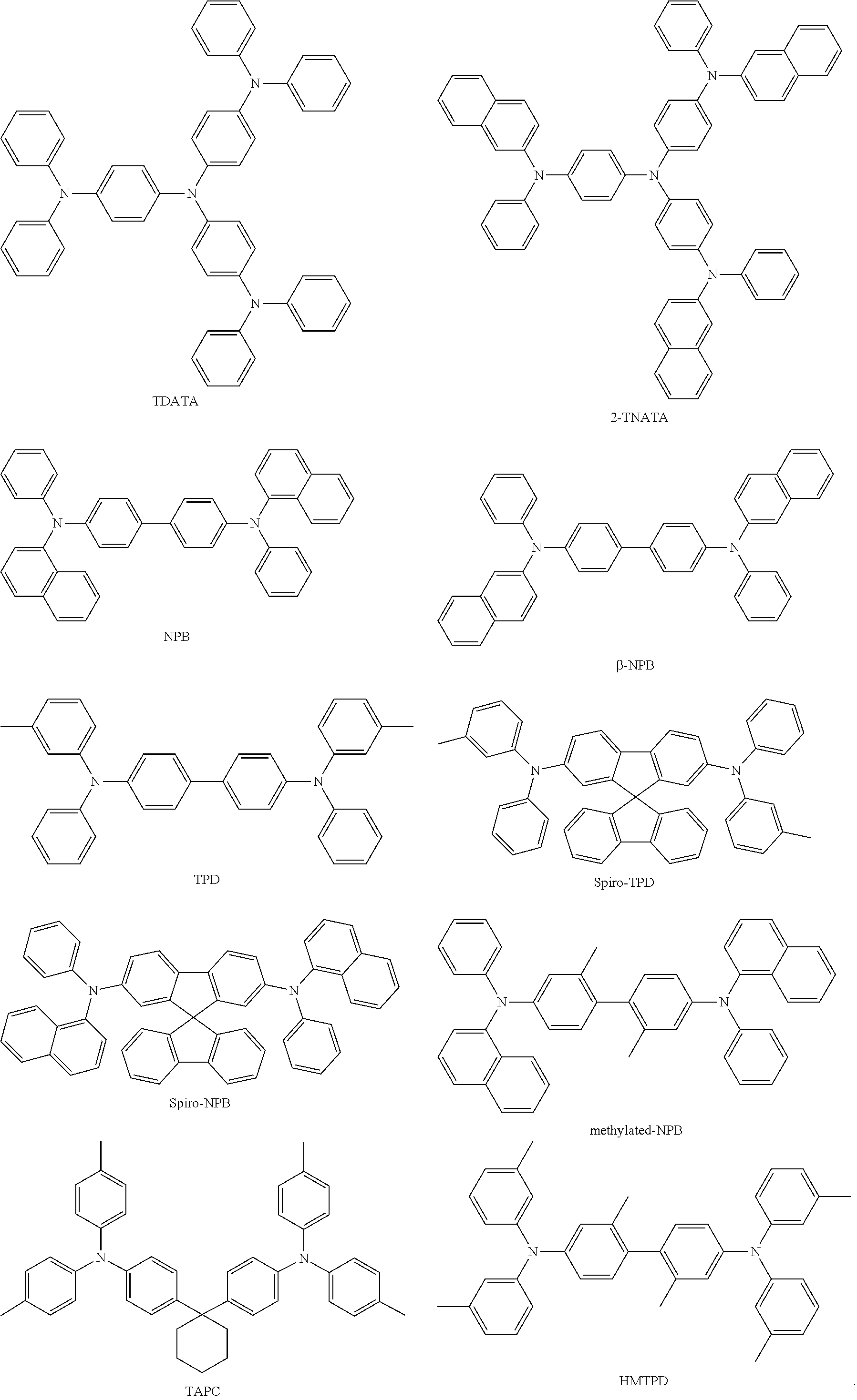

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, suitable or satisfactory hole transporting characteristics may be obtained without a substantial increase in driving voltage.

The emission auxiliary layer may increase light-emission efficiency by compensating for an optical resonance distance according to the wavelength of light emitted by the emission layer, and the electron blocking layer may block or reduce the leakage of electrons from the emission layer to the hole transport region. Materials that may be included in the hole transport region may be included in the emission auxiliary layer and the electron blocking layer.

p-Dopant

The hole transport region may further include, in addition to the materials as described above, a charge-generation material for improving conductive properties (e.g., electrically conductive properties). The charge-generation material may be uniformly or non-uniformly dispersed in the hole transport region (for example, in the form of a single layer consisting of a charge-generation material).

The charge-generation material may be, for example, a p-dopant.

For example, a LUMO energy level of the p-dopant may be −3.5 eV or less.

In an embodiment, the p-dopant may include a quinone derivative, a cyano group-containing compound, a compound containing element EL1 and element EL2, or any combination thereof.

Examples of the quinone derivative may include TCNQ, F4-TCNQ, and the like.

Examples of the cyano group-containing compound may include HAT-CN, and a compound represented by Formula 221:

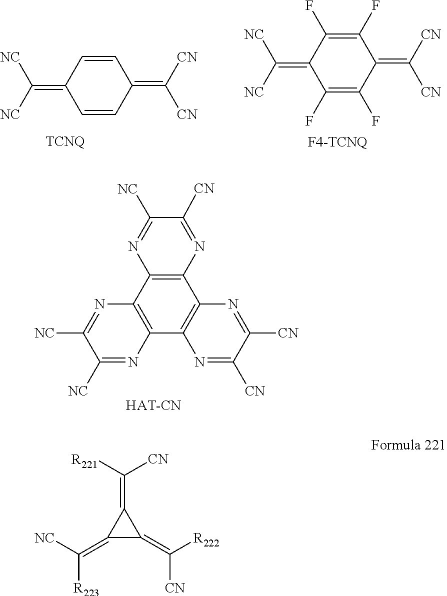

wherein, in Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and

at least one selected from R₂₂₁ to R₂₂₃ may each independently be a C₃-C₆₀ carbocyclic group or a C₁-C₆₀ heterocyclic group, each substituted with a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be metal, metalloid, or any combination thereof, and element EL2 may be non-metal, metalloid, or any combination thereof.

Examples of the metal may include: an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); and a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Examples of the metalloid may include silicon (Si), antimony (Sb), and tellurium (Te).

Examples of the non-metal may include oxygen (O) and halogen (for example, F, Cl, Br, I, etc.).

For example, the compound containing element EL1 and element EL2 may include metal oxide, metal halide (for example, metal fluoride, metal chloride, metal bromide, metal iodide, etc.), metalloid halide (for example, metalloid fluoride, metalloid chloride, metalloid bromide, metalloid iodide, etc.), metal telluride, or any combination thereof.

Examples of the metal oxide may include tungsten oxide (for example, WO, W₂O₃, WO₂, WO₃, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), and rhenium oxide (for example, ReO₃, etc.).

Examples of the metal halide may include alkali metal halide, alkaline earth metal halide, transition metal halide, post-transition metal halide, and lanthanide metal halide.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, and CsI.

Examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, and BaI₂.

Examples of the transition metal halide may include titanium halide (for example, TiF₄, TiCl₄, TiBr₄, Til₄, etc.), zirconium halide (for example, ZrF₄, ZrCl₄, ZrBr₄, ZrI₄, etc.), hafnium halide (for example, HfF₄, HfCl₄, HfBr₄, HfI₄, etc.), vanadium halide (for example, VF₃, VCl₃, VBr₃, VI₃, etc.), niobium halide (for example, NbF₃, NbCl₃, NbBr₃, NbI₃, etc.), tantalum halide (for example, TaF₃, TaCl₃, TaBr₃, TaI₃, etc.), chromium halide (for example, CrF₃, CrO₃, CrBr₃, CrI₃, etc.), molybdenum halide (for example, MoF₃, MoCl₃, MoBr₃, MoI₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), iron halide (for example, FeF₂, FeCl₂, FeBr₂, FeI₂, etc.), ruthenium halide (for example, RuF₂, RuCl₂, RuBr₂, RuI₂, etc.), osmium halide (for example, OsF₂, OsCl₂, OsBr₂, OsI₂, etc.), cobalt halide (for example, CoF₂, COCl₂, CoBr₂, CoI₂, etc.), rhodium halide (for example, RhF₂, RhCl₂, RhBr₂, RhI₂, etc.), iridium halide (for example, IrF₂, IrCl₂, IrBr₂, IrI₂, etc.), nickel halide (for example, NiF₂, NiCl₂, NiBr₂, NiI₂, etc.), palladium halide (for example, PdF₂, PdCl₂, PdBr₂, PdI₂, etc.), platinum halide (for example, PtF₂, PtCl₂, PtBr₂, PtI₂, etc.), copper halide (for example, CuF, CuCl, CuBr, CuI, etc.), silver halide (for example, AgF, AgCl, AgBr, AgI, etc.), and gold halide (for example, AuF, AuCl, AuBr, AuI, etc.).

Examples of the post-transition metal halide may include zinc halide (for example, ZnF₂, ZnCl₂, ZnBr₂, ZnI₂, etc.), indium halide (for example, InI₃, etc.), and tin halide (for example, SnI₂, etc.).

Examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, and SmI₃.

Examples of the metalloid halide may include antimony halide (for example, SbCl₅, etc.).

Examples of the metal telluride may include alkali metal telluride (for example, Li₂Te, Na₂Te, K₂Te, Rb₂Te, Cs₂Te, etc.), alkaline earth metal telluride (for example, BeTe, MgTe, CaTe, SrTe, BaTe, etc.), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal telluride (for example, ZnTe, etc.), and lanthanide metal telluride (for example, LaTe, CeTe, PrTe, NdTe, PmTe, EuTe, GdTe, TbTe, DyTe, HoTe, ErTe, TmTe, YbTe, LuTe, etc.).

### Emission Layer in Interlayer 130

When the light-emitting device 10 is a full-color light-emitting device, the emission layer may be patterned into a red emission layer, a green emission layer, and/or a blue emission layer, according to a subpixel. In one or more embodiments, the emission layer may have a stacked structure of two or more layers of a red emission layer, a green emission layer, and a blue emission layer, in which the two or more layers contact (e.g., physically contact) each other or are spaced apart from each other to emit white light. In one or more embodiments, the emission layer may have a structure in which two or more materials of a red light-emitting material, a green light-emitting material, and a blue light-emitting material are mixed together with each other in a single layer, and thus emit white light.

The emission layer may include a host and a dopant. The dopant may include a phosphorescent dopant, a fluorescent dopant, or any combination thereof.

An amount of the dopant in the emission layer may be in a range of about 0.01 part by weight to about 15 parts by weight based on 100 parts by weight of the host.

In one or more embodiments, the emission layer may include a quantum dot.

In one or more embodiments, the emission layer may include a delayed fluorescence material. The delayed fluorescence material may act as a host or a dopant in the emission layer.

A thickness of the emission layer may be in a range of about 100 Å to about 1,000 Å, for example, about 200 Å to about 600 Å. When the thickness of the emission layer is within this range, excellent light-emission characteristics may be obtained without a substantial increase in driving voltage.

### Host

The host may include a compound represented by Formula 301:

[Ar₃₀₁]_{xb11}-[(L₃₀₁)_{xb1}-R₃₀₁]_{xb21} Formula 301

wherein, in Formula 301,

Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xb11 may be 1, 2, or 3,

xb1 may be an integer from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer from 1 to 5, and

Q₃₀₁ to Q₃₀₃ are each the same as described herein in connection with Q₁.

For example, when xb11 in Formula 301 is 2 or more, two or more of Ar₃₀₁ (s) may be linked to each other via a single bond.

In one or more embodiments, the host may include a compound represented by Formula 301-1, a compound represented by Formula 301-2, or any combination thereof:

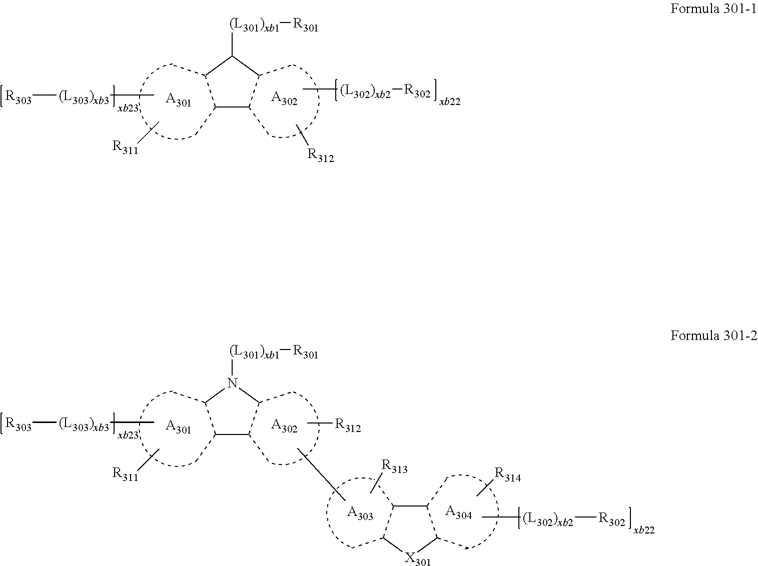

wherein, in Formulae 301-1 and 301-2,

ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

X₃₀₁ may be O, S, N-[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ are respectively the same as those described herein,

L₃₀₂ to L₃₀₄ are each independently the same as described herein in connection with L₃₀₁,

xb2 to xb4 are each independently the same as described herein in connection with xb1, and

R₃₀₂ to R₃₀₅ and R₃₁₁ to R₃₁₄ are each the same as described herein with respect to R₃₀₁.

In one or more embodiments, the host may include an alkali earth metal complex, a post-transition metal complex, or any combination thereof. For example, the host may include a Be complex (for example, Compound H55), an Mg complex, a Zn complex, or any combination thereof.

In one or more embodiments, the host may include one of Compounds H1 to H124, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof:

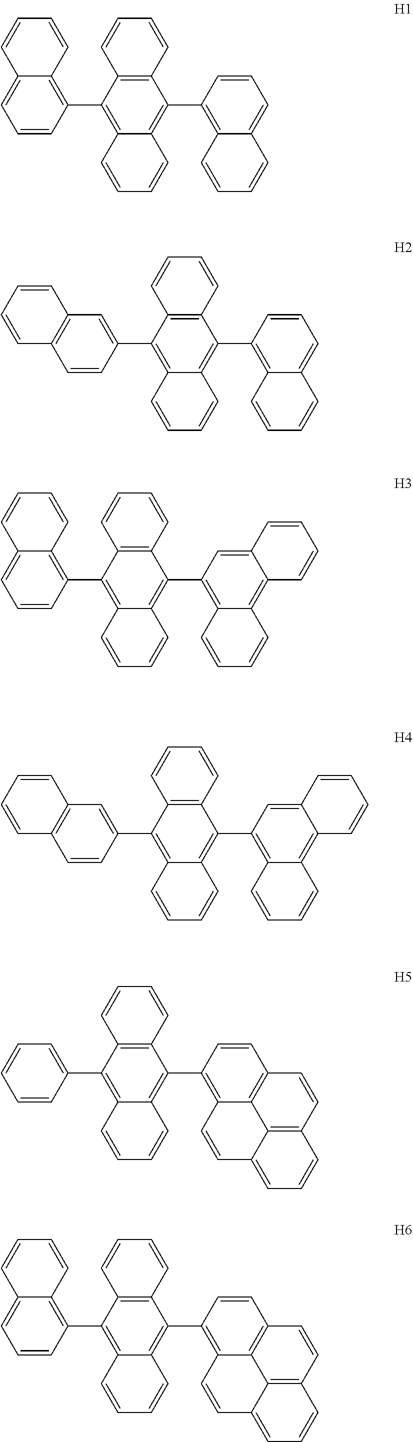

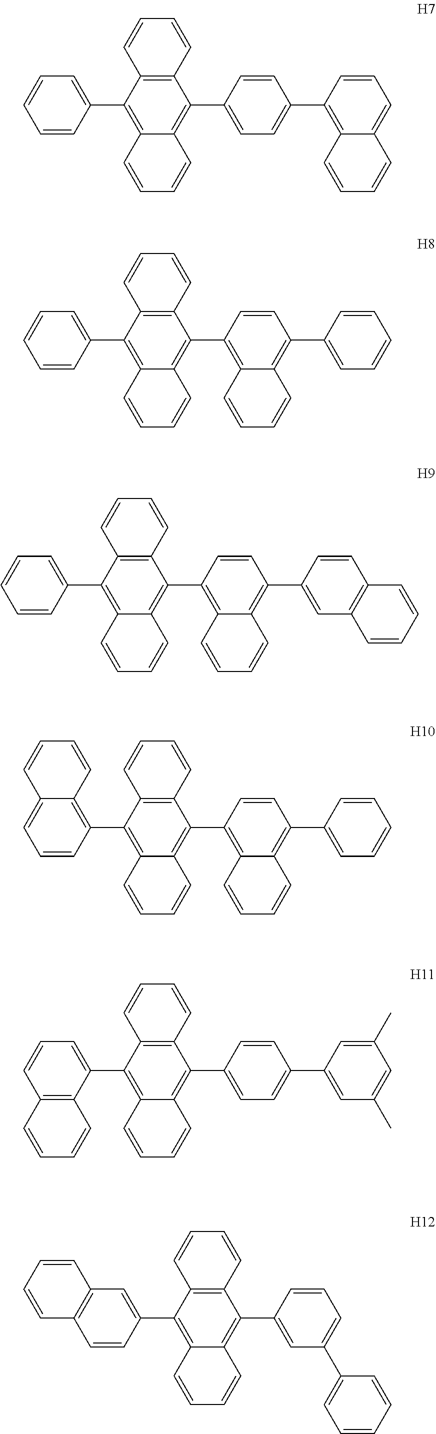

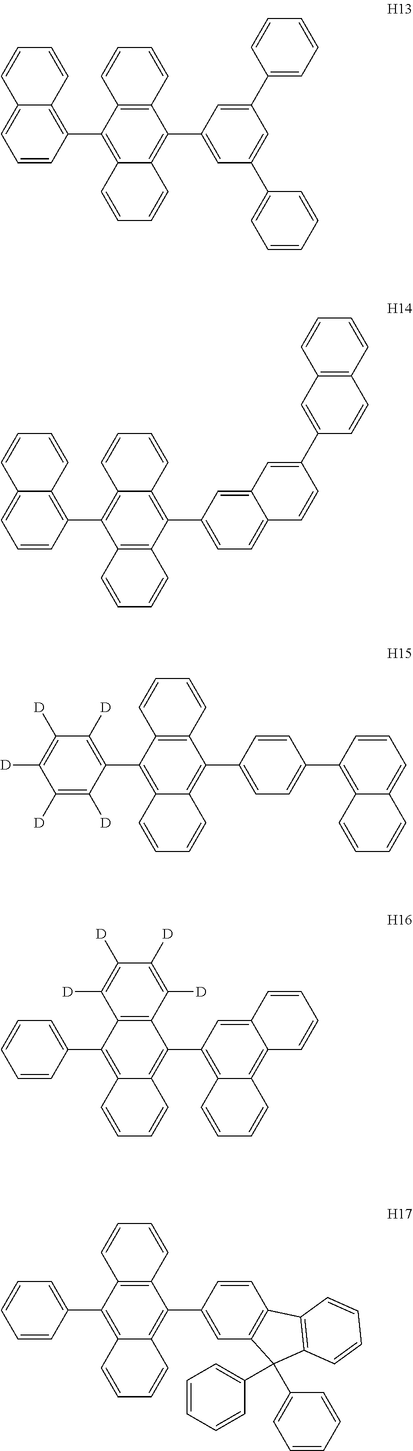

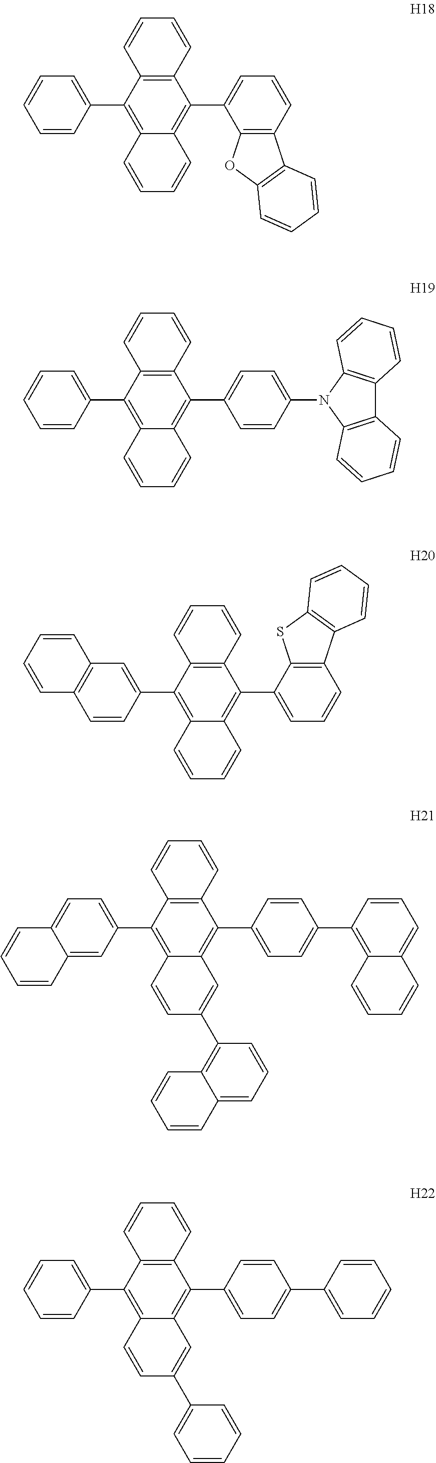

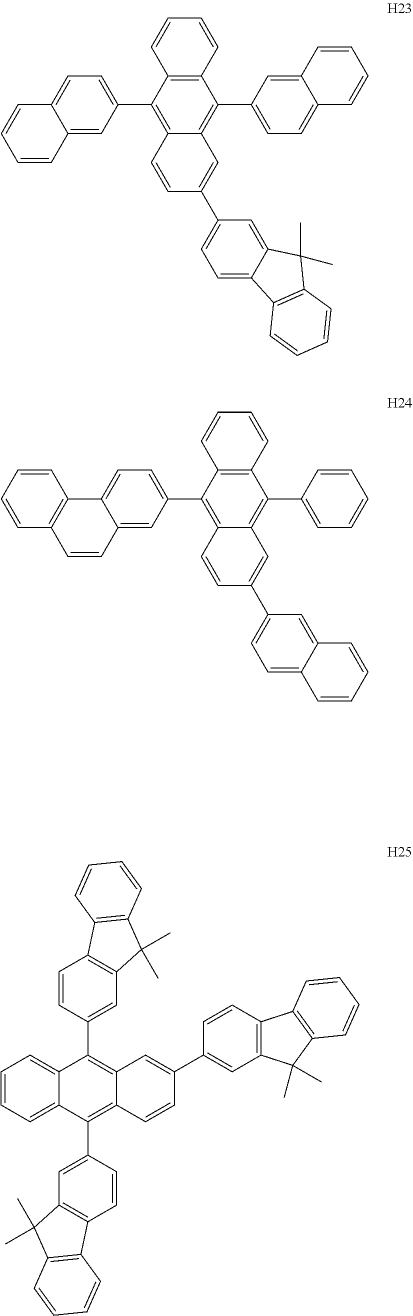

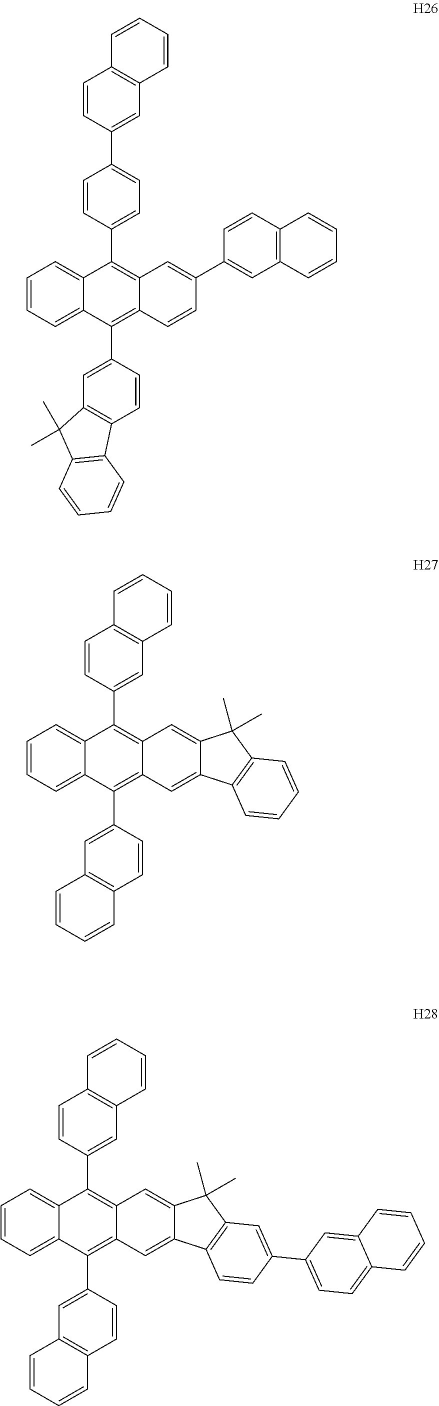

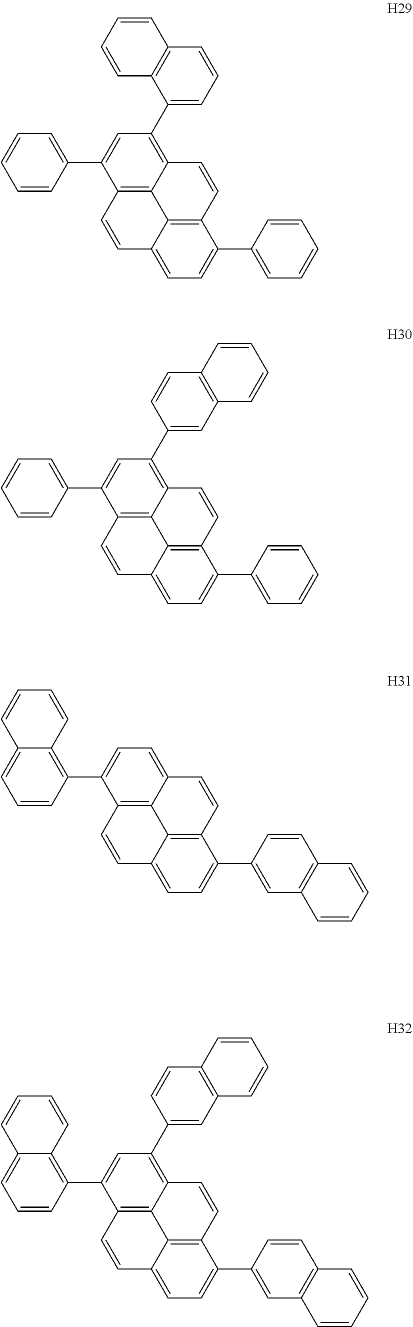

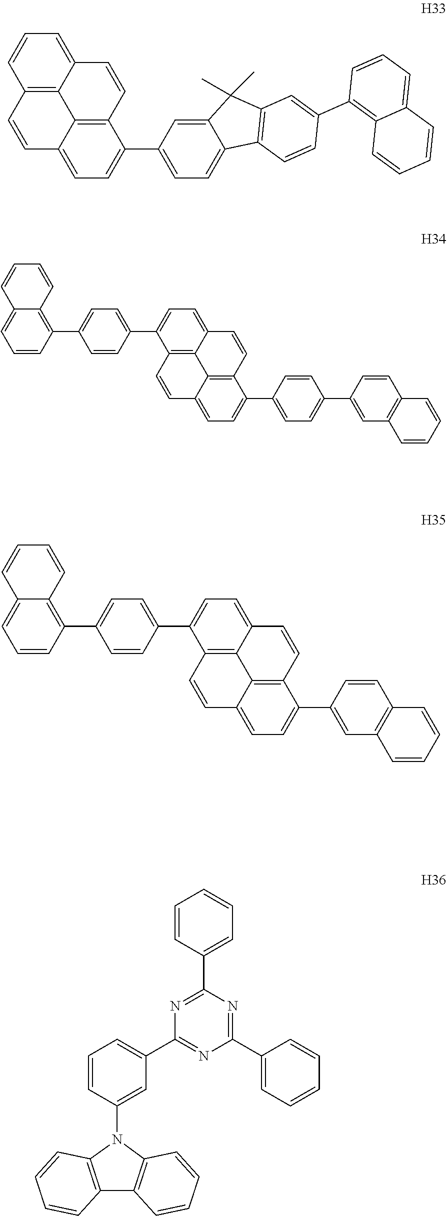

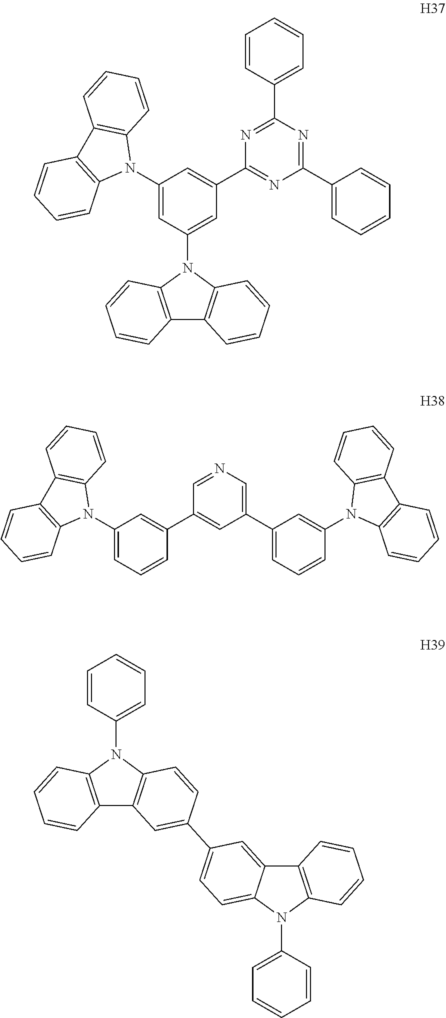 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1]

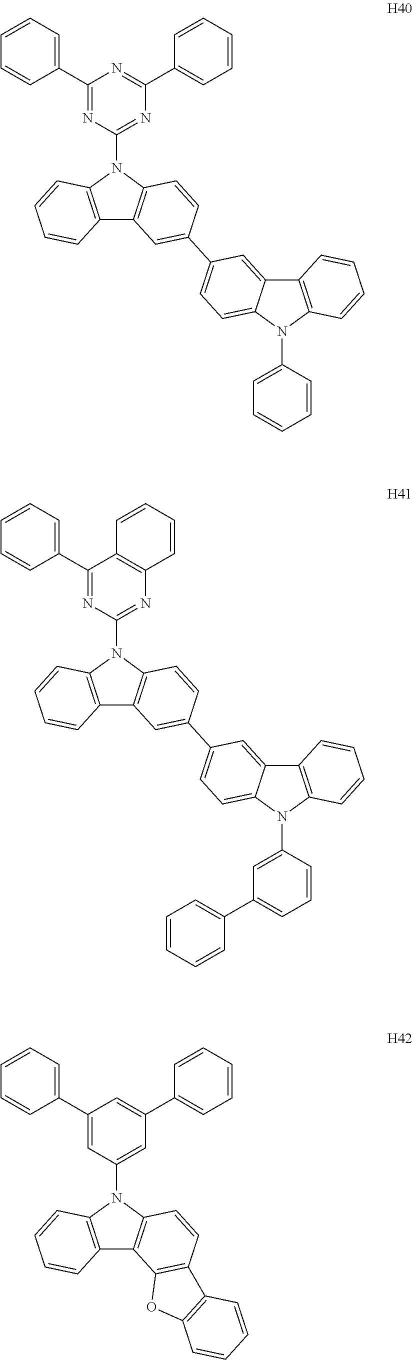 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

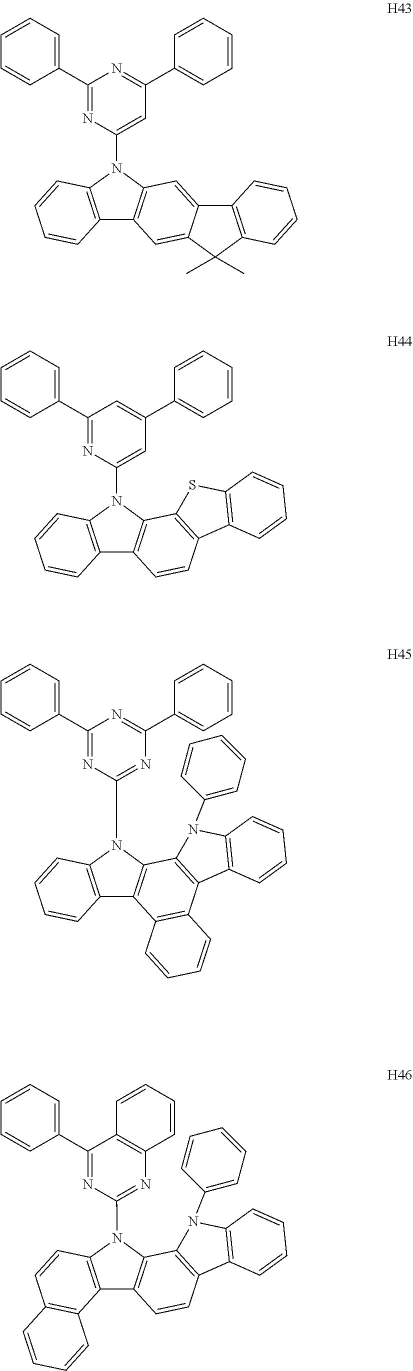 [SMILES: CC1(C)c2ccccc2-c2cc3c(cc21)c1ccccc1n3-c1cc(-c2ccccc2)nc(-c2ccccc2)n1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

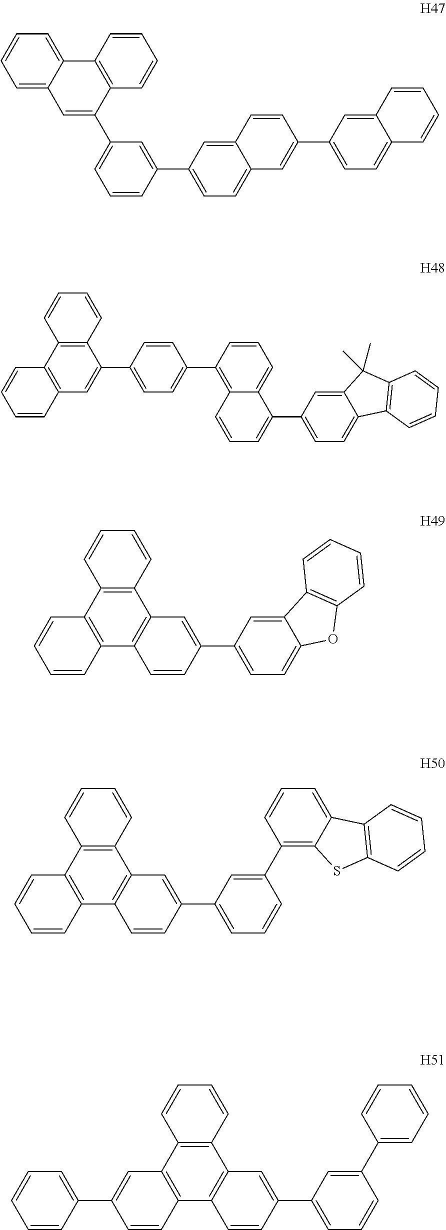

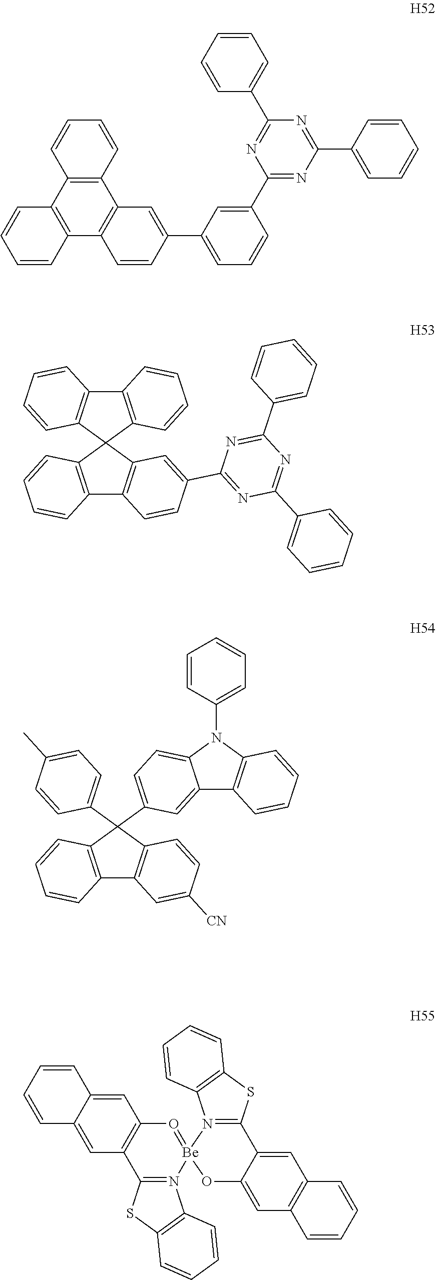

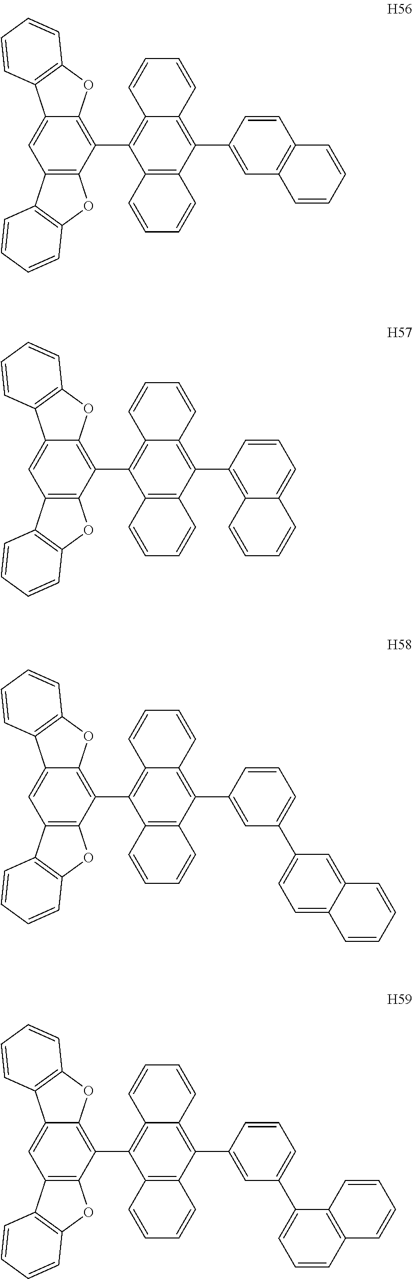 [SMILES: c1ccc2cc(-c3c4ccccc4c(-c4c5oc6ccccc6c5cc5c4oc4ccccc45)c4ccccc34)ccc2c1 ; c1ccc2c(-c3c4ccccc4c(-c4c5oc6ccccc6c5cc5c4oc4ccccc45)c4ccccc34)cccc2c1 ; c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1]

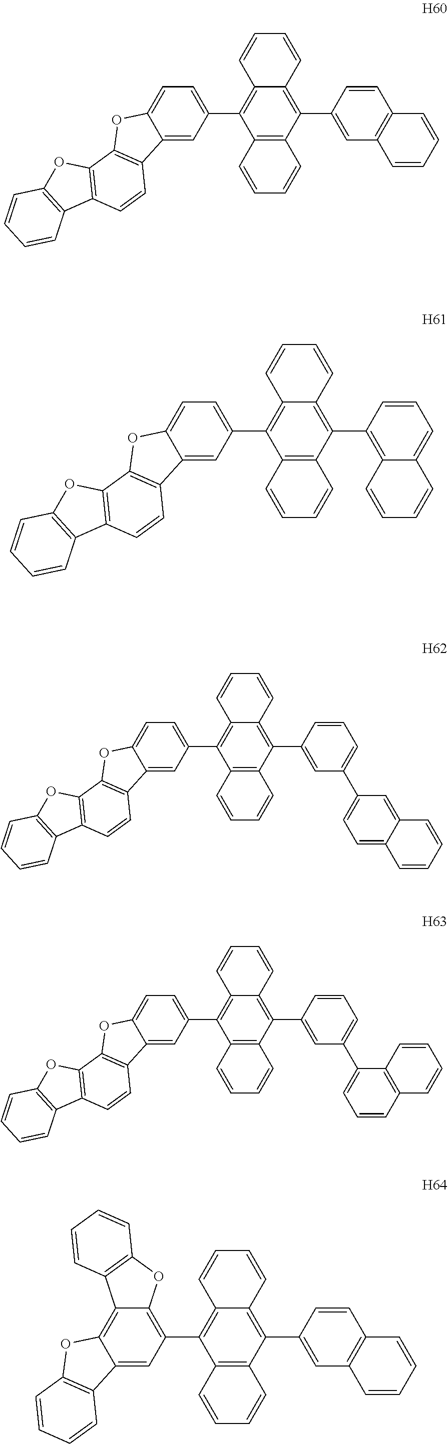 [SMILES: c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1]

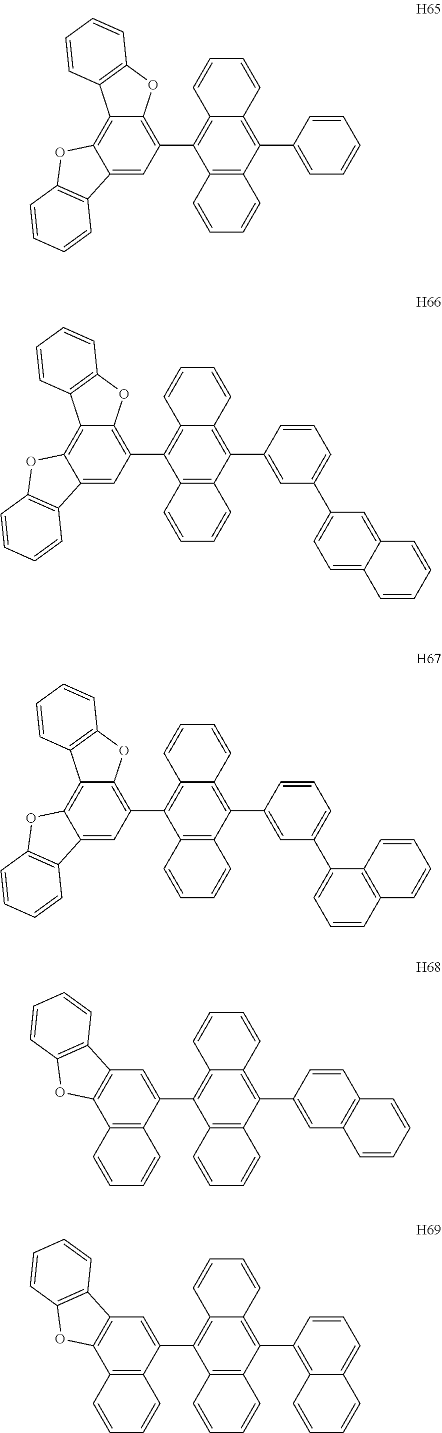 [SMILES: c1ccc(-c2c3ccccc3c(-c3cc4c5ccccc5oc4c4c3oc3ccccc34)c3ccccc23)cc1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1]

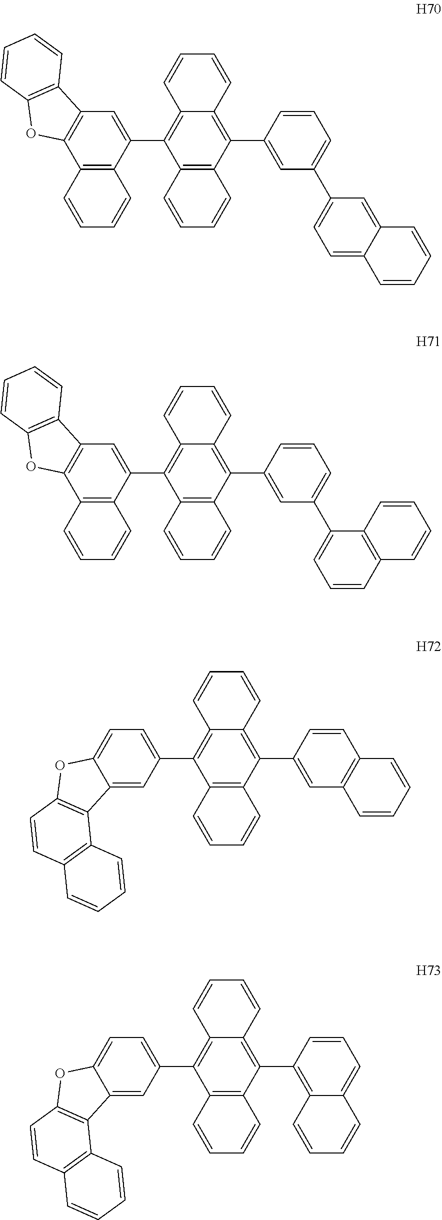 [SMILES: c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1 ; c1ccc2c(-c3c4ccccc4c(-c4ccc5oc6ccc7ccccc7c6c5c4)c4ccccc34)cccc2c1]

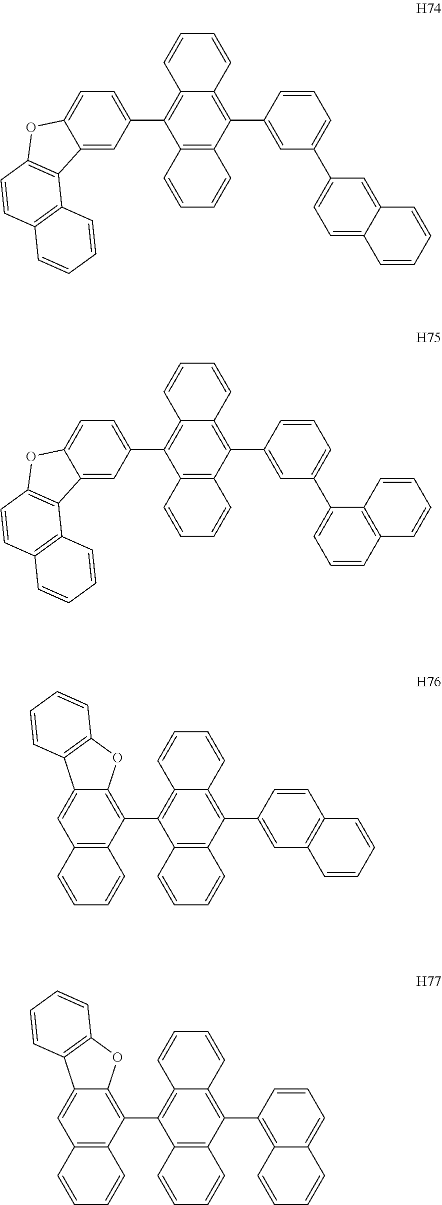 [SMILES: c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1 ; c1ccc2cc(-c3c4ccccc4c(-c4c5ccccc5cc5c4oc4ccccc45)c4ccccc34)ccc2c1 ; c1ccc2c(-c3c4ccccc4c(-c4c5ccccc5cc5c4oc4ccccc45)c4ccccc34)cccc2c1]

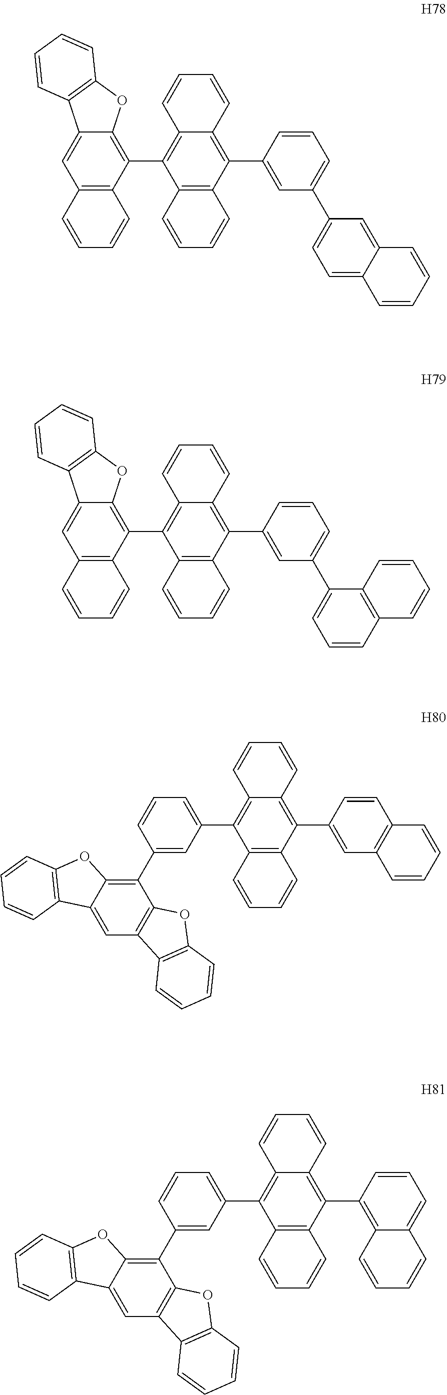 [SMILES: c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1]

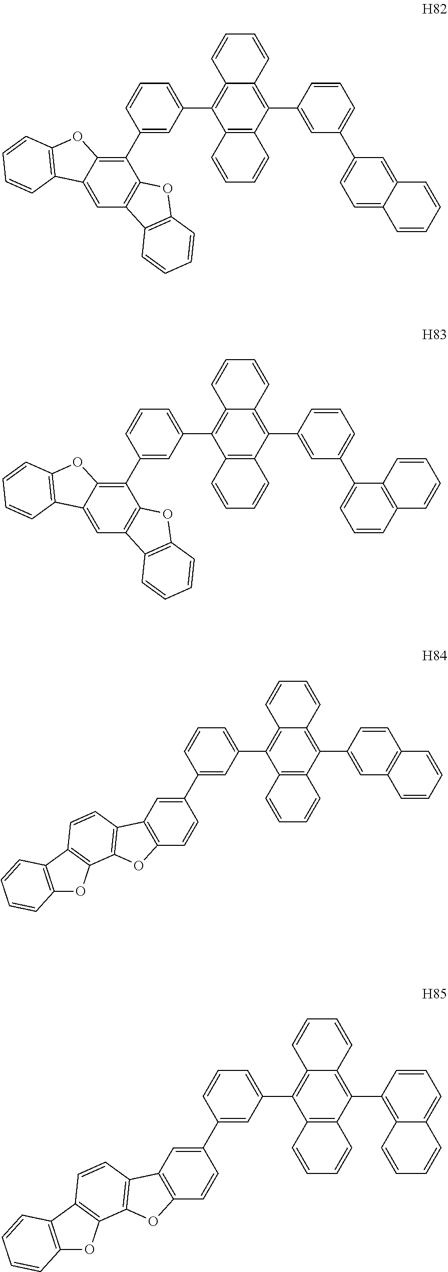 [SMILES: c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1]

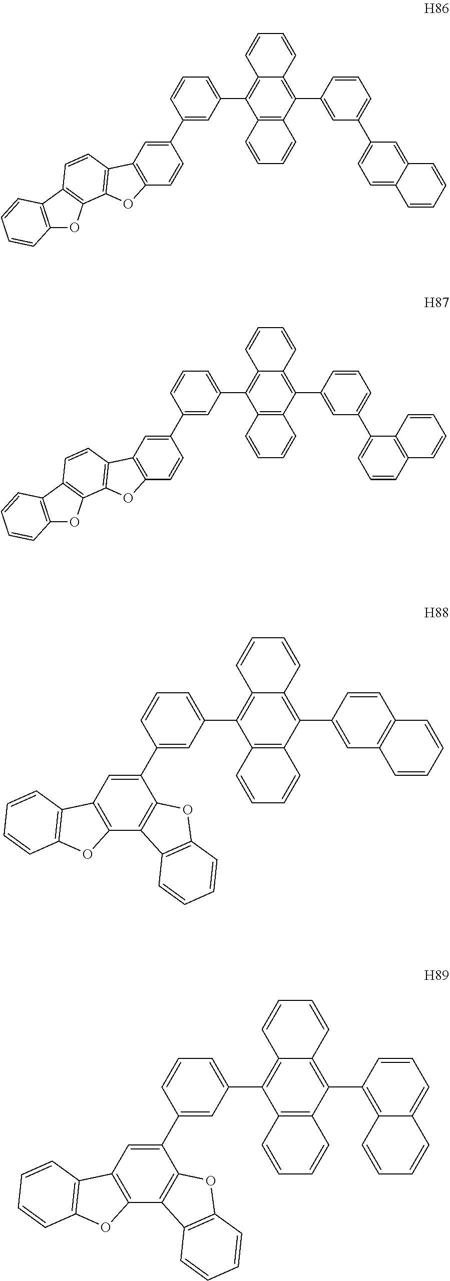 [SMILES: c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1]

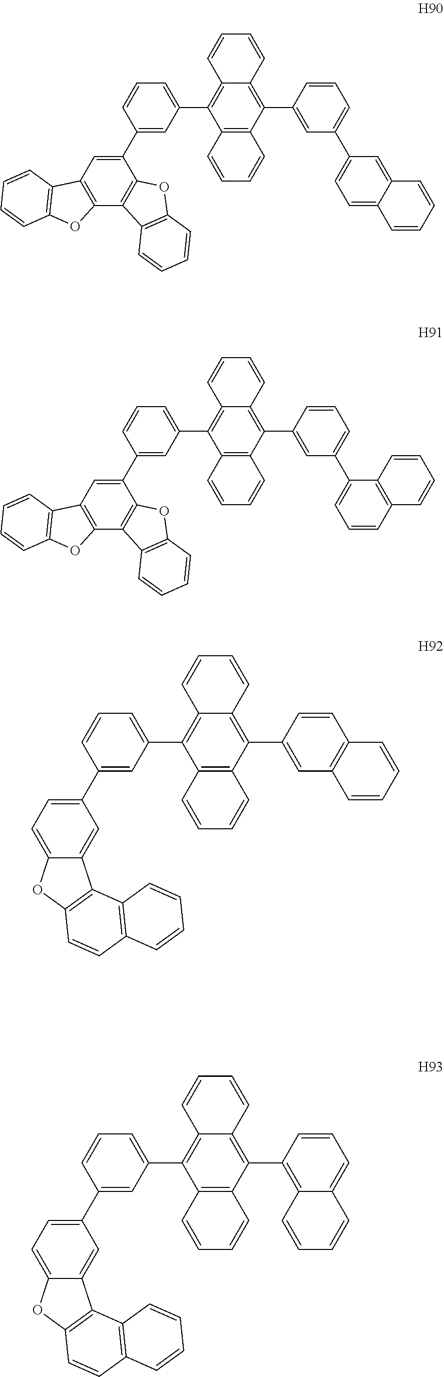 [SMILES: c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1]

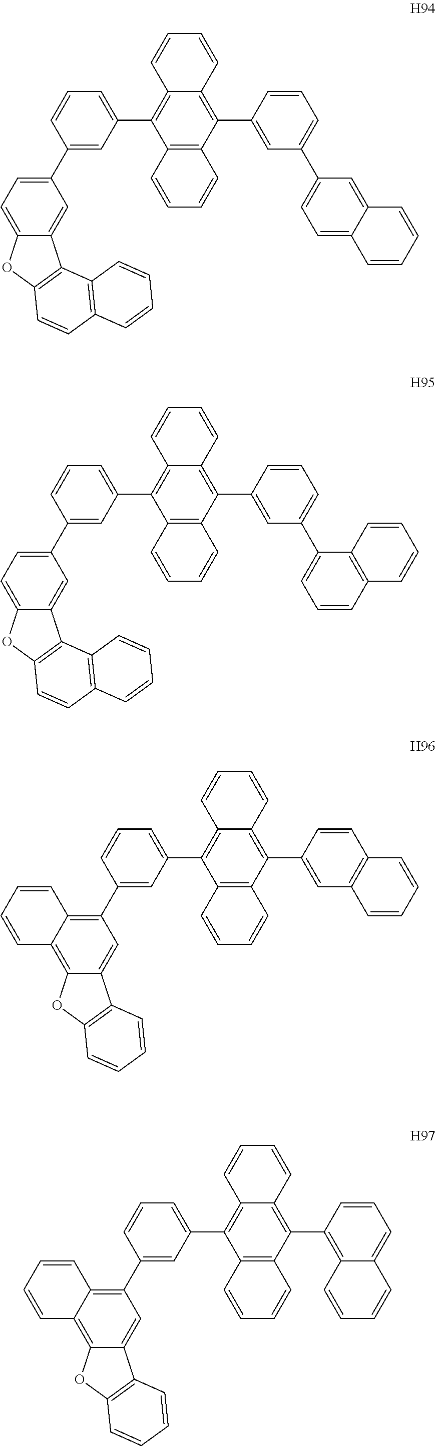 [SMILES: c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1]

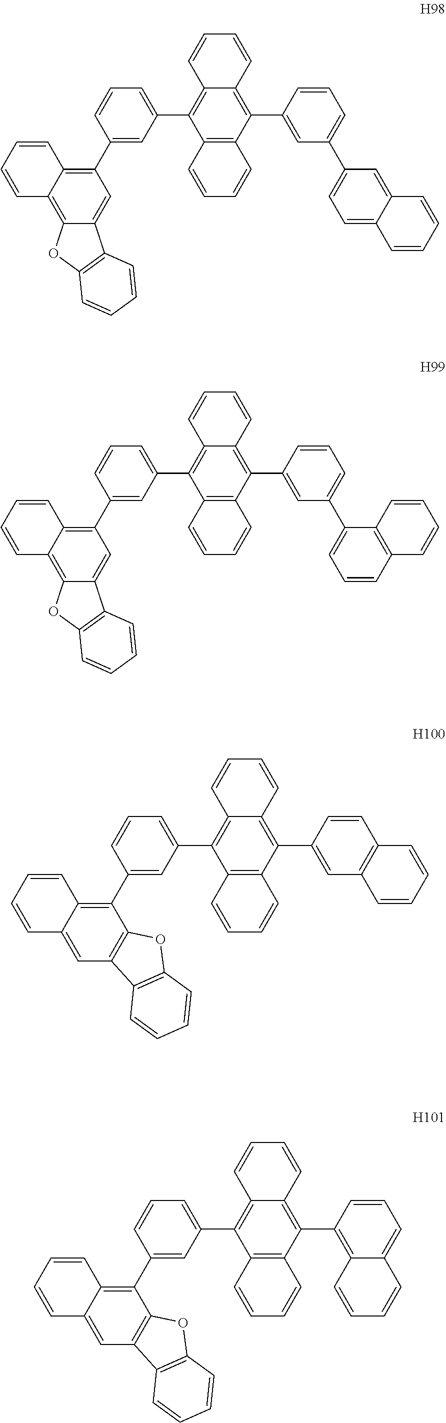 [SMILES: c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1]

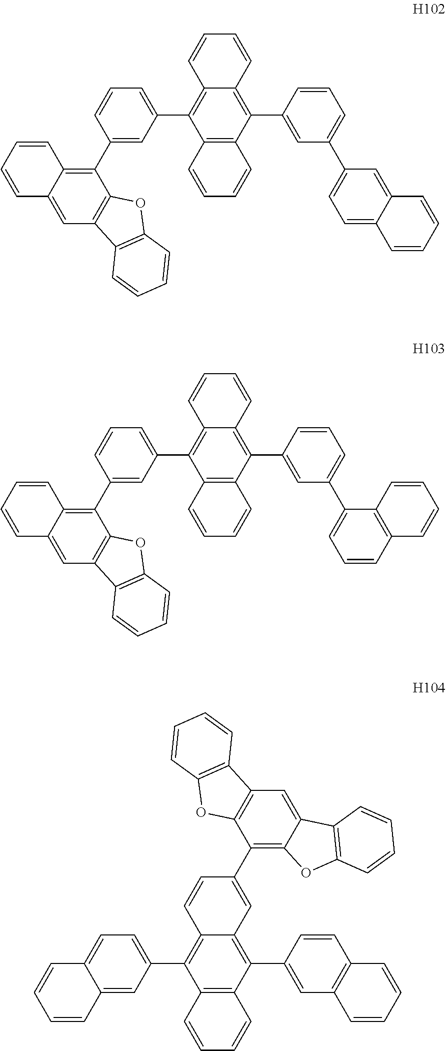 [SMILES: c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

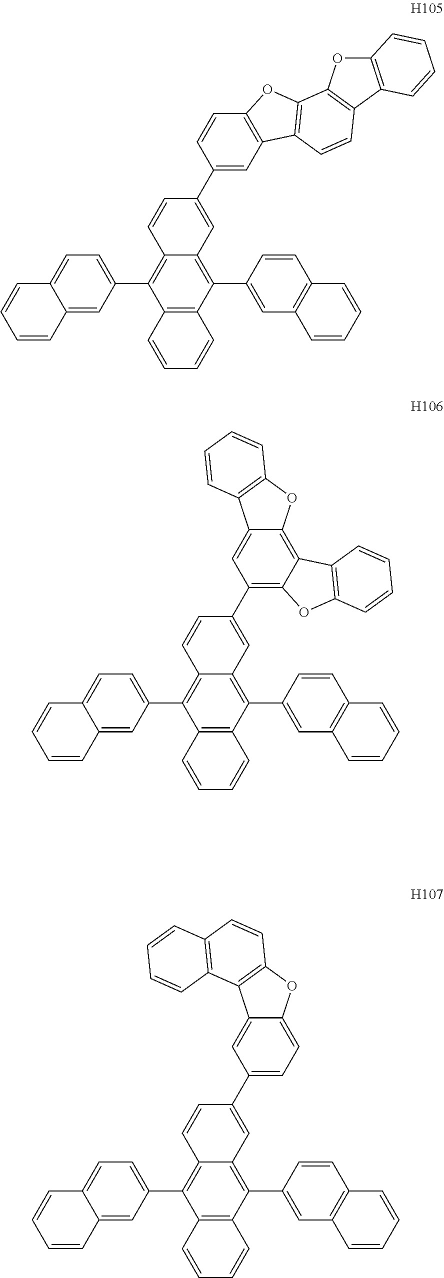 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1]

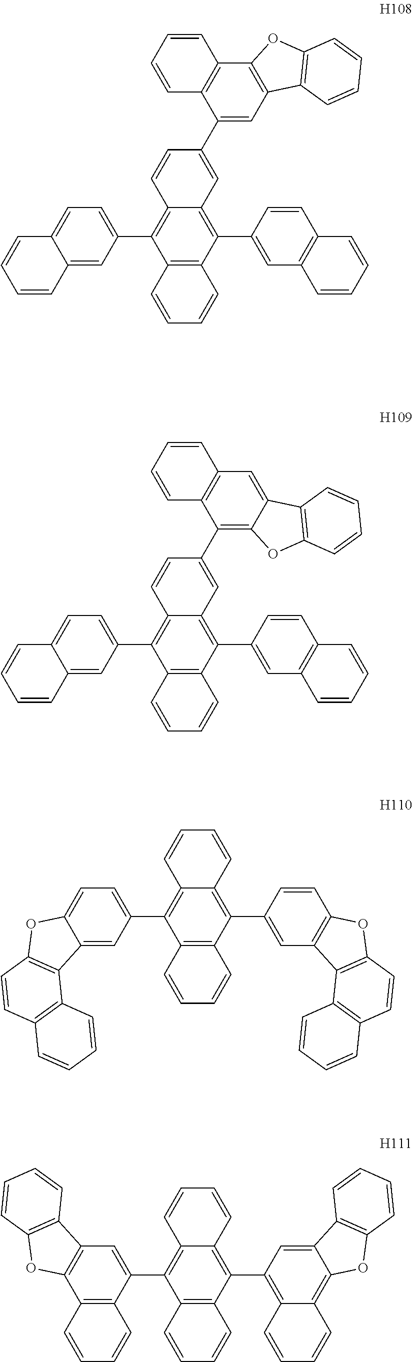 [SMILES: c1ccc2cc(-c3c4ccccc4c(-c4ccc5ccccc5c4)c4cc(-c5cc6c7ccccc7oc6c6ccccc56)ccc34)ccc2c1 ; c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12 ; c1ccc2c(c1)oc1c3ccccc3c(-c3c4ccccc4c(-c4cc5c6ccccc6oc5c5ccccc45)c4ccccc34)cc21]

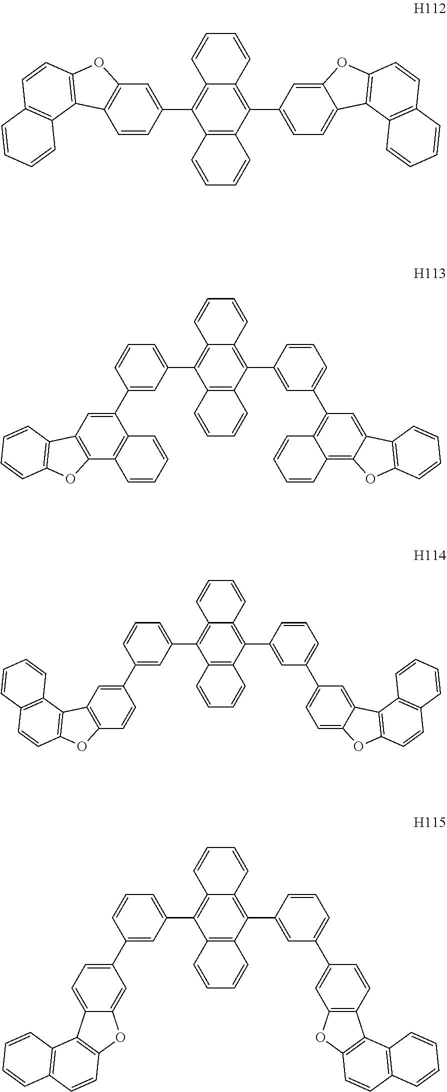 [SMILES: c1ccc2c(c1)ccc1oc3cc(-c4c5ccccc5c(-c5ccc6c(c5)oc5ccc7ccccc7c56)c5ccccc45)ccc3c12 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1]

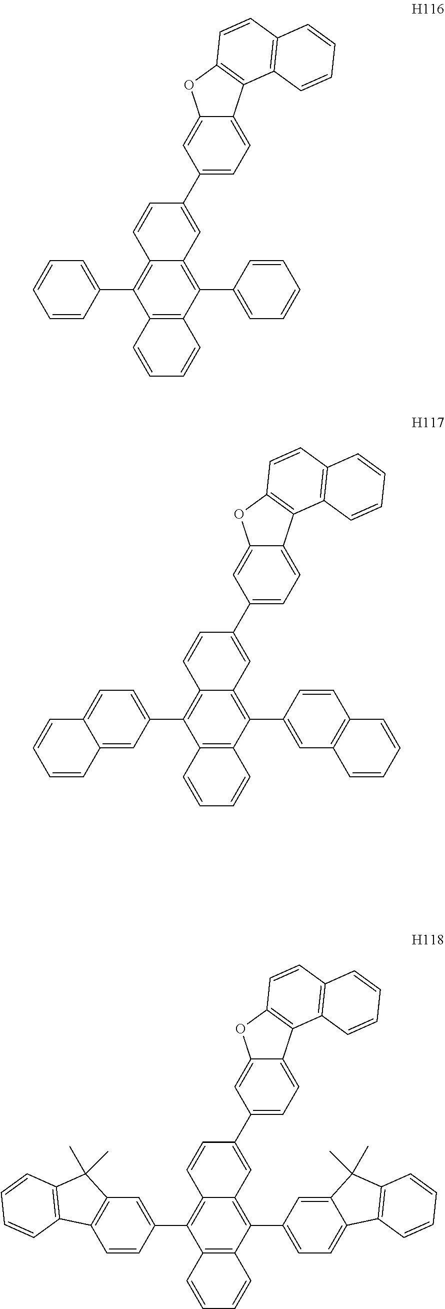 [SMILES: c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

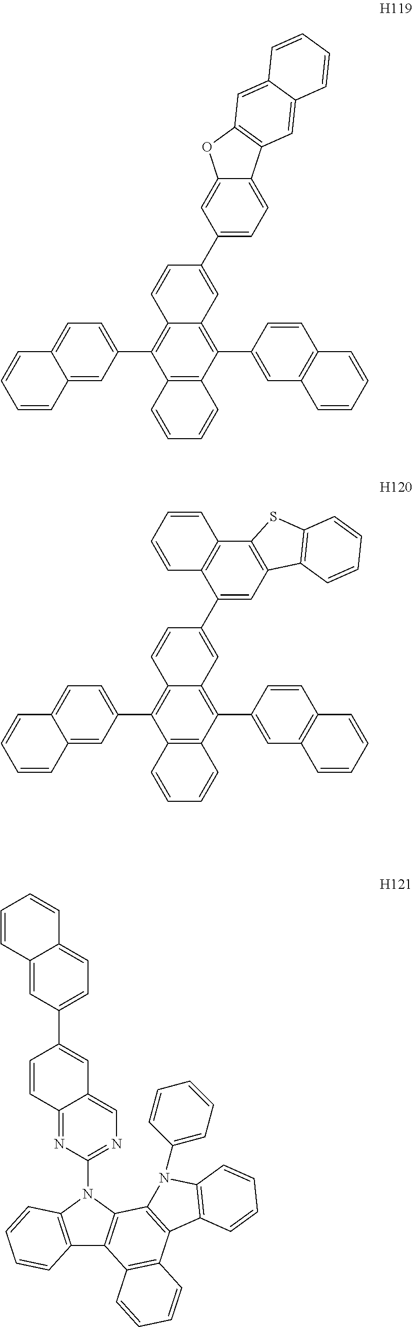 [SMILES: c1ccc2cc(-c3c4ccccc4c(-c4ccc5ccccc5c4)c4cc(-c5ccc6c(c5)oc5cc7ccccc7cc56)ccc34)ccc2c1 ; c1ccc2cc(-c3c4ccccc4c(-c4ccc5ccccc5c4)c4cc(-c5cc6c7ccccc7sc6c6ccccc56)ccc34)ccc2c1 ; c1ccc(-n2c3ccccc3c3c4ccccc4c4c5ccccc5n(-c5ncc6cc(-c7ccc8ccccc8c7)ccc6n5)c4c32)cc1]

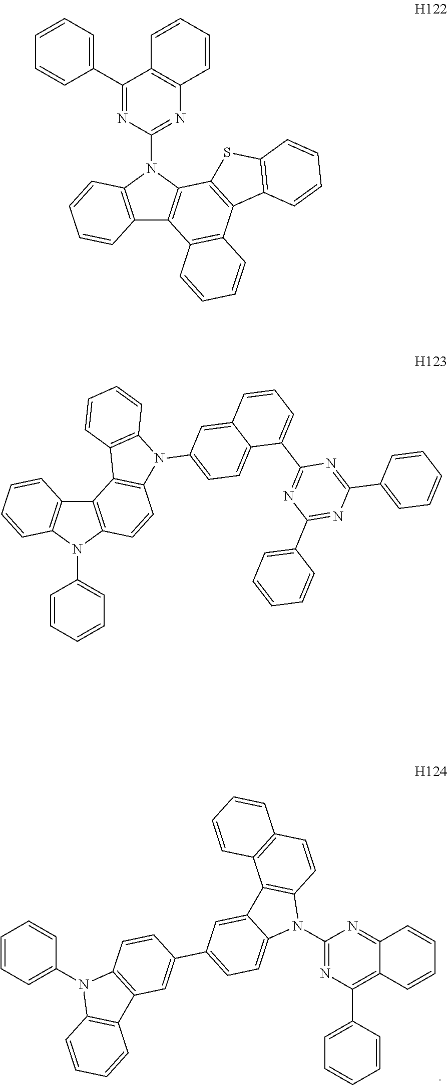 [SMILES: c1ccc(-c2nc(-n3c4ccccc4c4c5ccccc5c5c6ccccc6sc5c43)nc3ccccc23)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

### Phosphorescent Dopant

The phosphorescent dopant may include at least one transition metal as a center metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

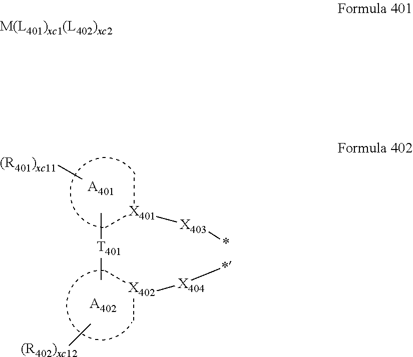

wherein, in Formulae 401 and 402,

M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),

L₄₀₁ may be a ligand represented by Formula 402, and xc1 may be 1, 2, or 3, wherein, when xc1 is 2 or more, two or more of L₄₀₁ (s) may be identical to or different from each other,

L₄₀₂ may be an organic ligand, and xc2 may be 0, 1, 2, 3, or 4, wherein, when xc2 is 2 or more, two or more of L₄₀₂(s) may be identical to or different from each other,

X₄₀₁ and X₄₀₂ may each independently be nitrogen or carbon,

ring A₄₀₁ and ring A₄₀₂ may each independently be a C₃-C₆₀ carbocyclic group or a C₁-C₆₀ heterocyclic group,

T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *═C(Q₄₁₁)=*′,

X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),

Q₄₁₁ to Q₄₁₄ are each the same as described herein in connection with Q₁,

R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),

Q₄₀₁ to Q₄₀₃ are each the same as described herein in connection with Q₁,

xc11 and xc12 may each independently be an integer from 0 to 10, and

* and *′ in Formula 402 each indicate a binding site to M in Formula 401.

For example, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 401 is 2 or more, two ring A₄₀₁(s) in two or more of L₄₀₁(s) may optionally be linked to each other via T₄₀₂, which is a linking group, and two ring A₄₀₂(s) may be optionally be linked to each other via T₄₀₃, which is a linking group (see Compounds PD1 to PD4 and PD7). T₄₀₂ and T₄₀₃ are each the same as described herein in connection with T₄₀₁.

L₄₀₂ in Formula 401 may be an organic ligand. For example, L₄₀₂ may include a halogen group, a diketone group (for example, an acetylacetonate group), a carboxylic acid group (for example, a picolinate group), —C(═O), an isonitrile group, a —CN group, a phosphorus group (for example, a phosphine group, a phosphite group, etc.), or any combination thereof.

The phosphorescent dopant may include, for example, one of compounds PD1 to PD39, or any combination thereof:

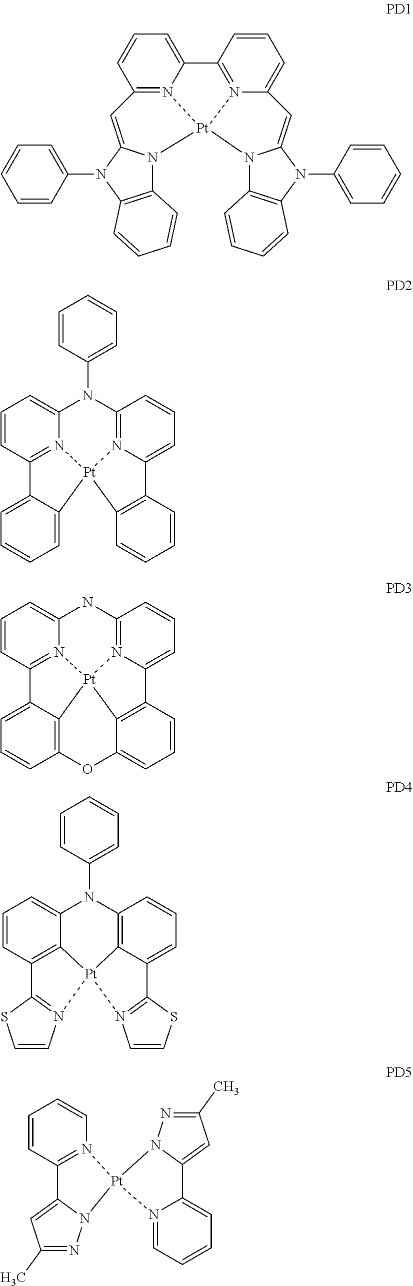

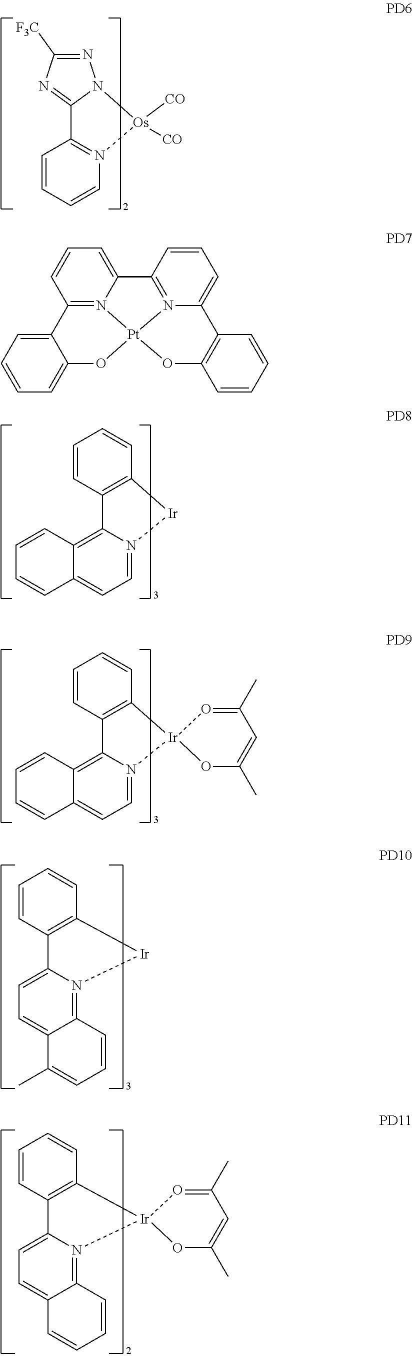

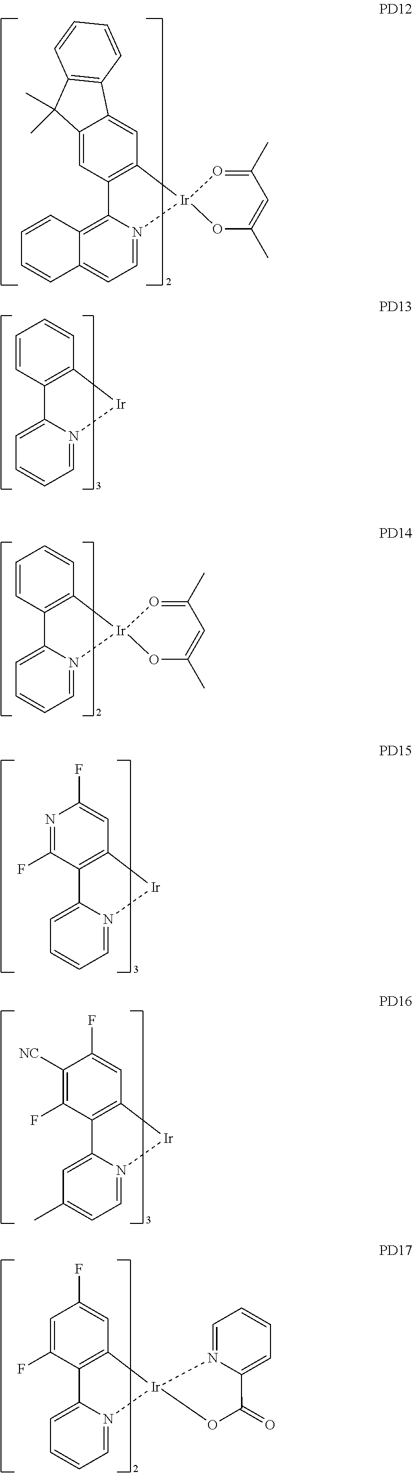

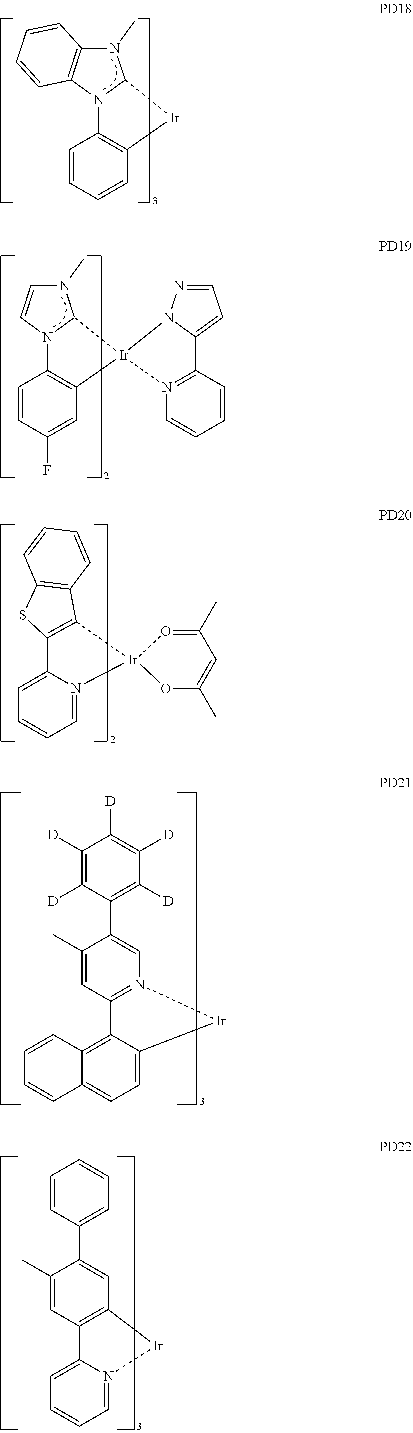

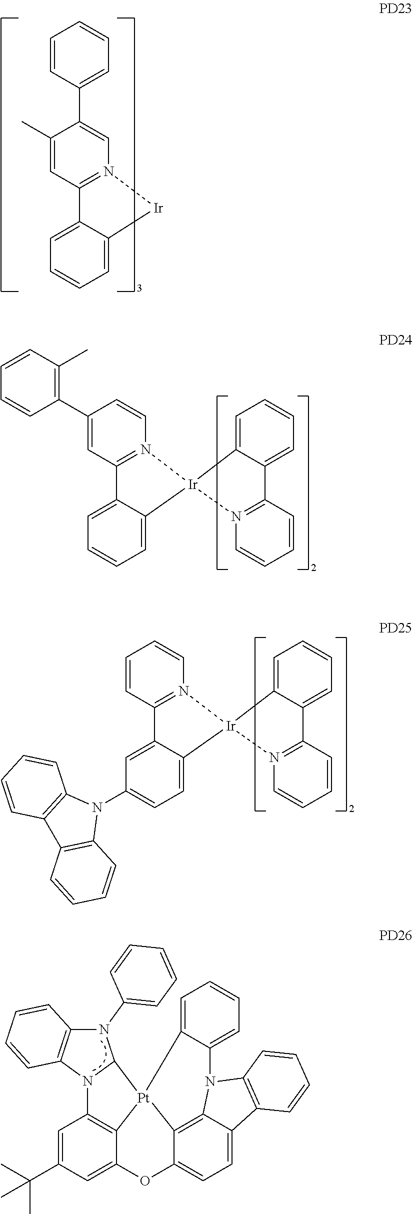

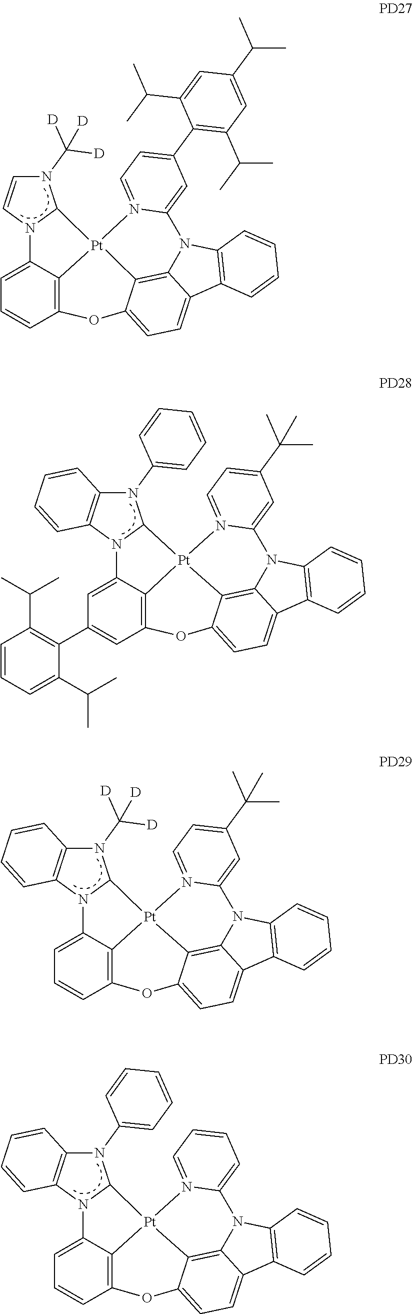

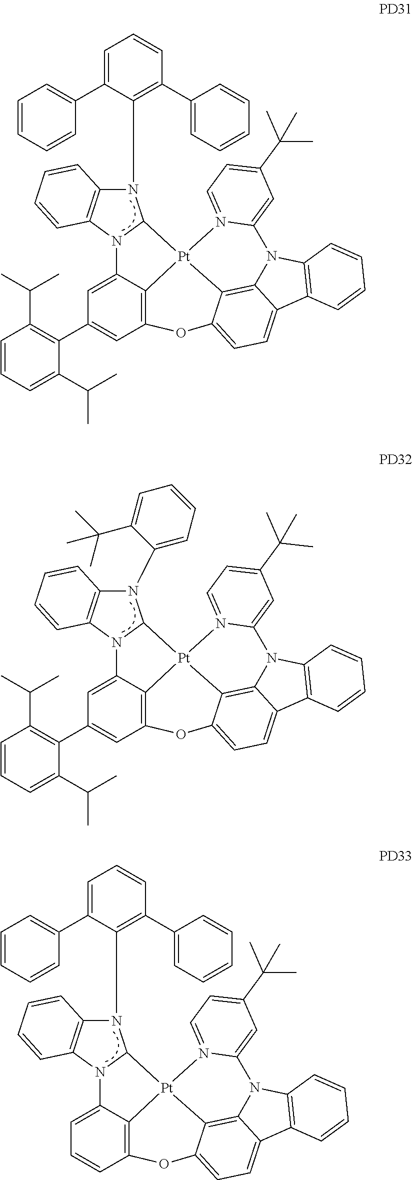

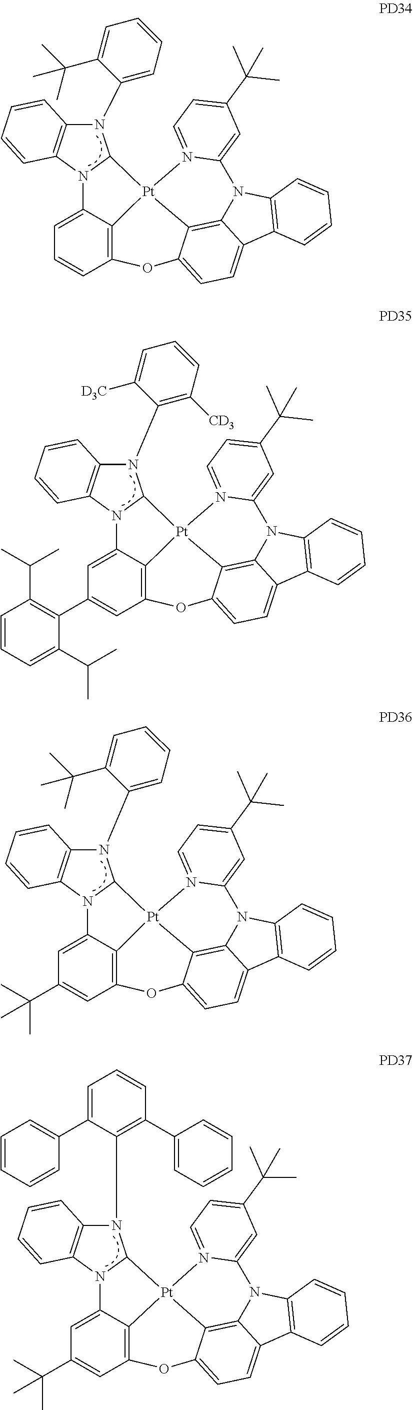

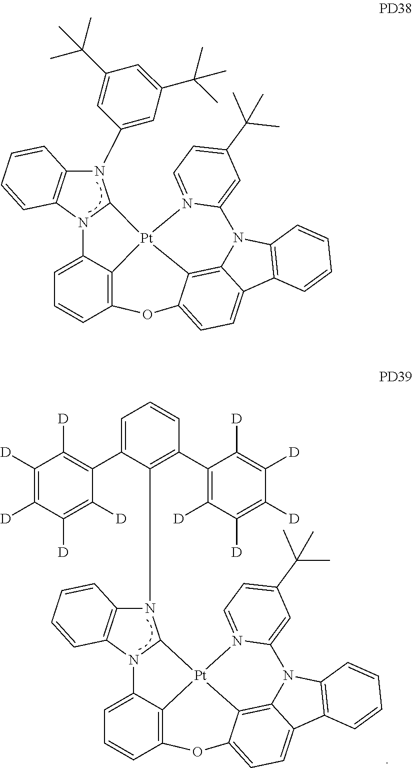

### Fluorescent Dopant

The fluorescent dopant may include an amine group-containing compound, a styryl group-containing compound, or any combination thereof.

For example, the fluorescent dopant may include a compound represented by Formula 501:

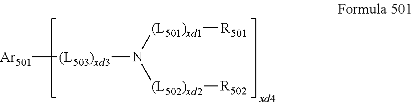

wherein, in Formula 501,

Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xd1 to xd3 may each independently be 0, 1, 2, or 3, and

xd4 may be 1, 2, 3, 4, 5, or 6.

For example, Ar₅₀₁ in Formula 501 may be a condensed cyclic group (for example, an anthracene group, a chrysene group, a pyrene group, etc.) in which three or more monocyclic groups are condensed together.

In one or more embodiments, xd4 in Formula 501 may be 2.

For example, the fluorescent dopant may include one of Compounds FD1 to FD36, DPVBi, DPAVBi, or any combination thereof:

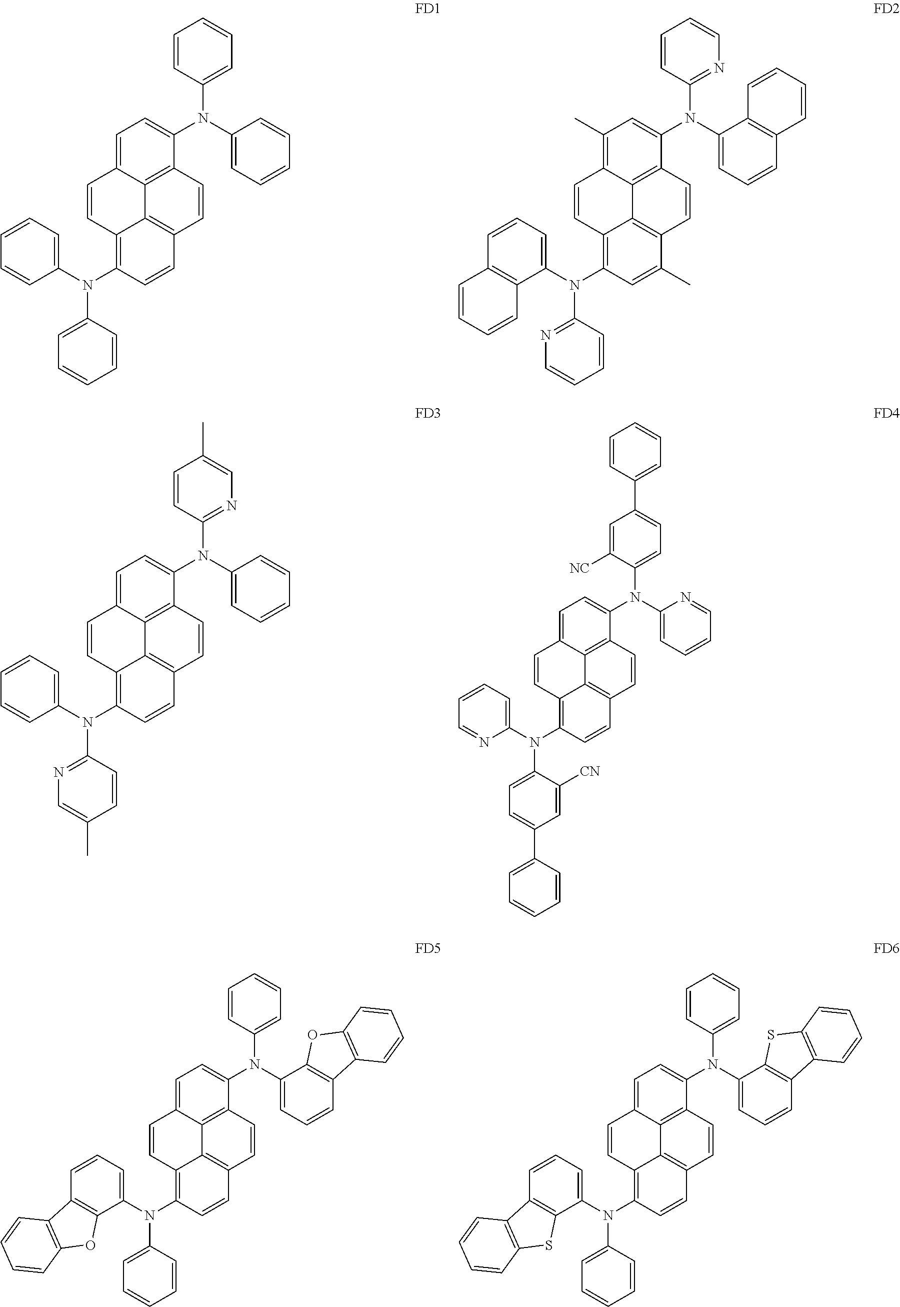

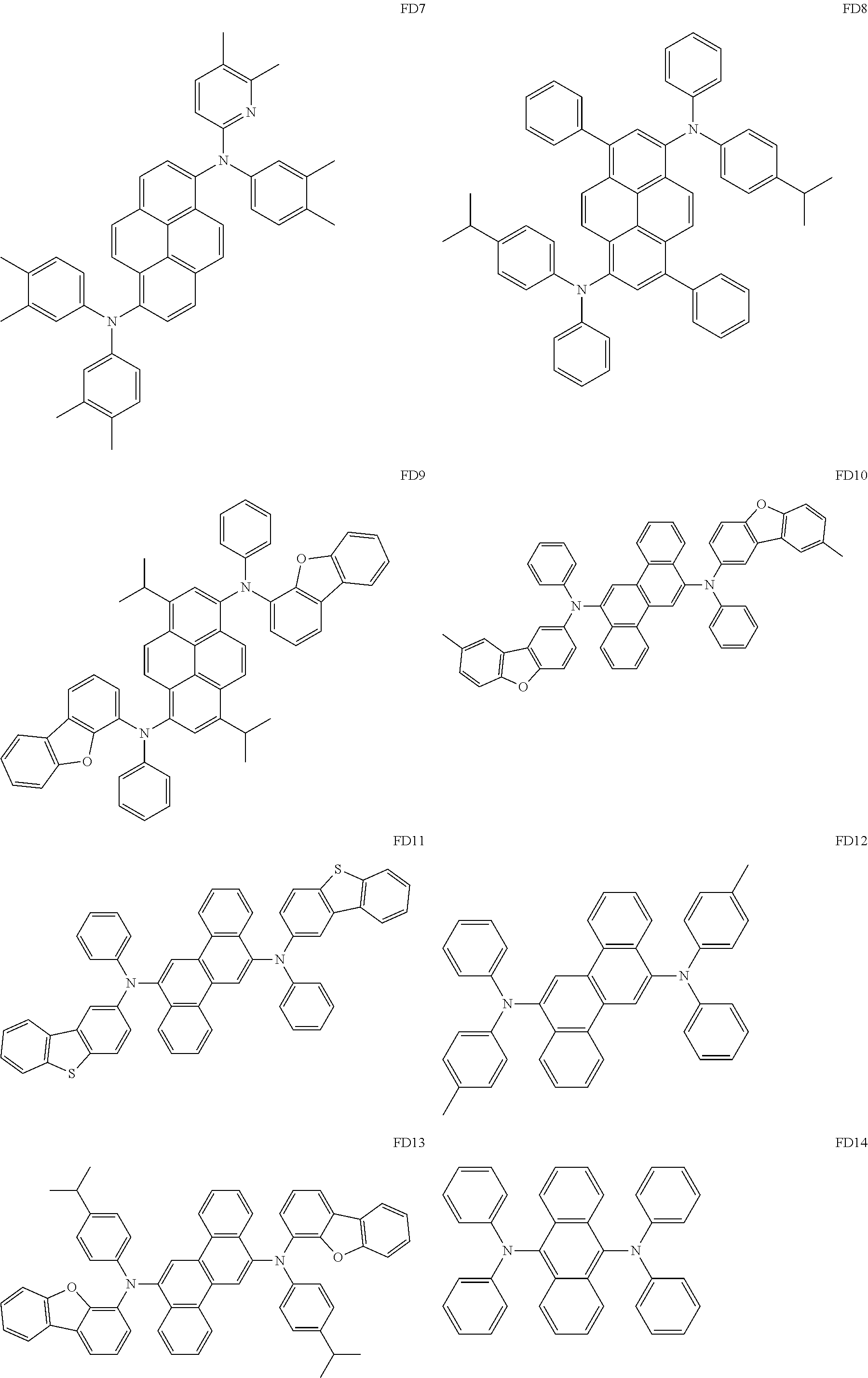

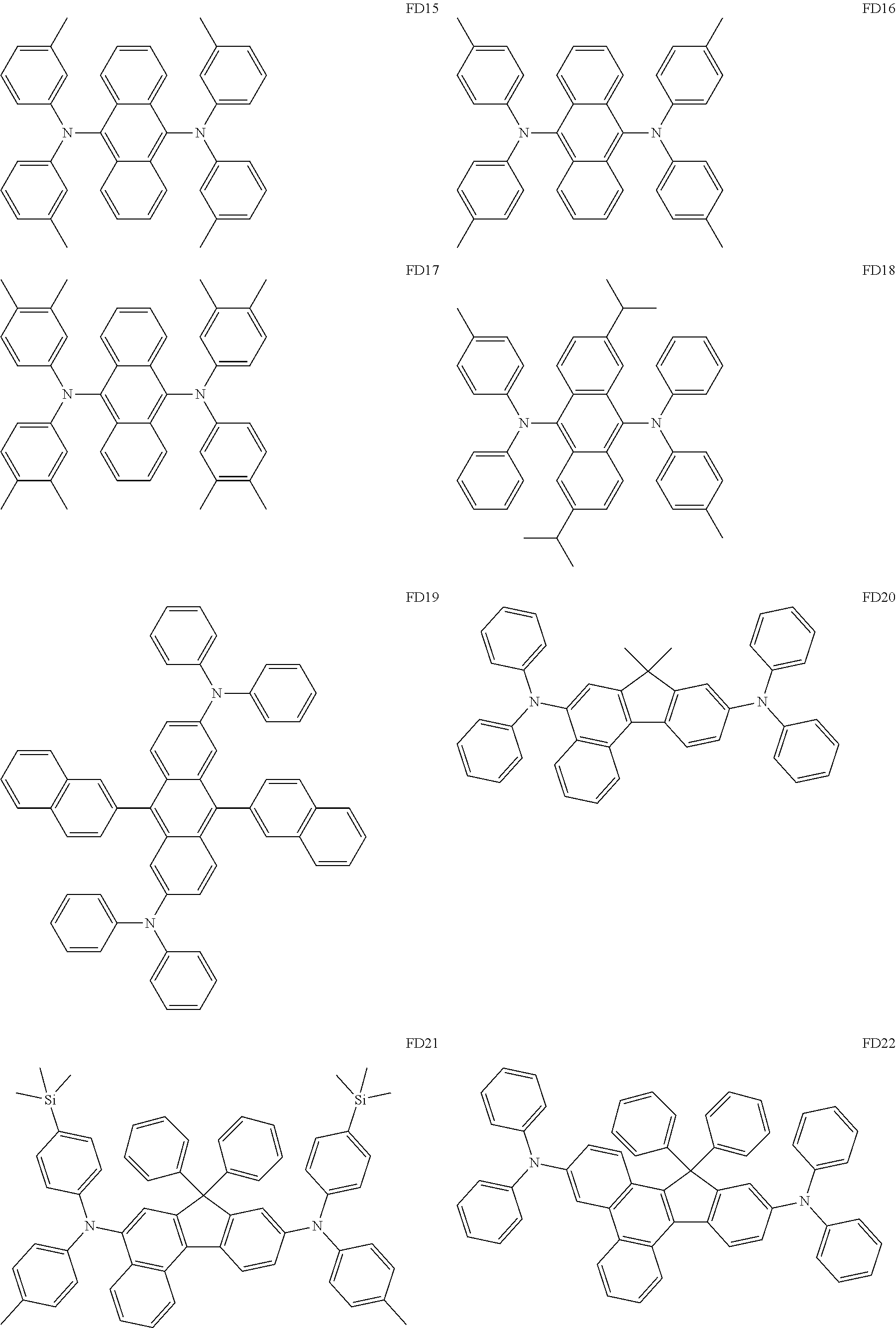

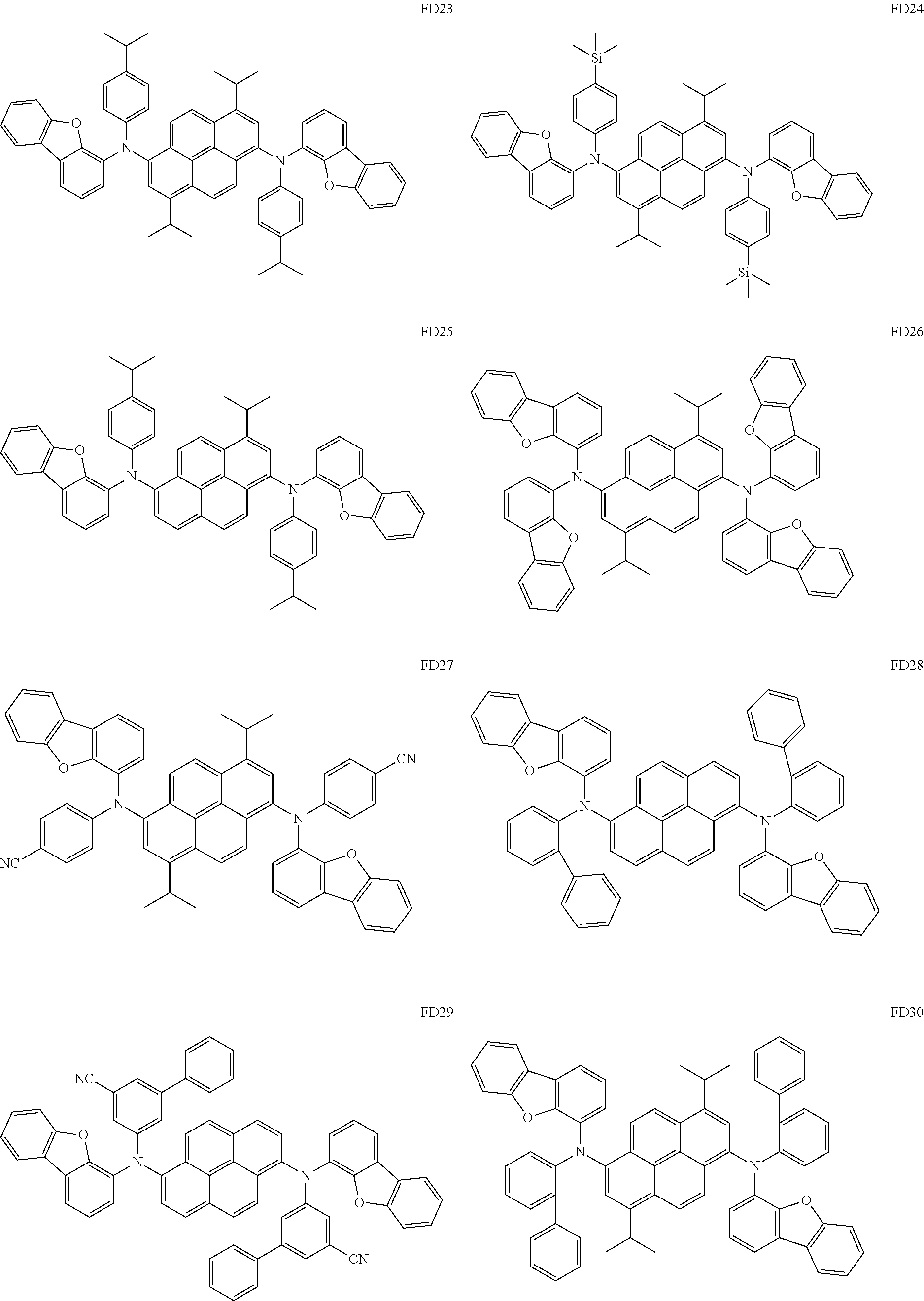

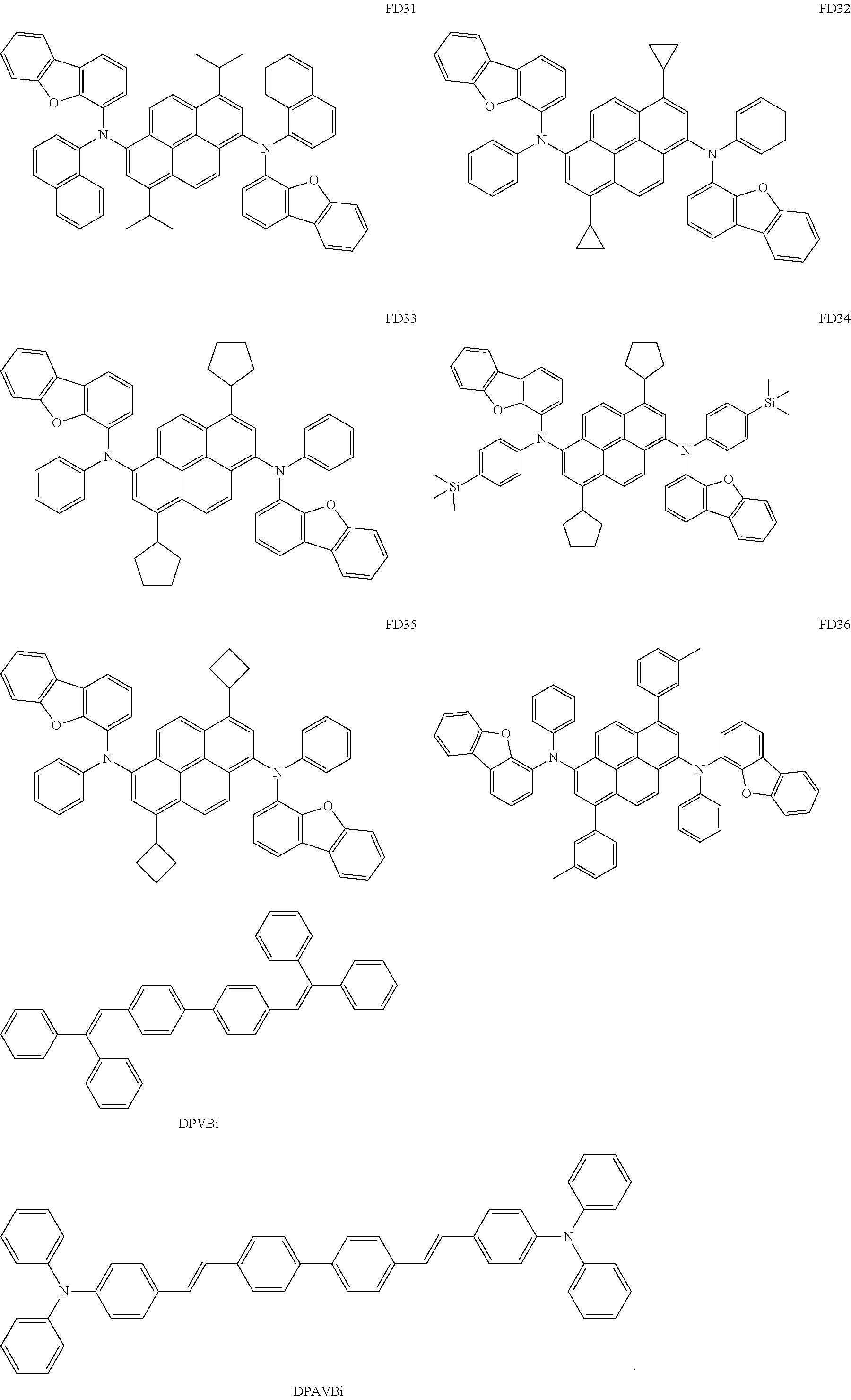

### Delayed Fluorescence Material

The emission layer may include a delayed fluorescence material.

In the present specification, the delayed fluorescence material may be selected from compounds capable of emitting delayed fluorescence by a delayed fluorescence emission mechanism.

The delayed fluorescence material included in the emission layer may act as a host or a dopant depending on the type (or kind) of other materials included in the emission layer.

In an embodiment, a difference between a triplet energy level (eV) of the delayed fluorescence material and a singlet energy level (eV) of the delayed fluorescence material may be equal to or greater than 0 eV and equal to or less than 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence material may effectively occur, and thus, the luminescence efficiency of the light-emitting device 10 may be improved.

For example, the delayed fluorescence material may include i) a material including at least one electron donor (for example, a π electron-rich C₃-C₆₀ cyclic group, such as a carbazole group) and at least one electron acceptor (for example, a sulfoxide group, a cyano group, or a π electron-deficient nitrogen-containing C₁-C₆₀ cyclic group), and ii) a material including a C₈-C₆₀ polycyclic group in which two or more cyclic groups are condensed together while sharing boron (B).

Examples of the delayed fluorescence material may include at least one selected from Compounds DF1 to DF9:

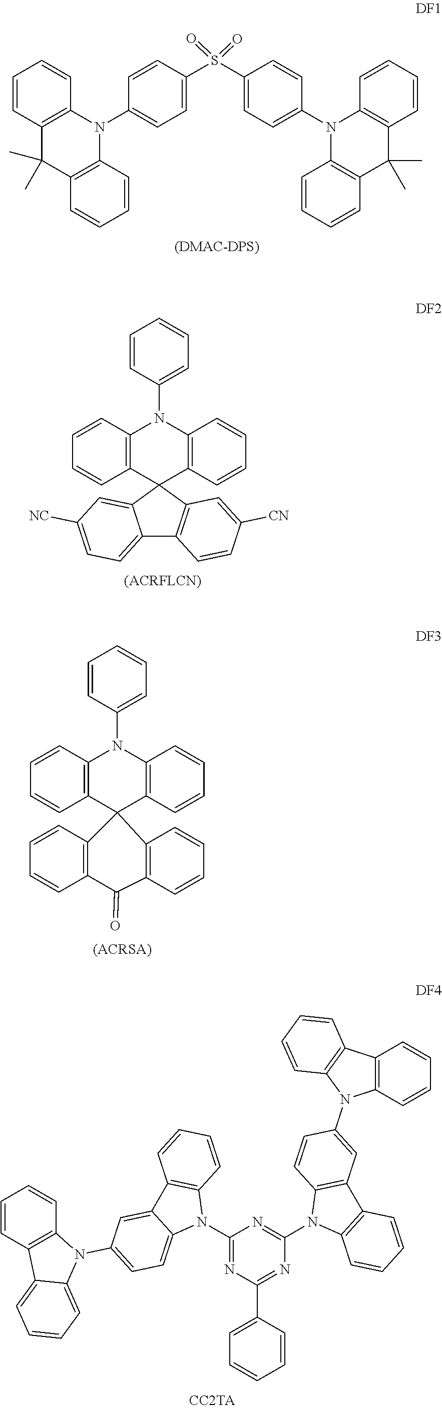

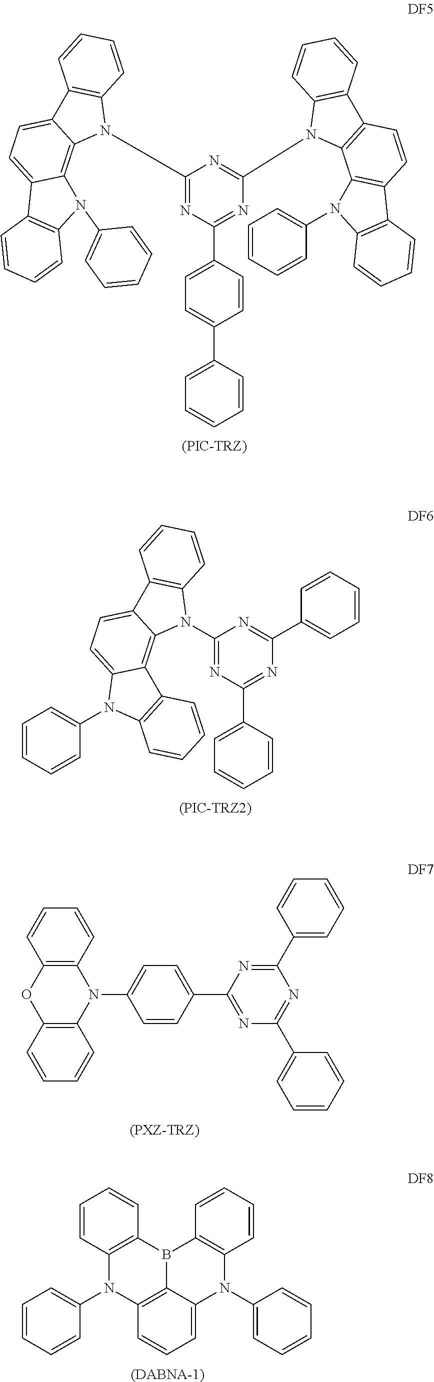

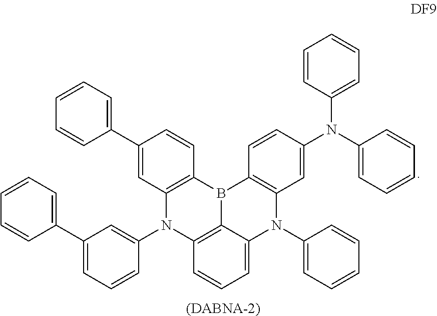

### Quantum Dot

The emission layer may include a quantum dot.

The term “quantum dot,” as used herein, refers to a crystal of a semiconductor compound, and may include any suitable material capable of emitting light of various suitable emission wavelengths according to the size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, and/or any suitable process similar thereto.

The wet chemical process is a method including mixing together a precursor material with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled through a process which costs lower, and is easier than vapor deposition methods, such as metal organic chemical vapor deposition (MOCVD) or molecular beam epitaxy (MBE),

The quantum dot may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Examples of the Group II-VI semiconductor compound may include: a binary compound, such as CdS, CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or any combination thereof.

Examples of the Group III-V semiconductor compound may include: a binary compound, such as GaN, GaP, GaAs, GaSb, AlN, AlP, AlAs, AlSb, InN, InP, InAs, and/or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, and/or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, and/or InAlPSb; or any combination thereof. In an embodiment, the Group III-V semiconductor compound may further include a Group II element. Examples of the Group III-V semiconductor compound further including the Group II element may include InZnP, InGaZnP, and InAlZnP.

Examples of the Group III-VI semiconductor compound may include: a binary compound, such as GaS, GaSe, Ga₂Se₃, GaTe, InS, InSe, In₂S₃, In₂Se₃, and/or InTe; a ternary compound, such as InGaS₃ and/or InGaSes; or any combination thereof.

Examples of the Group I-III-VI semiconductor compound may include: a ternary compound, such as AgInS, AgInS₂, CuInS, CuInS₂, CuGaO₂, AgGaO₂, or AgAlO₂; or any combination thereof.

Examples of the Group IV-VI semiconductor compound may include: a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, and/or SnPbTe; a quaternary compound, such as SnPbSSe, SnPbSeTe, and/or SnPbSTe; or any combination thereof.

The Group IV element or compound may include: a single element compound, such as Si and/or Ge; a binary compound, such as SiC and/or SiGe; or any combination thereof.

Each element included in a multi-element compound, such as the binary compound, the ternary compound, and the quaternary compound, may be present at a uniform concentration or non-uniform concentration in a particle.

The quantum dot may have a single structure in which the concentration of each element in the quantum dot is uniform (e.g., substantially uniform), or a core-shell dual structure. For example, a material included in the core and a material included in the shell may be different from each other.

The shell of the quantum dot may act as a protective layer that prevents or reduces chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases along a direction toward the center of the core.

Examples of the shell of the quantum dot may include an oxide of metal, metalloid, or non-metal, a semiconductor compound, or a combination thereof. Examples of the oxide of metal, metalloid, or non-metal may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO; a ternary compound, such as MgAl₂O₄, CoFe₂O₄, NiFe₂O₄, and/or CoMn₂O₄; or any combination thereof. Examples of the semiconductor compound may include, as described herein, a Group II-VI semiconductor compound; a Group III-V semiconductor compound; a Group III-VI semiconductor compound; a Group I-III-VI semiconductor compound; a Group IV-VI semiconductor compound; or any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of an emission wavelength spectrum of the quantum dot may be about 45 nm or less, for example, about 40 nm or less, for example, about 30 nm or less, and within these ranges, color purity and/or color reproducibility may be improved. In addition, because light through the quantum dot is emitted in all directions (e.g., substantially all directions), a wide viewing angle may be improved.

In addition, the quantum dot may be in the form of a spherical particle, a pyramidal particle, a multi-arm particle, a cubic nanoparticle, a nanotube particle, a nanowire particle, a nanofiber particle, and/or a nanoplate particle.

Because the energy band gap may be adjusted by controlling the size of the quantum dot, light having various suitable wavelength bands may be obtained from the emission layer including the quantum dot. Accordingly, by using quantum dots of different sizes, a light-emitting device that emits light of various suitable wavelengths may be implemented. In an embodiment, the size of the quantum dot may be selected to emit red, green and/or blue light. In addition, the size of the quantum dot may be configured to emit white light by combination of light of various suitable colors.

### Electron Transport Region in Interlayer 130

The electron transport region may have i) a single-layered structure consisting of a single layer consisting of a single material, ii) a single-layered structure consisting of a single layer consisting of a plurality of different materials, or iii) a multi-layered structure including a plurality of layers including different materials.

The electron transport region may include a buffer layer, a hole blocking layer, an electron control layer, an electron transport layer, an electron injection layer, or any combination thereof.

For example, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, the layers of each structure being stacked sequentially from the emission layer.

The electron transport region (for example, the buffer layer, the hole blocking layer, the electron control layer, or the electron transport layer in the electron transport region) may include a metal-free compound including at least one π electron-deficient nitrogen-containing C₁-C₆₀ cyclic group.

For example, the electron transport region may include a compound represented by Formula 601:

[Ar₆₀₁]ₓₑ₁₁-[(L₆₀₁)ₓₑ₁₁-R₆₀₁]ₓₑ₂₁ Formula 601

wherein, in Formula 601,

Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ are each the same as described herein in connection with Q₁,

xe21 may be 1, 2, 3, 4, or 5, and

at least one selected from Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In one or more embodiments, Ar₆₀₁ in Formula 601 may be a substituted or unsubstituted anthracene group.

In one or more embodiments, the electron transport region may include a compound represented by Formula 601-1:

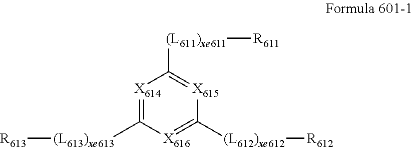

wherein, in Formula 601-1,

X₆₁₄ may be N or C(R₆₁₄), X₆₁₅ may be N or C(R₆₁₅), X₆₁₆ may be N or C(R₆₁₆), and at least one selected from X₆₁₄ to X₆₁₆ may be N,

L₆₁₁ to L₆₁₃ are each the same as described herein in connection with L₆₀₁,

xe611 to xe613 are each the same as described herein in connection with xe1,

R₆₁₁ to R₆₁₃ are each the same as described herein in connection with R₆₀₁, and

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

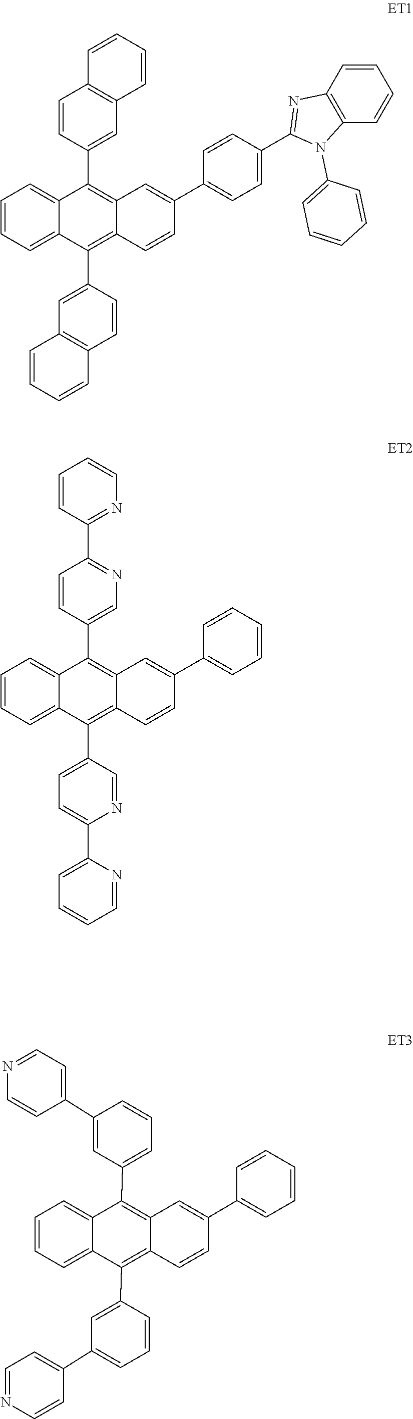 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

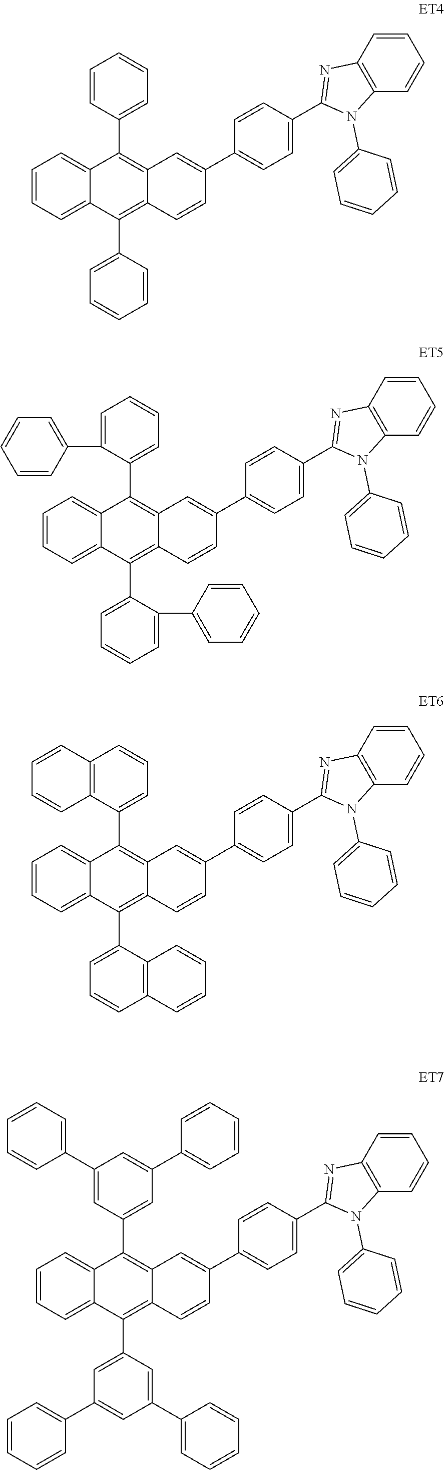 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

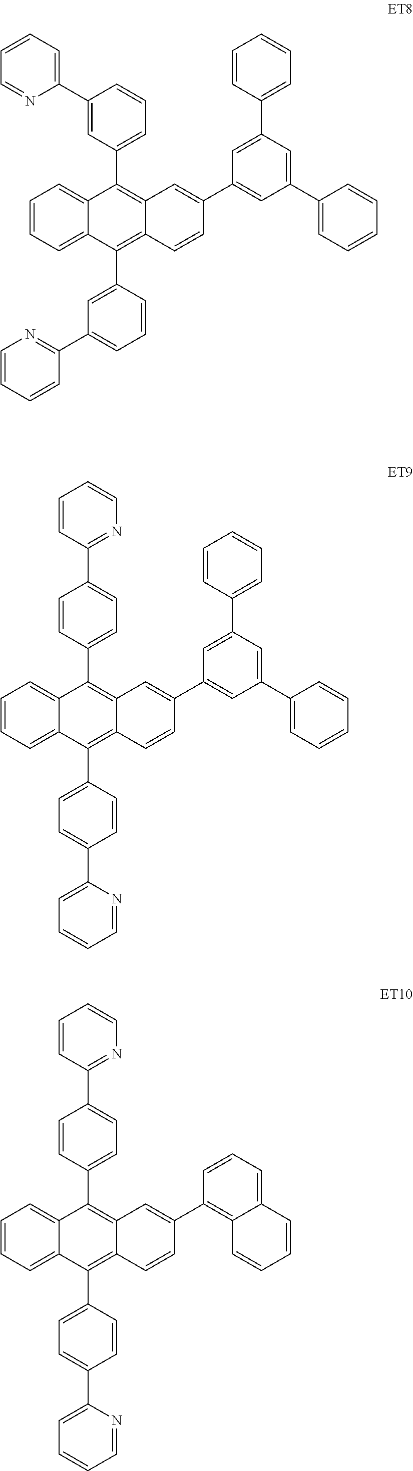 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

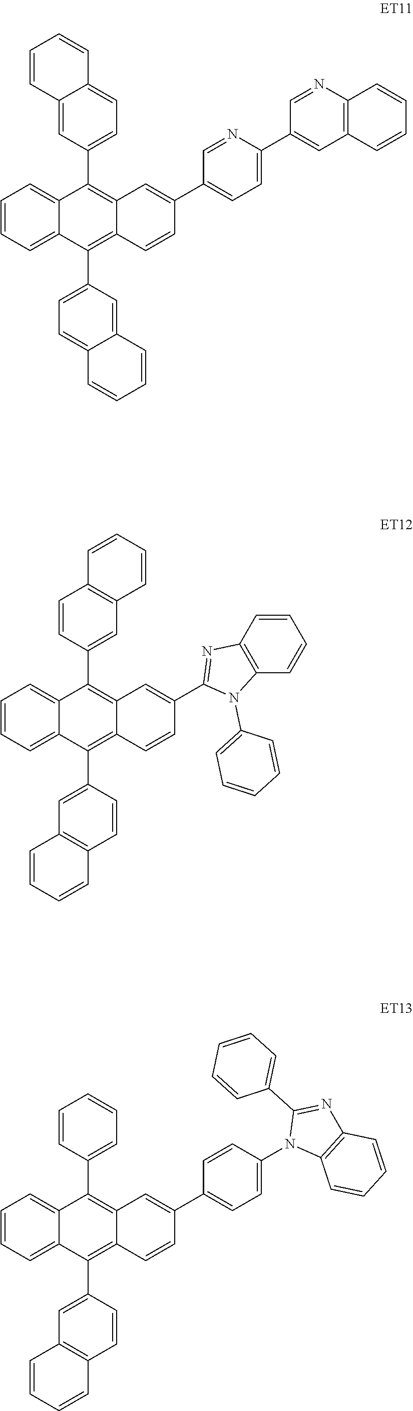 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

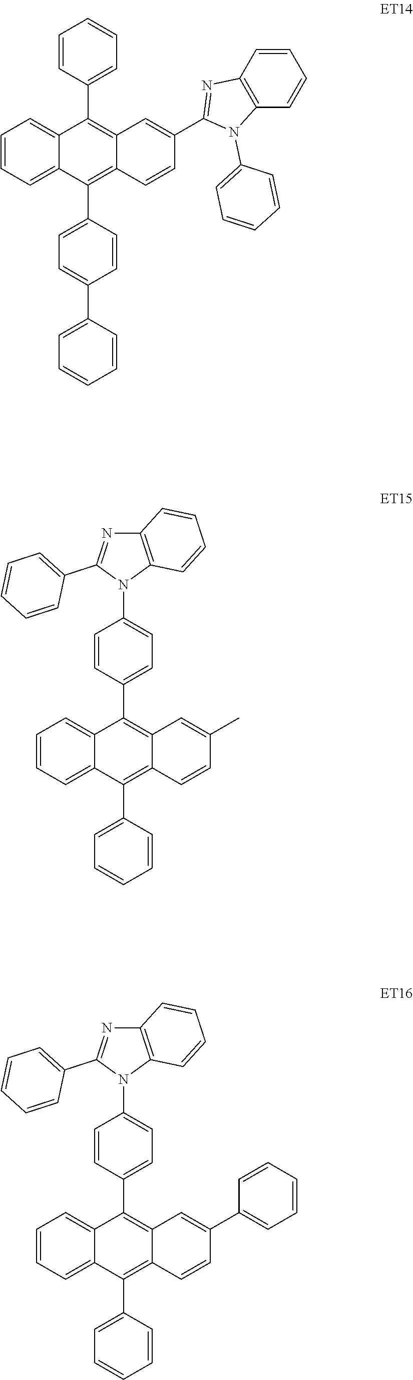 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

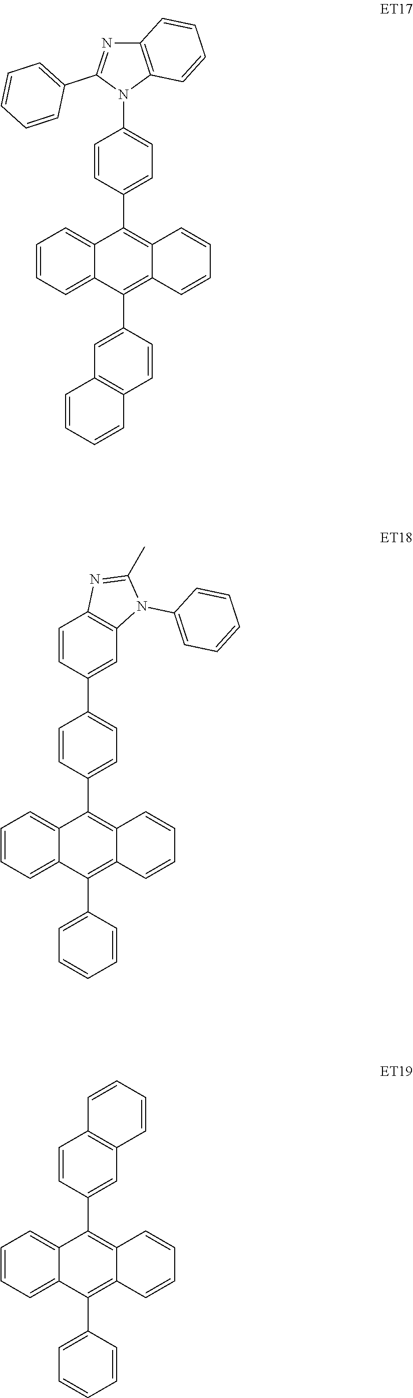

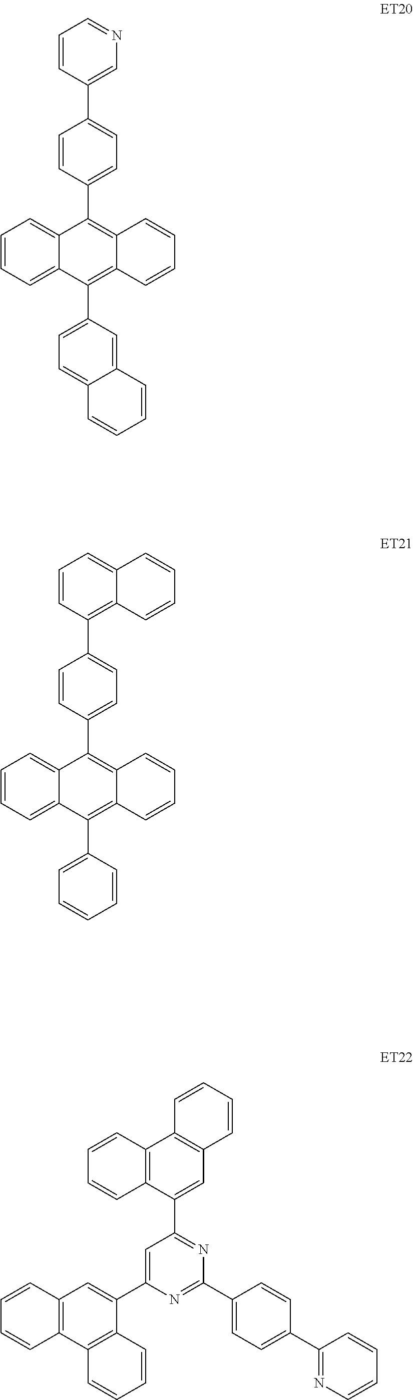

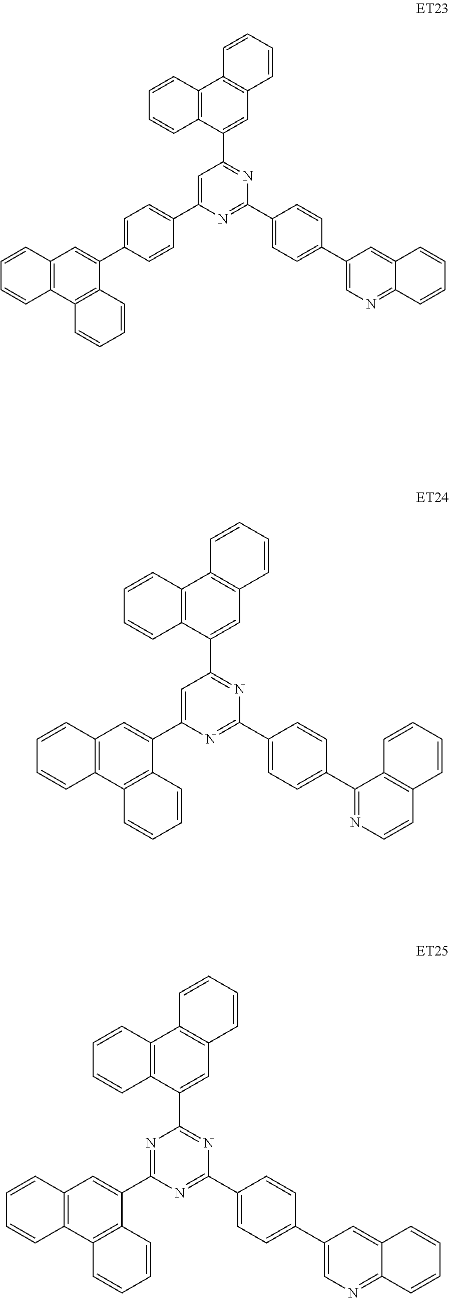 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

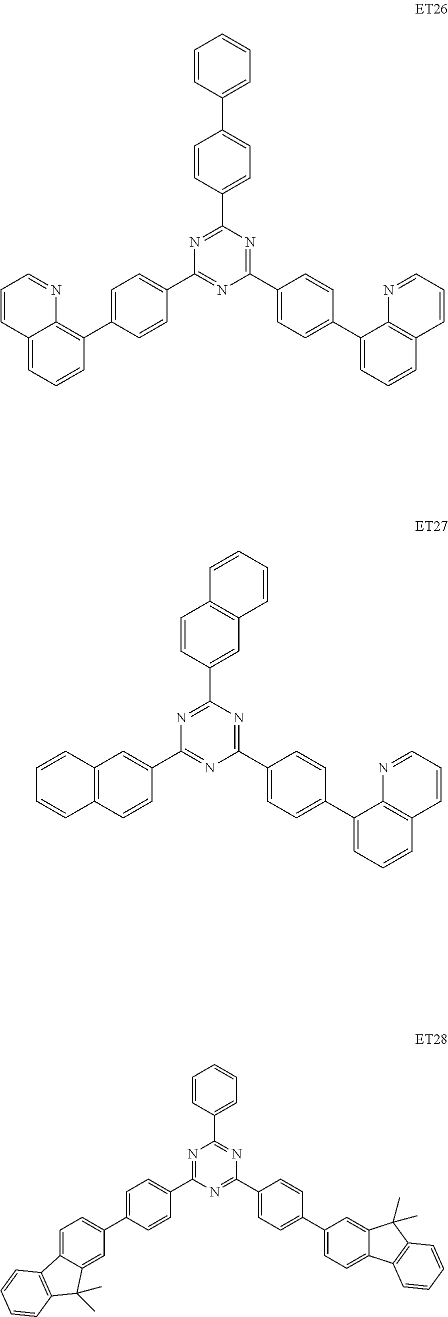 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

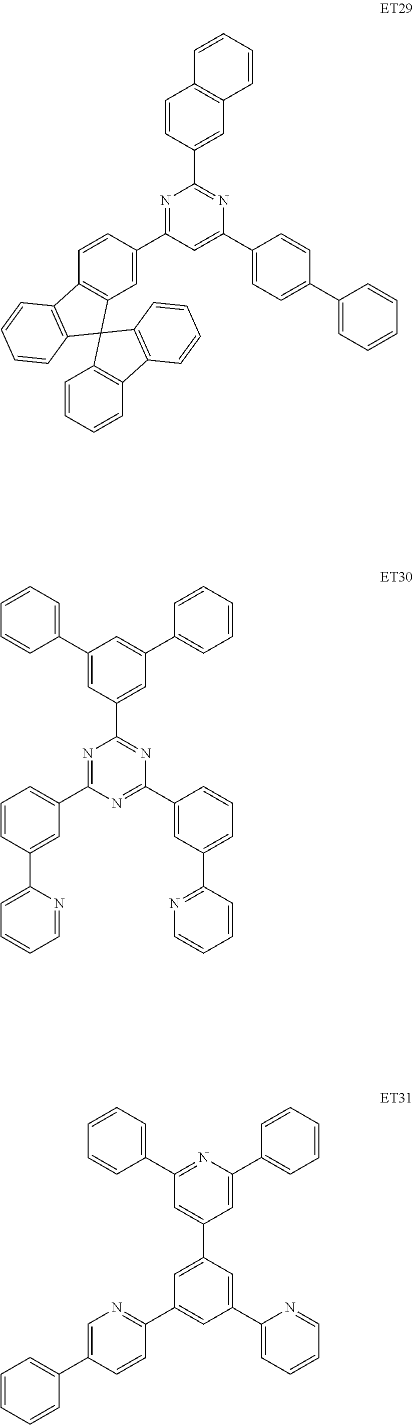 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1]

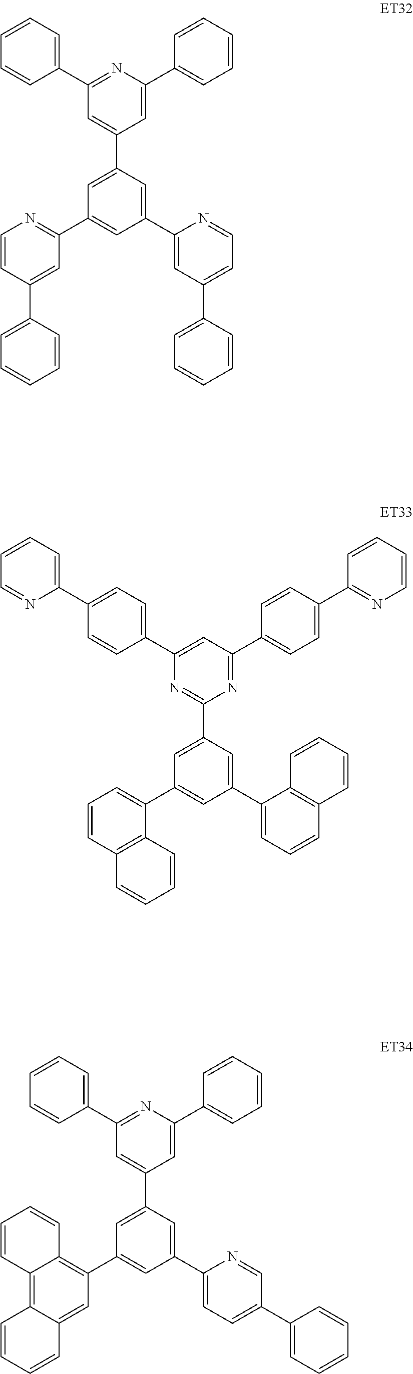 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

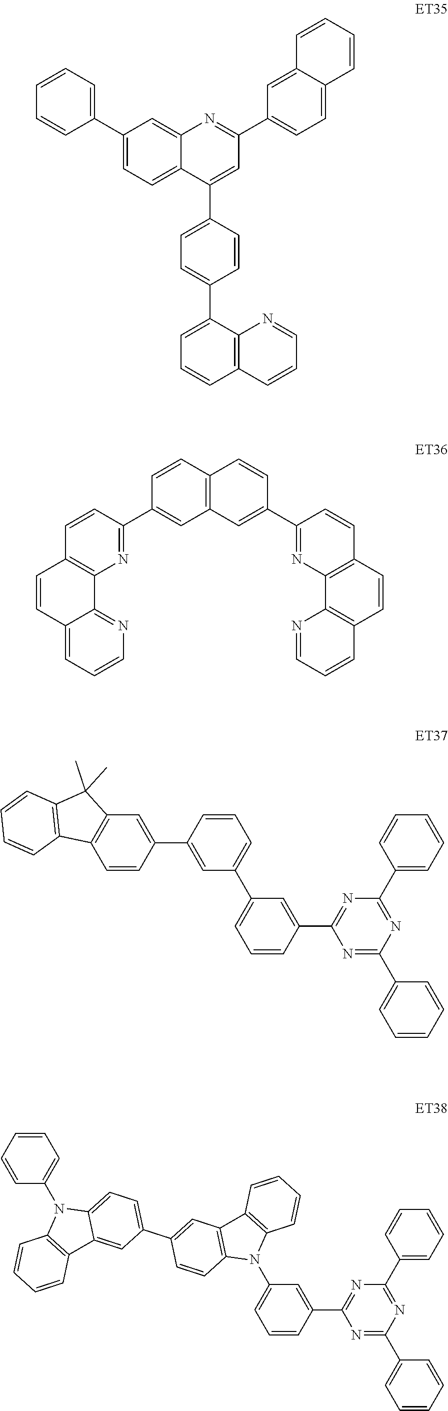 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

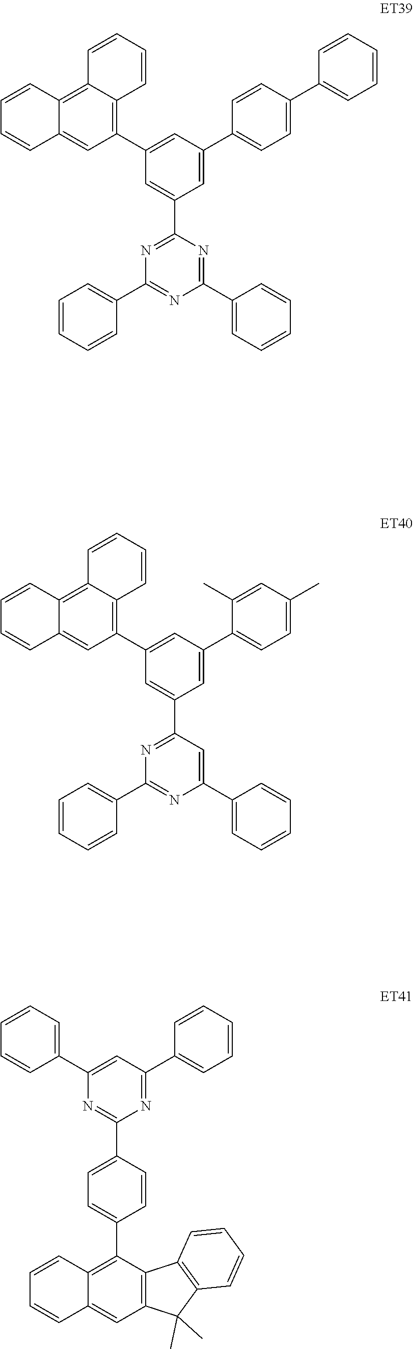 [SMILES: c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1]

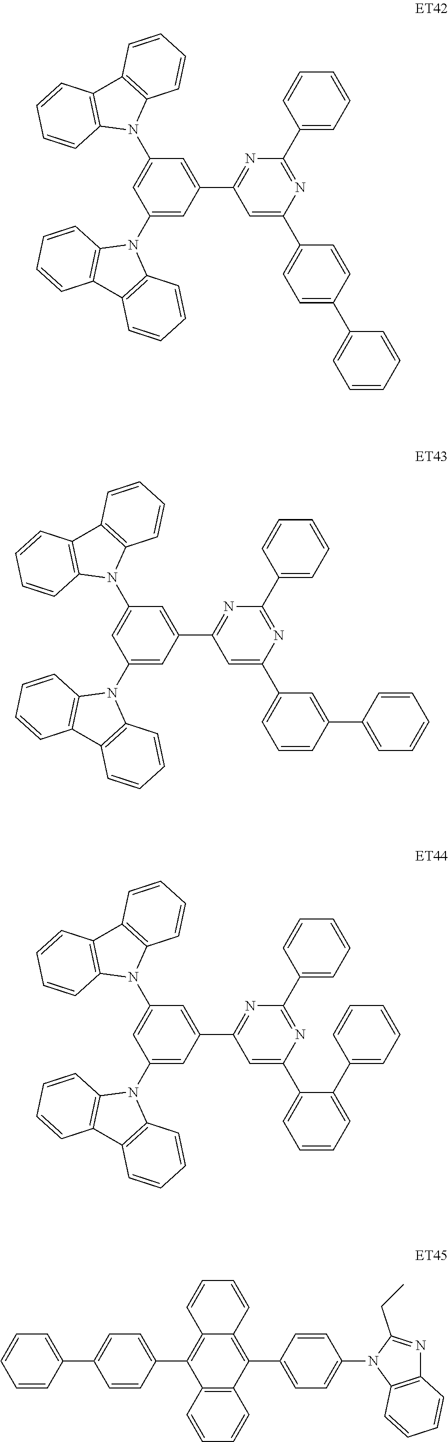 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

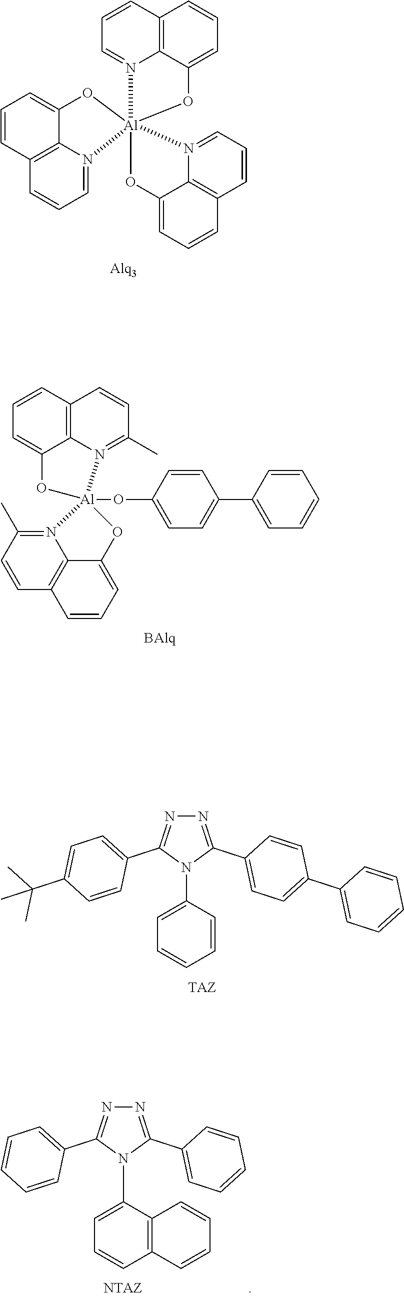

A thickness of the electron transport region may be in a range of about 100 Å to about 5,000 Å, for example, about 160 Å to about 4,000 Å. When the electron transport region includes a buffer layer, a hole blocking layer, an electron control layer, an electron transport layer, or any combination thereof, a thickness of the buffer layer, the hole blocking layer, or the electron control layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron transport region are within these ranges, suitable or satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, and/or a Cs ion, and a metal ion of the alkaline earth metal complex may be a Be ion, a Mg ion, a Ca ion, a Sr ion, and/or a Ba ion. A ligand coordinated with the metal ion of the alkali metal complex or the alkaline earth-metal complex may include hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

For example, the metal-containing material may include a Li complex. The Li complex may include, for example, Compound ET-D1 (LiQ) and/or ET-D2:

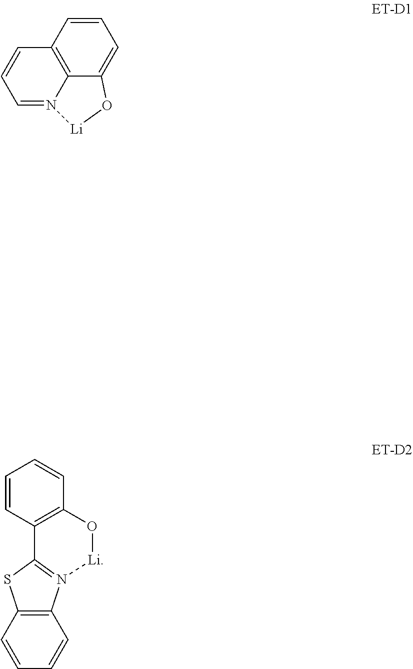

The electron transport region may include an electron injection layer that facilitates the injection of electrons from the second electrode 150. The electron injection layer may be in direct contact (e.g., physical contact) with the second electrode 150.

The electron injection layer may have i) a single-layered structure consisting of a single layer consisting of a single material, ii) a single-layered structure consisting of a single layer consisting of a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, iodides, etc.), and/or tellurides of the alkali metal, the alkaline earth metal, and the rare earth metal, or any combination thereof.

The alkali metal-containing compound may include: alkali metal oxides, such as Li₂O, Cs₂O, and/or K₂O; alkali metal halides, such as LiF, NaF, CsF, KF, LiI, NaI, CsI, and/or KI; or any combination thereof. The alkaline earth metal-containing compound may include an alkaline earth metal compound, such as BaO, SrO, CaO, BaₓSr₁₋ₓO (wherein x is a real number satisfying the condition of 0<x<1), and/or BaₓCa₁₋ₓO (wherein x is a real number satisfying the condition of 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one selected from ions of the alkali metal, the alkaline earth metal, and the rare earth metal and ii), as a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In an embodiment, the electron injection layer may include (e.g., consist of) i) an alkali metal-containing compound (for example, alkali metal halide), ii) a) an alkali metal-containing compound (for example, alkali metal halide); and b) an alkali metal, an alkaline earth metal, a rare earth metal, or any combination thereof. For example, the electron injection layer may be a KI:Yb co-deposited layer, an RbI:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

A thickness of the electron injection layer may be in a range of about 1 Å to about 100 Å, for example, about 3 Å to about 90 Å. When the thickness of the electron injection layer is within this range, suitable or satisfactory electron injection characteristics may be obtained without a substantial increase in driving voltage.

### Second Electrode 150

The second electrode 150 is on the interlayer 130 as described above. The second electrode 150 may be a cathode, which is an electron injection electrode, and as a material for forming the second electrode 150, a metal, an alloy, an electrically conductive compound, or any combination thereof, each having a low work function, may be used.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be outside the first electrode 110, and/or a second capping layer may be outside the second electrode 150. In an embodiment, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in the emission layer of the interlayer 130 of the light-emitting device 10 may be extracted toward the outside through the first electrode 110, which is a semi-transmissive electrode or a transmissive electrode, and the first capping layer. Light generated in the emission layer of the interlayer 130 of the light-emitting device 10 may be extracted toward the outside through the second electrode 150, which is a semi-transmissive electrode or a transmissive electrode, and the second capping layer.

The first capping layer and the second capping layer may increase external luminescence efficiency according to the principle of constructive interference. Accordingly, the light extraction efficiency of the light-emitting device 10 may be increased, so that the luminescence efficiency of the light-emitting device 10 may be improved.

Each of the first capping layer and the second capping layer may include a material having a refractive index of 1.6 or more (at a wavelength of 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof. In an embodiment, at least one selected from the first capping layer and the second capping layer may each independently include an amine group-containing compound.

For example, at least one selected from the first capping layer and the second capping layer may each independently include a compound represented by Formula 201, a compound represented by Formula 202, or any combination thereof.

In one or more embodiments, at least one selected from the first capping layer and the second capping layer may each independently include one selected from Compounds HT28 to HT33, one selected from Compounds CP1 to CP6, β-NPB, or any combination thereof:

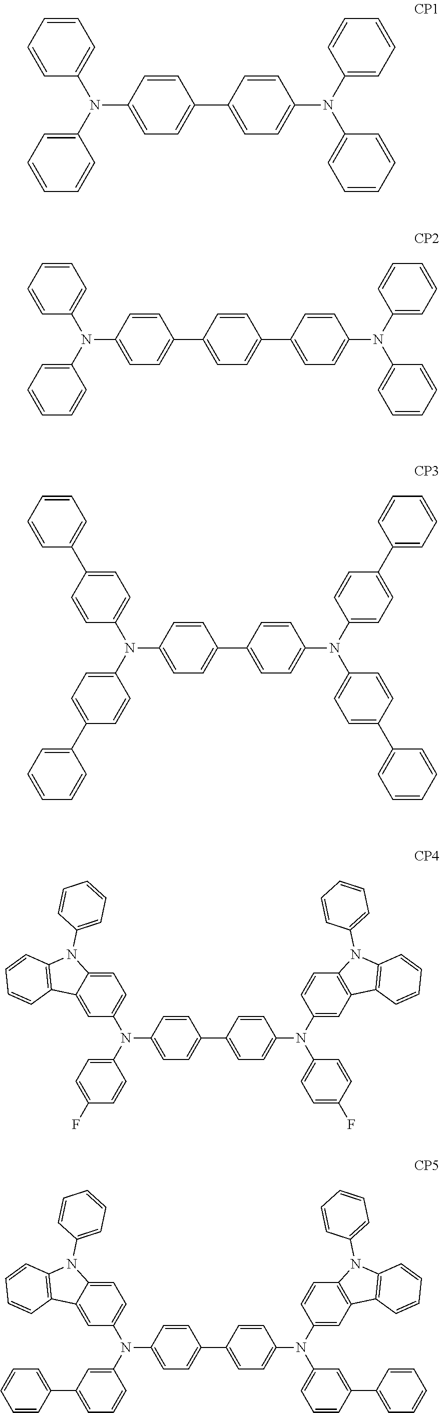 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

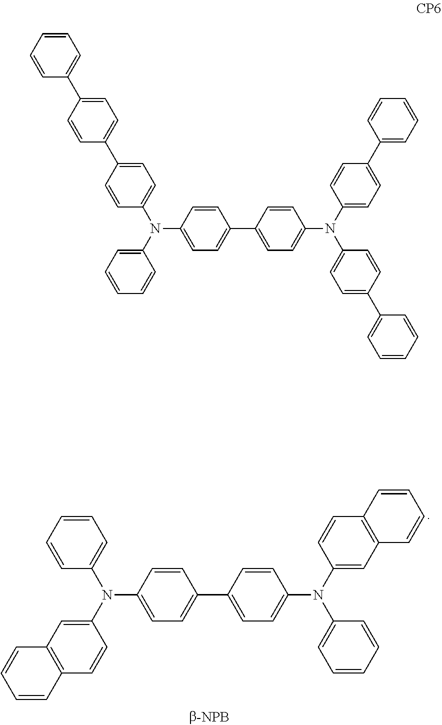

### Film

The amine compound represented by Formula 1 may be included in various suitable films. Accordingly, another aspect of embodiments of the present disclosure provides a film including the amine compound represented by Formula 1. The film may be, for example, an optical member (or a light control means) (for example, a color filter, a color conversion member, a capping layer, a light extraction efficiency enhancement layer, a selective light absorbing layer, a polarizing layer, a quantum dot-containing layer, etc.), a light-blocking member (for example, a light reflective layer, a light absorbing layer, etc.), and/or a protective member (for example, an insulating layer, a dielectric layer, etc.).

### Electronic Apparatus

The light-emitting device may be included in various suitable electronic apparatuses. For example, the electronic apparatus including the light-emitting device may be a light-emitting apparatus, an authentication apparatus, and/or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be in at least one traveling direction of light emitted from the light-emitting device. For example, the light emitted from the light-emitting device may be blue light or white light. The light-emitting device is the same as described above. In an embodiment, the color conversion layer may include a quantum dot. The quantum dots may be, for example, the same as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel defining film may be located among the subpixel areas to define each of the subpixel areas.

The color filter may further include a plurality of color filter areas and light-shielding patterns located among the color filter areas, and the color conversion layer may further include a plurality of color conversion areas and light-shielding patterns located among the color conversion areas.

The color filter areas (or the color conversion areas) may include a first area that emits a first color light, a second area that emits a second color light, and/or a third area that emits a third color light, and the first color light, the second color light, and/or the third color light may have different maximum emission wavelengths. For example, the first color light may be red light, the second color light may be green light, and the third color light may be blue light. For example, the color filter areas (or the color conversion areas) may include quantum dots. In an embodiment, the first area may include red quantum dots, the second area may include green quantum dots, and the third area may not include quantum dots. The quantum dot is the same as described herein. The first area, the second area, and/or the third area may each further include a scatterer.

For example, the light-emitting device may emit a first light, the first area may absorb the first light to emit a first-first color light, the second area may absorb the first light to emit a second-first color light, and the third area may absorb the first light to emit a third-first color light. In this regard, the first-first color light, the second-first color light, and the third-first color light may have different maximum emission wavelengths. In an embodiment, the first light may be blue light, the first-first color light may be red light, the second-first color light may be green light, and the third-first color light may be blue light.

The electronic apparatus may further include a thin-film transistor, in addition to the light-emitting device as described above. The thin-film transistor may include a source electrode, a drain electrode, and an activation layer, wherein one selected from the source electrode and the drain electrode may be electrically connected to one selected from the first electrode and the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may further include a sealing portion for sealing the light-emitting device. The sealing portion may be between the color filter and/or the color conversion layer and the light-emitting device. The sealing portion allows light from the light-emitting device to be extracted to the outside, and concurrently (e.g., simultaneously) prevents or reduces penetration of ambient air and/or moisture into the light-emitting device. The sealing portion may be a sealing substrate including a transparent glass substrate and/or a plastic substrate. The sealing portion may be a thin-film encapsulation layer including at least one layer of an organic layer and/or an inorganic layer. When the sealing portion is a thin-film encapsulating layer, the electronic apparatus may be flexible.

Various suitable functional layers may be additionally on the sealing portion, in addition to the color filter and/or the color conversion layer, according to the use of the electronic apparatus. Examples of the functional layers may include a touch screen layer, a polarizing layer, and the like. The touch screen layer may be a pressure-sensitive touch screen layer, a capacitive touch screen layer, and/or an infrared touch screen layer. The authentication apparatus may be, for example, a biometric authentication apparatus that authenticates an individual by using biometric information of a living body (for example, fingertips, pupils, etc.).

The authentication apparatus may further include, in addition to the light-emitting device as described above, a biometric information collector.

The electronic apparatus may be applied to various suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, and/or endoscope displays), fish finders, various suitable measuring instruments, meters (for example, meters for a vehicle, an aircraft, and/or a vessel), projectors, and/or the like.

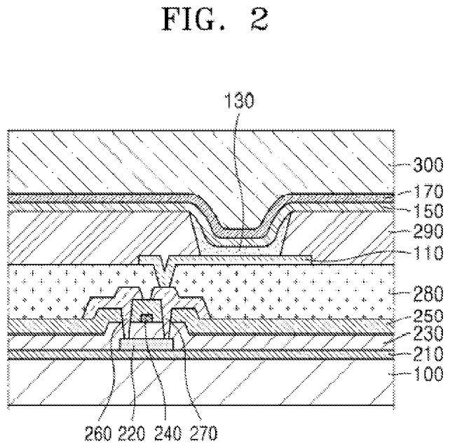

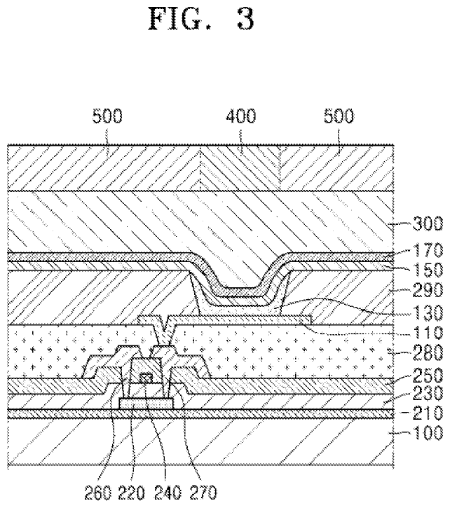

### Description of FIGS. 2 and 3

FIG. 2 is a cross-sectional view of a light-emitting apparatus according to an embodiment.

The light-emitting apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, and/or a metal substrate. A buffer layer 210 may be on the substrate 100. The buffer layer 210 may prevent or reduce penetration of impurities through the substrate 100 and may provide a flat surface on the substrate 100.

The TFT may be on the buffer layer 210. The TFT may include an activation layer 220, a gate electrode 240, a source electrode 260, and a drain electrode 270.

The activation layer 220 may include an inorganic semiconductor such as silicon and/or polysilicon, an organic semiconductor, and/or an oxide semiconductor, and may include a source region, a drain region, and a channel region.

A gate insulating film 230 for insulating the activation layer 220 from the gate electrode 240 may be on the activation layer 220, and the gate electrode 240 may be on the gate insulating film 230.

An interlayer insulating film 250 may be on the gate electrode 240. The interlayer insulating film 250 may be between the gate electrode 240 and the source electrode 260 and between the gate electrode 240 and the drain electrode 270 to provide insulation therebetween.

The source electrode 260 and the drain electrode 270 may be on the interlayer insulating film 250. The interlayer insulating film 250 and the gate insulating film 230 may expose the source region and the drain region of the activation layer 220, and the source electrode 260 and the drain electrode 270 may be in contact (e.g., physical contact) with the exposed portions of the source region and the drain region of the activation layer 220.

The TFT is electrically connected to a light-emitting device to drive the light-emitting device, and is covered and protected by a passivation layer 280. The passivation layer 280 may include an inorganic insulating film, an organic insulating film, or any combination thereof. A light-emitting device is provided on the passivation layer 280. The light-emitting device may include a first electrode 110, an interlayer 130, and a second electrode 150.

The first electrode 110 may be on the passivation layer 280. The passivation layer 280 may expose a certain region of the drain electrode 270 without fully covering the drain electrode 270, and the first electrode 110 may be connected to the exposed region of the drain electrode 270.

A pixel defining layer 290 including an insulating material may be on the first electrode 110. The pixel defining layer 290 may expose a certain region of the first electrode 110, and the interlayer 130 may be formed in the exposed region of the first electrode 110. The pixel defining layer 290 may be a polyimide and/or polyacrylic organic film. In some embodiments, at least some layers of the interlayer 130 may extend beyond the upper portion of the pixel defining layer 290 in the form of a common layer.

The second electrode 150 may be on the interlayer 130, and a capping layer 170 may be additionally on the second electrode 150. The capping layer 170 may cover the second electrode 150.

The encapsulation portion 300 may be on the capping layer 170. The encapsulation portion 300 may be on a light-emitting device to protect the light-emitting device from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, etc.), an epoxy-based resin (for example, aliphatic glycidyl ether (AGE), etc.), or any combination thereof; or a combination of the inorganic film and the organic film.

FIG. 3 is a cross-sectional view of a light-emitting apparatus according to another embodiment.

The light-emitting apparatus of FIG. 3 is substantially the same as the light-emitting apparatus of FIG. 2, except that a light-shielding pattern 500 and a functional region 400 are additionally on the encapsulation portion 300. The functional region 400 may be i) a color filter area, ii) a color conversion area, or iii) a combination of the color filter area and the color conversion area. In an embodiment, the light-emitting device included in the light-emitting apparatus of FIG. 3 may be a tandem light-emitting device.

### Manufacturing Method

The layers included in the hole transport region, the emission layer, and the layers included in the electron transport region may be formed in a certain region by using various suitable methods such as vacuum deposition, spin coating, casting, Langmuir-Blodgett (LB) deposition, ink-jet printing, laser-printing, laser-induced thermal imaging, and/or the like.

When the layers included in the hole transport region, the emission layer, and the layers included in the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group,” as used herein, refers to a cyclic group consisting of carbon only as a ring-forming atom and having 3 to 60 carbon atoms, and the term “C₁-C₆₀ heterocyclic group,” as used herein, refers to a cyclic group that has 1 to 60 carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed together with each other. For example, the C₁-C₆₀ heterocyclic group may have 3 to 61 ring-forming atoms.

The term “cyclic group,” as used herein, may include both the C₃-C₆₀ carbocyclic group and the C₁-C₆₀ heterocyclic group.

The term “π electron-rich C₃-C₆₀ cyclic group,” as used herein, refers to a cyclic group that has 3 to 60 carbon atoms and does not include *—N═*′ as a ring-forming moiety. The term “π electron-deficient nitrogen-containing C₁-C₆₀ cyclic group,” as used herein, refers to a heterocyclic group that has 1 to 60 carbon atoms and includes *—N═*′ as a ring-forming moiety.

For example,

the C₃-C₆₀ carbocyclic group may be i) a T1 group or ii) a condensed cyclic group in which at least two T1 groups are condensed together with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) a T2 group, ii) a condensed cyclic group in which at least two T2 groups are condensed together with each other, or iii) a condensed cyclic group in which at least one T2 group and at least one T1 group are condensed together with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

the π electron-rich C₃-C₆₀ cyclic group may be i) a T1 group, ii) a condensed cyclic group in which at least two T1 groups are condensed together with each other, iii) a T3 group, iv) a condensed cyclic group in which at least two T3 groups are condensed together with each other, or v) a condensed cyclic group in which at least one T3 group and at least one T1 group are condensed together with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),

the π electron-deficient nitrogen-containing C₁-C₆₀ cyclic group may be i) a T4 group, ii) a condensed cyclic group in which at least two T4 groups are condensed together with each other, iii) a condensed cyclic group in which at least one T4 group and at least one T1 group are condensed together with each other, iv) a condensed cyclic group in which at least one T4 group and at least one T3 group are condensed together with each other, or v) a condensed cyclic group in which at least one T4 group, at least one T1 group, and at least one T3 group are condensed together with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,

the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,

the T3 group may be a furan group, a thiophene group, a 1H-pyrrole group, a silole group, or a borole group, and

the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group,” “the C₃-C₆₀ carbocyclic group,” “the C₁-C₆₀ heterocyclic group,” “the π electron-rich C₃-C₆₀ cyclic group,” or “the π electron-deficient nitrogen-containing C₁-C₆₀ cyclic group,” as used herein, refer to a group condensed to any cyclic group, a monovalent group, or a polyvalent group (for example, a divalent group, a trivalent group, a tetravalent group, etc.) according to the structure of a formula for which the corresponding term is used. For example, the “benzene group” may be a benzo group, a phenyl group, a phenylene group, or the like, which may be easily understood by one of ordinary skill in the art according to the structure of a formula including the “benzene group.”

Examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and examples of the divalent C₃-C₆₀ carbocyclic group and the monovalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group,” as used herein, refers to a linear or branched aliphatic hydrocarbon monovalent group that has 1 to 60 carbon atoms, and examples thereof include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group,” as used herein, refers to a divalent group having substantially the same structure as the C₁-C₆₀ alkyl group.

The term “C₂-C₆₀ alkenyl group,” as used herein, refers to a monovalent hydrocarbon group having at least one carbon-carbon double bond at a main chain (e.g., in the middle) or at a terminal end (e.g., the terminus) of the C₂-C₆₀ alkyl group, and examples thereof include an ethenyl group, a propenyl group, and a butenyl group. The term “C₂-C₆₀ alkenylene group,” as used herein, refers to a divalent group having substantially the same structure as the C₂-C₆₀ alkenyl group.

The term “C₂-C₆₀ alkynyl group,” as used herein, refers to a monovalent hydrocarbon group having at least one carbon-carbon triple bond at a main chain (e.g., in the middle) or at a terminal end (e.g., the terminus) of the C₂-C₆₀ alkyl group, and examples thereof include an ethynyl group and a propynyl group. The term “C₂-C₆₀ alkynylene group,” as used herein, refers to a divalent group having substantially the same structure as the C₂-C₆₀ alkynyl group.

The term “C₁-C₆₀ alkoxy group,” as used herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group,” as used herein, refers to a monovalent saturated hydrocarbon cyclic group having 3 to 10 carbon atoms, and examples thereof include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group,” as used herein, refers to a divalent group having substantially the same structure as the C₃-C₁₀ cycloalkyl group.

The term “C₁-C₁₀ heterocycloalkyl group,” as used herein, refers to a monovalent cyclic group that further includes, in addition to a carbon atom, at least one heteroatom as a ring-forming atom and has 1 to 10 carbon atoms, and examples thereof include a 1,2,3,4-oxatriazolidinyl group, a tetrahydrofuranyl group, and a tetrahydrothiophenyl group. The term “C₁-C₁₀ heterocycloalkylene group,” as used herein, refers to a divalent group having substantially the same structure as the C₁-C₁₀ heterocycloalkyl group.

The term “C₃-C₁₀ cycloalkenyl group,” as used herein, refers to a monovalent cyclic group that has 3 to 10 carbon atoms and at least one carbon-carbon double bond in the ring thereof and no aromaticity (e.g., not aromatic), and examples thereof include a cyclopentenyl group, a cyclohexenyl group, and a cycloheptenyl group. The term “C₃-C₁₀ cycloalkenylene group,” as used herein, refers to a divalent group having substantially the same structure as the C₃-C₁₀ cycloalkenyl group.

The term “C₁-C₁₀ heterocycloalkenyl group,” as used herein, refers to a monovalent cyclic group that has, in addition to a carbon atom, at least one heteroatom as a ring-forming atom, 1 to 10 carbon atoms, and at least one double bond in the cyclic structure thereof. Examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group,” as used herein, refers to a divalent group having substantially the same structure as the C₁-C₁₀ heterocycloalkenyl group.

The term “C₆-C₆₀ aryl group,” as used herein, refers to a monovalent group having a carbocyclic aromatic system of 6 to 60 carbon atoms, and the term “C₆-C₆₀ arylene group,” as used herein, refers to a divalent group having a carbocyclic aromatic system of 6 to 60 carbon atoms. Examples of the C₆-C₆₀ aryl group include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the rings may be condensed together with each other.

The term “C₁-C₆₀ heteroaryl group,” as used herein, refers to a monovalent group having a heterocyclic aromatic system of 1 to 60 carbon atoms, further including, in addition to carbon atoms, at least one heteroatom, as ring-forming atoms. The term “C₁-C₆₀ heteroarylene group,” as used herein, refers to a divalent group having a heterocyclic aromatic system of 1 to 60 carbon atoms, further including, in addition to carbon atoms, at least one heteroatom, as ring-forming atoms. Examples of the C₁-C₆₀ heteroaryl group include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the rings may be condensed together with each other.

The term “monovalent non-aromatic condensed polycyclic group,” as used herein, refers to a monovalent group (for example, having 8 to 60 carbon atoms) having two or more rings condensed to each other, only carbon atoms as ring-forming atoms, and no aromaticity in its entire molecular structure (e.g., is not aromatic when considered as a whole). Examples of the monovalent non-aromatic condensed polycyclic group include an indenyl group, a fluorenyl group, a spiro-bifluorenyl group, a benzofluorenyl group, an indenophenanthrenyl group, and an indenoanthracenyl group. The term “divalent non-aromatic condensed polycyclic group,” as used herein, refers to a divalent group having substantially the same structure as the monovalent non-aromatic condensed polycyclic group.

The term “monovalent non-aromatic condensed heteropolycyclic group,” as used herein, refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure (e.g., is not aromatic when considered as a whole). Examples of the monovalent non-aromatic condensed heteropolycyclic group include a pyrrolyl group, a thiophenyl group, a furanyl group, an indolyl group, a benzoindolyl group, a naphtho indolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group,” as used herein, refers to a divalent group having substantially the same structure as the monovalent non-aromatic condensed heteropolycyclic group.

The term “C₆-C₆₀ aryloxy group,” as used herein, refers to —OA₁₀₂ (wherein A₁₀₂ is the C₆-C₆₀ aryl group), and the term “C₆-C₆₀ arylthio group,” as used herein, refers to —SA₁₀₃ (wherein A₁₀₃ is the C₆-C₆₀ aryl group).

The term “C₇-C₆₀ arylalkyl group,” as used herein, refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group,” as used herein, refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ,” as used herein, refers to:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃ and Q₃₁ to Q₃₃ used herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom,” as used herein, refers to any atom other than a carbon atom. Examples of the heteroatom include O, S, N, P, Si, B, Ge, Se, or any combination thereof.

The term “the third-row transition metal,” as used herein, includes hafnium (Hf), tantalum (Ta), tungsten (W), rhenium (Re), osmium (Os), iridium (Ir), platinum (Pt), and gold (Au).

“Ph,” as used herein, refers to a phenyl group, “Me,” as used herein, refers to a methyl group, “Et,” as used herein, refers to an ethyl group, “tert-Bu” or “Bu^{t},” as used herein, refers to a tert-butyl group, and “OMe,” as used herein, refers to a methoxy group.

The term “biphenyl group,” as used herein, refers to “a phenyl group substituted with a phenyl group.” In other words, the “biphenyl group” is a substituted phenyl group having a C₆-C₆₀ aryl group as a substituent.

The term “terphenyl group,” as used herein, refers to “a phenyl group substituted with a biphenyl group”. In other words, the “terphenyl group” is a substituted phenyl group having, as a substituent, a C₆-C₆₀ aryl group substituted with a C₆-C₆₀ aryl group.

* and *′, as used herein, unless defined otherwise, each refer to a binding site to a neighboring atom in a corresponding formula or moiety.

Hereinafter, compounds according to embodiments and light-emitting devices according to embodiments will be described in more detail with reference to Synthesis Examples and Examples. The wording “B was used instead of A” used in describing Synthesis Examples means that an identical molar equivalent of B was used in place of A.

### EXAMPLES

### Synthesis Examples

### Synthesis Example 1: Synthesis of Compound 13

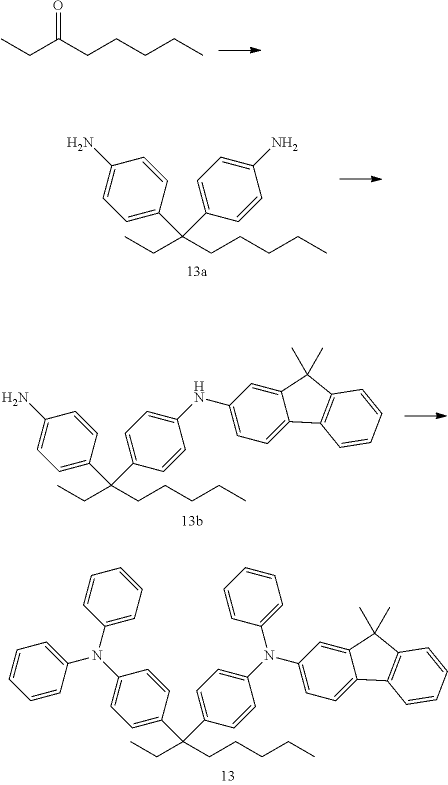

### Synthesis of Intermediate 13a

Octan-3-one (1.0 eq.), aniline (6.0 eq.), and HCl (20 eq.) were stirred under reflux at 160° C. for 48 hours. After cooling, a 0.5 M aqueous sodium bicarbonate solution was slowly added dropwise thereto at 0° C. until the pH reached 7. After the resultant mixture was washed three time with brine and water, the resulting organic layer was dried using MgSO₄ and then dried under reduced pressure. The resulting product was subjected to column chromatography to obtain Intermediate 13a. (Yield: 35%)

### Synthesis of Intermediate 13b

Intermediate 13a (1.0 eq.), 2-bromo-9,9-dimethyl-9H-fluorene (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 13b. (Yield: 40%)

### Synthesis of Compound 13

Intermediate 13b (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 13. (Yield: 85%, HRMS (EI): calcd.: 716.4130; found: 716.4131.)

### Synthesis Example 2: Synthesis of Compound 17

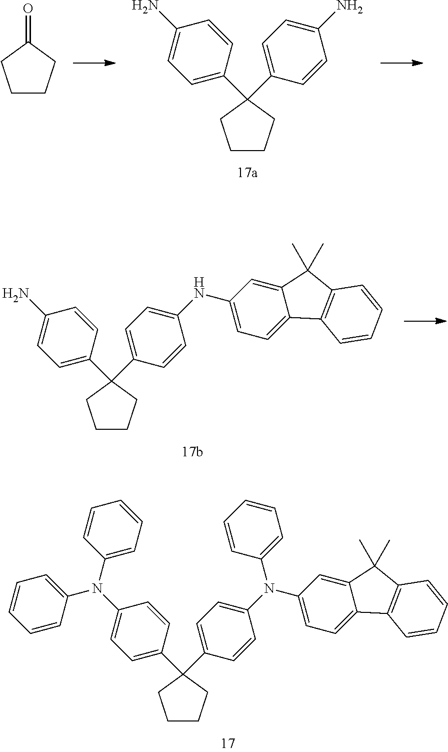 [SMILES: O=C1CCCC1 ; Nc1ccc(C2(c3ccc(N)cc3)CCCC2)cc1 ; CC1(C)c2ccccc2-c2ccc(Nc3ccc(C4(c5ccc(N)cc5)CCCC4)cc3)cc21 ; CC1(C)c2ccccc2-c2ccc(N(c3ccccc3)c3ccc(C4(c5ccc(N(c6ccccc6)c6ccccc6)cc5)CCCC4)cc3)cc21]

### Synthesis of Intermediate 17a

Cyclopentanone (1.0 eq.), aniline (6.0 eq.), and HCl (6.0 eq.) were stirred under reflux at 160° C. for 48 hours. After cooling, a 0.5 M aqueous sodium bicarbonate solution was slowly added dropwise thereto at 0° C. until the pH reached 7. After the resultant mixture was washed three time with brine and water, the resulting organic layer was dried using MgSO₄ and then dried under reduced pressure. The resulting product was subjected to column chromatography to obtain Intermediate 17a. (Yield: 37%)

### Synthesis of Intermediate 17b

Intermediate 17a (1.0 eq.), 2-bromo-9,9-dimethyl-9H-fluorene (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 17b. (Yield: 42%)

### Synthesis of Compound 17

Intermediate 17b (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 17. (Yield: 85%, HRMS (EI): calcd.: 672.3504; found: 672.3506.)

### Synthesis Example 3: Synthesis of Compound 21

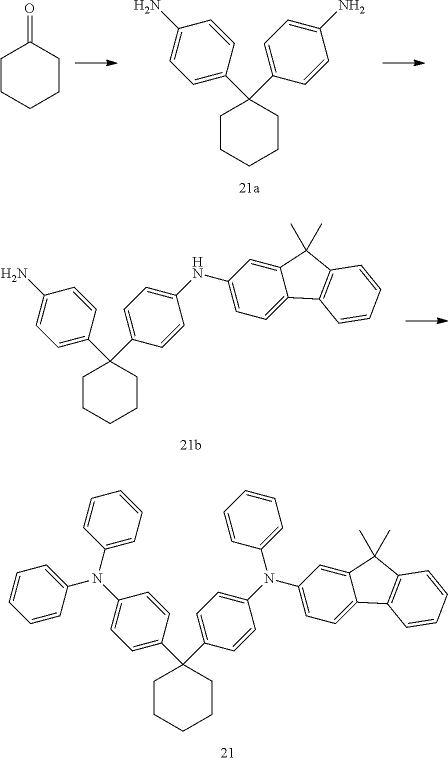 [SMILES: O=C1CCCCC1 ; Nc1ccc(C2(c3ccc(N)cc3)CCCCC2)cc1 ; CC1(C)c2ccccc2-c2ccc(Nc3ccc(C4(c5ccc(N)cc5)CCCCC4)cc3)cc21 ; CC1(C)c2ccccc2-c2ccc(N(c3ccccc3)c3ccc(C4(c5ccc(N(c6ccccc6)c6ccccc6)cc5)CCCCC4)cc3)cc21]

### Synthesis of Intermediate 21a

Cyclohexanone (1.0 eq.), aniline (6.0 eq.), and HCl (6.0 eq.) were stirred under reflux at 160° C. for 48 hours. After cooling, a 0.5 M aqueous sodium bicarbonate solution was slowly added dropwise thereto at 0° C. until the pH reached 7. After the resultant mixture was washed three time with brine and water, the resulting organic layer was dried using MgSO₄ and then dried under reduced pressure. The resulting product was subjected to column chromatography to obtain Intermediate 21a. (Yield: 55%)

### Synthesis of Intermediate 21b

Intermediate 21a (1.0 eq.), 2-bromo-9,9-dimethyl-9H-fluorene (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 21 b. (Yield: 42%)

### Synthesis of Compound 21

Intermediate 21 b (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 21. (Yield: 85%, HRMS (EI): calcd.: 686.3661; found: 686.3665.)

### Synthesis Example 4: Synthesis of Compound 25

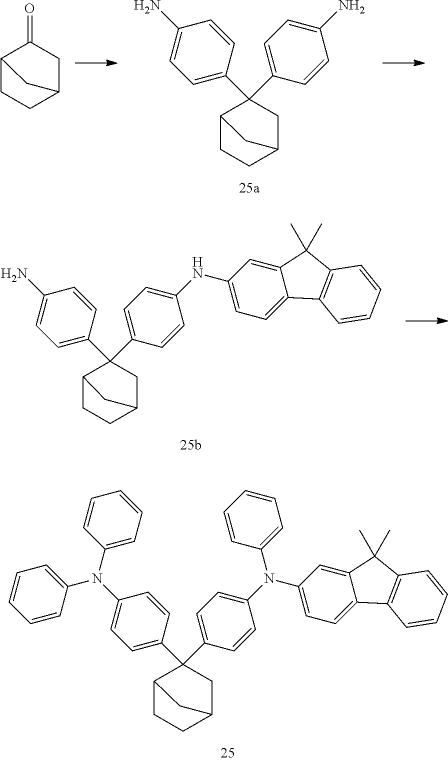 [SMILES: O=C1CC2CCC1C2 ; Nc1ccc(C2(c3ccc(N)cc3)CC3CCC2C3)cc1 ; CC1(C)c2ccccc2-c2ccc(Nc3ccc(C4(c5ccc(N)cc5)CC5CCC4C5)cc3)cc21 ; CC1(C)c2ccccc2-c2ccc(N(c3ccccc3)c3ccc(C4(c5ccc(N(c6ccccc6)c6ccccc6)cc5)CC5CCC4C5)cc3)cc21]

### Synthesis of Intermediate 25a

Norcamphor (1.0 eq.), aniline (6.0 eq.), and HCl (6.0 eq.) were stirred under reflux at 160° C. for 48 hours. After cooling, a 0.5 M aqueous sodium bicarbonate solution was slowly added dropwise thereto at 0° C. until the pH reached 7. After the resultant mixture was washed three time with brine and water, the resulting organic layer was dried using MgSO₄ and then dried under reduced pressure. The resulting product was subjected to column chromatography to obtain Intermediate 25a. (Yield: 25%)

### Synthesis of Intermediate 25b

Intermediate 25a (1.0 eq.), 2-bromo-9,9-dimethyl-9H-fluorene (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 25b. (Yield: 35%)

### Synthesis of Compound 25

Intermediate 25b (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 25. (Yield: 85%, HRMS (EI): calcd.: 698.3661; found: 698.3664.)

### Synthesis Example 5: Synthesis of Compound 26

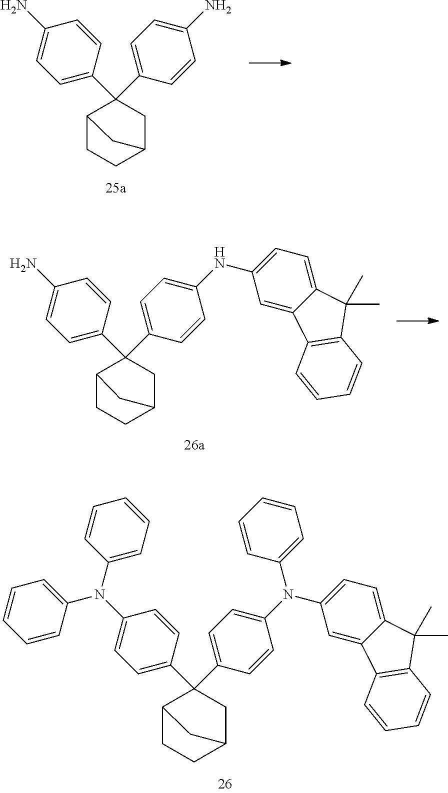 [SMILES: Nc1ccc(C2(c3ccc(N)cc3)CC3CCC2C3)cc1 ; CC1(C)c2ccccc2-c2cc(Nc3ccc(C4(c5ccc(N)cc5)CC5CCC4C5)cc3)ccc21 ; CC1(C)c2ccccc2-c2cc(N(c3ccccc3)c3ccc(C4(c5ccc(N(c6ccccc6)c6ccccc6)cc5)CC5CCC4C5)cc3)ccc21]

### Synthesis of Intermediate 26a

Intermediate 25a (1.0 eq.), 3-bromo-9,9-dimethyl-9H-fluorene (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 26a. (Yield: 40%)

### Synthesis of Compound 26

Intermediate 26a (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 26. (Yield: 86%, HRMS (EI): calcd.: 698.3661; found: 698.3664.)

### Synthesis Example 6: Synthesis of Compound 27

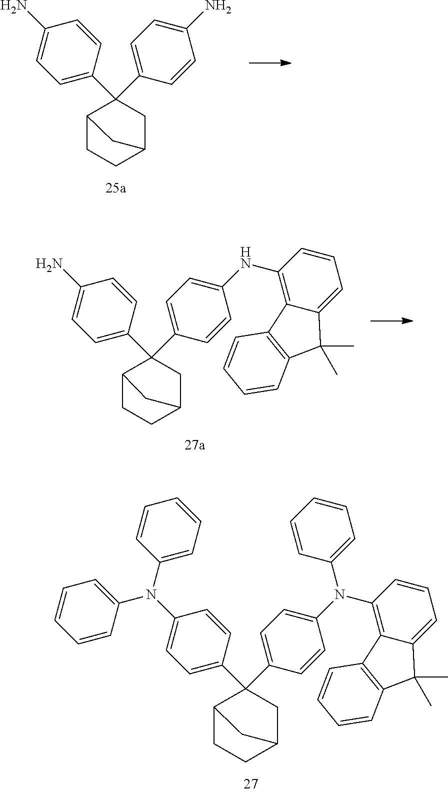 [SMILES: Nc1ccc(C2(c3ccc(N)cc3)CC3CCC2C3)cc1 ; CC1(C)c2ccccc2-c2c(Nc3ccc(C4(c5ccc(N)cc5)CC5CCC4C5)cc3)cccc21 ; CC1(C)c2ccccc2-c2c(N(c3ccccc3)c3ccc(C4(c5ccc(N(c6ccccc6)c6ccccc6)cc5)CC5CCC4C5)cc3)cccc21]

### Synthesis of Intermediate 27a

Intermediate 25a (1.0 eq.), 4-bromo-9,9-dimethyl-9H-fluorene (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 27a. (Yield: 40%)

### Synthesis of Compound 27

Intermediate 27a (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 27. (Yield: 86%, HRMS (EI): calcd.: 698.3661; found: 698.3663.)

### Synthesis Example 7: Synthesis of Compound 29

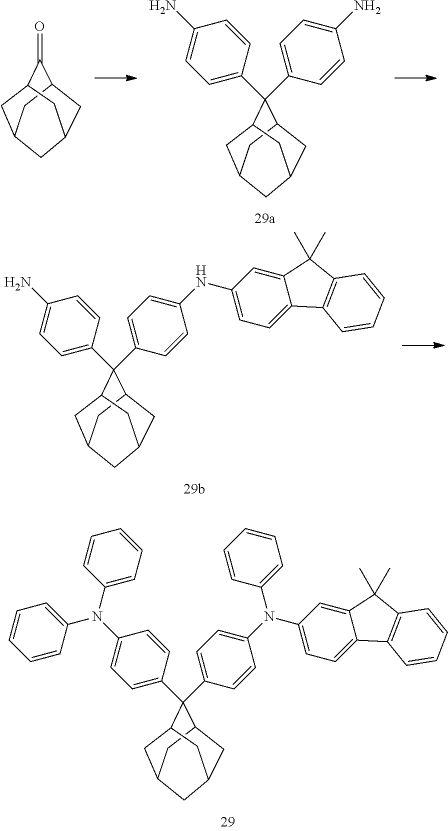 [SMILES: O=C1C2CC3CC(C2)CC1C3 ; Nc1ccc(C2(c3ccc(N)cc3)C3CC4CC(C3)CC2C4)cc1 ; CC1(C)c2ccccc2-c2ccc(Nc3ccc(C4(c5ccc(N)cc5)C5CC6CC(C5)CC4C6)cc3)cc21 ; CC1(C)c2ccccc2-c2ccc(N(c3ccccc3)c3ccc(C4(c5ccc(N(c6ccccc6)c6ccccc6)cc5)C5CC6CC(C5)CC4C6)cc3)cc21]

### Synthesis of Intermediate 29a

Adamantan-2-one (1.0 eq.), aniline (6.0 eq.), and HCl (6.0 eq.) were stirred under reflux at 160° C. for 48 hours. After cooling, a 0.5 M aqueous sodium bicarbonate solution was slowly added dropwise thereto at 0° C. until the pH reached 7. After the resultant mixture was washed three time with brine and water, the resulting organic layer was dried using MgSO₄ and then dried under reduced pressure. The resulting product was subjected to column chromatography to obtain Intermediate 29a. (Yield: 25%)

### Synthesis of Intermediate 29b

Intermediate 29a (1.0 eq.), 2-bromo-9,9-dimethyl-9H-fluorene (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 29b. (Yield: 35%)

### Synthesis of Compound 29

Intermediate 29b (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 29. (Yield: 85%, HRMS (EI): calcd.: 738.3974; found: 738.3975.)

### Synthesis Example 8: Synthesis of Compound 59

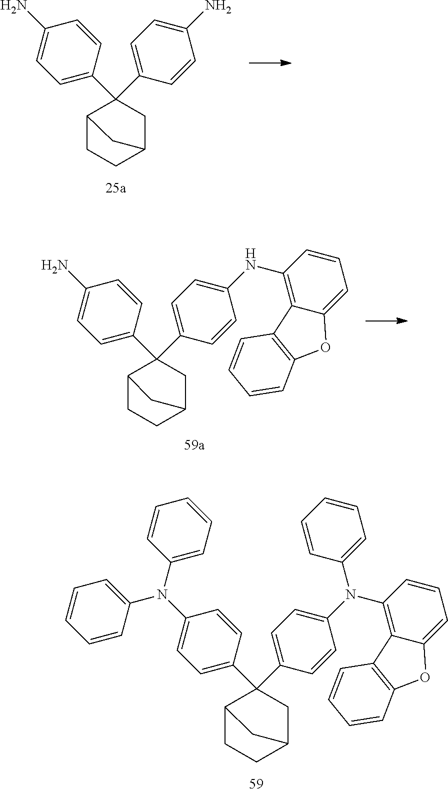 [SMILES: Nc1ccc(C2(c3ccc(N)cc3)CC3CCC2C3)cc1 ; Nc1ccc(C2(c3ccc(Nc4cccc5oc6ccccc6c45)cc3)CC3CCC2C3)cc1 ; c1ccc(N(c2ccccc2)c2ccc(C3(c4ccc(N(c5ccccc5)c5cccc6oc7ccccc7c56)cc4)CC4CCC3C4)cc2)cc1]

### Synthesis of Intermediate 59a

Intermediate 25a (1.0 eq.), 1-bromodibenzofuran (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 59a. (Yield: 40%)

### Synthesis of Compound 59

Intermediate 59a (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 59. (Yield: 86%, HRMS (EI): calcd.: 672.3141; found: 672.3143.)

### Synthesis Example 9: Synthesis of Compound 121

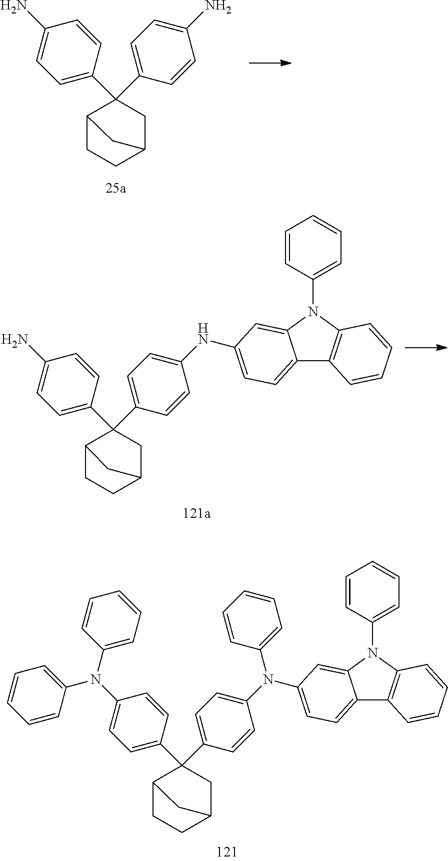 [SMILES: Nc1ccc(C2(c3ccc(N)cc3)CC3CCC2C3)cc1 ; Nc1ccc(C2(c3ccc(Nc4ccc5c6ccccc6n(-c6ccccc6)c5c4)cc3)CC3CCC2C3)cc1 ; c1ccc(N(c2ccccc2)c2ccc(C3(c4ccc(N(c5ccccc5)c5ccc6c7ccccc7n(-c7ccccc7)c6c5)cc4)CC4CCC3C4)cc2)cc1]

### Synthesis of Intermediate 121a

Intermediate 25a (1.0 eq.), 2-bromo-9-phenyl-9H-carbazole (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 121a. (Yield: 40%)

### Synthesis of Compound 121

Intermediate 121a (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 121. (Yield: 86%, HRMS (EI): calcd.: 747.3613; found: 747.3615.)

### Synthesis Example 10: Synthesis of Compound 182

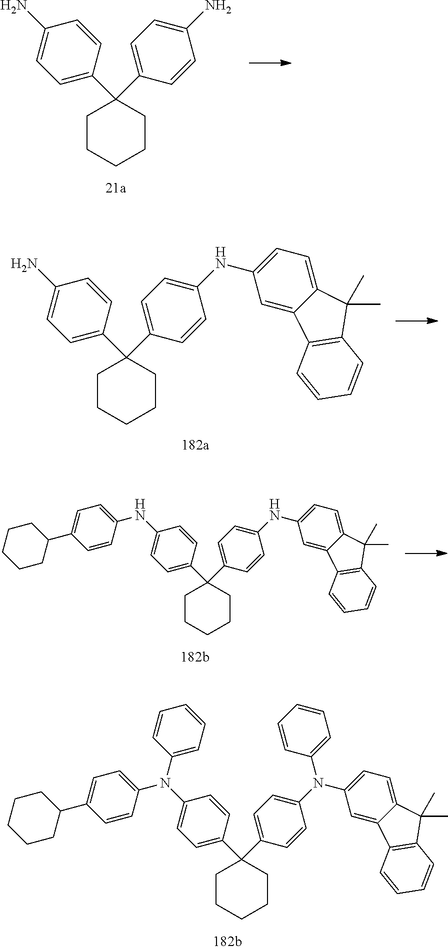 [SMILES: Nc1ccc(C2(c3ccc(N)cc3)CCCCC2)cc1 ; CC1(C)c2ccccc2-c2cc(Nc3ccc(C4(c5ccc(N)cc5)CCCCC4)cc3)ccc21 ; CC1(C)c2ccccc2-c2cc(Nc3ccc(C4(c5ccc(Nc6ccc(C7CCCCC7)cc6)cc5)CCCCC4)cc3)ccc21 ; CC1(C)c2ccccc2-c2cc(N(c3ccccc3)c3ccc(C4(c5ccc(N(c6ccccc6)c6ccc(C7CCCCC7)cc6)cc5)CCCCC4)cc3)ccc21]

### Synthesis of Intermediate 182a

Intermediate 21a (1.0 eq.), 2-bromo-9,9-dimethyl-9H-fluorene (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 182a. (Yield: 42%)

### Synthesis of Intermediate 182b

Intermediate 182a (1.0 eq.), 1-bromo-4-cyclohexylbenzene (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 182b. (Yield: 40%)

### Synthesis of Compound 182

Intermediate 182b (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 182. (Yield: 85%, HRMS (EI): calcd.: 768.4443; found: 768.4445.)

### Synthesis Example 11: Synthesis of Compound 118

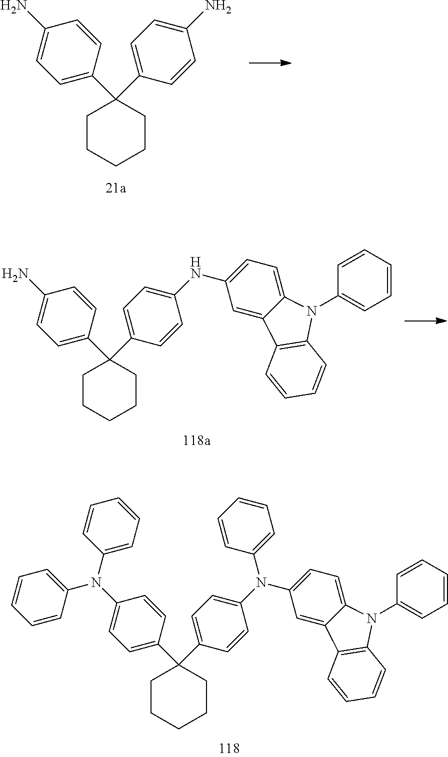 [SMILES: Nc1ccc(C2(c3ccc(N)cc3)CCCCC2)cc1 ; Nc1ccc(C2(c3ccc(Nc4ccc5c(c4)c4ccccc4n5-c4ccccc4)cc3)CCCCC2)cc1 ; c1ccc(N(c2ccccc2)c2ccc(C3(c4ccc(N(c5ccccc5)c5ccc6c(c5)c5ccccc5n6-c5ccccc5)cc4)CCCCC3)cc2)cc1]

### Synthesis of Intermediate 118a

Intermediate 21a (1.0 eq.), 3-bromo-9-phenyl-9H-carbazole (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 118a. (Yield: 42%)

### Synthesis of Compound 118

Intermediate 118a (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 118. (Yield: 85%, HRMS (EI): calcd.: 735.3613; found: 735.3615.)

### Synthesis Example 12: Synthesis of Compound 122

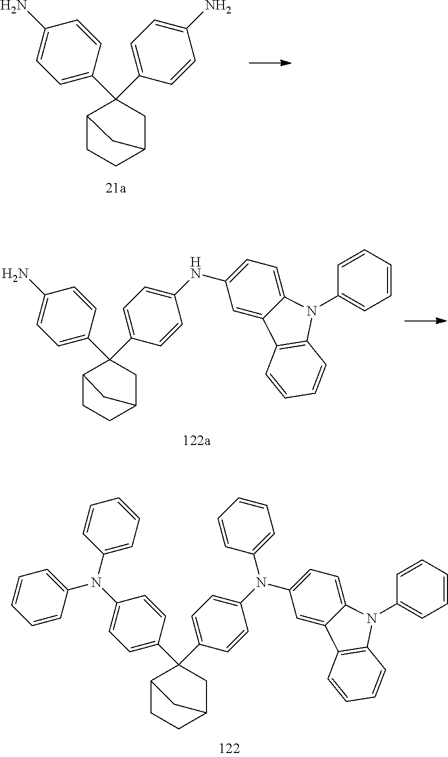 [SMILES: Nc1ccc(C2(c3ccc(N)cc3)CC3CCC2C3)cc1 ; Nc1ccc(C2(c3ccc(Nc4ccc5c(c4)c4ccccc4n5-c4ccccc4)cc3)CC3CCC2C3)cc1 ; c1ccc(N(c2ccccc2)c2ccc(C3(c4ccc(N(c5ccccc5)c5ccc6c(c5)c5ccccc5n6-c5ccccc5)cc4)CC4CCC3C4)cc2)cc1]

### Synthesis of Intermediate 122a

Intermediate 25a (1.0 eq.), 3-bromo-9-phenyl-9H-carbazole (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 122a. (Yield: 40%)

### Synthesis of Compound 122

Intermediate 122a (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 122. (Yield: 85%, HRMS (EI): calcd.: 747.3613; found: 747.3615.)

### Synthesis Example 13: Synthesis of Compound 126

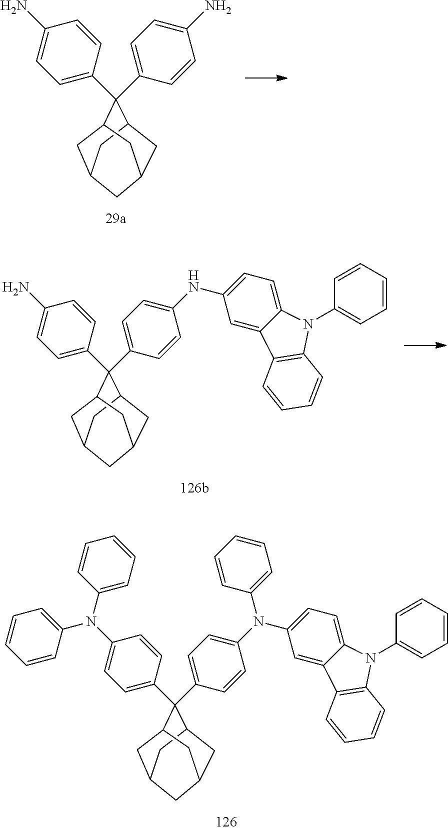 [SMILES: Nc1ccc(C2(c3ccc(N)cc3)C3CC4CC(C3)CC2C4)cc1 ; Nc1ccc(C2(c3ccc(Nc4ccc5c(c4)c4ccccc4n5-c4ccccc4)cc3)C3CC4CC(C3)CC2C4)cc1 ; c1ccc(N(c2ccccc2)c2ccc(C3(c4ccc(N(c5ccccc5)c5ccc6c(c5)c5ccccc5n6-c5ccccc5)cc4)C4CC5CC(C4)CC3C5)cc2)cc1]

### Synthesis of Intermediate 126b

Intermediate 29a (1.0 eq.), 3-bromo-9-phenyl-9H-carbazole (1.0 eq), palladium acetate (0.03 eq.), 2,2′-bis(diphenylphosphino)-1,1′-binaphthyl (0.06 eq.), and sodium tert-butoxide (6.0 eq.) were dissolved in toluene and then stirred at 75° C. for 3 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Intermediate 126b. (Yield: 40%)

### Synthesis of Compound 126

Intermediate 126b (1 eq.), 4-iodobenzene (5.0 eq.), tris(dibenzylideneacetone)dipalladium (0) (0.03 eq.), tri-tert-butylphosphine (0.06 eq.), and sodium tert-butoxide (2.0 eq.) were dissolved in toluene and then stirred at 80° C. for 2 hours under a nitrogen atmosphere. After the resultant mixture was cooled and washed three times using ethyl acetate and water, the resulting organic layer was dried using MgSO₄ and then dried under reduced pressure. The resulting product was subjected to column chromatography to obtain Compound 126. (Yield: 80%, HRMS (EI): calcd.: 787.3926; found: 787.3928.)

### Evaluation Example 1

The highest occupied molecular orbital (HOMO) energy and lowest unoccupied molecular orbital (LUMO) energy of the compounds used in Synthesis Examples 1 to 13 and Comparative Examples were measured, and the results thereof are shown in Table 2.

In more detail, the HOMO and LUMO energies were measured by the methods of Table 1, and the band gap indicates the absolute value of the difference between the LUMO energy level (eV) and the HOMO energy level (eV).

TABLE 1

| | |
|---|---|
| HOMO energy level | By using cyclic voltammetry (CV) (electrolyte: |
| evaluation method | 0.1M Bu₄NPF₆/solvent: dimethylforamide |
| | (DMF)/electrode: 3-electrode system (working |
| | electrode: GC, referenceelectrode: Ag/AgCl, |
| | and auxiliary electrode: Pt)), the potential (V)- |
| | current (A) graph of each compound was |
| | obtained, and then, from the oxidation onset |
| | of the graph, the HOMO energy level of each |
| | compound was calculated. |
| LUMO energy level | By using cyclic voltammetry (CV) (electrolyte: |
| evaluation method | 0.1M Bu₄NPF₆/solvent: dimethylforamide |
| | (DMF)/electrode: 3-electrode system (working |
| | electrode: GC, reference electrode: Ag/AgCl, |
| | and auxiliary electrode: Pt)), the potential (V)- |
| | current (A) graph of each compound was |
| | obtained, and then, from the reduction onset |
| | of the graph, the LUMO energy level of each |
| | compound was calculated. |

TABLE 2

| | HOMO | LUMO | Band gap |
|---|---|---|---|
| Compound | (eV) | (eV) | (eV) |
| Compound | −5.12 | −2.00 | 3.12 |
| 13 | | | |
| Compound | −5.12 | −2.00 | 3.12 |
| 17 | | | |
| Compound | −5.18 | −1.87 | 3.31 |
| 21 | | | |
| Compound | −5.13 | −2.05 | 3.08 |
| 25 | | | |
| Compound | −5.12 | −2.03 | 3.09 |
| 26 | | | |
| Compound | −5.14 | −2.05 | 3.09 |
| 27 | | | |
| Compound | −5.15 | −1.84 | 3.31 |
| 29 | | | |
| Compound | −5.17 | −1.82 | 3.35 |
| 59 | | | |
| Compound | −5.14 | −2.02 | 3.12 |
| 121 | | | |
| Compound | −5.15 | −1.89 | 3.26 |
| 182 | | | |
| Compound | −5.12 | −2.01 | 3.11 |
| 118 | | | |
| Compound | −5.11 | −2.00 | 3.11 |
| 122 | | | |
| Compound | −5.11 | −2.01 | 3.10 |
| 126 | | | |
| NPB | −5.09 | −2.00 | 3.09 |
| Compound | −5.11 | −1.88 | 3.23 |
| A | | | |
| Compound | −5.27 | −1.70 | 3.57 |
| B | | | |
| Compound | −5.21 | −1.68 | 3.53 |
| C | | | |
| Compound | −5.23 | −1.66 | 3.57 |
| D | | | |
| Compound | −5.11 | −2.00 | 3.11 |
| E | | | |
| Compound | −5.21 | −1.67 | 3.54 |
| F | | | |

### Comparative Example 1

As an anode, a Corning 15 Ω/cm² (1,200 Å) ITO glass substrate was cut to a size of 50 mm×50 mm×0.7 mm, sonicated with isopropyl alcohol and pure water each for 5 minutes, and then cleaned by exposure to ultraviolet rays and ozone for 30 minutes. Then, the resultant glass substrate was loaded onto a vacuum deposition apparatus.

2-TNATA was vacuum-deposited on the substrate to form a hole injection layer having a thickness of 600 Å, and then, 4,4′-bis[N-(1-naphthyl)-N-phenylamino]biphenyl (hereinafter, referred to as NPB), which is a hole transport material, was vacuum-deposited thereon as a hole transport compound to form a hole transport layer having a thickness of 300 Å.

9,10-di(naphthalen-2-yl)anthracene (hereinafter, referred to as ADN) as a blue fluorescent host and 4,4′-bis[2-(4-(N,N-diphenylamino)phenyl)vinyl]biphenyl (hereinafter, referred to as DPAVBi) as a blue fluorescent dopant were co-deposited on the hole transport layer at a weight ratio of 98:2 to form an emission layer having a thickness of 300 Å.

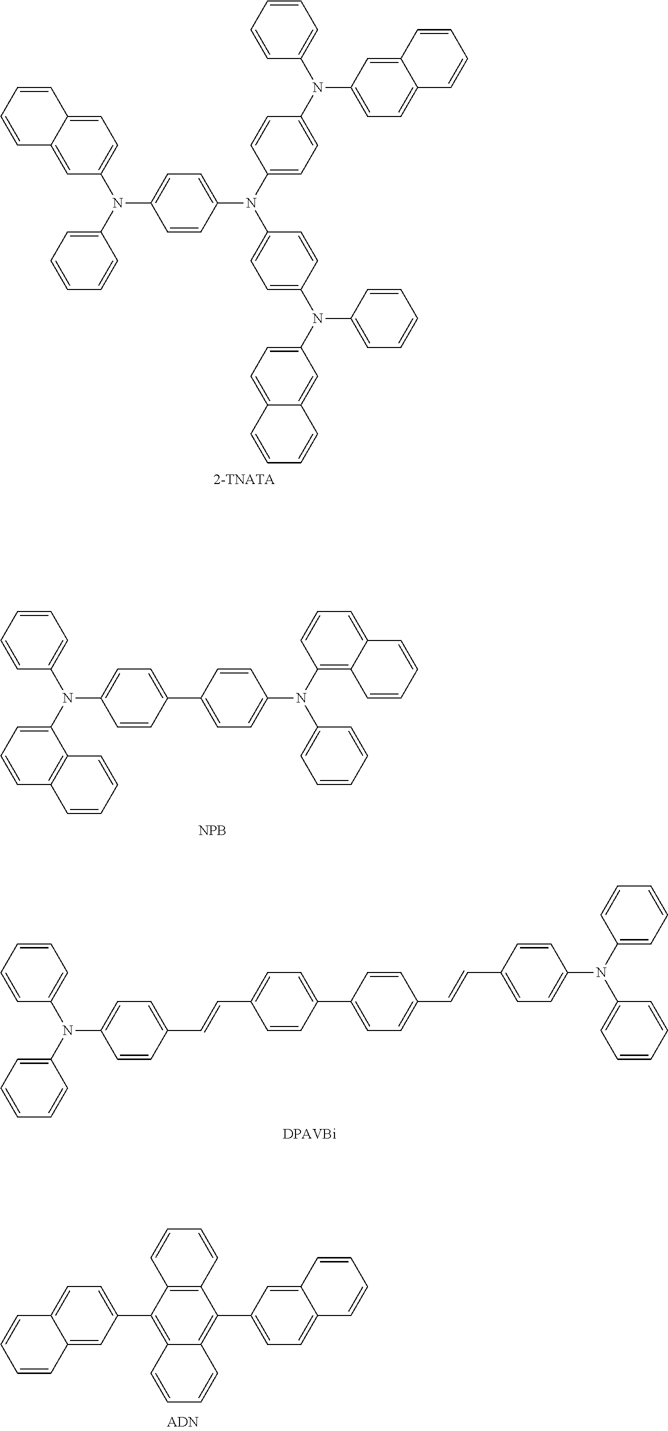

Then, Alq₃ was deposited on the emission layer to form an electron transport layer having a thickness of 300 Å, LiF, which is a halogenated alkali metal, was deposited on the electron transport layer to form an electron injection layer having a thickness of 10 Å, and Al was vacuum-deposited thereon to form an Al electrode having a thickness of 3,000 Å (cathode) to form an LiF/Al electrode, thereby completing the manufacture of an organic electroluminescent (EL) device.

### Comparative Example 2

An organic EL device was manufactured in substantially the same manner as in Comparative Example 1, except that Compound A was used instead of NPB in forming a hole transport layer.

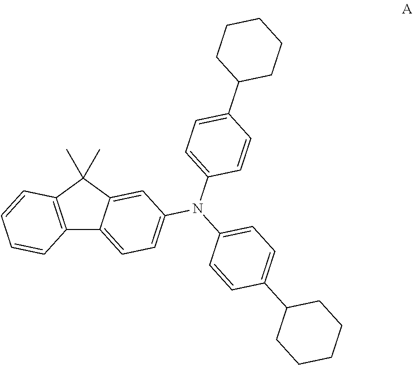 [SMILES: CC1(C)c2ccccc2-c2ccc(N(c3ccc(C4CCCCC4)cc3)c3ccc(C4CCCCC4)cc3)cc21]

### Comparative Example 3

An organic EL device was manufactured in substantially the same manner as in Comparative Example 1, except that Compound B was used instead of NPB in forming a hole transport layer.

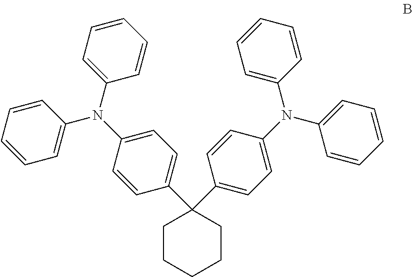 [SMILES: c1ccc(N(c2ccccc2)c2ccc(C3(c4ccc(N(c5ccccc5)c5ccccc5)cc4)CCCCC3)cc2)cc1]

### Comparative Example 4

An organic EL device was manufactured in substantially the same manner as in Comparative Example 1, except that Compound C was used instead of NPB in forming a hole transport layer.

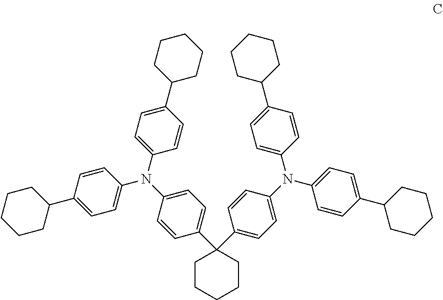 [SMILES: c1cc(N(c2ccc(C3CCCCC3)cc2)c2ccc(C3(c4ccc(N(c5ccc(C6CCCCC6)cc5)c5ccc(C6CCCCC6)cc5)cc4)CCCCC3)cc2)ccc1C1CCCCC1]

### Comparative Example 5

An organic EL device was manufactured in substantially the same manner as in Comparative Example 1, except that Compound D was used instead of NPB in forming a hole transport layer.

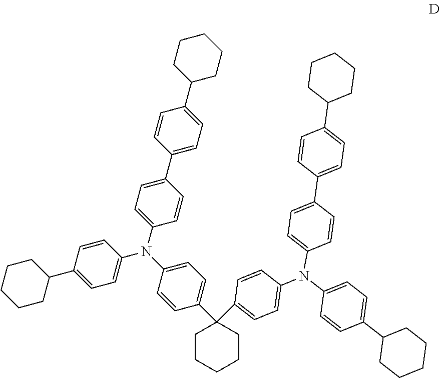 [SMILES: c1cc(C2CCCCC2)ccc1-c1ccc(N(c2ccc(C3CCCCC3)cc2)c2ccc(C3(c4ccc(N(c5ccc(-c6ccc(C7CCCCC7)cc6)cc5)c5ccc(C6CCCCC6)cc5)cc4)CCCCC3)cc2)cc1]

### Comparative Example 6

An organic EL device was manufactured in substantially the same manner as in Comparative Example 1, except that Compound E was used instead of NPB in forming a hole transport layer.

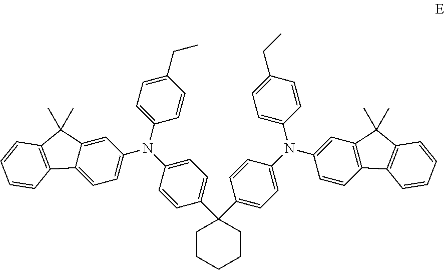 [SMILES: CCc1ccc(N(c2ccc(C3(c4ccc(N(c5ccc(CC)cc5)c5ccc6c(c5)C(C)(C)c5ccccc5-6)cc4)CCCCC3)cc2)c2ccc3c(c2)C(C)(C)c2ccccc2-3)cc1]

### Comparative Example 7

An organic EL device was manufactured in substantially the same manner as in Comparative Example 1, except that Compound F was used instead of NPB in forming a hole transport layer.

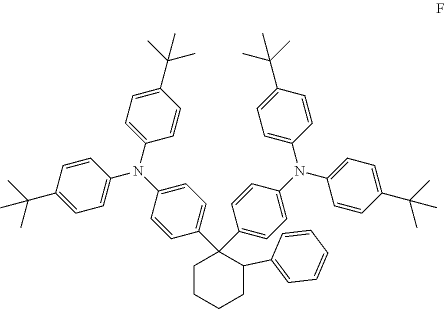 [SMILES: CC(C)(C)c1ccc(N(c2ccc(C(C)(C)C)cc2)c2ccc(C3(c4ccc(N(c5ccc(C(C)(C)C)cc5)c5ccc(C(C)(C)C)cc5)cc4)CCCCC3c3ccccc3)cc2)cc1]

### Example 1

An organic EL device was manufactured in substantially the same manner as in Comparative Example 1, except that Compound 13 was used instead of NPB in forming a hole transport layer.

### Example 2

An organic EL device was manufactured in substantially the same manner as in Comparative Example 1, except that Compound 17 was used instead of NPB in forming a hole transport layer.

### Example 3

An organic EL device was manufactured in substantially the same manner as in Comparative Example 1, except that Compound 21 was used instead of NPB in forming a hole transport layer.

### Example 4

An organic EL device was manufactured in substantially the same manner as in Comparative Example 1, except that Compound 25 was used instead of NPB in forming a hole transport layer.

### Example 5

An organic EL device was manufactured in substantially the same manner as in Comparative Example 1, except that Compound 26 was used instead of NPB in forming a hole transport layer.

### Example 6

An organic EL device was manufactured in substantially the same manner as in Comparative Example 1, except that Compound 27 was used instead of NPB in forming a hole transport layer.

### Example 7

An organic EL device was manufactured in substantially the same manner as in Comparative Example 1, except that Compound 29 was used instead of NPB in forming a hole transport layer.

### Example 8

An organic EL device was manufactured in substantially the same manner as in Comparative Example 1, except that Compound 59 was used instead of NPB in forming a hole transport layer.

### Example 9

An organic EL device was manufactured in substantially the same manner as in Comparative Example 1, except that Compound 121 was used instead of NPB in forming a hole transport layer.

### Example 10

An organic EL device was manufactured in substantially the same manner as in Comparative Example 1, except that Compound 182 was used instead of NPB in forming a hole transport layer.

### Example 11

An organic EL device was manufactured in substantially the same manner as in Comparative Example 1, except that Compound 118 was used instead of NPB in forming a hole transport layer.

### Example 12

An organic EL device was manufactured in substantially the same manner as in Comparative Example 1, except that Compound 122 was used instead of NPB in forming a hole transport layer.

### Example 13

An organic EL device was manufactured in substantially the same manner as in Comparative Example 1, except that Compound 126 was used instead of NPB in forming a hole transport layer.

### Evaluation Example 2 (Evaluation of Characteristics of Light-Emitting Devices)

To evaluate the characteristics of the organic EL devices according to Examples and Comparative Examples, the driving voltage (V) at the current density of 50 mA/cm², luminance (cd/m²), luminescence efficiency (Cd/A), and half lifespan (T50) at 100 mA/cm² of each organic EL device were measured using a Keithley MU236 and a luminance meter PR650, and the results thereof are shown in Table 3. In Table 3, the half lifespan is a measure of the time (hr) taken until the luminance reaches 50% of the initial luminance.

TABLE 3

| | | | | | | | Half |
|---|---|---|---|---|---|---|---|
| | | Driving | Current | | | | lifespan |
| | | voltage | density | Luminance | Efficiency | Emission | (hr @ 100 |
| | Hole transport layer | (V) | (mA/cm²) | (cd/m²) | (cd/A) | color | mA/cm²) |
| Comparative | NPB | 7.01 | 50 | 2645 | 5.29 | Blue | 258 |
| Example 1 | | | | | | | |
| Comparative | Compound A | 5.0 | 50 | 3150 | 6.8 | Blue | 300 |
| Example 2 | | | | | | | |
| Comparative | Compound B | 4.4 | 50 | 3200 | 7.0 | Blue | 325 |
| Example 3 | | | | | | | |
| Comparative | Compound C | 4.6 | 50 | 3125 | 7.2 | Blue | 305 |
| Example 4 | | | | | | | |
| Comparative | Compound D | 4.5 | 50 | 3130 | 6.3 | Blue | 307 |
| Example 5 | | | | | | | |
| Comparative | Compound E | 4.5 | 50 | 3120 | 6.2 | Blue | 320 |
| Example 6 | | | | | | | |
| Comparative | Compound F | 4.4 | 50 | 3250 | 7.1 | Blue | 303 |
| Example 7 | | | | | | | |
| Example 1 | Compound 13 | 4.4 | 50 | 3400 | 7.5 | Blue | 380 |
| Example 2 | Compound 17 | 3.9 | 50 | 3600 | 7.5 | Blue | 400 |
| Example 3 | Compound 21 | 3.9 | 50 | 3620 | 7.5 | Blue | 430 |
| Example 4 | Compound 25 | 4.0 | 50 | 3625 | 7.6 | Blue | 435 |
| Example 5 | Compound 26 | 4.2 | 50 | 3450 | 7.4 | Blue | 390 |
| Example 6 | Compound 27 | 4.0 | 50 | 3500 | 7.5 | Blue | 390 |
| Example 7 | Compound 29 | 4.1 | 50 | 3630 | 7.5 | Blue | 410 |
| Example 8 | Compound 59 | 4.2 | 50 | 3500 | 7.4 | Blue | 390 |
| Example 9 | Compound 121 | 4.3 | 50 | 3550 | 7.4 | Blue | 395 |
| Example 10 | Compound 182 | 4.0 | 50 | 3605 | 7.5 | Blue | 400 |
| Example 11 | Compound 118 | 3.9 | 50 | 3650 | 7.3 | Blue | 450 |
| Example 12 | Compound 122 | 4.0 | 50 | 3680 | 7.4 | Blue | 455 |
| Example 13 | Compound 126 | 4.05 | 50 | 3620 | 7.2 | Blue | 445 |

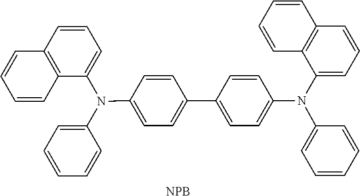 [SMILES: c1ccc(N(c2ccc(-c3ccc(N(c4ccccc4)c4cccc5ccccc45)cc3)cc2)c2cccc3ccccc23)cc1 ; "NPB"]

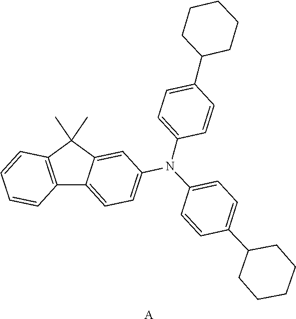

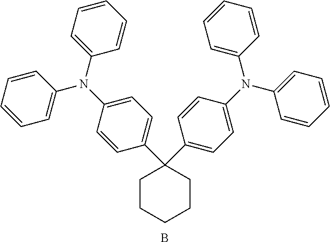

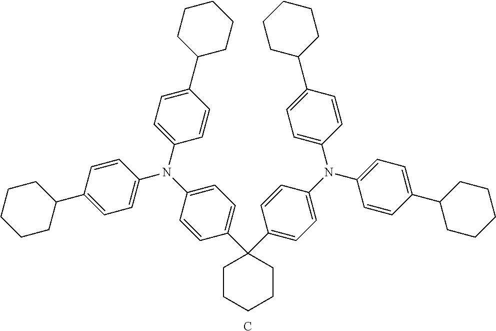

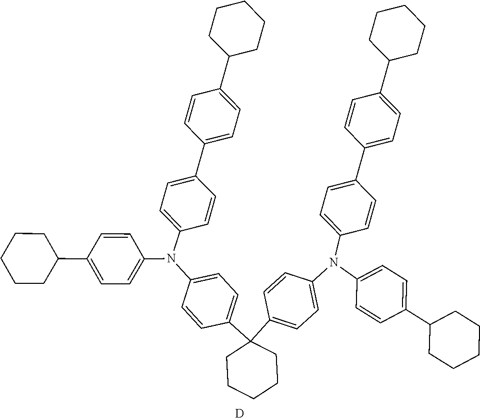

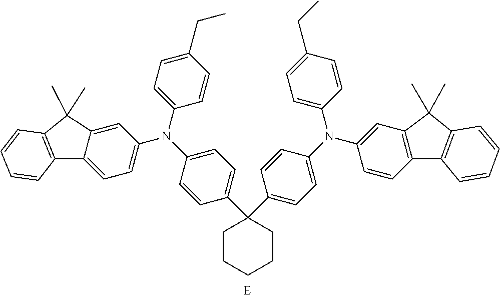

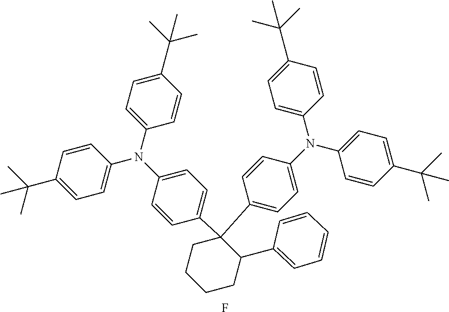

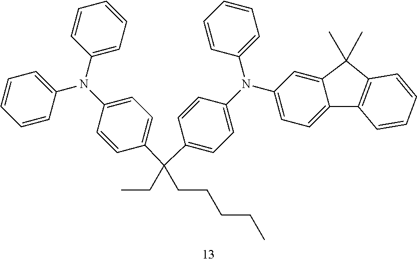 [SMILES: CCCCCC(CC)(c1ccc(N(c2ccccc2)c2ccccc2)cc1)c1ccc(N(c2ccccc2)c2ccc3c(c2)C(C)(C)c2ccccc2-3)cc1 ; "13"]

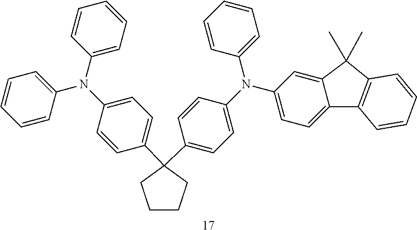

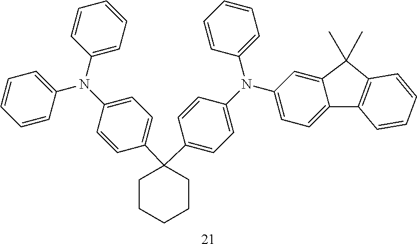

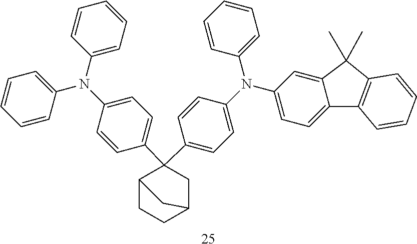

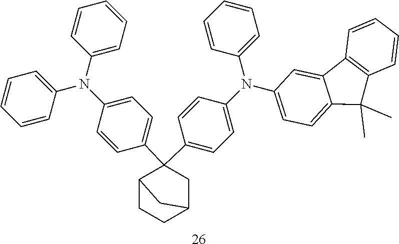

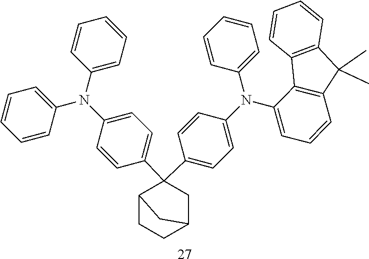

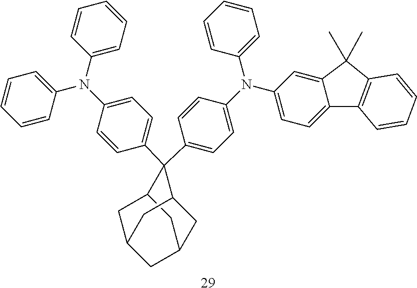 [SMILES: CC1(C)c2ccccc2-c2ccc(N(c3ccccc3)c3ccc(C4(c5ccc(N(c6ccccc6)c6ccccc6)cc5)C5CC6CC(C5)CC4C6)cc3)cc21 ; "29"]

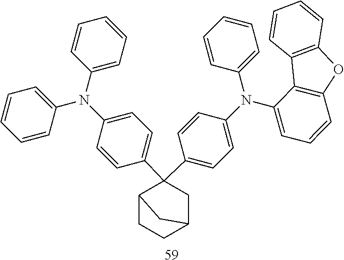

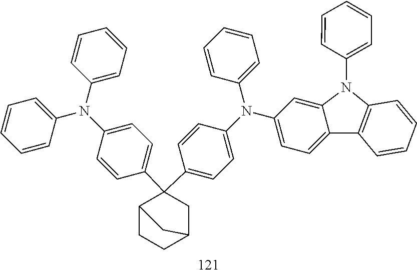

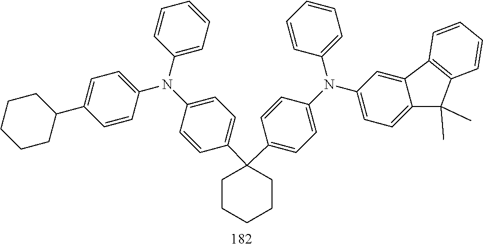

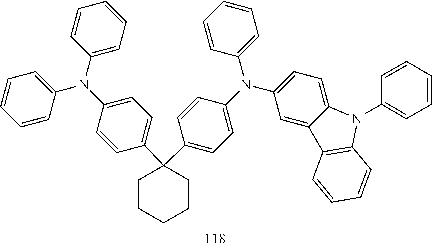

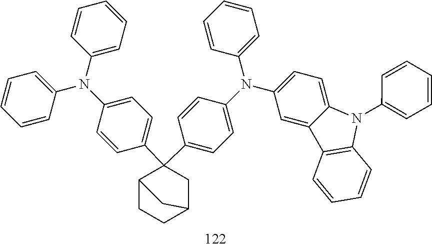

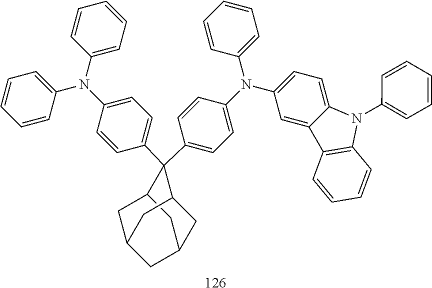 [SMILES: c1ccc(N(c2ccccc2)c2ccc(C3(c4ccc(N(c5ccccc5)c5ccc6c(c5)c5ccccc5n6-c5ccccc5)cc4)C4CC5CC(C4)CC3C5)cc2)cc1 ; "126"]

According to Table 3, the organic EL devices of the Examples using the amine compound represented by Formula 1 show excellent I-V-L (current-voltage-luminance) characteristics with improved luminance, driving voltage, and/or luminescence efficiency, compared to the organic EL devices of the Comparative Examples. For example, when comparing the half lifespans in the Examples and the Comparative Examples, it can be seen that the organic EL devices of the Examples have significantly improved lifespan compared to the organic EL devices of the Comparative Examples.

According to the one or more embodiments, by including an amine compound represented by Formula 1, a light-emitting device may have excellent characteristics in terms of luminescence efficiency and lifespan without a significant increase in driving voltage. Accordingly, by using the light-emitting device, a high-quality electronic apparatus may be manufactured.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the present disclosure as defined by the following claims, and equivalents thereof.

What is claimed is:

1. A light-emitting device comprising:
a first electrode;
a second electrode facing the first electrode;
an interlayer between the first electrode and the second electrode and comprising an emission layer; and
at least one amine compound represented by Formula 1: 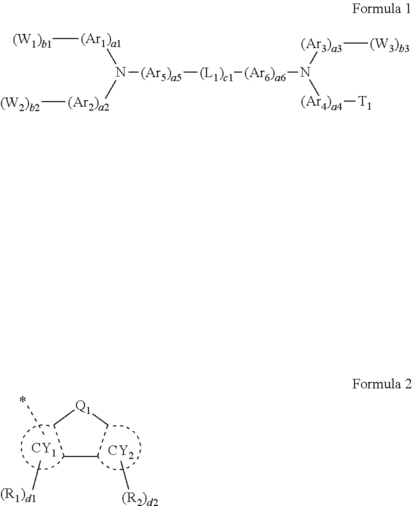
wherein, in Formulae 1 and 2,
L₁ is *—C(X₁)(X₂)—*′,
X₁ and X₂ are each independently:
hydrogen, deuterium, —F, —Cl, —Br, —I, a hydroxyl group, or a cyano group; or
a C₁-C₂₀ alkyl group unsubstituted or substituted with at least one R₁₀ₐ or a C₃-C₂₀ cycloalkyl group unsubstituted or substituted with at least one R₁₀ₐ,
X₁ and X₂ are optionally linked to each other to form a C₃-C₂₀ cycloalkane group unsubstituted or substituted with at least one R₁₀ₐ,
c1 is an integer from 1 to 3,
Ar₁ to Ar₆ are each independently a single bond, a C₅-C₆₀ carbocyclic group unsubstituted or substituted with at least one R₁₀ₐ, or a C₁-C₆₀ heterocyclic group unsubstituted or substituted with at least one R₁₀ₐ,
a1 to a6 are each independently an integer from 1 to 3,
W₁ to W₃ are each independently a C₅-C₆₀ carbocyclic group unsubstituted or substituted with at least one R₁₀ₐ or a C₁-C₆₀ heterocyclic group unsubstituted or substituted with at least one R₁₀ₐ,
b1 to b3 are each independently an integer from 1 to 10,
T₁ is a group represented by Formula 2,
in Formula 2,
CY₁ and CY₂ are each independently a C₅-C₆₀ carbocyclic group or a C₁-C₆₀ heterocyclic group,
Q₁ is O, S, N(X₁₁), C(X₁₁)(X₁₂), or Si(X₁₁)(X₁₂),
R₁, R₂, X₁₁, and X₁₂ are each independently hydrogen or the same as described in connection with R₁₀ₐ,
X₁₁ and X₁₂ are optionally linked to each other to form a C₃-C₆₀ carbocyclic group or a C₁-C₆₀ heterocyclic group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, or any combination thereof,
d1 and d2 are each independently an integer from 0 to 10,
in Formula 1, a group represented by *—(Ar₄)ₐ₄-T₁ is different from a group represented by *—(Ar₁)ₐ₁—(W₁)_{b1} and a group represented by *—(Ar₂)ₐ₂—(W₂)_{b2},
* and *′ each indicate a binding site to a neighboring atom,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The light-emitting device of claim 1, wherein the first electrode is an anode,
the second electrode is a cathode,
the interlayer further comprises a hole transport region between the emission layer and the first electrode,
the interlayer further comprises an electron transport region between the emission layer and the second electrode,
the hole transport region comprises a hole injection layer, a hole transport layer, an emission auxiliary layer, an electron blocking layer, or any combination thereof, and
the electron transport region comprises a buffer layer, a hole blocking layer, an electron control layer, an electron transport layer, an electron injection layer, or any combination thereof.

3. The light-emitting device of claim 2, wherein the hole transport region comprises the at least one amine compound represented by Formula 1.

4. The light-emitting device of claim 2, wherein the hole transport region comprises at least one selected from a hole injection layer, a hole transport layer, and an electron blocking layer, and
the at least one selected from the hole injection layer, the hole transport layer, and the electron blocking layer comprises the at least one amine compound represented by Formula 1.

5. The light-emitting device of claim 1, wherein the emission layer emits blue light having a maximum emission wavelength of about 430 nm to about 490 nm.

6. An electronic apparatus comprising the light-emitting device of claim 1.

7. The electronic apparatus of claim 6, further comprising a thin-film transistor,
wherein the thin-film transistor comprises a source electrode and a drain electrode, and
the first electrode of the light-emitting device is electrically connected to the source electrode or the drain electrode.

8. The electronic apparatus of claim 6, further comprising a color filter, a color conversion layer, a touch screen layer, a polarizing layer, or any combination thereof.

9. An amine compound represented by Formula 1: 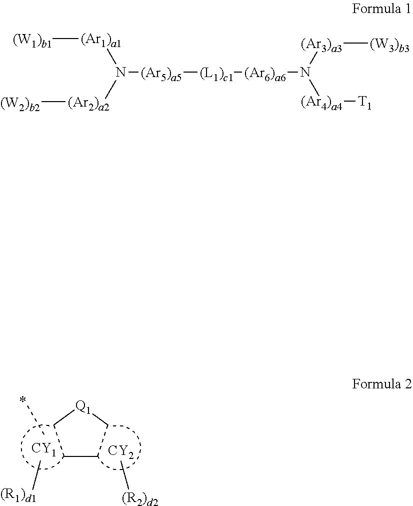
wherein, in Formulae 1 and 2,
L₁ is *—C(X₁)(X₂)—*′,
X₁ and X₂ are each independently:
hydrogen, deuterium, —F, —Cl, —Br, —I, a hydroxyl group, or a cyano group; or
a C₁-C₂₀ alkyl group unsubstituted or substituted with at least one R₁₀ₐ or a C₃-C₂₀ cycloalkyl group unsubstituted or substituted with at least one R₁₀ₐ,
X₁ and X₂ are optionally linked to each other to form a C₃-C₂₀ cycloalkane group unsubstituted or substituted with at least one R₁₀ₐ,
c1 is an integer from 1 to 3,
Ar₁ to Ar₆ are each independently a single bond, a C₅-C₆₀ carbocyclic group unsubstituted or substituted with at least one R₁₀ₐ, or a C₁-C₆₀ heterocyclic group unsubstituted or substituted with at least one R₁₀ₐ,
a1 to a6 are each independently an integer from 1 to 3,
W₁ to W₃ are each independently a C₅-C₆₀ carbocyclic group unsubstituted or substituted with at least one R₁₀ₐ or a C₁-C₆₀ heterocyclic group unsubstituted or substituted with at least one R₁₀ₐ,
b1 to b3 are each independently an integer from 1 to 10,
T₁ is a group represented by Formula 2,
in Formula 2,
CY₁ and CY₂ are each independently a C₅-C₆₀ carbocyclic group or a C₁-C₆₀ heterocyclic group,
Q₁ is O, S, N(X₁₁), C(X₁₁)(X₁₂), or Si(X₁₁)(X₁₂),
R₁, R₂, X₁₁, and X₁₂ are each independently hydrogen or the same as described in connection with R₁₀ₐ,
X₁₁ and X₁₂ are optionally linked to each other to form a C₃-C₆₀ carbocyclic group or a C₁-C₆₀ heterocyclic group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, or any combination thereof,
d1 and d2 are each independently an integer from 0 to 10,
in Formula 1, a group represented by *—(Ar₄)ₐ₄-T₁ is different from a group represented by *—(Ar₁)ₐ₁—(W₁)_{b1} and a group represented by *—(Ar₂)ₐ₂—(W₂)_{b2},
* and *′ each indicate a binding site to a neighboring atom,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

10. The amine compound of claim 9, wherein X₁ and X₂ are each independently: hydrogen; a C₁-C₁₀ alkyl group unsubstituted or substituted with deuterium, —F, a cyano group, or any combination thereof; or a C₃-C₁₀ cycloalkyl group unsubstituted or substituted with deuterium, —F, a cyano group, a C₁-C₁₀ alkyl group, or any combination thereof.

11. The amine compound of claim 9, wherein c1 is 1 or 2.

12. The amine compound of claim 9, wherein a group represented by *-(L₁)_{c1}- is one selected from *—(CH₂)—*′, *—CH(CH₃)—*′, *—CH(CH₂CH₃)—*′, *—CH[(CH₂)₂CH₃]—*′, *—CH[(CH₂)₃CH₃]—*′, *—CH[(CH₂)₄CH₃]—*′, *—C(CH₃)₂—*′, *—C(CH₃)(CH₂CH₃)—*′, *—C(CH₃)[(CH₂)₂CH₃]—*′, *—C(CH₃)[(CH₂)₃CH₃]—*′, *—C(CH₃)[(CH₂)₄CH₃]—*′, *—C(CH₂CH₃)₂—*′, *—C(CH₂CH₃)[(CH₂)₂CH₃]—*′, *—C(CH₂CH₃)[(CH₂)₃CH₃]—*′, *—C(CH₂CH₃)[(CH₂)₄CH₃]—*′, and a group represented by one selected from Formulae L-1 to L-8: 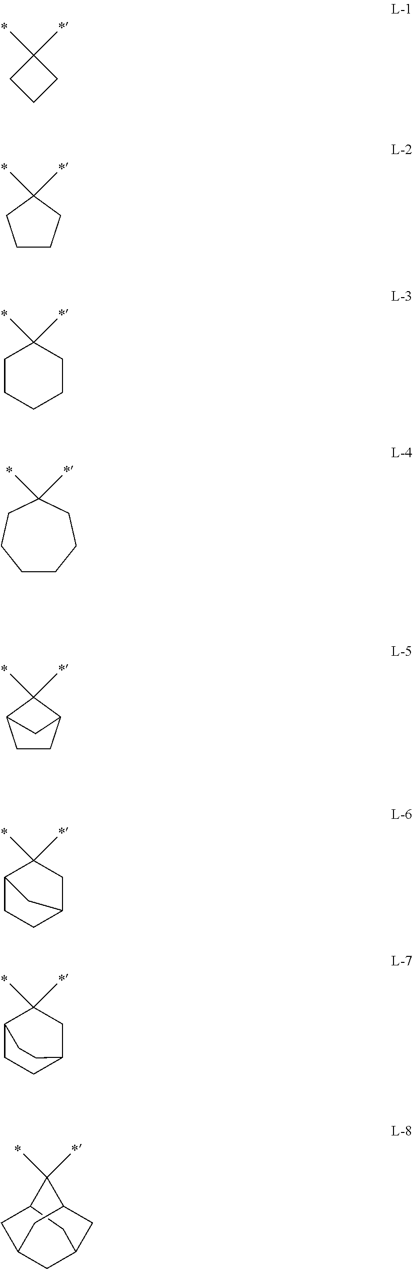
wherein, in Formulae L-1 to L-8, * and *′ each indicate a binding site to a neighboring atom.

13. The amine compound of claim 9, wherein Ar₁ to Ar₆ are each independently one selected from a single bond and a group represented by one selected from Formulae 2-1 to 2-3: 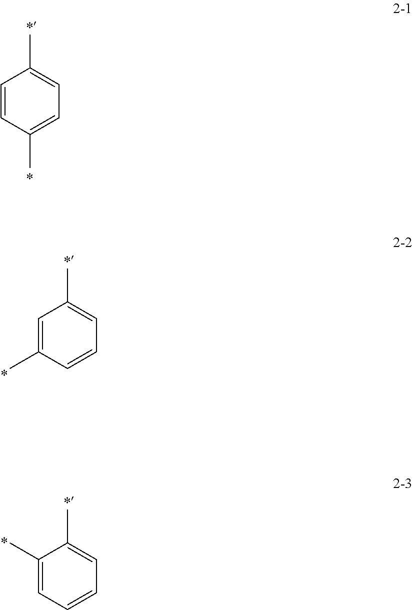
wherein, in Formulae 2-1 to 2-3, * and *′ each indicate a binding site to a neighboring atom.

14. The amine compound of claim 9, wherein a1 to a6 are each independently 1 or 2.

15. The amine compound of claim 9, wherein W₁ to W₃ are each independently a phenyl group or a naphthyl group, each unsubstituted or substituted with deuterium, —F, a cyano group, a nitro group, a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a cyclopentyl group, a cyclohexyl group, a cycloheptyl group, a cyclooctyl group, an adamantanyl group, a norbornanyl group, a bicyclo[1.1.1]pentyl group, a bicyclo[2.1.1]hexyl group, a bicyclo[2.2.2]octyl group, or any combination thereof.

16. The amine compound of claim 9, wherein b1 to b3 are each independently an integer from 1 to 5.

17. The amine compound of claim 9, wherein CY₁ and CY₂ are each independently a benzene group or a naphthalene group.

18. The amine compound of claim 9, wherein R₁ and R₂ are each independently: hydrogen, deuterium, —F, a cyano group, or a nitro group; or a C₁-C₁₀ alkyl group, a C₃-C₁₀ cycloalkyl group, or a C₁-C₁₀ alkoxy group, each unsubstituted or substituted with deuterium, —F, a cyano group, a nitro group, or any combination thereof, and
d1 and d2 are each independently an integer from 0 to 5.

19. The amine compound of claim 9, wherein T₁ is a group represented by one selected from Formulae 4-1 to 4-4: 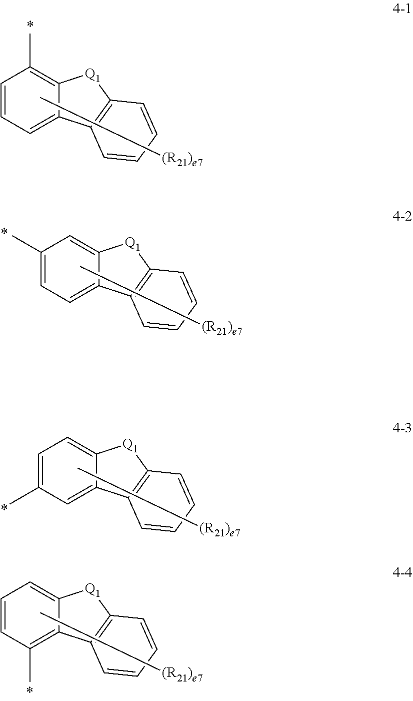
wherein, in Formulae 4-1 to 4-4,
Q₁ is the same as Q₁ described in claim 9,
R₂₁ is the same as described in connection with R₁ in claim 9,
e7 is an integer from 0 to 7, and
* indicates a binding site to a neighboring atom.

20. The light-emitting device of claim 9, wherein the amine compound is represented by Formula 1A: 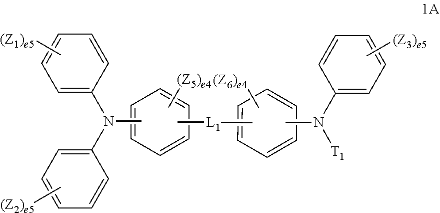
wherein, in Formula 1A,
L₁ and T₁ are respectively the same as L₁ and Tᵢ described in claim 9,
Z₁ to Z₃, Z₅, and Z₆ are each the same as described in connection with R₁₀ₐ in claim 9,
e4 is an integer from 0 to 4, and
e5 is an integer from 0 to 5.
